(19) United States
(12) Patent Application Publication
(10) Pub. No.: US 2026/0232682 A1
(43) Pub. Date: Aug. 13, 2026

(54) GSPT1 DEGRADER COMPOUNDS

(71) Applicant: Captor Therapeutics S.A., Wroclaw (PL)

(72) Inventors: Sylvain COTTENS, Witterswil (CH); Przemyslaw GLAZA, Osieczna (PL); Joanna MAJKUT, Oborniki Slaskie (PL); Krzysztofa ODRZYWÓL, Wroclaw (PL); Roman PLUTA, Kraków (PL); Michal Jerzy WALCZAK, Wroclaw (PL)

(21) Appl. No.: 19/154,211

(22) PCT Filed: Feb. 6, 2024
(86) PCT No.: PCT/EP2024/052941

§ 371 Date: Aug. 6, 2025

(30) Foreign Application Priority Data
Feb. 7, 2023 (WO) PCT/PL2023/000008

(51) Int. Cl. A61K 31/5377; A61K 31/4709; A61K 31/497; A61K 31/501; A61K 31/513; A61P 35/00; C07B 59/00; C07D 401/04; C07D 401/14; C07D 405/14; C07D 409/14; C07D 413/14

(52) U.S. Cl. CPC A61K 31/5377; A61K 31/4709; A61K 31/497; A61K 31/501; A61K 31/513; A61P 35/00; C07B 59/002; C07D 401/04; C07D 401/14; C07D 405/14; C07D 409/14; C07D 413/14

(57) ABSTRACT

The present invention provides a compound of formula (I) and a pharmaceutical composition comprising the same, and their use in the treatment of cancer.

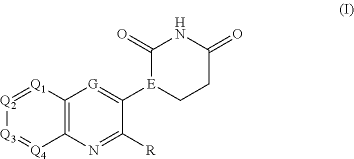

### FIELD OF THE INVENTION

The present invention relates to compounds which can modulate cellular concentrations of disease-related protein—the translation termination factor GSPT1, and their applications.

### BACKGROUND

The Ubiquitin-Proteasome System (UPS) is responsible for the maintenance of healthy and well-balanced proteome. In the process of ubiquitination, ubiquitin units are covalently attached to the protein, forming a polyubiquitin chain, which marks the protein for degradation via the proteasome. Ubiquitination is central to the regulation of nearly all cellular processes and is also tightly regulated itself. Ubiquitin ligases such as cereblon (CRBN) facilitate ubiquitination of different proteins in vivo and contribute to precise regulation of the system. Upon recognition, the ubiquitin ligases mediate the attachment of ubiquitin moieties to the target protein, which label it for degradation by the proteasome.

The idea of selective target protein degradation (TPD) by modulation of UPS was first described in 1999 (US2002173049 A1 (PROTEINIX INC) 21 Nov. 2002). The implementation of this concept has been demonstrated for clinically approved thalidomide analogs, as binding of the thalidomide analogs to the CRL4^{CRBN} E3 ligase causes recruitment of selected target proteins, leading to their ubiquitination and subsequent proteasomal degradation. The recent scientific and clinical progress in TPD has been recently reviewed by Faust T B et al. Annu. Rev. Cancer Biol. 2021. 5:181-201.

### Cereblon Modulating Agents in the Treatment of Cancer

Cereblon (CRBN) is a protein which associates with DDB1 (damaged DNA binding protein 1), CUL4 (Cullin-4), and RBX1 (RING-Box Protein 1). Collectively, the proteins form a ubiquitin ligase complex, which belongs to Cullin RING Ligase (CRL) protein family and is referred to as CRL4^{CRBN}. Thalidomide, a drug approved for treatment of multiple myeloma in the late 1990s, binds to cereblon and modulates the substrate specificity of the CRL4^{CRBN} ubiquitin ligase complex. This mechanism underlies the pleiotropic effect of thalidomide on both immune cells and cancer cells (Lu G et al. Science. 2014 Jan. 17; 343(6168): 305-9).

The clinical applicability of cereblon modulating agents (CMAs) in numerous hematologic malignancies, such as multiple myeloma, myelodysplastic syndromes lymphomas and leukemia, has been demonstrated (Le Roy A et al. Front Immunol. 2018; 9: 977). The antitumor activity of CMAs is mediated by:
inhibition of cancer cell proliferation and induction of apoptosis,
disruption of trophic support from tumor stroma,
stimulation of immune cells, resulting in proliferation of T-cells, cytokine production and activation of NK (natural killer) cells.

Thalidomide's success in cancer therapy stimulated efforts towards development of analogues with higher potency and fewer detrimental side effects. As a result, various drug candidates were produced, including lenalidomide, pomalidomide, iberdomide, avadomide, eragidomide and CC-885. For the discussion of these compounds, see—for example—U.S. Pat. No. 5,635,517 B2, WO2008039489 A2, WO2017197055 A1, WO2018237026 A1, WO2017197051 A1, U.S. Pat. No. 8,518,972 B2, EP 2057143 B1, WO2019014100 A1, WO2004103274 A2, and Surka Ch et al. Blood. 2021 Feb. 4; 137(5):661-677.

Neosubstrate degradation profile of cereblon modulating agents mediate the phenotypic and clinical outcome in a context specific manner. For example, downregulation of lymphoid transcription factors IKZF1 (IKAROS Family Zinc Finger 1) and IKZF3 (IKAROS Family Zinc Finger 3) mediates clinical efficacy of lenalidomide and pomalidomide in multiple myeloma. Simultaneously, downregulation of IKZF1 and IKZ3 has been shown to contribute to the occurrence of serious side effects, that reduce the dose of the drug that can be administered to the patient suffering from myelodysplastic syndromes. Side effects occurring during the treatment with lenalidomide include neutropenia, leukopenia, thrombocytopenia, anemia, and hemorrhagic disorders (Stahl M et al. Cancer. 2017 May 15; 123(10):1703-1713). Thus, it is desired to advance the development of the cereblon modulating agents in order to achieve a desired substrate specificity of the CRL4^{CRBN} ubiquitin ligase complex to reach a desired efficacy and safety profile depending on the clinical context (Sievers Q L et al. Science. 2018 Nov. 2; 362(6414).

### GSPT1-Targeted Strategy in Eliminating Tumor Cells

GSPT1 is a translation termination factor downregulation of which may activate an integrated stress response leading to cancer cell death. It has been demonstrated that GSPT1 depletion plays a significant functional role in the anti-AML activity of eragidomide, which is now under the clinical development. GSPT1 degradation activates the GCN1/GCN2/elF2α/ATF4 axis of the integrated stress response, and subsequent induction of acute apoptosis in AML (Surka Ch et al. Blood. 2021 Feb. 4; 137(5):661-677).

### SUMMARY OF INVENTION

The invention provides compounds which can modulate levels of target disease-related protein—GSPT1 in vitro. The compounds of the invention exhibit high selectivity, since they exhibit no or poor affinity against IKZF1, IKZF2 nor CK1α in contrast to lenalidomide and pomalidomide, preferentially resulting in a unique phenotypic profile.

The invention relates to the developing of a drug candidate that inhibits the development of cancer and/or increases the effectiveness of currently available therapies. The small molecule drug efficacy relies on the induced degradation of the preferentially-targeted protein. A protein which is preferentially targeted by the compounds of the present invention is GSPT1, which plays an important role in the process of carcinogenesis and its progression.

The invention provides compounds which cause preferential degradation of GSPT1, with increased stability in comparison to known GSPT1 degraders.

The compounds of the present invention potently inhibit growth of several cancer types including hepatocellular carcinoma (Hep3B), neuroblastoma (Kelly), leukemia (KG-1). IMiDs like pomalidomide or lenalidomide are inactive in these assays. The compounds of the present invention work through degradation of GSPT1 as they are classified as inactive in Hep3B GSPT1 G575N cell line (GSPT1 degradation-resistant cell line). The compounds of the present invention also demonstrate improved chemical stability.

The developed GSPT1 degrading drug candidates can be applied to treatment of novel cancer types, where known IMiDs are not applicable.

To minimize occurrence of the potential adverse side effects resulting from the degradation of IKZF1 or IKZF3, drug candidates which target GSPT1 preferentially are presented, with no or minor activity against indicated proteins as compared to known current IMiD drugs.

In accordance with a first aspect of the invention, there is provided a of formula (I):

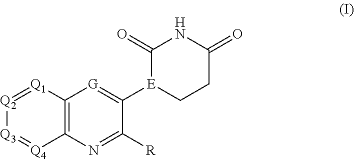

wherein
   R⁶ is hydrogen, unsubstituted C₁-C₄ alkyl, haloalkyl, —OR¹ or —N(R¹)₂;
   E is CH, CD, CF, C—(C₁-C₃ alkyl) or N;
   G is CR¹ or N;
   one of Q₁, Q₂, Q₃, and Q₄ is CR⁵ and the other three of Q₁, Q₂, Q₃, and Q₄ are each independently N or CR⁴; wherein when G is CR¹ then at least one of Q₁, Q₂, Q₃, and Q₄ is CR⁴; and
when G is N then at least two of Q₁, Q₂, Q₃, and Q₄ are CR⁴;
   each R¹ is independently hydrogen, unsubstituted C₁-C₄ alkyl, or C₁-C₄ haloalkyl; each R⁴ is independently hydrogen, unsubstituted alkyl, haloalkyl, halogen, —CN, —OR¹ or —N(R¹)₂;
   R⁶ is selected from:

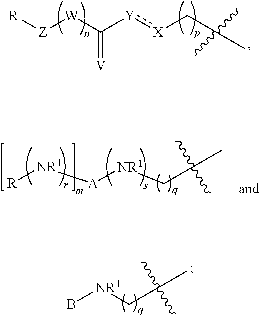

wherein [symbol] is a single bond or a double bond, wherein
when [symbol] is a single bond, then X and Y are each independently O, NR¹, CHR¹ or CD₂;
wherein when one of X and Y is O or NR¹, then the other of X and Y is CHR¹ or CD₂; and
   when [symbol] is a double bond, then X and Y are each CR¹;
W is CH₂ or C═O;
Z is C═O, NR¹ or C(R²)₂; wherein each R² is independently hydrogen, unsubstituted alkyl, halogen, OR¹ or N(R¹)₂;
V is O or S;
each R is independently cycloalkyl, heterocycloalkyl, aryl, heteroaryl, alkyl, or benzyl wherein each cycloalkyl, heterocycloalkyl, aryl, heteroaryl, alkyl or benzyl is optionally substituted with one or more R³, wherein each R³ is independently selected from halogen, R⁷, —CH₂-heterocycloalkyl, —CN, —OH, OR⁷, —NH₂, —NR¹R⁷, —NHC(O)R⁷, —NHC(O)OR⁷, —NHC(O)NR¹R⁷ and —C(O)R⁷; or wherein two R³ together with the carbon atoms to which they are attached form a cycloalkyl, heterocycloalkyl or heteroaryl ring;
or wherein R and R¹, together with the N atom to which they are attached when Z is NR¹, form a heterocycloalkyl or heteroaryl ring which is optionally substituted with one or more groups selected from halogen, unsubstituted alkyl, haloalkyl, —OH, —O(haloalkyl), —O(unsubstituted alkyl) and —N(R¹)₂;
each R¹ is independently alkyl, haloalkyl, heteroaryl, aryl, benzyl, cycloalkyl or heterocycloalkyl;
A is heteroaryl, heterocycloalkyl, cycloalkyl or cycloalkenyl;
B is —SO₂NHR or 8-12 membered bicyclic heteroaryl substituted with one or more R⁸, wherein each R⁸ is independently selected from halogen, alkyl, —O(haloalkyl), —O(unsubstituted alkyl), aryl, and CH₂-heterocycloalkyl;
n is 0 or 1;
m is 1 or 2;
r is 0 or 1;
s is 0 or 1;
each p is independently 0, 1, 2 or 3; and
each q is independently 1, 2, 3 or 4;
wherein:
   one or more CH₂ groups of any cycloalkyl, heterocycloalkyl or cycloalkenyl are optionally replaced by a C═O group;
   in E, R³, R⁷ and R⁸, each alkyl is independently optionally substituted with one or more groups selected from halogen, —OH, —O(haloalkyl), —O(unsubstituted alkyl) and —N(R¹)₂; and
   in A, R³, R⁷ and R⁸, each cycloalkyl, heterocycloalkyl, cycloalkenyl, aryl and heteroaryl is independently optionally substituted with one or more groups selected from halogen, unsubstituted alkyl, haloalkyl, —OH, —O(haloalkyl), —O(unsubstituted alkyl) and —N(R¹)₂;
   provided that:
(a) when R⁵ is

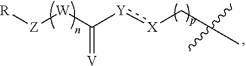

W is C═O, n is 1 and p is 0; then Z is NR¹ or C(R²)₂; and
(b) when R⁵ is

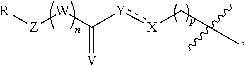

X is CHR¹, Y is NH, V is O, n is 0, p is 0, Z is NH or C(halogen)₂, and R is aryl; then at least one of E and G is N.

In some embodiments of the first aspect of the invention, when R⁵ is

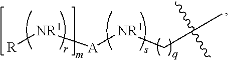

then E is CH, CD, CF or C—(C₁-C₃ alkyl).

In some such embodiments when R⁵ is

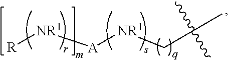

then E is CH.

In some embodiments of the first aspect of the invention, at least one of r and s is 1. In some such embodiments, r is 1. In some embodiments, s is 1.

In some embodiments of the first aspect of the invention, when [symbol] is a single bond and one of X and Y is CHR¹, then the other of X and Y is O or NR¹.

In some embodiments of the first aspect of the invention, each alkyl in E, R³, R⁷ and R⁸ is independently optionally substituted with one or more groups selected from halogen, —OH, —O(haloalkyl), and —O(unsubstituted alkyl). in some embodiments, each alkyl in E, R³, R⁷ and R⁸ is independently optionally substituted with one or more groups selected from halogen and —OH.

In other embodiments of the first aspect of the invention, each alkyl in E, R³, R⁷ and R⁸ is unsubstituted.

In some embodiments of the first aspect of the invention, the C₁-C₃ alkyl of E is unsubstituted C₁-C₃ alkyl.

In some embodiments of the first aspect of the invention, E is CH, CD or N. In some such embodiments, E is CH or N. In some such embodiments, E is N.

In other embodiments of the first aspect of the invention, E is CH, CD, CF or C—(C₁-C₃ alkyl). In some such embodiments, E is CH, CD or C—(C₁-C₃ alkyl). In some such embodiments, E is CH.

In some embodiments of the first aspect of the invention, in A, R³, R⁷ and R⁸ any cycloalkyl, heterocycloalkyl, cycloalkenyl, aryl and heteroaryl is optionally substituted with one or more groups selected from halogen, unsubstituted alkyl, haloalkyl, —O(haloalkyl) and —O(unsubstituted alkyl).

In some embodiments of the first aspect of the invention, the heterocycloalkyl or heteroaryl ring formed by R and R¹, together with the N atom to which they are attached when Z is NR¹, is optionally substituted with one or more groups selected from halogen, unsubstituted alkyl, haloalkyl, —O(haloalkyl) and —O(unsubstituted alkyl).

In some embodiments of the first aspect of the invention, each R³ is independently selected from halogen, R⁷, —CH₂-heterocycloalkyl, —CN, —OH, —OR⁷, —NH₂, —NR¹R⁷, —NHC(O)R⁷, —NHC(O)OR⁷, —NHC(O)NR¹R⁷ and —C(O)R⁷. In some such embodiments, each R³ is independently selected from halogen, R⁷, —CH₂-heterocycloalkyl, —CN, —OR⁷, —NH₂, —NHC(O)R⁷ and —C(O)R⁷. In some embodiments, each R³ is independently selected from halogen, R⁷, —CH₂-heterocycloalkyl, —CN, —OR⁷, —NH₂, —NHC(O)alkyl and —C(O)alkyl. In some embodiments, each R³ is independently selected from halogen, R⁷, —CH₂-heterocycloalkyl, —CN, —O(haloalkyl), —O(alkyl), —NH₂, —NHC(O)alkyl and —C(O)alkyl; In some embodiments of the first aspect of the invention, each R¹ is independently hydrogen or unsubstituted C₁-C₄ alkyl.

In some embodiments of the first aspect of the invention, each R⁴ is independently hydrogen, unsubstituted alkyl, haloalkyl, halogen, —OR¹ or —N(R¹)₂. In some such embodiments, each R⁴ is independently hydrogen, unsubstituted alkyl, halogen, —OR¹ or —N(R¹)₂

In some embodiments of the first aspect of the invention, R⁶ is unsubstituted C₁-C₄ alkyl, haloalkyl, OR¹ or N(R¹)₂. In some embodiments, R⁶ is OR¹ or N(R¹)₂. In other embodiments, R⁶ is unsubstituted C₁-C₄ alkyl or haloalkyl. In some such embodiments, R⁶ is unsubstituted C₁-C₄ alkyl or C₁-C₄ haloalkyl. In some embodiments, R⁶ is unsubstituted C₁-C₄ alkyl. In some embodiments, R⁶ is methyl.

In some embodiments of the first aspect of the invention, when R⁵ is

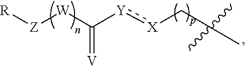

X is CHR¹, Y is NR¹, V is O, n is 0, p is 0, Z is NR¹ or C(halogen)₂, and R is aryl; then at least one of E and G is N.

In some embodiments of the first aspect of the invention, G is CR¹. In some such embodiments, G is CH. In other embodiments, G is N.

In some embodiments of the first aspect of the invention, at least two of Q₁, Q₂, Q₃, and Q₄ are CR⁴.

In some embodiments of the first aspect of the invention, three of Q₁, Q₂, Q₃, and Q₄ are CR⁴. In some such embodiments, Q₁ is CR⁵ and Q₂, Q₃, and Q₄ are each independently CR⁴. In other embodiments, Q₂ is CR⁵ and Q₁, Q₃, and Q₄ are each independently CR⁴. In other embodiments, Q₃ is CR⁵ and Q₁, Q₂, and Q₄ are each independently CR⁴. In other embodiments, Q₄ is CR⁵ and Q₁, Q₂, and Q₃ are each independently CR⁴.

In some embodiments of the first aspect of the invention, R⁵ is

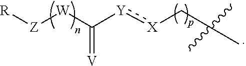

In some embodiments, the compound is selected from:

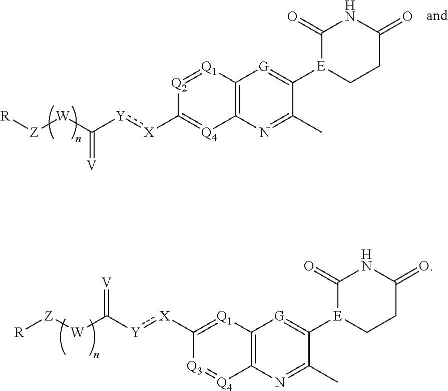

In some embodiments, R⁵ is

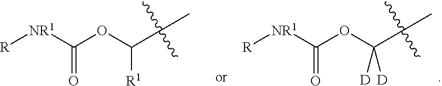

In some such embodiments, R⁵ is selected from

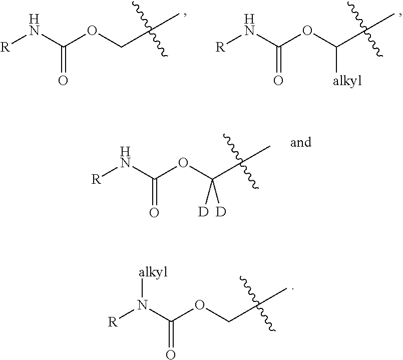

In some embodiments of the first aspect of the invention, G is CH and E is CH, CD, CF or C—(C₁-C₃ alkyl). In some such embodiments, the compound is selected from:

| Compound No. | Structure |
|---|---|
| 4 | 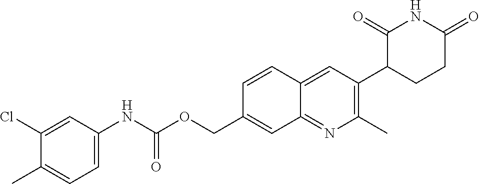 [SMILES: Cc1ccc(NC(=O)OCc2ccc3cc(C4CCC(=O)NC4=O)c(C)nc3c2)cc1Cl] |
| 5 | 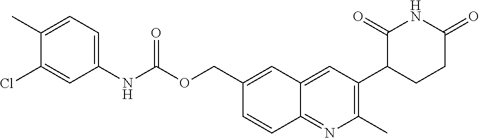 [SMILES: Cc1ccc(NC(=O)OCc2ccc3nc(C)c(C4CCC(=O)NC4=O)cc3c2)cc1Cl] |
| 6 | 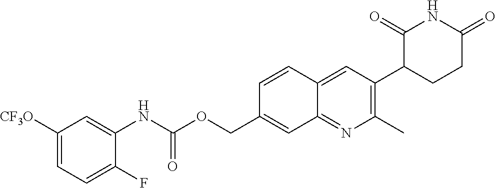 |
| 7 | 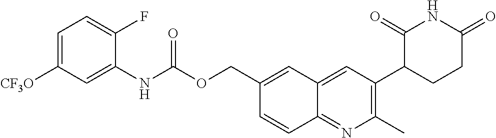 |
| 8 | 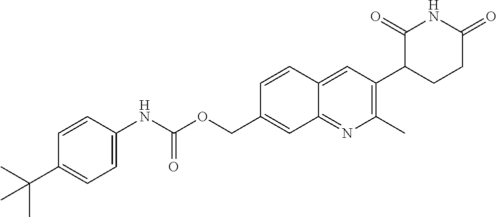 [SMILES: Cc1nc2cc(COC(=O)Nc3ccc(C(C)(C)C)cc3)ccc2cc1C1CCC(=O)NC1=O] |
| 9 | 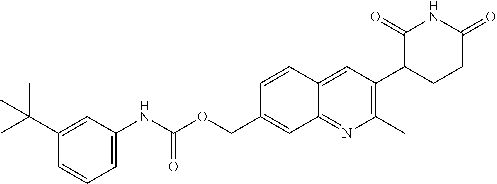 [SMILES: Cc1nc2cc(COC(=O)Nc3cccc(C(C)(C)C)c3)ccc2cc1C1CCC(=O)NC1=O] |
| 10 | 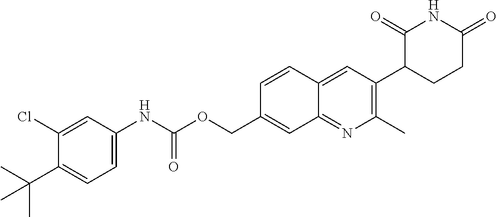 [SMILES: Cc1nc2cc(COC(=O)Nc3ccc(C(C)(C)C)c(Cl)c3)ccc2cc1C1CCC(=O)NC1=O] |
| 11 | 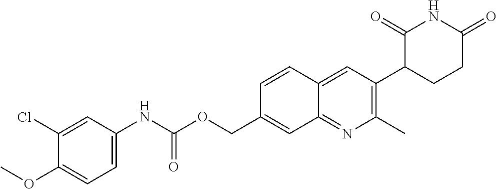 [SMILES: COc1ccc(NC(=O)OCc2ccc3cc(C4CCC(=O)NC4=O)c(C)nc3c2)cc1Cl] |
| 12 | 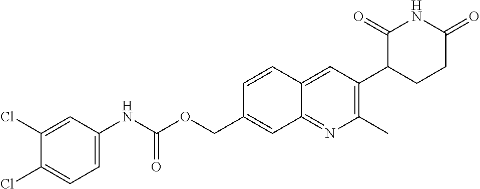 [SMILES: Cc1nc2cc(COC(=O)Nc3ccc(Cl)c(Cl)c3)ccc2cc1C1CCC(=O)NC1=O] |
| 14 | 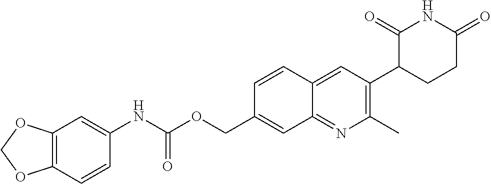 [SMILES: Cc1nc2cc(COC(=O)Nc3ccc4c(c3)OCO4)ccc2cc1C1CCC(=O)NC1=O] |
| 15 | 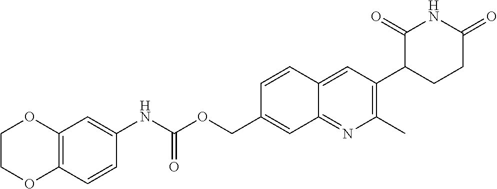 [SMILES: Cc1nc2cc(COC(=O)Nc3ccc4c(c3)OCCO4)ccc2cc1C1CCC(=O)NC1=O] |
| 17 | 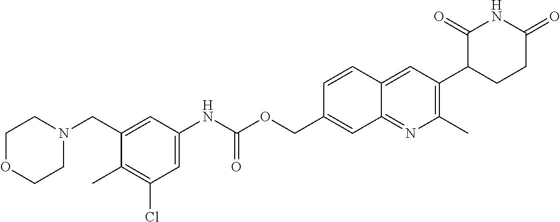 [SMILES: Cc1nc2cc(COC(=O)Nc3cc(Cl)c(C)c(CN4CCOCC4)c3)ccc2cc1C1CCC(=O)NC1=O] |
| 18 | 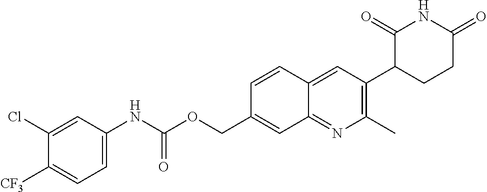 [SMILES: Cc1nc2cc(COC(=O)Nc3ccc(C(F)(F)F)c(Cl)c3)ccc2cc1C1CCC(=O)NC1=O] |
| 19 | 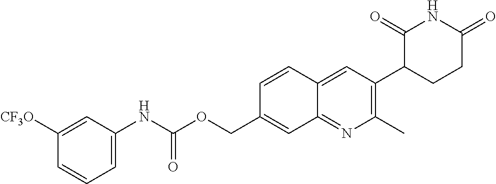 |
| 20 | 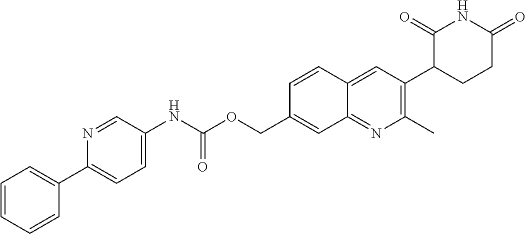 [SMILES: Cc1nc2cc(COC(=O)Nc3ccc(-c4ccccc4)nc3)ccc2cc1C1CCC(=O)NC1=O] |
| 21 | 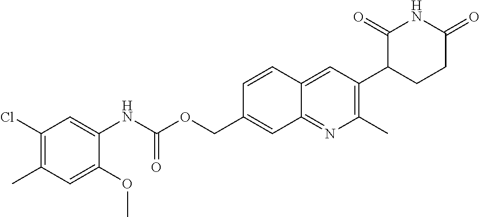 [SMILES: COc1cc(C)c(Cl)cc1NC(=O)OCc1ccc2cc(C3CCC(=O)NC3=O)c(C)nc2c1] |
| 22 | 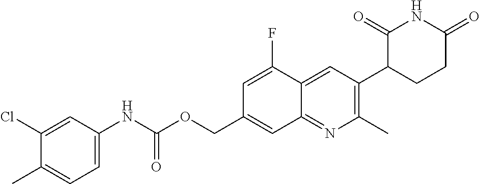 [SMILES: Cc1ccc(NC(=O)OCc2cc(F)c3cc(C4CCC(=O)NC4=O)c(C)nc3c2)cc1Cl] |
| 28 | 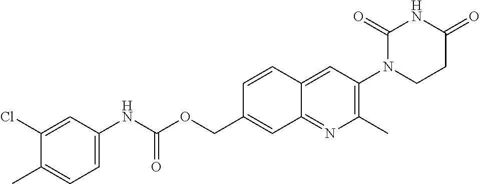 [SMILES: Cc1ccc(NC(=O)OCc2ccc3cc(N4CCC(=O)NC4=O)c(C)nc3c2)cc1Cl] |
| 29 | 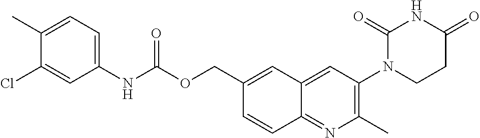 [SMILES: Cc1ccc(NC(=O)OCc2ccc3nc(C)c(N4CCC(=O)NC4=O)cc3c2)cc1Cl] |
| 30 | 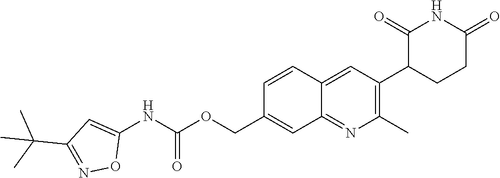 [SMILES: Cc1nc2cc(COC(=O)Nc3cc(C(C)(C)C)no3)ccc2cc1C1CCC(=O)NC1=O] |
| 37 | 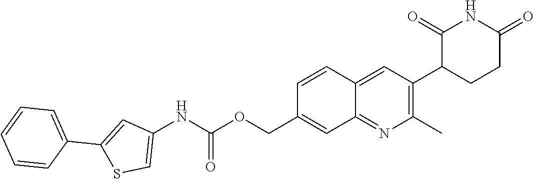 [SMILES: Cc1nc2cc(COC(=O)Nc3csc(-c4ccccc4)c3)ccc2cc1C1CCC(=O)NC1=O] |
| 38 | 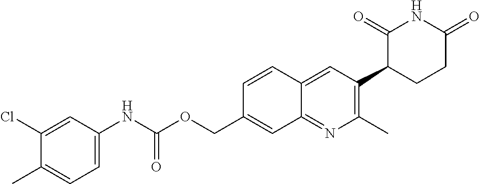 [SMILES: Cc1ccc(NC(=O)OCc2ccc3cc([C@@H]4CCC(=O)NC4=O)c(C)nc3c2)cc1Cl] |
| 39 | 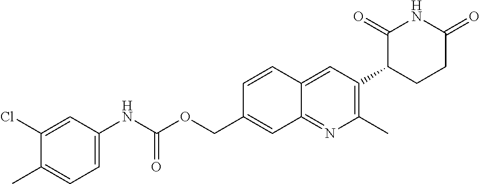 [SMILES: Cc1ccc(NC(=O)OCc2ccc3cc([C@H]4CCC(=O)NC4=O)c(C)nc3c2)cc1Cl] |
| 42 | 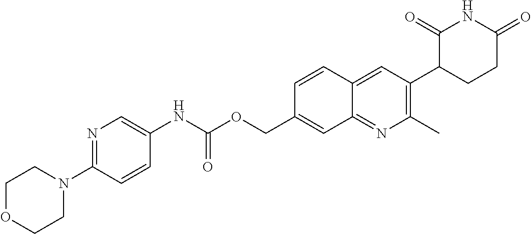 [SMILES: Cc1nc2cc(COC(=O)Nc3ccc(N4CCOCC4)nc3)ccc2cc1C1CCC(=O)NC1=O] |
| 44 | 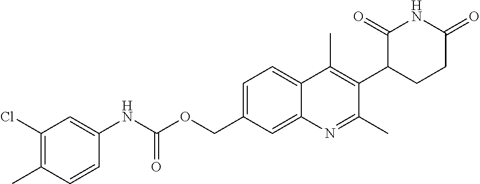 [SMILES: Cc1ccc(NC(=O)OCc2ccc3c(C)c(C4CCC(=O)NC4=O)c(C)nc3c2)cc1Cl] |
| 45 | 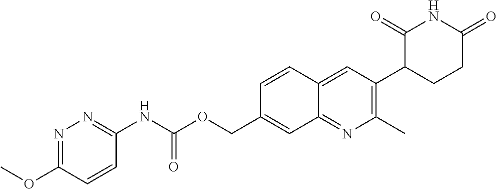 [SMILES: COc1ccc(NC(=O)OCc2ccc3cc(C4CCC(=O)NC4=O)c(C)nc3c2)nn1] |
| 46 | 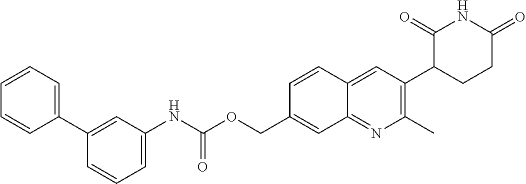 [SMILES: Cc1nc2cc(COC(=O)Nc3cccc(-c4ccccc4)c3)ccc2cc1C1CCC(=O)NC1=O] |
| 47 | 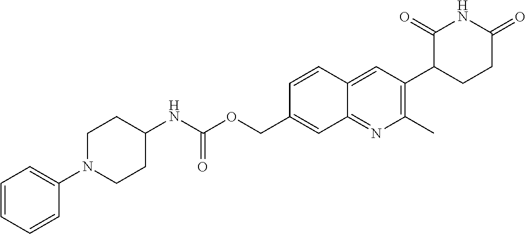 [SMILES: Cc1nc2cc(COC(=O)NC3CCN(c4ccccc4)CC3)ccc2cc1C1CCC(=O)NC1=O] |
| 49 | 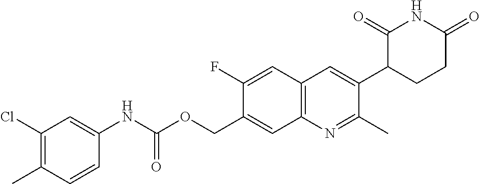 [SMILES: Cc1ccc(NC(=O)OCc2cc3nc(C)c(C4CCC(=O)NC4=O)cc3cc2F)cc1Cl] |
| 50 | 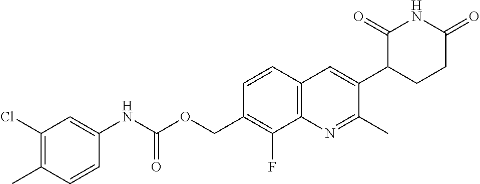 [SMILES: Cc1ccc(NC(=O)OCc2ccc3cc(C4CCC(=O)NC4=O)c(C)nc3c2F)cc1Cl] |
| 54 | 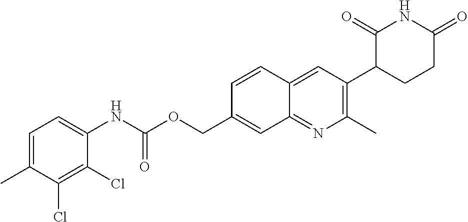 [SMILES: Cc1ccc(NC(=O)OCc2ccc3cc(C4CCC(=O)NC4=O)c(C)nc3c2)c(Cl)c1Cl] |
| 55 | 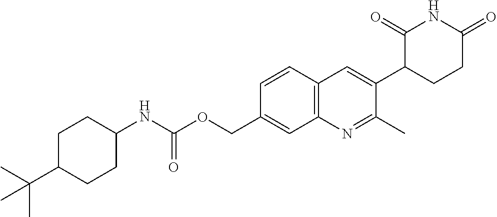 [SMILES: Cc1nc2cc(COC(=O)NC3CCC(C(C)(C)C)CC3)ccc2cc1C1CCC(=O)NC1=O] |
| 58 | 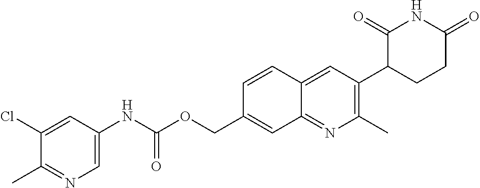 [SMILES: Cc1ncc(NC(=O)OCc2ccc3cc(C4CCC(=O)NC4=O)c(C)nc3c2)cc1Cl] |
| 59 | 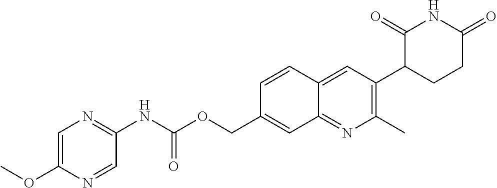 [SMILES: COc1cnc(NC(=O)OCc2ccc3cc(C4CCC(=O)NC4=O)c(C)nc3c2)cn1] |
| 60 | 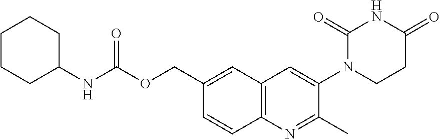 [SMILES: Cc1nc2ccc(COC(=O)NC3CCCCC3)cc2cc1N1CCC(=O)NC1=O] |
| 68 | 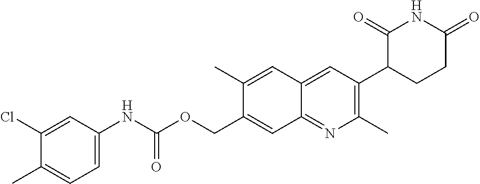 [SMILES: Cc1ccc(NC(=O)OCc2cc3nc(C)c(C4CCC(=O)NC4=O)cc3cc2C)cc1Cl] |
| 69 | 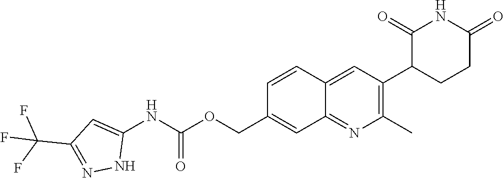 [SMILES: Cc1nc2cc(COC(=O)Nc3cc(C(F)(F)F)n[nH]3)ccc2cc1C1CCC(=O)NC1=O] |
| 70 | 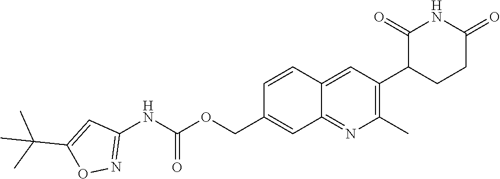 [SMILES: Cc1nc2cc(COC(=O)Nc3cc(C(C)(C)C)on3)ccc2cc1C1CCC(=O)NC1=O] |
| 71 | 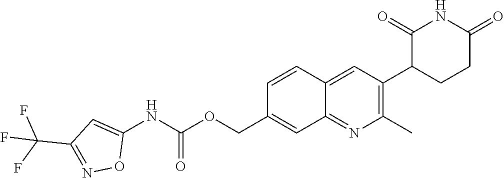 [SMILES: Cc1nc2cc(COC(=O)Nc3cc(C(F)(F)F)no3)ccc2cc1C1CCC(=O)NC1=O] |
| 74 | 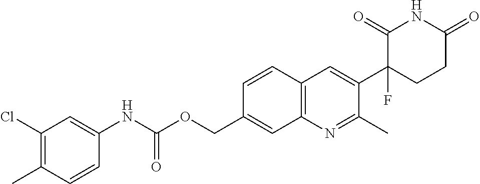 [SMILES: Cc1ccc(NC(=O)OCc2ccc3cc(C4(F)CCC(=O)NC4=O)c(C)nc3c2)cc1Cl] |
| 75 | 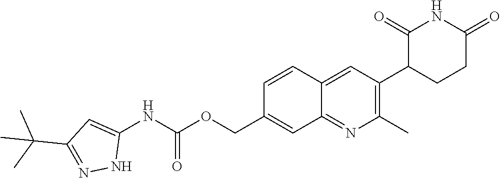 [SMILES: Cc1nc2cc(COC(=O)Nc3cc(C(C)(C)C)n[nH]3)ccc2cc1C1CCC(=O)NC1=O] |
| 80 | 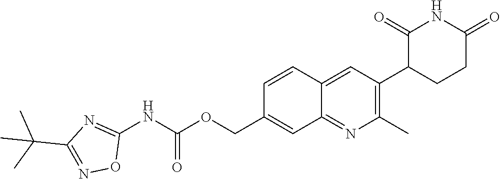 [SMILES: Cc1nc2cc(COC(=O)Nc3nc(C(C)(C)C)no3)ccc2cc1C1CCC(=O)NC1=O] |
| 81 | 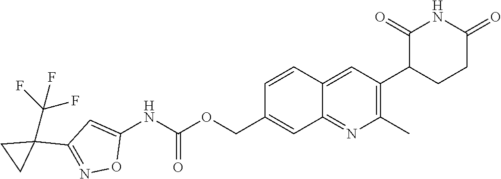 [SMILES: Cc1nc2cc(COC(=O)Nc3cc(C4(C(F)(F)F)CC4)no3)ccc2cc1C1CCC(=O)NC1=O] |
| 82 | 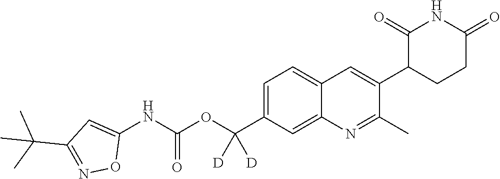 [SMILES: [2H]C([2H])(OC(=O)Nc1cc(C(C)(C)C)no1)c1ccc2cc(C3CCC(=O)NC3=O)c(C)nc2c1] |

In some embodiments of the first aspect of the invention, R⁵ is

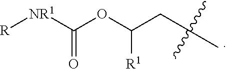

In some such embodiments, R⁵ is

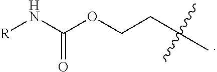

In some embodiments of the first aspect of the invention, the compound is selected from

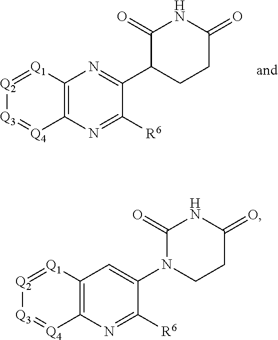

optionally wherein R⁶ is methyl. In some such embodiments, the compound is selected from

| Compound No. | Structure |
|---|---|
| 66 | 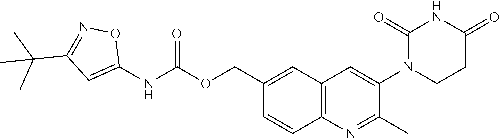 [SMILES: Cc1nc2ccc(COC(=O)Nc3cc(C(C)(C)C)no3)cc2cc1N1CCC(=O)NC1=O] |
| 72 | 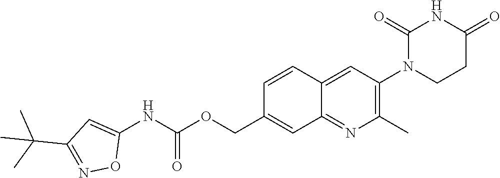 [SMILES: Cc1nc2cc(COC(=O)Nc3cc(C(C)(C)C)no3)ccc2cc1N1CCC(=O)NC1=O] |

In some embodiments of the first aspect of the invention, R⁵ is

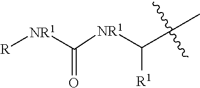

and the compound is selected from

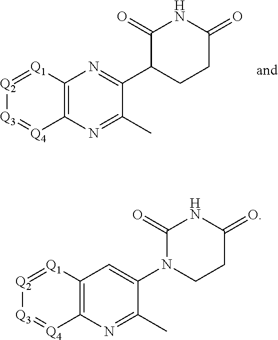

In some such embodiments, the compound is:

| Compound No. | Structure |
|---|---|
| 27 | 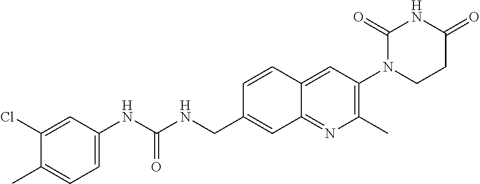 [SMILES: Cc1ccc(NC(=O)NCc2ccc3cc(N4CCC(=O)NC4=O)c(C)nc3c2)cc1Cl] |
| 41 | 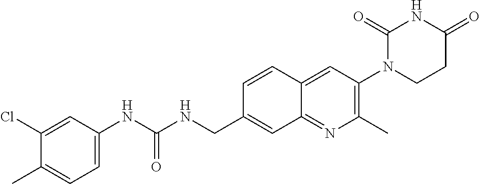 [SMILES: Cc1ccc(NC(=O)NCc2ccc3cc(N4CCC(=O)NC4=O)c(C)nc3c2)cc1Cl] |
| 51 | 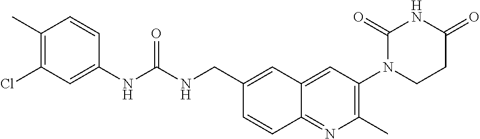 [SMILES: Cc1ccc(NC(=O)NCc2ccc3nc(C)c(N4CCC(=O)NC4=O)cc3c2)cc1Cl] |
| 52 | 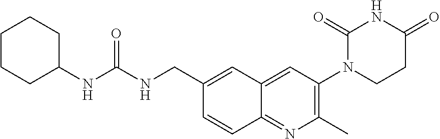 [SMILES: Cc1nc2ccc(CNC(=O)NC3CCCCC3)cc2cc1N1CCC(=O)NC1=O] |
| 64 | 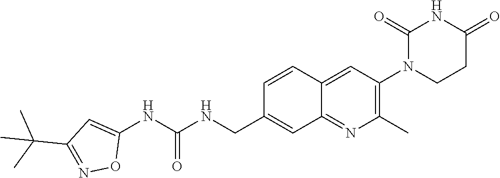 [SMILES: Cc1nc2cc(CNC(=O)Nc3cc(C(C)(C)C)no3)ccc2cc1N1CCC(=O)NC1=O] |
| 67 | 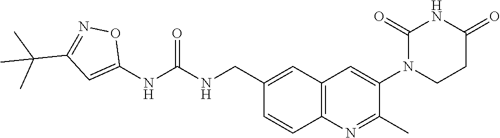 [SMILES: Cc1nc2ccc(CNC(=O)Nc3cc(C(C)(C)C)no3)cc2cc1N1CCC(=O)NC1=O] |

In other embodiments of the first aspect of the invention, R⁵ is

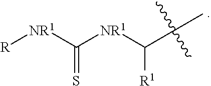

In some such embodiments, the compound is:

| Compound No. | Structure |
|---|---|
| 2 | 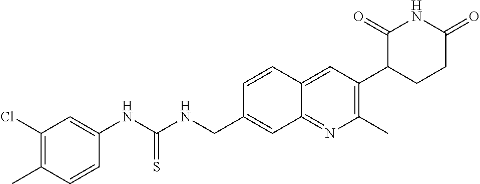 [SMILES: Cc1ccc(NC(=S)NCc2ccc3cc(C4CCC(=O)NC4=O)c(C)nc3c2)cc1Cl] |

In some embodiments of the first aspect of the invention, R⁵ is

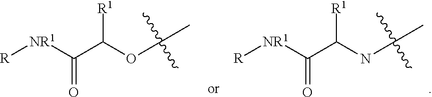

In some such embodiments, R⁵ is selected from:

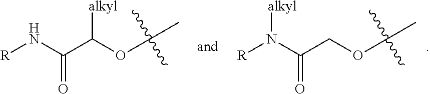

In some such embodiments, G and E are CH. In some such embodiments, the compound is selected from:

| Compound No. | Structure |
|---|---|
| 34 | 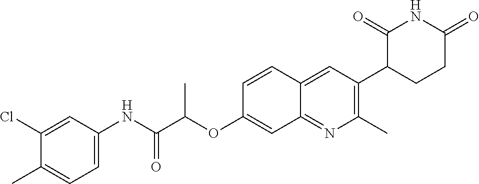 [SMILES: Cc1ccc(NC(=O)C(C)Oc2ccc3cc(C4CCC(=O)NC4=O)c(C)nc3c2)cc1Cl] |
| 36 | 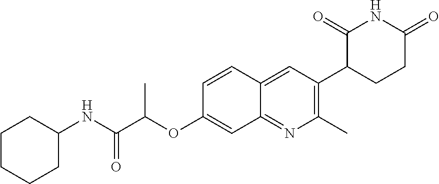 [SMILES: Cc1nc2cc(OC(C)C(=O)NC3CCCCC3)ccc2cc1C1CCC(=O)NC1=O] |

In some embodiments of the first aspect of the invention, R⁵ is:

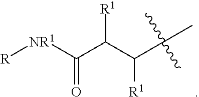

In some such embodiments, R⁵ is:

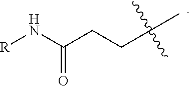

In some embodiments, the compound is:

| Compound No. | Structure |
|---|---|
| 32 | 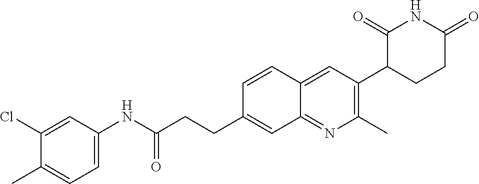 [SMILES: Cc1ccc(NC(=O)CCc2ccc3cc(C4CCC(=O)NC4=O)c(C)nc3c2)cc1Cl] |

In some embodiments of the first aspect of the invention, R⁵ is

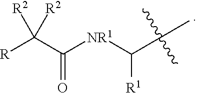

In other embodiments, R⁵ is

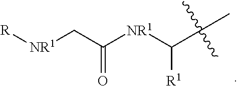

In some such embodiments, R⁵ is

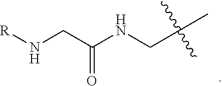

In some such embodiments, the compound is selected from:

| Compound No. | Structure |
|---|---|
| 26 | 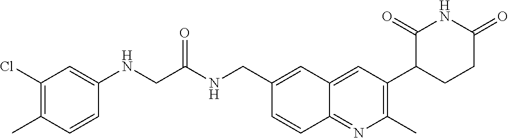 [SMILES: Cc1ccc(NCC(=O)NCc2ccc3nc(C)c(C4CCC(=O)NC4=O)cc3c2)cc1Cl] |

In other embodiments of the first aspect of the invention, R⁵ is

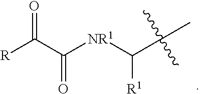

In some such embodiments, the compound is selected from:

| Compound No. | Structure |
|---|---|
| 1 | 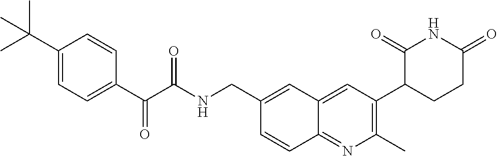 [SMILES: Cc1nc2ccc(CNC(=O)C(=O)c3ccc(C(C)(C)C)cc3)cc2cc1C1CCC(=O)NC1=O] |
| 23 | 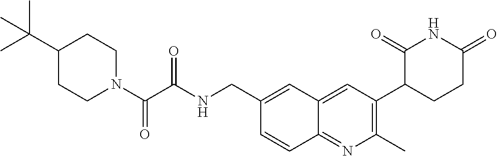 [SMILES: Cc1nc2ccc(CNC(=O)C(=O)N3CCC(C(C)(C)C)CC3)cc2cc1C1CCC(=O)NC1=O] |

In other embodiments of the first aspect of the invention, R⁵ is

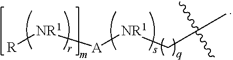

In some such embodiments, the compound is selected from:

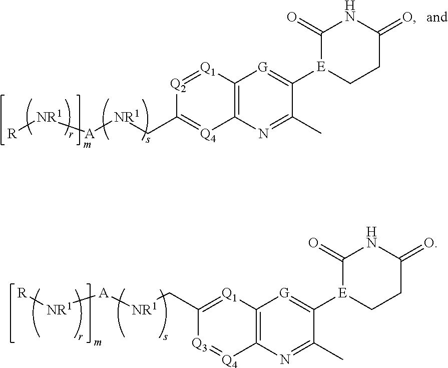

In some embodiments of the first aspect of the invention, R⁵ is

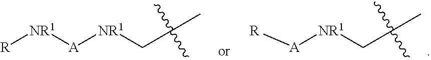

In some embodiments of the first aspect of the invention, A is 5- or 6-membered heteroaryl having 1-3 heteroatoms, 5- or 6-membered heterocycloalkyl having 1-3 heteroatoms, or 4- to 6-membered cycloalkenyl; wherein one or more CH₂ groups of the heterocycloalkyl or cycloalkenyl are optionally replaced by a C═O group. In some such embodiments, A is 5-membered heteroaryl having 2 or 3 heteroatoms. In other embodiments, A is 4-membered cycloalkenyl in which one or more CH₂ groups are replaced by a C═O group.

In some embodiments, the compound is selected from:

| Compound No. | Structure |
|---|---|
| 3 | 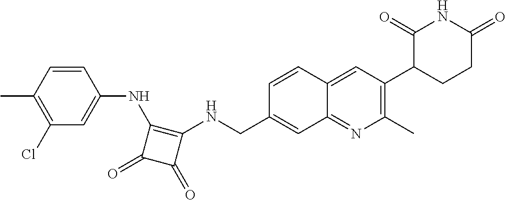 [SMILES: Cc1ccc(Nc2c(NCc3ccc4cc(C5CCC(=O)NC5=O)c(C)nc4c3)c(=O)c2=O)cc1Cl] |
| 13 | 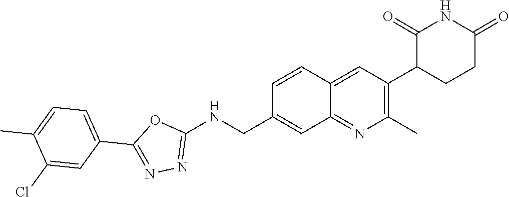 [SMILES: Cc1ccc(-c2nnc(NCc3ccc4cc(C5CCC(=O)NC5=O)c(C)nc4c3)o2)cc1Cl] |

In other embodiments of the first aspect of the invention, R⁵ is

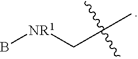

In some embodiments of the first aspect of the invention, B is —SO₂NHR or 8-12 membered bicyclic heteroaryl substituted with one or more groups selected from CI, Me, ^{t}Bu, OCF₃, OMe, CH₂-morpholino, and phenyl. In some such embodiments, B is 8-12 membered bicyclic heteroaryl substituted with one or more groups selected from CI, Me, ^{t}Bu, OCF₃, OMe, CH₂-morpholino, and phenyl.

In some embodiments of the first aspect of the invention, R is cycloalkyl, heterocycloalkyl, aryl or heteroaryl optionally substituted with one or more R³, or two R³ together with the carbon atoms to which they are attached form a cycloalkyl, heterocycloalkyl or heteroaryl ring.

In some embodiments of the first aspect of the invention, each R³ is independently selected from CI, Me, ^{t}Bu, CF₃, OCF₃, OMe, CH₂-heterocycloalkyl, phenyl, CN, F, NH₂, NHC(O)Me, C(O)Me, cyclopropyl, cyclopropyl substituted with haloalkyl, morpholino, benzyl, pyridyl or ethyl; or two R³ together with the carbon atoms to which they are attached form a cycloalkyl, heterocycloalkyl or heteroaryl ring.

In some embodiments of the first aspect of the invention, each R³ is independently selected from CI, Me, ^{t}Bu, CF₃, OCF₃, OMe, CH₂-heterocycloalkyl or phenyl; or wherein two R³ together with the carbon atoms to which they are attached form a heterocycloalkyl ring. In some such embodiments, each R³ is independently selected from CI, Me, ^{t}Bu, CF₃, OCF₃, OMe, —CH₂-morpholino or phenyl; or two R³ together with the carbon atoms to which they are attached form a heterocycloalkyl ring.

In some embodiments of the first aspect of the invention, wherein R is selected from:

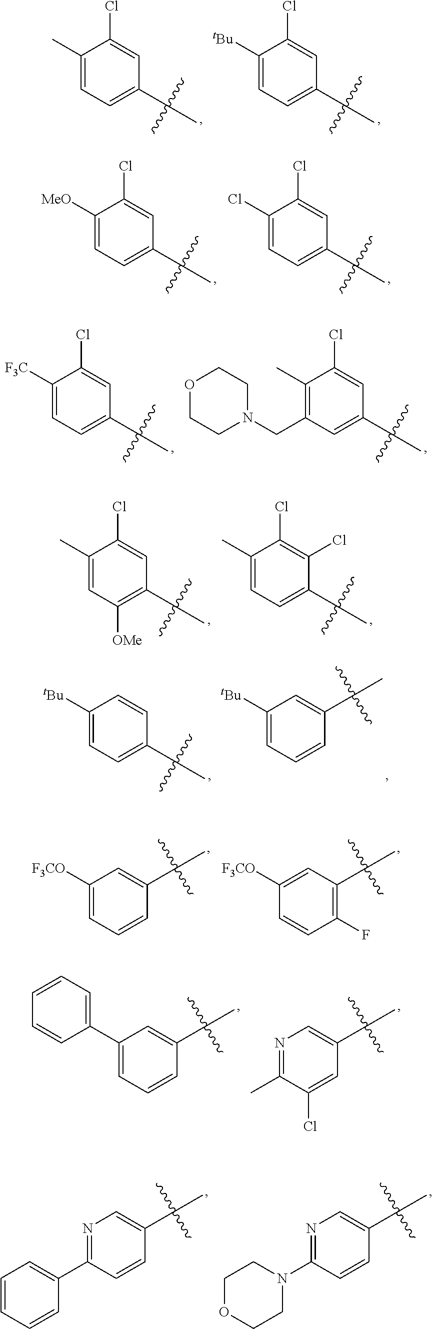

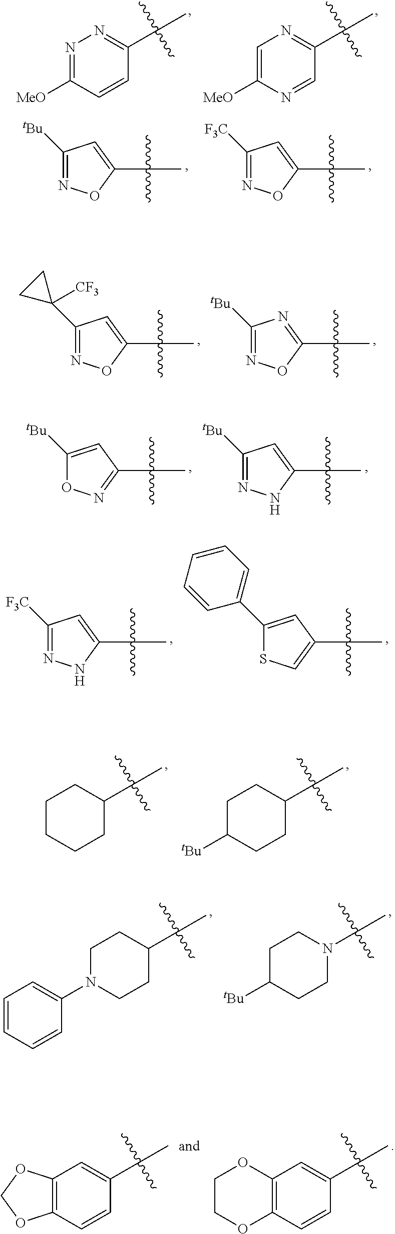

In some embodiments of the first aspect of the invention, R is selected from:

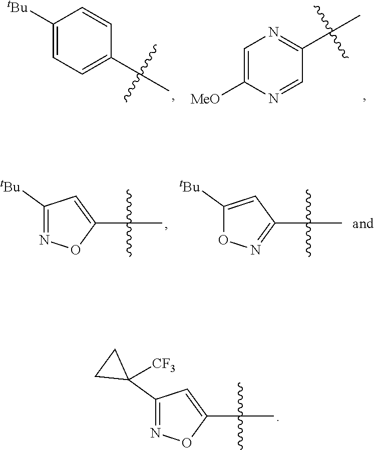 [SMILES: *c1ccc(C(C)(C)C)cc1 ; *c1cnc(OC)cn1 ; *c1cc(C(C)(C)C)no1 ; *c1cc(C(C)(C)C)on1 ; *c1cc(C2(C(F)(F)F)CC2)no1]

In some such embodiments, when R is

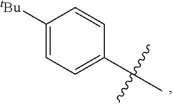 [SMILES: *c1ccc(C(C)(C)C)cc1]

then Q₂ is CR⁵ and Q₁, Q₃, and Q₄ are each independently CR⁴. In some embodiments, when R is

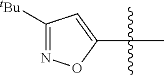 [SMILES: *c1cc(C(C)(C)C)no1]

Q₃ is CR⁵ and Q₁, Q₂, and Q₄ are each independently CR⁴; then R⁵ is

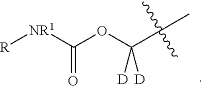

In some embodiments of the first aspect of the invention, when R is

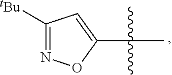 [SMILES: *c1cc(C(C)(C)C)no1]

Q₂ is CR⁵ and Q₁, Q₃, and Q₄ are each independently CR⁴; then R⁵ is

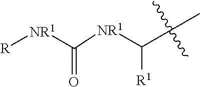

In some embodiments of the first aspect of the invention, R is selected from

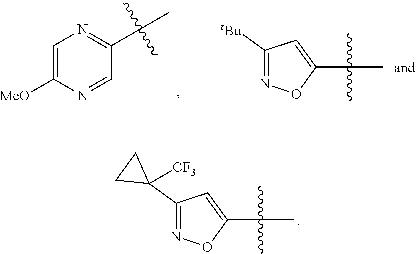 [SMILES: *c1cnc(OC)cn1 ; *c1cc(C(C)(C)C)no1 ; *c1cc(C2(C(F)(F)F)CC2)no1]

In some embodiments, R is selected from:

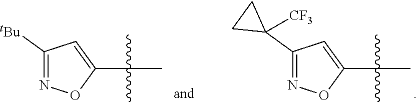 [SMILES: *c1cc(C(C)(C)C)no1 ; *c1cc(C2(C(F)(F)F)CC2)no1]

In some embodiments of the first aspect of the invention, R is selected from:

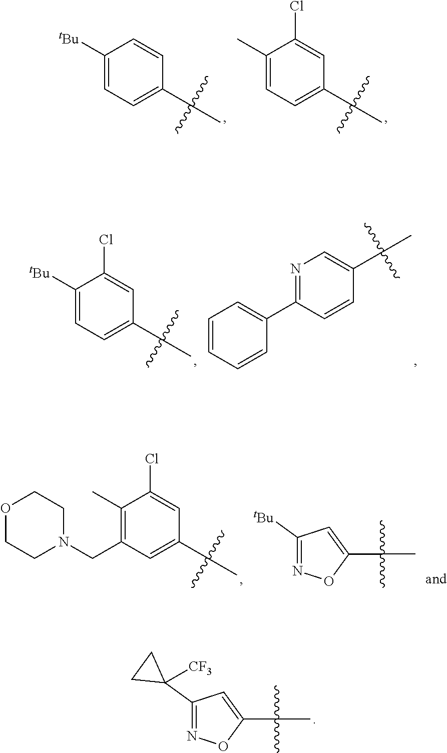

In some such embodiments, when R is

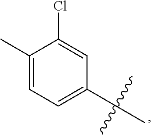 [SMILES: *c1ccc(C)c(Cl)c1]

then G is C-(unsubstituted C₁-C₄ alkyl).

In some embodiments, when R is

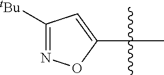 [SMILES: *c1cc(C(C)(C)C)no1]

then R⁵ is

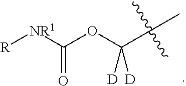

In some embodiments of the first aspect of the invention, each R² is independently hydrogen or halogen.

In some embodiments of the first aspect of the invention, each R⁴ is independently hydrogen, halogen or alkyl.

In some embodiments of the first aspect of the invention, each R¹ is independently hydrogen or methyl

In some embodiments of the first aspect of the invention, each NR¹ is NH.

In some embodiments of the first aspect of the invention the compound is selected from:

| Compound No. | Structure |
|---|---|
| 1 | 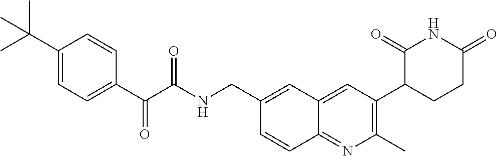 [SMILES: Cc1nc2ccc(CNC(=O)C(=O)c3ccc(C(C)(C)C)cc3)cc2cc1C1CCC(=O)NC1=O] |
| 2 | 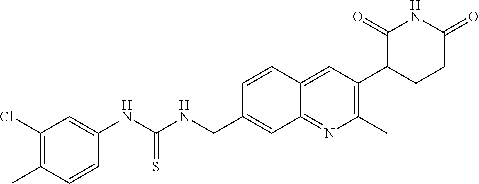 [SMILES: Cc1ccc(NC(=S)NCc2ccc3cc(C4CCC(=O)NC4=O)c(C)nc3c2)cc1Cl] |
| 3 | 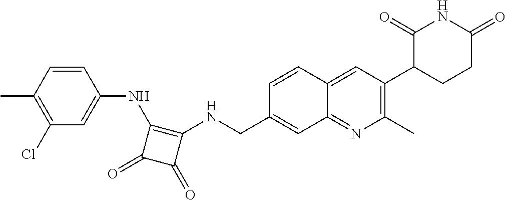 [SMILES: Cc1ccc(Nc2c(NCc3ccc4cc(C5CCC(=O)NC5=O)c(C)nc4c3)c(=O)c2=O)cc1Cl] |
| 4 | 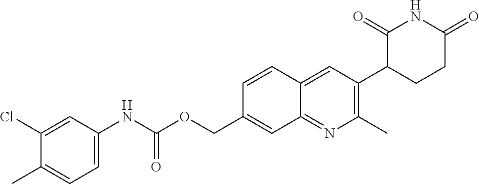 [SMILES: Cc1ccc(NC(=O)OCc2ccc3cc(C4CCC(=O)NC4=O)c(C)nc3c2)cc1Cl] |
| 5 | 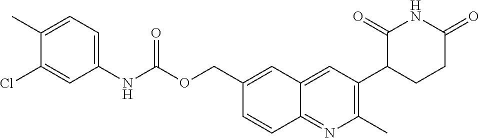 [SMILES: Cc1ccc(NC(=O)OCc2ccc3nc(C)c(C4CCC(=O)NC4=O)cc3c2)cc1Cl] |
| 6 | 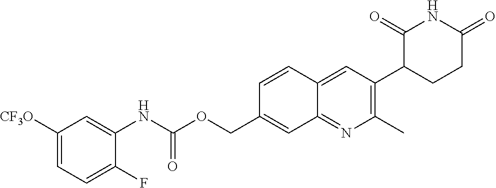 |
| 7 | 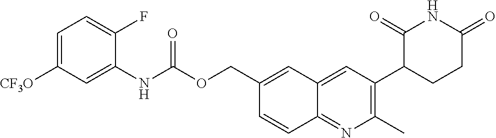 |
| 8 | 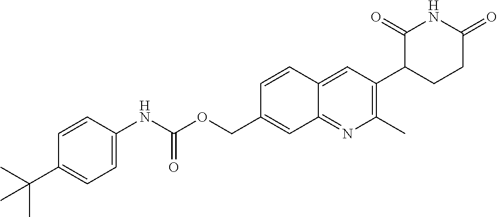 [SMILES: Cc1nc2cc(COC(=O)Nc3ccc(C(C)(C)C)cc3)ccc2cc1C1CCC(=O)NC1=O] |
| 9 | 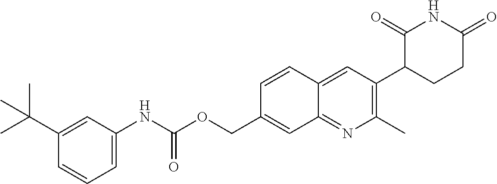 [SMILES: Cc1nc2cc(COC(=O)Nc3cccc(C(C)(C)C)c3)ccc2cc1C1CCC(=O)NC1=O] |
| 10 | 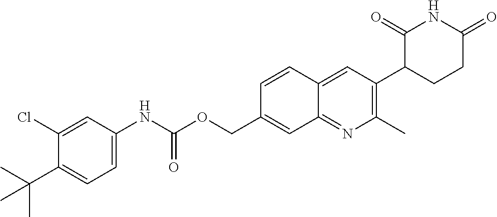 [SMILES: Cc1nc2cc(COC(=O)Nc3ccc(C(C)(C)C)c(Cl)c3)ccc2cc1C1CCC(=O)NC1=O] |
| 11 | 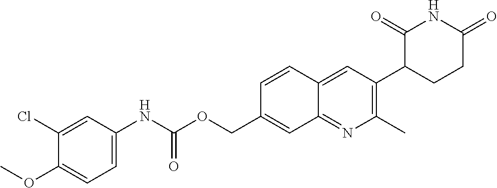 [SMILES: COc1ccc(NC(=O)OCc2ccc3cc(C4CCC(=O)NC4=O)c(C)nc3c2)cc1Cl] |
| 12 | 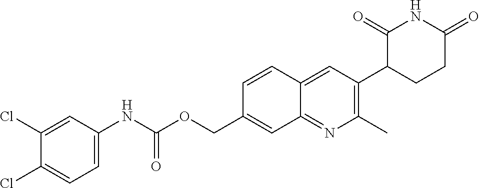 [SMILES: Cc1nc2cc(COC(=O)Nc3ccc(Cl)c(Cl)c3)ccc2cc1C1CCC(=O)NC1=O] |
| 13 | 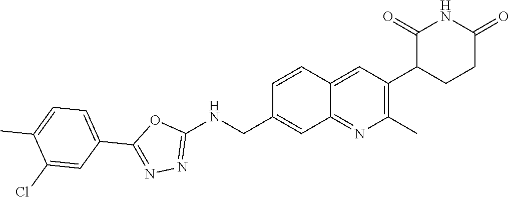 [SMILES: Cc1ccc(-c2nnc(NCc3ccc4cc(C5CCC(=O)NC5=O)c(C)nc4c3)o2)cc1Cl] |
| 14 | 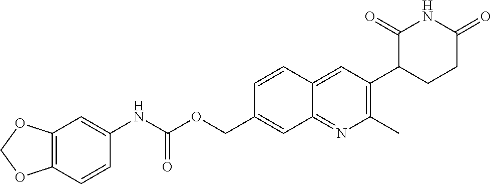 [SMILES: Cc1nc2cc(COC(=O)Nc3ccc4c(c3)OCO4)ccc2cc1C1CCC(=O)NC1=O] |
| 15 | 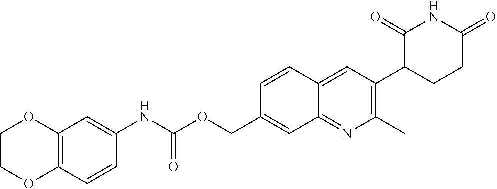 [SMILES: Cc1nc2cc(COC(=O)Nc3ccc4c(c3)OCCO4)ccc2cc1C1CCC(=O)NC1=O] |
| 17 | 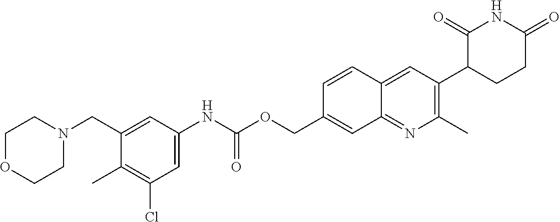 [SMILES: Cc1nc2cc(COC(=O)Nc3cc(Cl)c(C)c(CN4CCOCC4)c3)ccc2cc1C1CCC(=O)NC1=O] |
| 18 | 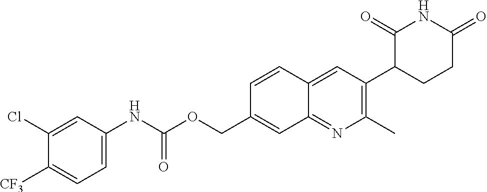 [SMILES: Cc1nc2cc(COC(=O)Nc3ccc(C(F)(F)F)c(Cl)c3)ccc2cc1C1CCC(=O)NC1=O] |
| 19 | 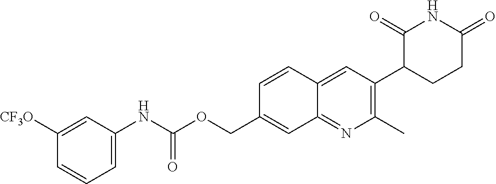 |
| 20 | 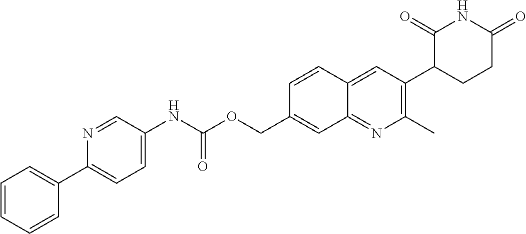 [SMILES: Cc1nc2cc(COC(=O)Nc3ccc(-c4ccccc4)nc3)ccc2cc1C1CCC(=O)NC1=O] |
| 21 | 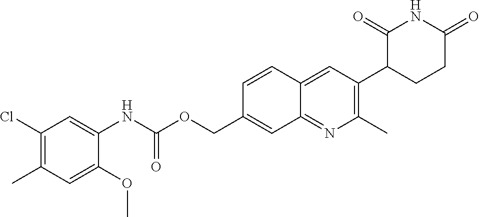 [SMILES: COc1cc(C)c(Cl)cc1NC(=O)OCc1ccc2cc(C3CCC(=O)NC3=O)c(C)nc2c1] |
| 22 | 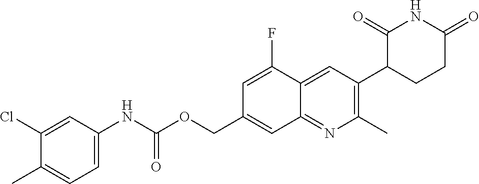 [SMILES: Cc1ccc(NC(=O)OCc2cc(F)c3cc(C4CCC(=O)NC4=O)c(C)nc3c2)cc1Cl] |
| 23 | 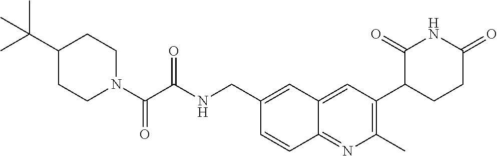 [SMILES: Cc1nc2ccc(CNC(=O)C(=O)N3CCC(C(C)(C)C)CC3)cc2cc1C1CCC(=O)NC1=O] |
| 26 | 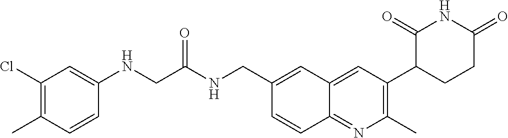 [SMILES: Cc1ccc(NCC(=O)NCc2ccc3nc(C)c(C4CCC(=O)NC4=O)cc3c2)cc1Cl] |
| 27 | 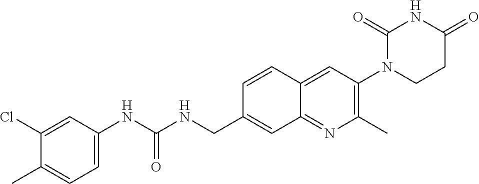 [SMILES: Cc1ccc(NC(=O)NCc2ccc3cc(N4CCC(=O)NC4=O)c(C)nc3c2)cc1Cl] |
| 28 | 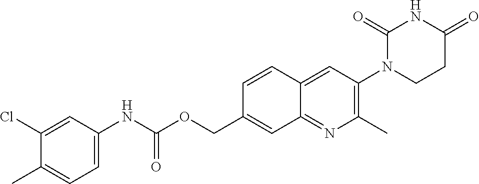 [SMILES: Cc1ccc(NC(=O)OCc2ccc3cc(N4CCC(=O)NC4=O)c(C)nc3c2)cc1Cl] |
| 29 | 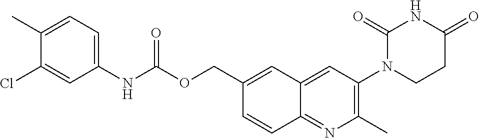 [SMILES: Cc1ccc(NC(=O)OCc2ccc3nc(C)c(N4CCC(=O)NC4=O)cc3c2)cc1Cl] |
| 30 | 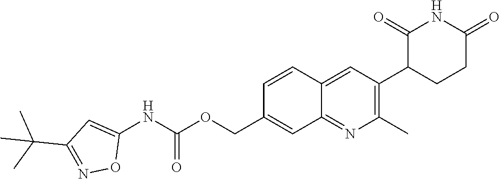 [SMILES: Cc1nc2cc(COC(=O)Nc3cc(C(C)(C)C)no3)ccc2cc1C1CCC(=O)NC1=O] |
| 32 | 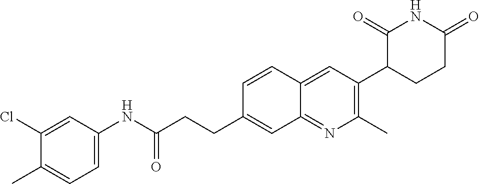 [SMILES: Cc1ccc(NC(=O)CCc2ccc3cc(C4CCC(=O)NC4=O)c(C)nc3c2)cc1Cl] |
| 34 | 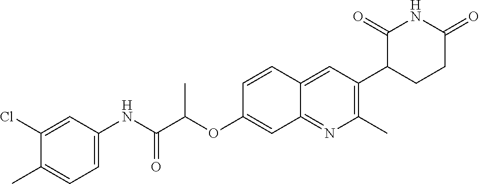 [SMILES: Cc1ccc(NC(=O)C(C)Oc2ccc3cc(C4CCC(=O)NC4=O)c(C)nc3c2)cc1Cl] |
| 36 | 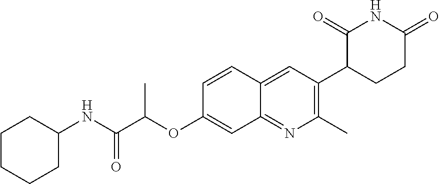 [SMILES: Cc1nc2cc(OC(C)C(=O)NC3CCCCC3)ccc2cc1C1CCC(=O)NC1=O] |
| 37 | 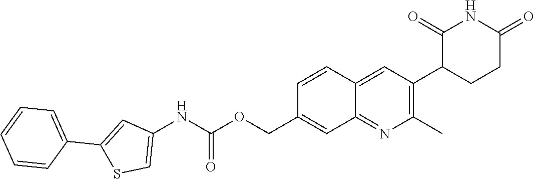 [SMILES: Cc1nc2cc(COC(=O)Nc3csc(-c4ccccc4)c3)ccc2cc1C1CCC(=O)NC1=O] |
| 38 | 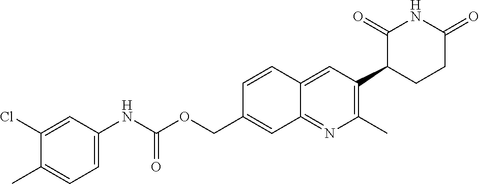 [SMILES: Cc1ccc(NC(=O)OCc2ccc3cc([C@@H]4CCC(=O)NC4=O)c(C)nc3c2)cc1Cl] |
| 39 | 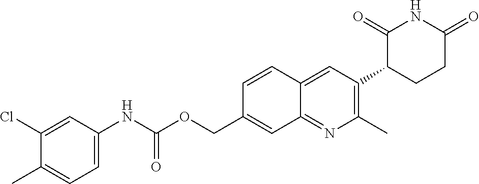 [SMILES: Cc1ccc(NC(=O)OCc2ccc3cc([C@H]4CCC(=O)NC4=O)c(C)nc3c2)cc1Cl] |
| 41 | 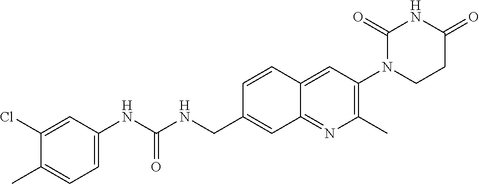 [SMILES: Cc1ccc(NC(=O)NCc2ccc3cc(N4CCC(=O)NC4=O)c(C)nc3c2)cc1Cl] |
| 42 | 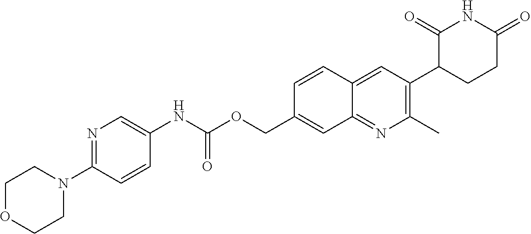 [SMILES: Cc1nc2cc(COC(=O)Nc3ccc(N4CCOCC4)nc3)ccc2cc1C1CCC(=O)NC1=O] |
| 44 | 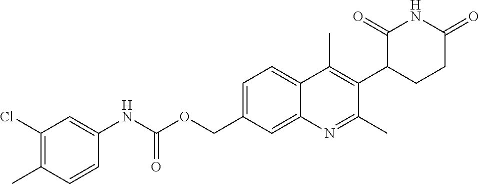 [SMILES: Cc1ccc(NC(=O)OCc2ccc3c(C)c(C4CCC(=O)NC4=O)c(C)nc3c2)cc1Cl] |
| 45 | 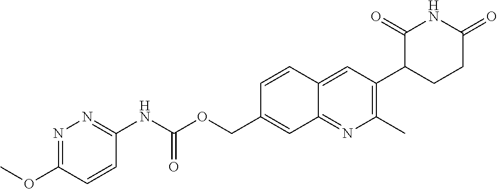 [SMILES: COc1ccc(NC(=O)OCc2ccc3cc(C4CCC(=O)NC4=O)c(C)nc3c2)nn1] |
| 46 | 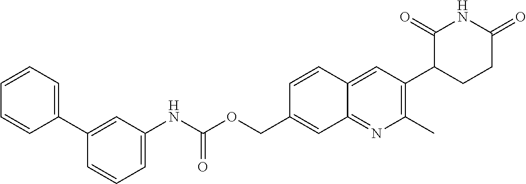 [SMILES: Cc1nc2cc(COC(=O)Nc3cccc(-c4ccccc4)c3)ccc2cc1C1CCC(=O)NC1=O] |
| 47 | 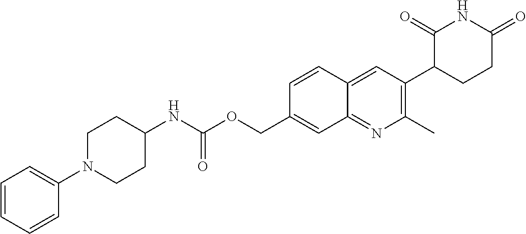 [SMILES: Cc1nc2cc(COC(=O)NC3CCN(c4ccccc4)CC3)ccc2cc1C1CCC(=O)NC1=O] |
| 49 | 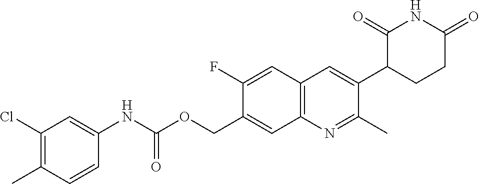 [SMILES: Cc1ccc(NC(=O)OCc2cc3nc(C)c(C4CCC(=O)NC4=O)cc3cc2F)cc1Cl] |
| 50 | 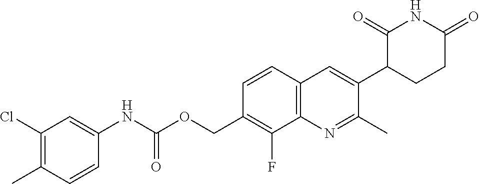 [SMILES: Cc1ccc(NC(=O)OCc2ccc3cc(C4CCC(=O)NC4=O)c(C)nc3c2F)cc1Cl] |
| 51 | 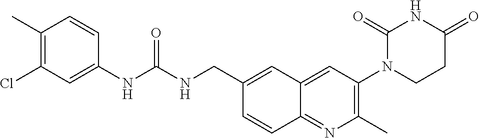 [SMILES: Cc1ccc(NC(=O)NCc2ccc3nc(C)c(N4CCC(=O)NC4=O)cc3c2)cc1Cl] |
| 52 | 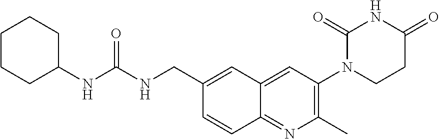 [SMILES: Cc1nc2ccc(CNC(=O)NC3CCCCC3)cc2cc1N1CCC(=O)NC1=O] |
| 54 | 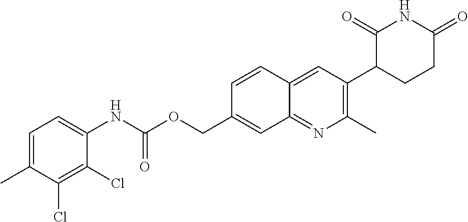 [SMILES: Cc1ccc(NC(=O)OCc2ccc3cc(C4CCC(=O)NC4=O)c(C)nc3c2)c(Cl)c1Cl] |
| 55 | 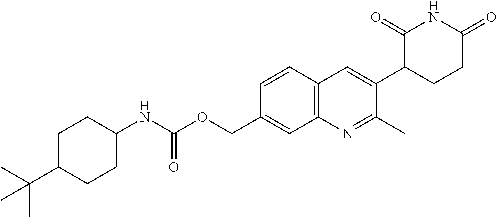 [SMILES: Cc1nc2cc(COC(=O)NC3CCC(C(C)(C)C)CC3)ccc2cc1C1CCC(=O)NC1=O] |
| 58 | 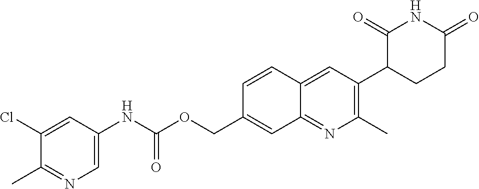 [SMILES: Cc1ncc(NC(=O)OCc2ccc3cc(C4CCC(=O)NC4=O)c(C)nc3c2)cc1Cl] |
| 59 | 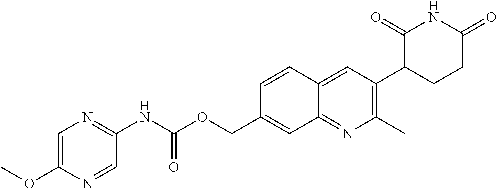 [SMILES: COc1cnc(NC(=O)OCc2ccc3cc(C4CCC(=O)NC4=O)c(C)nc3c2)cn1] |
| 60 | 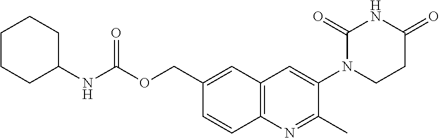 [SMILES: Cc1nc2ccc(COC(=O)NC3CCCCC3)cc2cc1N1CCC(=O)NC1=O] |
| 64 | 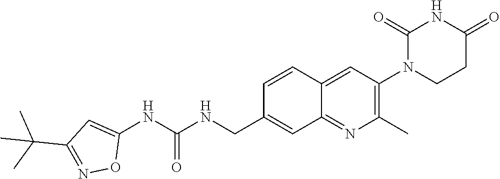 [SMILES: Cc1nc2cc(CNC(=O)Nc3cc(C(C)(C)C)no3)ccc2cc1N1CCC(=O)NC1=O] |
| 66 | 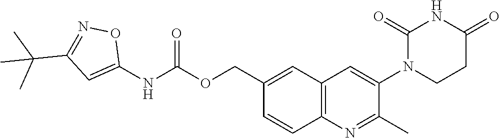 [SMILES: Cc1nc2ccc(COC(=O)Nc3cc(C(C)(C)C)no3)cc2cc1N1CCC(=O)NC1=O] |
| 67 | 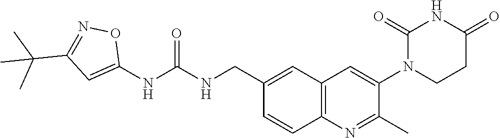 [SMILES: Cc1nc2ccc(CNC(=O)Nc3cc(C(C)(C)C)no3)cc2cc1N1CCC(=O)NC1=O] |
| 68 | 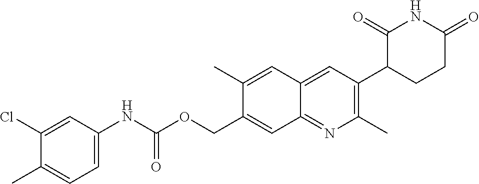 [SMILES: Cc1ccc(NC(=O)OCc2cc3nc(C)c(C4CCC(=O)NC4=O)cc3cc2C)cc1Cl] |
| 69 | 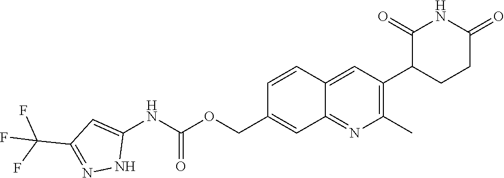 [SMILES: Cc1nc2cc(COC(=O)Nc3cc(C(F)(F)F)n[nH]3)ccc2cc1C1CCC(=O)NC1=O] |
| 70 | 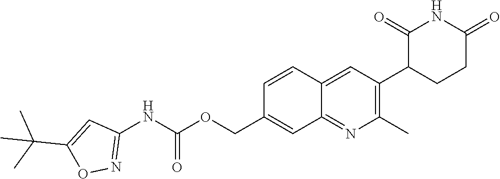 [SMILES: Cc1nc2cc(COC(=O)Nc3cc(C(C)(C)C)on3)ccc2cc1C1CCC(=O)NC1=O] |
| 71 | 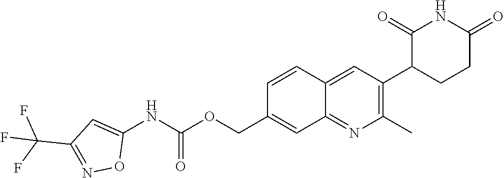 [SMILES: Cc1nc2cc(COC(=O)Nc3cc(C(F)(F)F)no3)ccc2cc1C1CCC(=O)NC1=O] |
| 72 | 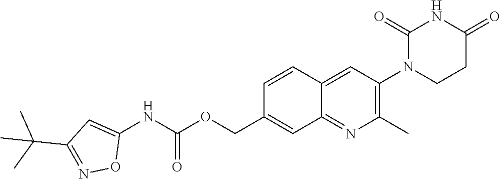 [SMILES: Cc1nc2cc(COC(=O)Nc3cc(C(C)(C)C)no3)ccc2cc1N1CCC(=O)NC1=O] |
| 74 | 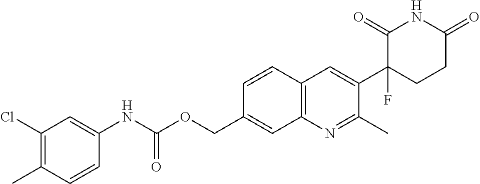 [SMILES: Cc1ccc(NC(=O)OCc2ccc3cc(C4(F)CCC(=O)NC4=O)c(C)nc3c2)cc1Cl] |
| 75 | 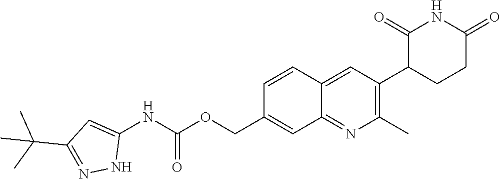 [SMILES: Cc1nc2cc(COC(=O)Nc3cc(C(C)(C)C)n[nH]3)ccc2cc1C1CCC(=O)NC1=O] |
| 80 | 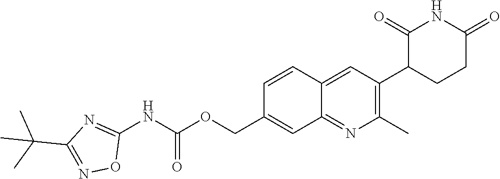 [SMILES: Cc1nc2cc(COC(=O)Nc3nc(C(C)(C)C)no3)ccc2cc1C1CCC(=O)NC1=O] |
| 81 | 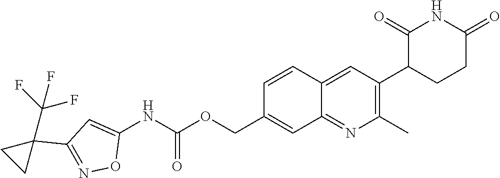 [SMILES: Cc1nc2cc(COC(=O)Nc3cc(C4(C(F)(F)F)CC4)no3)ccc2cc1C1CCC(=O)NC1=O] |
| 82 | 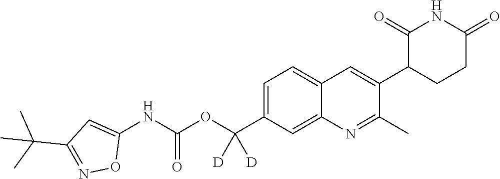 [SMILES: [2H]C([2H])(OC(=O)Nc1cc(C(C)(C)C)no1)c1ccc2cc(C3CCC(=O)NC3=O)c(C)nc2c1] |

In some embodiments, the compound is selected from Compounds 1, 2, 4, 5, 7, 8, 10, 13, 17, 18, 20, 21, 23, 26, 27, 28, 29, 30, 32, 34, 36, 39, 42, 44, 46, 47, 50, 51, 54, 55, 58, 59, 60, 64, 66, 67, 70, 72, 80, 81 and 82. In some such embodiments, the compound is selected from Compounds 1, 5, 7, 8, 10, 13, 17, 20, 23, 27, 29, 36, 42, 44, 47, 51, 59, 64, 66, 67, 70, 81 and 82.

In some embodiments, the compound is selected from Compounds 1, 4, 8, 11, 21, 22, 30, 32, 39, 42, 50, 54, 55, 58, 59, 66, 67, 69, 70, 80, 81 and 82. In some such embodiments, the compound is selected from Compounds 1, 59, 67, 70, 81 and 82.

In some embodiments, the compound is selected from Compounds 2, 3, 4, 6, 8, 9, 10, 11, 12, 13, 14, 15, 16, 17, 18, 19, 20, 21, 22, 27, 28, 30, 32, 34, 36, 37, 38, 39, 42, 44, 45, 46, 47, 49, 50, 54, 55, 58, 59, 64, 68, 72, 74, 75, 80, 81 and 82.

In some embodiments, the compound is selected from Compounds 4, 6, 8, 9, 10, 11, 12, 14, 15, 17, 18, 19, 20, 21, 22, 28, 29, 30, 32, 37, 39, 42, 44, 45, 46, 50, 51, 54, 55, 58, 59, 66, 67, 69, 70, 72, 75, 80, 81 and 82. In some such embodiments, the compound is selected from Compounds 8, 10, 11, 15, 17, 19, 20, 30, 39, 44, 58, 70, 81 and 82.

In a second aspect, the present invention provides a pharmaceutical composition comprising a compound of any embodiment of the first aspect of the invention.

In a third aspect, the present invention provides a compound of any of any embodiment of the first aspect, or a pharmaceutical composition of the second aspect, for use in medicine.

In a fourth aspect, the present invention provides a compound of any of any embodiment of the first aspect, or a pharmaceutical composition of the second aspect, for use in the treatment of cancer.

In some embodiments of the fourth aspect of the invention, the cancer is hepatocellular carcinoma, neuroblastoma, leukemia, acute myeloid leukemia (AML), acute promyelocytic leukemia (APL), multiple myeloma, breast cancer, prostate cancer, bladder cancer, kidney cancer, muscle cancer, ovarian cancer, skin cancer, pancreatic cancer, colon cancer, hematological cancer, cancer of connective tissue, placental cancer, bone cancer, uterine cancer, cervical cancer, choriocarcinoma, endometrial cancer, gastric cancer, or lung cancer.

As used herein, the chemical symbol “D” denotes deuterium, i.e. ²H.

As used herein the term “alkyl” is intended to include both linear and branched alkyl groups, both of which either may be unsubstituted, or may be substituted by one or more additional groups. An alkyl group as recited herein may be an unsubstituted alkyl group. In certain substituents of the compound of Formula (I) (for example R), an alkyl group (which may be linear or branched) may be substituted with one or more groups selected from halogen, R⁷, —CH₂-heterocycloalkyl, —CN, —OH, OR⁷, —NH₂, —NR¹R⁷, —NHC(O)R⁷, —NHC(O)OR⁷, —NHC(O)NR¹R⁷ and —C(O)R⁷; wherein each R⁷ is independently alkyl, haloalkyl, heteroaryl, aryl, benzyl, cycloalkyl or heterocycloalkyl; and each R¹ is independently hydrogen, unsubstituted C₁-C₄ alkyl, or C₁-C₄ haloalkyl. In other substituents of the compound of Formula (I) (for example E, R³, R⁷ and R⁸) an alkyl group (which may be linear or branched) may be substituted with one or more groups selected from halogen, —OH, —O(haloalkyl), —O(unsubstituted alkyl) and —N(R¹)₂, wherein each R¹ is independently hydrogen, unsubstituted C₁-C₄ alkyl, or C₁-C₄ haloalkyl. In some embodiments, the alkyl group is a C₁-C₁₂ alkyl, a C₁-C₁₀ alkyl, a C₁-C₈ alkyl, a C₁-C₆ alkyl, or a C₁-C₄ alkyl group. In some embodiments the alkyl group is a linear alkyl group. In some embodiments the alkyl group is an unsubstituted linear alkyl group. In some embodiments the alkyl group is a branched alkyl group. In some embodiments the alkyl group is an unsubstituted branched alkyl group.

As used herein the term “cycloalkyl” is intended to include both unsubstituted cycloalkyl groups, and cycloalkyl groups which are substituted by one or more additional groups. The term “cycloalkyl” is also intended to include monocyclic and bicyclic ring systems (including spirocyclic ring systems, in which the two rings share a single atom; fused bicyclic ring systems, in which the two rings share two adjacent atoms; and bridged bicyclic ring systems, in which the two rings share three or more atoms). A cycloalkyl group as recited herein may be an unsubstituted cycloalkyl group. In certain substituents of the compound of Formula (I) (for example R), a cycloalkyl group may be substituted with one or more groups selected from halogen, R⁷, —CH₂-heterocycloalkyl, —CN, —OH, OR⁷, —NH₂, —NR¹R⁷, —NHC(O)R⁷, —NHC(O)OR⁷, —NHC(O)NR¹R⁷ and —C(O)R⁷; wherein each R⁷ is independently alkyl, haloalkyl, heteroaryl, aryl, benzyl, cycloalkyl or heterocycloalkyl; and each R¹ is independently hydrogen, unsubstituted C₁-C₄ alkyl, or C₁-C₄ haloalkyl. In other substituents of the compound of Formula (I) (for example A, R³, R⁷ and R⁸), a cycloalkyl group may be substituted with one or more groups selected from halogen, unsubstituted alkyl, haloalkyl, —OH, —O(haloalkyl), —O(unsubstituted alkyl) and —N(R¹)₂, wherein each R¹ is independently hydrogen, unsubstituted C₁-C₄ alkyl, or C₁-C₄ haloalkyl. In some embodiments, one or more —CH₂— groups of the cycloalkyl ring may be replaced with a —C(O)— group. In some embodiments, the cycloalkyl group is a C₃-C₁₂ cycloalkyl, a C₄-C₁₂ cycloalkyl, a C₅-C₁₂ cycloalkyl, a C₃-C₁₀ cycloalkyl, a C₄-C₁₀ cycloalkyl, a C₅-C₁₀ cycloalkyl, a C₃-C₈ cycloalkyl, a C₄-C₈ cycloalkyl, a C₅-C₈ cycloalkyl, a C₃-C₆ cycloalkyl, a C₄-C₆ cycloalkyl, a C₅-C₆ cycloalkyl, a C₃-C₄ cycloalkyl, or a C₄-C₅ cycloalkyl group.

As used herein the term “cycloalkenyl” is intended to include both unsubstituted cycloalkenyl groups, and cycloalkenyl groups which are substituted by one or more additional groups. A cycloalkenyl group as recited herein may be an unsubstituted cycloalkenyl group. In certain substituents of the compound of Formula (I) (for example A, R³, R⁷ and R⁸), a cycloalkenyl group may be substituted by one or more groups selected from halogen, unsubstituted alkyl, haloalkyl, —OH, —O(haloalkyl), —O(unsubstituted alkyl) and —N(R¹)₂, wherein each R¹ is independently hydrogen, unsubstituted C₁-C₄ alkyl, or C₁-C₄ haloalkyl. In some embodiments, one or more —CH₂— groups of the cycloalkenyl ring may be replaced with a —C(O)— group. In some embodiments, the cycloalkenyl group is a C₄-C₁₂ cycloalkenyl, a C₅-C₁₂ cycloalkenyl, a C₄-C₁₀ cycloalkenyl, a C₅-C₁₀ cycloalkenyl, a C₄-C₈ cycloalkenyl, a C₅-C₈ cycloalkenyl, a C₄-C₆ cycloalkenyl, a C₅-C₆ cycloalkenyl, or a C₄-C₅ cycloalkenyl group.

As used herein the term “heterocycloalkyl” is intended to include both unsubstituted heterocycloalkyl groups, and heterocycloalkyl groups which are substituted by one or more additional groups. The term “heterocycloalkyl” is also intended to include monocyclic and bicyclic ring systems (including spirocyclic ring systems, in which the two rings share a single atom; fused bicyclic ring systems, in which the two rings share two adjacent atoms; and bridged bicyclic ring systems, in which the two rings share three or more atoms). In some embodiments, the heterocycloalkyl group is a monocyclic ring system, a spirocyclic ring system, or a fused bicyclic ring system. A heterocycloalkyl group as recited herein may be an unsubstituted heterocycloalkyl group. In certain substituents of the compound of Formula (I) (for example R), a heterocycloalkyl group may be substituted with one or more groups selected from halogen, R⁷, —CH₂-heterocycloalkyl, —CN, —OH, OR⁷, —NH₂, —NR¹R⁷, —NHC(O)R⁷, —NHC(O)OR⁷, —NHC(O)NR¹R⁷ and —C(O)R⁷; wherein each R⁷ is independently alkyl, haloalkyl, heteroaryl, aryl, benzyl, cycloalkyl or heterocycloalkyl; and each R¹ is independently hydrogen, unsubstituted C₁-C₄ alkyl, or C₁-C₄ haloalkyl. In other substituents of the compound of Formula (I) (for example A, R³, R⁷ and R⁸), a heterocycloalkyl group may be substituted by one or more groups selected from halogen, unsubstituted alkyl, haloalkyl, —OH, —O(haloalkyl), —O(unsubstituted alkyl) and —N(R¹)₂, wherein each R¹ is independently hydrogen, unsubstituted C₁-C₄ alkyl, or C₁-C₄ haloalkyl. In some embodiments, one or more —CH₂— groups of the heterocycloalkyl ring may be replaced with a —C(O)— group. In some embodiments, the heterocycloalkyl group is a C₃-C₁₂ heterocycloalkyl, a C₄-C₁₂ heterocycloalkyl, a C₅-C₁₂ heterocycloalkyl, a C₃-C₁₀ heterocycloalkyl, a C₄-C₁₀ heterocycloalkyl, a C₅-C₁₀ heterocycloalkyl, a C₃-C₈ heterocycloalkyl, a C₄-C₈ heterocycloalkyl, a C₅-C₈ heterocycloalkyl, a C₃-C₆ heterocycloalkyl, a C₄-C₆ heterocycloalkyl, a C₅-C₆ heterocycloalkyl, a C₃-C₄ heterocycloalkyl, or a C₄-C₅ heterocycloalkyl group.

As used herein the term “aryl” is intended to include both unsubstituted aryl groups, and aryl groups which are substituted by one or more additional groups. As recited herein an aryl group may be an unsubstituted aryl group. In certain substituents of the compound of Formula (I) (for example R), an aryl group may be substituted with one or more groups selected from halogen, R⁷, —CH₂— heterocycloalkyl, —CN, —OH, OR⁷, —NH₂, —NR¹R⁷, —NHC(O)R⁷, —NHC(O)OR⁷, —NHC(O)NR¹R⁷ and —C(O)R⁷; wherein each R⁷ is independently alkyl, haloalkyl, heteroaryl, aryl, benzyl, cycloalkyl or heterocycloalkyl; and each R¹ is independently hydrogen, unsubstituted C₁-C₄ alkyl, or C₁-C₄ haloalkyl. In other substituents of the compound of Formula (I) (for example A, R³, R⁷ and R⁸), an aryl group may be substituted by one or more groups selected from halogen, unsubstituted alkyl, haloalkyl, —OH, —O(haloalkyl), —O(unsubstituted alkyl) and —N(R¹)₂, wherein each R¹ is independently hydrogen, unsubstituted C₁-C₄ alkyl, or C₁-C₄ haloalkyl. In some embodiments, the aryl group is a C₆-C₁₀ aryl, a C₆-C₈ aryl, or a C₆ aryl.

As used herein the term “heteroaryl” is intended to include both unsubstituted heteroaryl groups, and heteroaryl groups which are substituted by one or more additional groups. As recited herein, a heteroaryl group may be an unsubstituted heteroaryl group. In certain substituents of the compound of Formula (I) (for example R), a heteroaryl group may be substituted with one or more groups selected from halogen, R⁷, —CH₂-heterocycloalkyl, —CN, —OH, OR⁷, —NH₂, —NR¹R⁷, —NHC(O)R⁷, —NHC(O)OR⁷, —NHC(O)NR¹R⁷ and —C(O)R⁷; wherein each R⁷ is independently alkyl, haloalkyl, heteroaryl, aryl, benzyl, cycloalkyl or heterocycloalkyl; and each R¹ is independently hydrogen, unsubstituted C₁-C₄ alkyl, or C₁-C₄ haloalkyl. In other substituents of the compound of Formula (I) (for example A, R³, R⁷ and R⁸), a heteroaryl group may be substituted by one or more groups selected from halogen, unsubstituted alkyl, haloalkyl, —OH, —O(haloalkyl), —O(unsubstituted alkyl) and —N(R¹)₂, wherein each R¹ is independently hydrogen, unsubstituted C₁-C₄ alkyl, or C₁-C₄ haloalkyl. In some embodiments, the heteroaryl group is a C₆-C₁₀ heteroaryl, a C₆-C₉ heteroaryl, a C₆-C₈ heteroaryl, or a C₆ heteroaryl.

As used herein the term “fused heterocycloalkyl-heteroaryl” is intended to mean a bicyclic ring system in which one ring is a heterocycloalkyl ring and the other is a heteroaryl ring, and in which the two rings share two adjacent atoms. Of the two adjacent atoms shared by the two rings, both may be carbon atoms; both may be heteroatoms (e. g. independently O, N or S); or one may be a carbon atom and the other a heteroatom (e. g. O, N or S). The fused heterocycloalkyl-heteroaryl may be unsubstituted or may be substituted by one or more additional groups.

As used herein the term “benzyl” is intended to include both unsubstituted benzyl groups, and benzyl groups which are substituted by one or more additional groups. As recited herein, a benzyl group may be an unsubstituted benzyl group. In certain substituents of the compound of Formula (I) (for example R), a benzyl group may be substituted with one or more groups selected from halogen, R⁷, —CH₂— heterocycloalkyl, —CN, —OH, OR⁷, —NH₂, —NR¹R⁷, —NHC(O)R⁷, —NHC(O)OR⁷, —NHC(O)NR¹R⁷ and —C(O)R⁷; wherein each R⁷ is independently alkyl, haloalkyl, heteroaryl, aryl, benzyl, cycloalkyl or heterocycloalkyl; and each R¹ is independently hydrogen, unsubstituted C₁-C₄ alkyl, or C₁-C₄ haloalkyl.

### BRIEF DESCRIPTION OF THE FIGURES

FIG. 1 shows the immunoblot analysis of Hep3B cells treated with DMSO or Compound 4, for 6 and 24 hours as indicated.

FIG. 2 is a graphical representation of the dose response curves showing % of GSPT1 protein degradation induced by Compound 4 in HEP3B cells.

### DETAILED DESCRIPTION OF THE INVENTION

As discussed above, the present invention provides compound of formula (I):

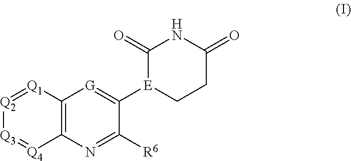

wherein
   R⁶ is hydrogen, unsubstituted C₁-C₄ alkyl, haloalkyl, —OR¹ or —N(R¹)₂;
   E is CH, CD, CF, C—(C₁-C₃ alkyl) or N;
   G is CR¹ or N;
   one of Q₁, Q₂, Q₃, and Q₄ is CR⁵ and the other three of Q₁, Q₂, Q₃, and Q₄ are each independently N or CR⁴; wherein when G is CR¹ then at least one of Q₁, Q₂, Q₃, and Q₄ is CR⁴; and when G is N then at least two of Q₁, Q₂, Q₃, and Q₄ are CR⁴;
   each R¹ is independently hydrogen, unsubstituted C₁-C₄ alkyl, or C₁-C₄ haloalkyl; each R⁴ is independently hydrogen, unsubstituted alkyl, haloalkyl, halogen, —CN, —OR¹ or —N(R¹)₂;
   R⁵ is selected from:

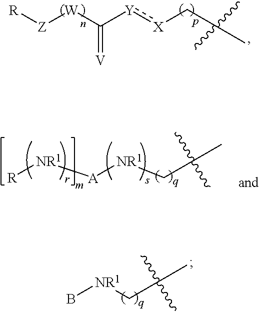

wherein [symbol] a single bond or a double bond, wherein
when [symbol] is a single bond, then X and Y are each independently O, NR¹, CHR¹ or CD₂;
wherein when one of X and Y is O or NR¹, then the other of X and Y is CHR¹ or CD₂; and
   when [symbol] is a double bond, then X and Y are each CR¹;
W is CH₂ or C═O;
Z is C═O, NR¹ or C(R²)₂; wherein each R² is independently hydrogen, unsubstituted alkyl, halogen, OR¹ or N(R¹)₂;
V is O or S;
each R is independently cycloalkyl, heterocycloalkyl, aryl, heteroaryl, alkyl, or benzyl wherein each cycloalkyl, heterocycloalkyl, aryl, heteroaryl, alkyl or benzyl is optionally substituted with one or more R³, wherein each R³ is independently selected from halogen, R⁷, —CH₂-heterocycloalkyl, —CN, —OH, OR⁷, —NH₂, —NR¹R⁷, —NHC(O)R⁷, —NHC(O)OR⁷, —NHC(O)NR¹R⁷ and —C(O)R⁷; or wherein two R³ together with the carbon atoms to which they are attached form a cycloalkyl, heterocycloalkyl or heteroaryl ring;
or wherein R and R¹, together with the N atom to which they are attached when Z is NR¹, form a heterocycloalkyl or heteroaryl ring which is optionally substituted with one or more groups selected from halogen, unsubstituted alkyl, haloalkyl, —OH, —O(haloalkyl), —O(unsubstituted alkyl) and —N(R¹)₂;
each R⁷ is independently alkyl, haloalkyl, heteroaryl, aryl, benzyl, cycloalkyl or heterocycloalkyl;
A is heteroaryl, heterocycloalkyl, cycloalkyl or cycloalkenyl;
B is —SO₂NHR or 8-12 membered bicyclic heteroaryl substituted with one or more R⁸, wherein each R⁸ is independently selected from halogen, alkyl, —O(haloalkyl), —O(unsubstituted alkyl), aryl, and CH₂-heterocycloalkyl;
n is 0 or 1;
m is 1 or 2;
r is 0 or 1;
s is 0 or 1;
each p is independently 0, 1, 2 or 3; and
each q is independently 1, 2, 3 or 4;
wherein:
   one or more CH₂ groups of any cycloalkyl, heterocycloalkyl or cycloalkenyl are optionally replaced by a C═O group;
   in E, R³, R⁷ and R⁸, each alkyl is independently optionally substituted with one or more groups selected from halogen, —OH, —O(haloalkyl), —O(unsubstituted alkyl) and —N(R¹)₂; and
   in A, R³, R⁷ and R⁸, each cycloalkyl, heterocycloalkyl, cycloalkenyl, aryl and heteroaryl is independently optionally substituted with one or more groups selected from halogen, unsubstituted alkyl, haloalkyl, —OH, —O(haloalkyl), —O(unsubstituted alkyl) and —N(R¹)₂;
   provided that:
(a) when R⁵ is

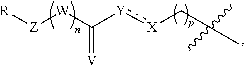

W is C═O, n is 1 and p is 0; then Z is NR¹ or C(R²)₂; and
(b) when R⁵ is

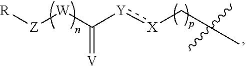

X is CHR¹, Y is NH, V is O, n is 0, p is 0, Z is NH or C(halogen)₂, and R is aryl; then at least one of E and G is N.

The present invention also provides a pharmaceutical composition comprising a compound of the invention.

The present invention also provides a compound or pharmaceutical composition of the invention for use in medicine.

The present invention also provides a compound or pharmaceutical composition of the invention for use in the treatment of cancer.

The compounds may be in the form of pharmaceutically acceptable salts or solvates.

As used herein, and unless otherwise specified, the term “pharmaceutically acceptable salt” refers to salts prepared from pharmaceutically acceptable non-toxic acids, including inorganic acids and organic acids. As used herein, and unless otherwise specified, the term “solvate” means a compound of the present invention or a salt thereof, that further includes a stoichiometric or non-stoichiometric amount of solvent bound by non-covalent intermolecular forces. Where the solvent is water, the solvate is a hydrate.

### EXAMPLES

The reagents and solvents were used as received from the commercial sources. Proton nuclear magnetic resonance (NMR) spectra were recorded on 500 MHz Bruker Avance spectrometer. The spectra are reported in terms of chemical shift (δ [ppm]), multiplicity (s=singlet, br s=broad singlet, d=doublet, t=triplet, q=quartet, p=quintet, m=multiplet), coupling constant (J [Hz]), and integration. Chemical shifts are reported in ppm relative to dimethyl sulfoxide-d₆ (δ 2.50) as indicated in NMR spectra data. The samples were prepared by dissolving a dry sample (0.2-2 mg) in an appropriate deuterated solvent (0.7-1 mL).

LCMS measurements were collected using either Shimadzu Nexera X2/MS-2020 or Advion Expression CMS coupled to liquid chromatograph. All masses reported are the m/z of the protonated parent ions unless otherwise stated. The sample was dissolved in an appropriate solvent (e.g. DMSO, ACN, water) and was injected directly into the column using an automated sample handler.

Purification of the products was performed using flash column chromatography (Interchim PuriFlash® 430, XS520, or 5.020 using PuriFlash® columns preloaded with SI-HP or C18-HP gels). Preparative TLC was performed using Analtech® Uniplate® glass backed silica gel GF plates. Preparative HPLC was performed using Thermo Fisher Scientific UltiMate™ 3000 instruments equipped with Hypersil™ ODS C18, ACE® C18-Amide or ACE® C18-PFP HPLC columns.

The chemical names were generated using ChemDraw Professional v. 18.2.0.48 from PerkinElmer Informatics, Inc.

Abbreviations used in the following examples are presented below in the alphabetical order:
ACN Acetonitrile
Boc Tert-butoxycarbonyl
DCE 1,2-Dichloroethane
DCM Dichloromethane
DIPEA N,N-Diisopropylethylamine
DMAP N,N-Dimethylpyridin-4-amine
DMF N,N-Dimethylformamide
DMSO Dimethyl sulfoxide
Et Ethyl
FA Formic acid
HATU 1-[Bis(dimethylamino)methylene]-1H-1,2,3-triazolo[4,5-b]pyridinium 3-oxide hexafluorophosphate
HPLC High performance liquid chromatography
LCMS Liquid chromatography mass spectrometry
LDA Lithium diisopropylamide
LiHMDS Lithium bis(trimethylsilyl)amide
NFSI N-Fluoro-N-(phenylsulfonyl)benzenesulfonamide
NMP N-Methyl-2-pyrrolidone
NMR Nuclear magnetic resonance
PyBOP (Benzotriazol-1-yloxy)tripyrrolidinophosphonium hexafluorophosphate
Rₜ Retention time
RT Room temperature
TEA Triethylamine
TFA Trifluoroacetic acid
TEBAC Benzyltriethylammonium chloride
THF Tetrahydrofuran

### General Procedures

The synthesis of the compounds can be summarized in the following general procedures as set out below:

### Example Method 1: Amide Formation—Method A

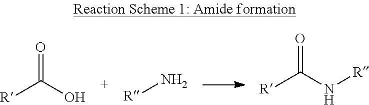

To a solution of the acid (1 equiv), amine (1-1.2 equiv), HATU (1-2 equiv) and/or DMAP (cat.) in appropriate solvent (e.g. DMF) was added DIPEA (3-6 equiv) and the reaction mixture was stirred under inert atmosphere at RT for 2-24 h. The volatiles were removed under reduced pressure and the crude product was isolated by flash column chromatography and/or preparative HPLC.

### Example Method 2: Amide Formation—Method B

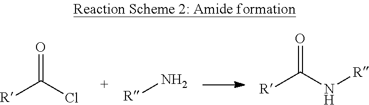

The amine (1 equiv) and base (3-6 equiv) were dissolved in an appropriate solvent (e.g. DMF). The solution of acid chloride (1.5-2.5 equiv) in the same solvent was added dropwise and the reaction mixture was stirred for 2-24 h at 20-80° C. The volatiles were removed under reduced pressure and the crude product was purified by flash column chromatography and/or preparative HPLC.

### Example Method 3: Carbamate Formation—Method A

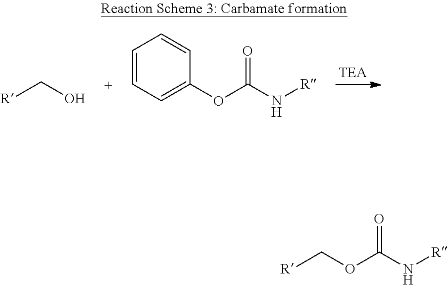

To a solution of an alcohol (1 equiv) and phenyl carbamate (1-1.5 equiv) in anhydrous solvent (e.g. DMF) was added TEA, DIPEA or 1-methyl-1H-imidazole (1-5 equiv) and reaction mixture was stirred at 20-50° C. for 1-24 h. The volatiles were removed under reduced pressure and the crude product was purified by flash column chromatography and/or preparative HPLC.

### Example Method 4: Carbamate Formation—Method B

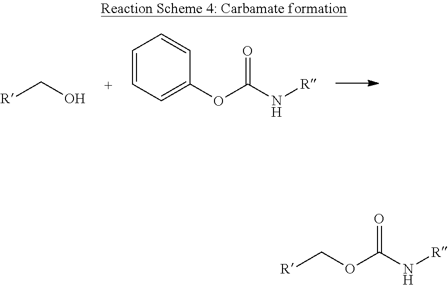

To a solution of an alcohol (1 equiv) and phenyl carbamate (1-1.5 equiv) in anhydrous solvent (e.g. DMF), was added sodium hydride (2-4 equiv, 60% suspension in mineral oil) in one portion and the resulting mixture was stirred at RT for 1-5 h. The reaction mixture was quenched by addition of formic acid or glacial acetic acid. The volatiles were removed under reduced pressure and the crude product was purified by flash column chromatography and/or preparative HPLC.

### Example Method 5: Carbamate Formation—Method C

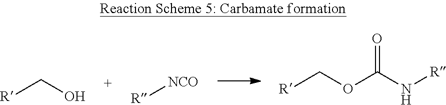

To a solution of an alcohol (1 equiv) in dry solvent (e.g. DMF, THF) at RT were added appropriate isocyanate (1-5 equiv) and TEA (2-4 equiv). The reaction mixture was stirred at RT for 1-18 h. The reaction mixture was quenched by addition of water. The volatiles were removed under reduced pressure and the crude product was purified by flash column chromatography and/or preparative HPLC.

### Example Method 6: Carbamate Synthesis from Amine and Phenyl Chloroformate

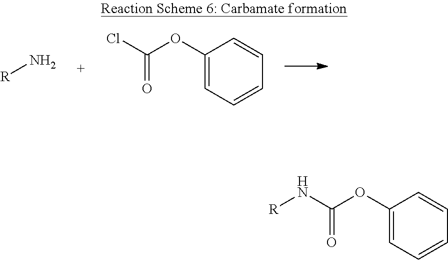

To a stirred solution of amine (1 equiv) in pyridine or in DCM and pyridine (2-4 equiv), cooled to 0° C., was added phenyl chloroformate (1-1.5 equiv) dropwise and the reaction was carried out for 0.5-18 h at RT. 10% Citric acid was added, the organic fraction was washed with water and brine, dried over Na₂SO₄ or MgSO₄ and evaporated. Product was purified by flash column chromatography.

### Example Method 7: Stille Coupling

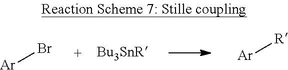

To a solution of aryl bromide (1 equiv) and palladium catalyst (0.05-0.15 equiv) in dry dioxane was added appropriate tributyltin derivative (1-4 equiv) under argon atmosphere and the solution was additionally bubbled with argon for 5-15 min. The reaction was then carried out at 95-115° C. in a sealed tube for 5-18 h. The product was purified by flash column chromatography directly after completion, or after work-up if necessary.

### Example Method 8: Michael Conjugate Addition

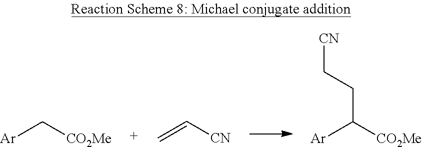

To a stirred solution of appropriate ester (1 equiv), potassium carbonate (1 equiv) and TEBAC (1 equiv) in DMF was added acrylonitrile (1-4 equiv). The reaction was carried out at 20-100° C. for 18-50 h, quenched by acetic acid and water. The product was extracted with ethyl acetate, the combined organic fractions were washed with water and brine, dried over Na₂SO₄ or MgSO₄ and evaporated. The product was purified by flash column chromatography.

### Example Method 9: Glutarimide Ring Formation

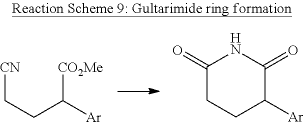

An appropriate substrate (1 equiv) was solubilized in glacial acetic acid and concentrated sulfuric acid (10-50 equiv) was added. The reaction mixture was stirred at 110° C. for 15-50 h, poured onto crushed ice and neutralized by slow addition of solid NaHCO₃. The solution was then extracted with ethyl acetate, the combined organic fractions were washed with water and brine, dried over Na₂SO₄ or MgSO₄ and evaporated. The crude product was used directly for the next step or further purified by flash column chromatography.

### Example 1: Synthesis of 2-(4-(tert-butyl)phenyl)-N-((3-(2,6-dioxopiperidin-3-yl)-2-methylquinolin-6-yl)methyl)-2-oxoacetamide (Compound 1)

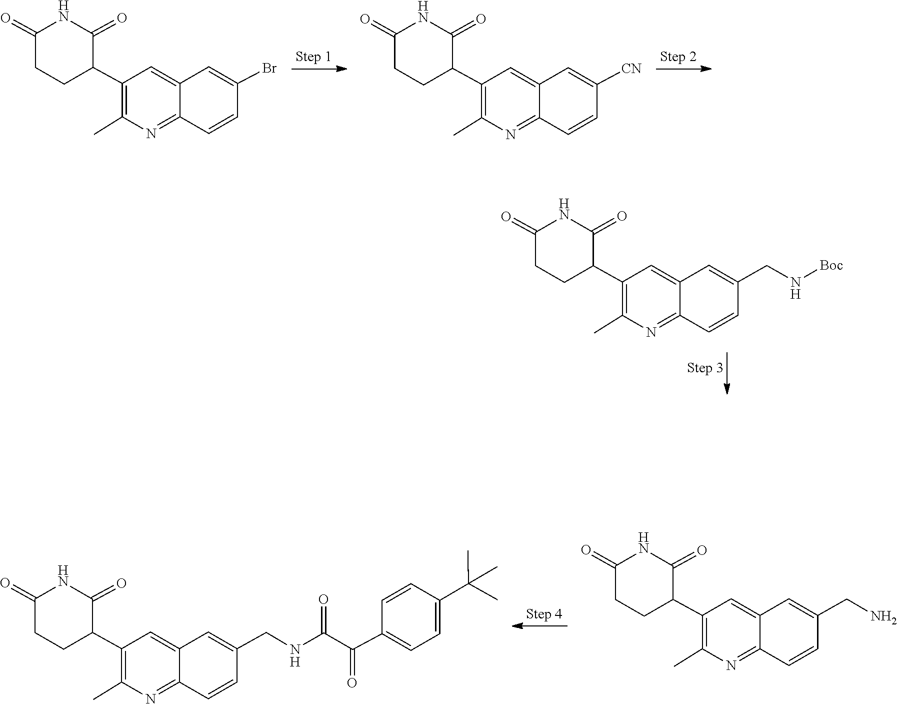 [SMILES: Cc1nc2ccc(Br)cc2cc1C1CCC(=O)NC1=O ; Cc1nc2ccc(C#N)cc2cc1C1CCC(=O)NC1=O ; Cc1nc2ccc(CNC(=O)OC(C)(C)C)cc2cc1C1CCC(=O)NC1=O ; Cc1nc2ccc(CNC(=O)C(=O)c3ccc(C(C)(C)C)cc3)cc2cc1C1CCC(=O)NC1=O ; Cc1nc2ccc(CN)cc2cc1C1CCC(=O)NC1=O]

Step 1: In a vial were placed 3-(6-bromo-2-methylquinolin-3-yl)piperidine-2,6-dione (300 mg, 0.9 mmol, 1 equiv), zinc cyanide (1 equiv) and tetrakis(triphenylphosphine)palladium(0) (0.1 equiv). DMF (10 mL) was added and the reaction mixture was stirred for 18 h at 100° C. The volatiles were removed under reduced pressure and the crude product was purified by flash column chromatography to give 3-(2,6-dioxopiperidin-3-yl)-2-methylquinoline-6-carbonitrile (230 mg, 91% yield).

The synthesis of 3-(6-bromo-2-methylquinolin-3-yl)piperidine-2,6-dione was described in WO2022253713A1.

LCMS (ESI+) m/z 280.0 [M+H]⁺

Step 2: To a solution of 3-(2,6-dioxopiperidin-3-yl)-2-methylquinoline-6-carbonitrile (43 mg, 0.154 mmol, 1 equiv) and di-tert-butyl dicarbonate (3.4 equiv) in DMF (2.3 mL) was added Raney Nickel (2 equiv) in THF (2.3 mL). The reaction mixture was stirred at RT for 24 h under hydrogen atmosphere (balloon). The reaction mixture was filtered through Celite® and concentrated under reduced pressure. The crude product was purified by flash column chromatography to give tert-butyl ((3-(2,6-dioxopiperidin-3-yl)-2-methylquinolin-6-yl)methyl)carbamate (35.5 mg, 60% yield).

LCMS (ESI+) m/z 384.1 [M+H]⁺

Step 3: tert-Butyl ((3-(2,6-dioxopiperidin-3-yl)-2-methylquinolin-6-yl)methyl)carbamate (20 mg, 0.052 mmol, 1 equiv) was dissolved in TFA (1 mL) and the solution was stirred at RT for 2 h. The volatiles were removed under reduced pressure to give 3-(6-(aminomethyl)-2-methylquinolin-3-yl)piperidine-2,6-dione trifluoroacetate (20.6 mg, 100% yield).

LCMS (ESI+) m/z 284.1 [M+H]⁺

Step 4: 2-(4-(tert-Butyl)phenyl)-N-((3-(2,6-dioxopiperidin-3-yl)-2-methylquinolin-6-yl)methyl)-2-oxoacetamide was synthesized using the general procedure shown in Reaction Scheme 2 and Example Method 2, above (49% yield), using 3-(6-(aminomethyl)-2-methylquinolin-3-yl)piperidine-2,6-dione trifluoroacetate (20.6 mg, 0.052 mmol, 1 equiv) and 2-(4-(tert-butyl)phenyl)-2-oxoacetyl chloride (2 equiv) as starting materials, DIPEA (5 equiv) as base and DMF as solvent.

LCMS (ESI+) m/z 472.2 [M+H]⁺

¹H NMR (500 MHz, DMSO-d₆) δ 10.92 (s, 1H), 9.52 (t, J=6.1 Hz, 1H), 8.11 (s, 1H), 7.96-7.93 (m, 2H), 7.92 (d, J=8.6 Hz, 1H), 7.78 (d, J=1.8 Hz, 1H), 7.67 (dd, J=8.7, 2.0 Hz, 1H), 7.63-7.58 (m, 2H), 4.62 (d, J=6.1 Hz, 2H), 4.29 (dd, J=12.5, 4.7 Hz, 1H), 2.88-2.78 (m, 1H), 2.65 (s, 3H), 2.62 (s, 1H), 2.43 (td, J=12.9, 4.2 Hz, 1H), 2.11 (dtd, J=13.4, 6.3, 5.7, 3.1 Hz, 1H), 1.31 (s, 9H).

### Example 2: Synthesis of 1-(3-chloro-4-methylphenyl)-3-((3-(2,6-dioxopiperidin-3-yl)-2-methylquinolin-7-yl)methyl)thiourea (Compound 2)

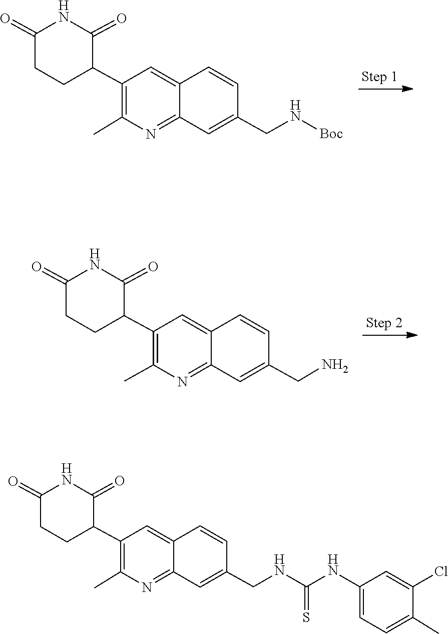 [SMILES: Cc1nc2cc(CNC(=O)OC(C)(C)C)ccc2cc1C1CCC(=O)NC1=O ; Cc1nc2cc(CN)ccc2cc1C1CCC(=O)NC1=O ; Cc1ccc(NC(=S)NCc2ccc3cc(C4CCC(=O)NC4=O)c(C)nc3c2)cc1Cl]

Step 1: tert-Butyl ((3-(2,6-dioxopiperidin-3-yl)-2-methylquinolin-7-yl)methyl)carbamate (60 mg, 0.156 mmol, 1 equiv) was dissolved in dry dioxane (1 mL) followed by addition of 4M HCl in dioxane (1 mL). The reaction mixture was stirred at RT for 30 min and the volatiles were removed under reduced pressure to give 3-(7-(aminomethyl)-2-methylquinolin-3-yl)piperidine-2,6-dione hydrochloride (40 mg, 80% yield).

The synthesis of tert-butyl ((3-(2,6-dioxopiperidin-3-yl)-2-methylquinolin-7-yl)methyl)carbamate was described in WO2022144416A1.

LCMS (ESI+) m/z 284.0 [M+H]⁺

Step 2: To an ice-cooled solution of 3-(7-(aminomethyl)-2-methylquinolin-3-yl)piperidine-2,6-dione hydrochloride (20 mg, 0.063 mmol, 1 equiv) in DCM (4 mL) was added TEA (2 equiv) followed by a slow addition of thiocarbonyldiimidazole (1.5 equiv). The reaction mixture was warmed up to RT and stirred for 18 h. The solution was cooled to 0° C., 3-chloro-4-methylaniline (1.5 equiv) was added and the reaction was continued at RT for 5 h. The volatiles were removed under reduced pressure and the crude product was purified by preparative HPLC to give 1-(3-chloro-4-methylphenyl)-3-((3-(2,6-dioxopiperidin-3-yl)-2-methylquinolin-7-yl)methyl)thiourea (3.1 mg, 11% yield).

LCMS (ESI+) m/z 467.1 [M+H]⁺

¹H NMR (500 MHz, DMSO-d₆) δ 10.92 (s, 1H), 9.77 (s, 1H), 8.44 (s, 1H), 8.08 (s, 1H), 7.87-7.79 (m, 2H), 7.67-7.63 (m, 1H), 7.50 (dd, J=8.4, 1.7 Hz, 1H), 7.32-7.23 (m, 2H), 4.93 (d, J=5.6 Hz, 2H), 4.28 (dd, J=12.4, 4.7 Hz, 1H), 2.82 (m, 1H), 2.65 (m, 4H), 2.42 (m, 1H), 2.29 (s, 3H), 2.12 (m, 1H).

### Example 3: Synthesis of 3-(7-(((2-((3-chloro-4-methylphenyl)amino)-3,4-dioxocyclobut-1-en-1-yl)amino)methyl)-2-methylquinolin-3-yl)piperidine-2,6-dione (Compound 3)

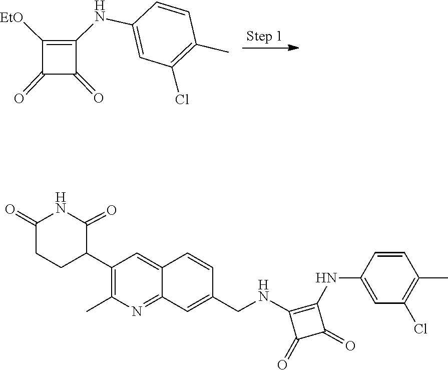 [SMILES: CCOc1c(Nc2ccc(C)c(Cl)c2)c(=O)c1=O ; Cc1ccc(Nc2c(NCc3ccc4cc(C5CCC(=O)NC5=O)c(C)nc4c3)c(=O)c2=O)cc1Cl]

Step 1: In a vial 3-(7-(aminomethyl)-2-methylquinolin-3-yl)piperidine-2,6-dione hydrochloride (20 mg, 0.062 mmol, 1 equiv) was dissolved in DMF (2 mL). TEA (5 equiv) was added followed by a solution of 3-((3-chloro-4-methylphenyl)amino)-4-ethoxycyclobut-3-ene-1,2-dione (1.1 equiv) in dry DMF (0.5 mL) and the reaction mixture was stirred at RT for 18 h. The volatiles were removed under reduced pressure and the crude product was purified by preparative HPLC to give 3-(7-(((2-((3-chloro-4-methylphenyl)amino)-3,4-dioxocyclobut-1-en-1-yl)amino)methyl)-2-methylquinolin-3-yl)piperidine-2,6-dione (2.2 mg, 6.4% yield).

LCMS (ESI+) m/z 503.1 [M+H]⁺

¹H NMR (500 MHz, DMSO-d₆) δ 10.92 (s, 1H), 9.93 (s, 1H), 8.34 (s, 1H), 8.12 (s, 1H), 7.93-7.84 (m, 2H), 7.65 (s, 1H), 7.53 (dd, J=8.3, 1.8 Hz, 1H), 7.29 (d, J=8.4 Hz, 1H), 7.21 (dd, J=8.2, 2.4 Hz, 1H), 5.01 (d, J=5.9 Hz, 2H), 4.29 (dd, J=12.5, 4.7 Hz, 1H), 2.82 (ddd, J=17.7, 12.9, 5.3 Hz, 1H), 2.65 (m, 4H), 2.42 (tt, J=12.9, 6.5 Hz, 1H), 2.27 (s, 3H), 2.16-2.08 (m, 1H).

### Example 4: Synthesis of (3-(2,6-dioxopiperidin-3-yl)-2-methylquinolin-7-yl)methyl (3-chloro-4-methylphenyl)carbamate (Compound 4)

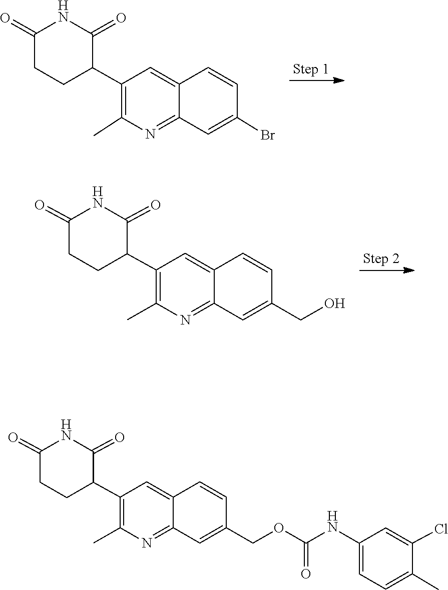 [SMILES: Cc1nc2cc(Br)ccc2cc1C1CCC(=O)NC1=O ; Cc1nc2cc(CO)ccc2cc1C1CCC(=O)NC1=O ; Cc1ccc(NC(=O)OCc2ccc3cc(C4CCC(=O)NC4=O)c(C)nc3c2)cc1Cl]

Step 1: 3-(7-(Hydroxymethyl)-2-methylquinolin-3-yl)piperidine-2,6-dione was synthesized using the general procedure shown in Reaction Scheme 7 and Example Method 7, above (85% yield), using 3-(7-bromo-2-methylquinolin-3-yl)piperidine-2,6-dione (112 mg, 0.336 mmol, 1 equiv) and (tributylstannyl)methanol (1.1 equiv) as starting materials, tetrakis(triphenylphosphine)palladium(0) (0.1 equiv) as catalyst and dioxane as solvent.

LCMS (ESI+) m/z 284.8 [M+H]⁺

¹H NMR (500 MHz, DMSO-d₆) δ 11.04 (s, 1H), 8.70 (s, 1H), 8.05 (s, 2H), 7.69 (d, J=8.3 Hz, 1H), 5.63 (s, 1H), 4.79 (s, 2H), 4.43 (dd, J=13.0, 4.5 Hz, 1H), 2.89-2.78 (m, 4H), 2.67 (dt, J=14.6, 2.6 Hz, 1H), 2.48-2.41 (m, 1H), 2.17 (dtd, J=12.9, 5.1, 2.6 Hz, 1H).

Step 2: (3-(2,6-Dioxopiperidin-3-yl)-2-methylquinolin-7-yl)methyl (3-chloro-4-methylphenyl)carbamate was synthesized using the general procedure shown in Reaction Scheme 5 and Example Method 5, above (40% yield), using 3-(7-(hydroxymethyl)-2-methylquinolin-3-yl)piperidine-2,6-dione (25 mg, 0.088 mmol, 1 equiv) and 2-chloro-4-isocyanato-1-methylbenzene (2 equiv) as starting materials and TEA (3 equiv) as base.

LCMS (ESI+) m/z 452.1 [M+H]⁺

¹H NMR (500 MHz, DMSO-d₆) δ 10.93 (s, 1H), 9.96 (s, 1H), 8.13 (s, 1H), 7.98-7.93 (m, 1H), 7.90 (d, J=8.4 Hz, 1H), 7.61 (d, J=2.2 Hz, 1H), 7.55 (dd, J=8.3, 1.7 Hz, 1H), 7.31 (dd, J=8.3, 2.2 Hz, 1H), 7.25 (d, J=8.5 Hz, 1H), 5.36 (s, 2H), 4.29 (dd, J=12.5, 4.7 Hz, 1H), 2.82 (ddd, J=17.2, 12.9, 5.3 Hz, 1H), 2.66 (s, 3H), 2.60 (t, J=3.6 Hz, 1H), 2.42 (qd, J=13.0, 4.4 Hz, 1H), 2.25 (s, 3H), 2.13 (dtd, J=12.9, 5.2, 3.0 Hz, 1H).

### Example 5: Synthesis of (3-(2,6-dioxopiperidin-3-yl)-2-methylquinolin-6-yl)methyl (3-chloro-4-methylphenyl)carbamate (Compound 5)

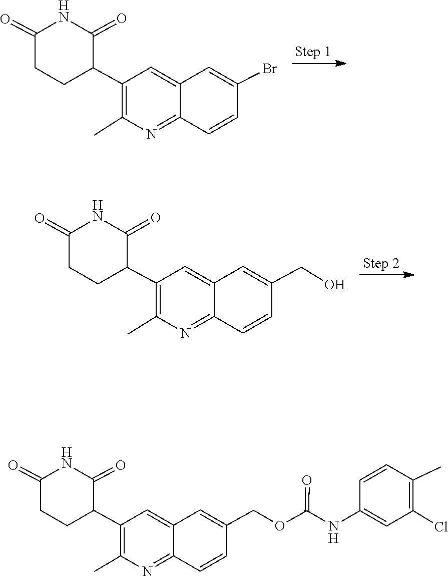 [SMILES: Cc1nc2ccc(Br)cc2cc1C1CCC(=O)NC1=O ; Cc1nc2ccc(CO)cc2cc1C1CCC(=O)NC1=O ; Cc1ccc(NC(=O)OCc2ccc3nc(C)c(C4CCC(=O)NC4=O)cc3c2)cc1Cl]

Step 1: 3-(6-(Hydroxymethyl)-2-methylquinolin-3-yl)piperidine-2,6-dione was synthesized using the general procedure shown in Reaction Scheme 7 and Example Method 7, above (93% yield), using 3-(6-bromo-2-methylquinolin-3-yl)piperidine-2,6-dione (150 mg, 0.45 mmol, 1 equiv) and (tributylstannyl)methanol (1.1 equiv) as starting materials, tetrakis(triphenylphosphine)palladium(0) (0.1 equiv) as catalyst and dioxane as solvent.

LCMS (ESI+) m/z 284.8 [M+H]⁺

Step 2: To the solution of 3-(6-(hydroxymethyl)-2-methylquinolin-3-yl)piperidine-2,6-dione (12 mg, 0.033 mmol, 1 equiv) in THF (1 mL) was added 1M solution of LiHMDS (0.099 mL, 0.099 mmol, 3 equiv) and the resulting solution was stirred for 15 min. Phenyl (3-chloro-4-methylphenyl)carbamate (2 equiv) in THF (1 mL) was added the reaction was continued for 4 h at RT. The reaction mixture was quenched by addition of glacial acetic acid. The volatiles were removed under reduced pressure and the crude product was purified by preparative HPLC to give (3-(2,6-dioxopiperidin-3-yl)-2-methylquinolin-6-yl)methyl (3-chloro-4-methylphenyl)carbamate (4.0 mg, 27% yield).

LCMS (ESI+) m/z 452.0 [M+H]⁺

¹H NMR (500 MHz, DMSO-d₆) δ 10.93 (s, 1H), 9.92 (s, 1H), 8.13 (s, 1H), 7.94 (d, J=8.6 Hz, 1H), 7.91 (d, J=1.5 Hz, 1H), 7.73 (dd, J=8.6, 1.9 Hz, 1H), 7.64-7.57 (m, 1H), 7.30 (dd, J=8.3, 2.1 Hz, 1H), 7.24 (d, J=8.5 Hz, 1H), 5.32 (s, 2H), 4.29 (dd, J=12.5, 4.7 Hz, 1H), 2.82 (ddd, J=17.7, 12.9, 5.3 Hz, 1H), 2.66 (s, 3H), 2.62 (dt, J=17.2, 3.3 Hz, 1H), 2.43 (qd, J=12.9, 4.2 Hz, 1H), 2.25 (s, 3H), 2.13 (ddt, J=11.0, 5.0, 3.0 Hz, 1H).

### Example 6: Synthesis of (3-(2,6-dioxopiperidin-3-yl)-2-methylquinolin-7-yl)methyl (2-fluoro-5-(trifluoromethoxy)phenyl)carbamate (Compound 6)

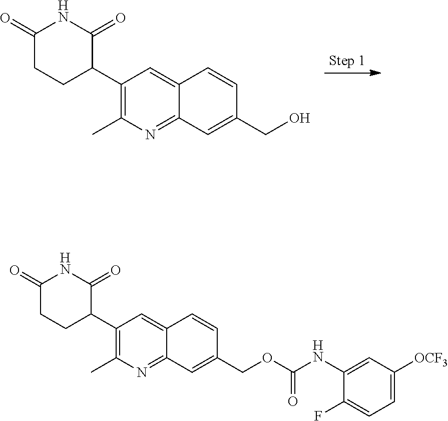 [SMILES: Cc1nc2cc(CO)ccc2cc1C1CCC(=O)NC1=O ; Cc1nc2cc(COC(=O)Nc3cc(OC(F)(F)F)ccc3F)ccc2cc1C1CCC(=O)NC1=O]

Step 1: (3-(2,6-Dioxopiperidin-3-yl)-2-methylquinolin-7-yl)methyl (2-fluoro-5-(trifluoromethoxy)phenyl)carbamate was synthesized using the general procedure shown in Reaction Scheme 4 and Example Method 4, above (37% yield), using 3-(7-(hydroxymethyl)-2-methylquinolin-3-yl)piperidine-2,6-dione (20.0 mg, 0.070 mmol, 1 equiv) and phenyl (2-fluoro-5-(trifluoromethoxy)phenyl)carbamate (1.1 equiv) as starting materials, sodium hydride as base and DMF as solvent.

LCMS (ESI+) m/z 506.1 [M+H]⁺

¹H NMR (500 MHz, DMSO-d₆) δ 10.93 (s, 1H), 9.95 (s, 1H), 8.13 (s, 1H), 8.01-7.97 (m, 1H), 7.90 (d, J=8.4 Hz, 1H), 7.85 (dd, J=6.8, 2.9 Hz, 1H), 7.56 (dd, J=8.4, 1.7 Hz, 1H), 7.39 (dd, J=10.4, 9.0 Hz, 1H), 7.14 (dt, J=8.8, 3.6 Hz, 1H), 5.40 (s, 2H), 4.30 (dd, J=12.5, 4.7 Hz, 1H), 2.82 (ddd, J=17.6, 12.9, 5.3 Hz, 1H), 2.66 (s, 3H), 2.60 (d, J=3.7 Hz, 1H), 2.46-2.38 (m, 1H), 2.13 (dtd, J=13.0, 5.2, 3.0 Hz, 1H).

### Example 7: Synthesis of (3-(2,6-dioxopiperidin-3-yl)-2-methylquinolin-6-yl)methyl (2-fluoro-5-(trifluoromethoxy)phenyl)carbamate (Compound 7)

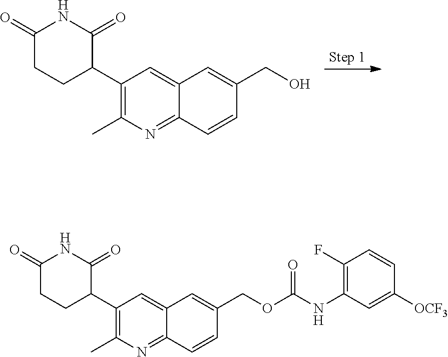 [SMILES: Cc1nc2ccc(CO)cc2cc1C1CCC(=O)NC1=O ; Cc1nc2ccc(COC(=O)Nc3cc(OC(F)(F)F)ccc3F)cc2cc1C1CCC(=O)NC1=O]

Step 1: (3-(2,6-Dioxopiperidin-3-yl)-2-methylquinolin-6-yl)methyl (2-fluoro-5-(trifluoromethoxy)phenyl)carbamate was synthesized using the general procedure shown in Reaction Scheme 4 and Example Method 4, above (45% yield), using 3-(6-(hydroxymethyl)-2-methylquinolin-3-yl)piperidine-2,6-dione (25.0 mg, 0.088 mmol, 1 equiv) and phenyl (2-fluoro-5-(trifluoromethoxy)phenyl)carbamate (1.1 equiv) as starting materials, sodium hydride as base, and DMF as solvent.

LCMS (ESI+) m/z 506.1 [M+H]⁺

¹H NMR (500 MHz, DMSO-d₆) δ 10.93 (s, 1H), 9.89 (s, 1H), 8.13 (s, 1H), 7.96-7.90 (m, 2H), 7.84 (dd, J=6.4, 3.0 Hz, 1H), 7.74 (dd, J=8.7, 1.9 Hz, 1H), 7.38 (dd, J=10.4, 9.0 Hz, 1H), 7.15-7.11 (m, 1H), 5.36 (s, 2H), 4.30 (dd, J=12.4, 4.8 Hz, 1H), 2.82 (ddd, J=17.7, 12.9, 5.4 Hz, 1H), 2.66 (s, 3H), 2.63-2.58 (m, 1H), 2.41 (td, J=12.8, 4.3 Hz, 1H), 2.13 (dtd, J=13.0, 5.2, 2.9 Hz, 1H).

### Example 8: Synthesis of (3-(2,6-dioxopiperidin-3-yl)-2-methylquinolin-7-yl)methyl (4-(tert-butyl)phenyl)carbamate (Compound 8)

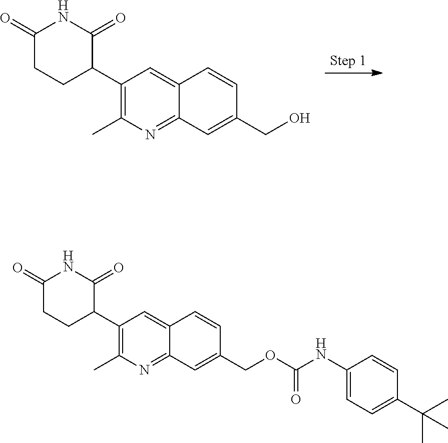 [SMILES: Cc1nc2cc(CO)ccc2cc1C1CCC(=O)NC1=O ; Cc1nc2cc(COC(=O)Nc3ccc(C(C)(C)C)cc3)ccc2cc1C1CCC(=O)NC1=O]

Step 1: (3-(2,6-Dioxopiperidin-3-yl)-2-methylquinolin-7-yl)methyl (4-(tert-butyl)phenyl)carbamate was synthesized using the general procedure shown in Reaction Scheme 4 and Example Method 4, above (39% yield), using 3-(7-(hydroxymethyl)-2-methylquinolin-3-yl)piperidine-2,6-dione (15.0 mg, 0.053 mmol, 1 equiv) and phenyl (4-(tert-butyl)phenyl)carbamate (1.1 equiv) as starting materials, sodium hydride as base, and DMF as solvent.

LCMS (ESI+) m/z 460.2 [M+H]⁺

¹H NMR (500 MHz, DMSO-d₆): δ 10.93 (s, 1H), 9.75 (s, 1H), 8.12 (s, 1H), 7.98-7.93 (m, 1H), 7.90 (d, J=8.4 Hz, 1H), 7.55 (dd, J=8.4, 1.7 Hz, 1H), 7.46-7.36 (m, 2H), 7.33-7.24 (m, 2H), 5.35 (s, 2H), 4.29 (dd, J=12.5, 4.7 Hz, 1H), 2.88-2.76 (m, 1H), 2.66 (s, 3H), 2.64-2.59 (m, 1H), 2.47-2.38 (m, 1H), 2.17-2.05 (m, 1H), 1.25 (s, 9H).

### Example 9: Synthesis of (3-(2,6-dioxopiperidin-3-yl)-2-methylquinolin-7-yl)methyl (3-(tert-butyl)phenyl)carbamate (Compound 9)

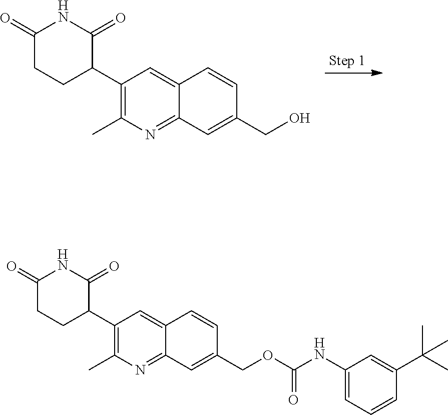 [SMILES: Cc1nc2cc(CO)ccc2cc1C1CCC(=O)NC1=O ; Cc1nc2cc(COC(=O)Nc3cccc(C(C)(C)C)c3)ccc2cc1C1CCC(=O)NC1=O]

Step 1: (3-(2,6-Dioxopiperidin-3-yl)-2-methylquinolin-7-yl)methyl (3-(tert-butyl)phenyl)carbamate was synthesized using the general procedure shown in Reaction Scheme 4 and Example Method 4, above (38% yield), using 3-(7-(hydroxymethyl)-2-methylquinolin-3-yl)piperidine-2,6-dione (15.0 mg, 0.053 mmol, 1 equiv) and phenyl (3-(tert-butyl)phenyl)carbamate (1.1 equiv) as starting materials, sodium hydride as base, and DMF as solvent.

LCMS (ESI+) m/z 460.2 [M+H]⁺

¹H NMR (500 MHz, DMSO-d₆): δ 10.93 (s, 1H), 9.76 (s, 1H), 8.13 (s, 1H), 7.98-7.92 (m, 1H), 7.90 (d, J=8.4 Hz, 1H), 7.59-7.49 (m, 2H), 7.37-7.27 (m, 1H), 7.20 (t, J=7.9 Hz, 1H), 7.08-6.99 (m, 1H), 5.36 (s, 2H), 4.29 (dd, J=12.5, 4.7 Hz, 1H), 2.88-2.77 (m, 1H), 2.69-2.64 (m, 3H), 2.64-2.59 (m, 1H), 2.47-2.37 (m, 1H), 2.16-2.08 (m, 1H), 1.25 (s, 9H).

### Example 10: Synthesis of (3-(2,6-dioxopiperidin-3-yl)-2-methylquinolin-7-yl)methyl (4-(tert-butyl)-3-chlorophenyl)carbamate (Compound 10)

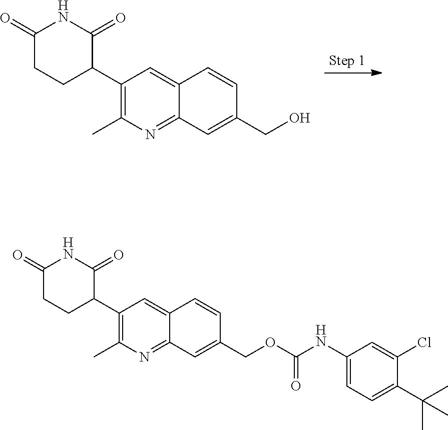 [SMILES: Cc1nc2cc(CO)ccc2cc1C1CCC(=O)NC1=O ; Cc1nc2cc(COC(=O)Nc3ccc(C(C)(C)C)c(Cl)c3)ccc2cc1C1CCC(=O)NC1=O]

Step 1: (3-(2,6-Dioxopiperidin-3-yl)-2-methylquinolin-7-yl)methyl (4-(tert-butyl)-3-chlorophenyl)carbamate was synthesized using the general procedure shown in Reaction Scheme 4 and Example Method 4, above (54% yield), using 3-(7-(hydroxymethyl)-2-methylquinolin-3-yl)piperidine-2,6-dione (12.0 mg, 0.042 mmol, 1 equiv) and phenyl (4-(tert-butyl)-3-chlorophenyl)carbamate (1.1 equiv) as starting materials, sodium hydride as base, and DMF as solvent.

LCMS (ESI+) m/z 494.2 [M+H]⁺

¹H NMR (500 MHz, DMSO-d₆): δ 10.93 (s, 1H), 9.98 (s, 1H), 8.13 (s, 1H), 7.98-7.93 (m, 1H), 7.90 (d, J=8.4 Hz, 1H), 7.62-7.57 (m, 1H), 7.55 (dd, J=8.4, 1.7 Hz, 1H), 7.41-7.32 (m, 2H), 5.36 (s, 2H), 4.29 (dd, J=12.5, 4.7 Hz, 1H), 2.87-2.76 (m, 1H), 2.66 (s, 3H), 2.64-2.58 (m, 1H), 2.47-2.37 (m, 1H), 2.18-2.07 (m, 1H), 1.41 (s, 9H).

### Example 11: Synthesis of (3-(2,6-dioxopiperidin-3-yl)-2-methylquinolin-7-yl)methyl (3-chloro-4-methoxyphenyl)carbamate (Compound 11)

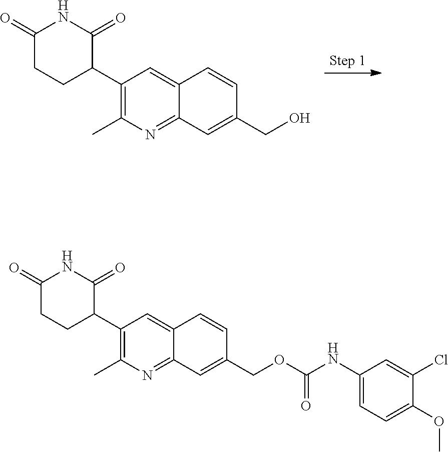 [SMILES: Cc1nc2cc(CO)ccc2cc1C1CCC(=O)NC1=O ; COc1ccc(NC(=O)OCc2ccc3cc(C4CCC(=O)NC4=O)c(C)nc3c2)cc1Cl]

Step 1: (3-(2,6-Dioxopiperidin-3-yl)-2-methylquinolin-7-yl)methyl (3-chloro-4-methoxyphenyl)carbamate was synthesized using the general procedure shown in Reaction Scheme 4 and Example Method 4, above (14% yield), using 3-(7-(hydroxymethyl)-2-methylquinolin-3-yl)piperidine-2,6-dione (15.0 mg, 0.053 mmol, 1 equiv) and phenyl (3-chloro-4-methoxyphenyl)carbamate (1.1 equiv) as starting materials, sodium hydride as base, and DMF as solvent.

LCMS (ESI+) m/z 468.1 [M+H]⁺

¹H NMR (500 MHz, DMSO-d₆): δ 10.93 (s, 1H), 9.84 (s, 1H), 8.13 (s, 1H), 7.97-7.93 (m, 1H), 7.90 (d, J=8.4 Hz, 1H), 7.63-7.57 (m, 1H), 7.55 (dd, J=8.4, 1.7 Hz, 1H), 7.40-7.33 (m, 1H), 7.10 (d, J=9.0 Hz, 1H), 5.35 (s, 2H), 4.29 (dd, J=12.4, 4.8 Hz, 1H), 3.80 (s, 3H), 2.86-2.78 (m, 1H), 2.66 (s, 3H), 2.64-2.55 (m, 1H), 2.42 (qd, J=12.9, 4.3 Hz, 1H), 2.17-2.08 (m, 1H).

### Example 12: Synthesis of (3-(2,6-dioxopiperidin-3-yl)-2-methylquinolin-7-yl)methyl (3,4-dichlorophenyl)carbamate (Compound 12)

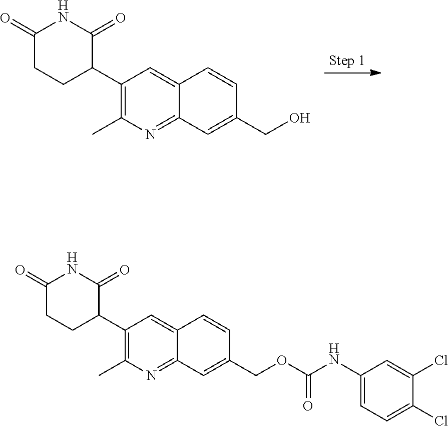 [SMILES: Cc1nc2cc(CO)ccc2cc1C1CCC(=O)NC1=O ; Cc1nc2cc(COC(=O)Nc3ccc(Cl)c(Cl)c3)ccc2cc1C1CCC(=O)NC1=O]

Step 1: (3-(2,6-Dioxopiperidin-3-yl)-2-methylquinolin-7-yl)methyl (3,4-dichlorophenyl)carbamate was synthesized using the general procedure shown in Reaction Scheme 5 and Example Method 5, above (39% yield), using 3-(7-(hydroxymethyl)-2-methylquinolin-3-yl)piperidine-2,6-dione (20 mg, 0.070 mmol, 1 equiv) and 1,2-dichloro-4-isocyanatobenzene (1.1 equiv) as starting materials and TEA as base, and THF as solvent.

LCMS (ESI+) m/z 472.0 [M+H]⁺

¹H NMR (500 MHz, DMSO-d₆) δ 10.93 (s, 1H), 10.20 (s, 1H), 8.13 (s, 1H), 7.97-7.94 (m, 1H), 7.91 (d, J=8.4 Hz, 1H), 7.80 (d, J=2.5 Hz, 1H), 7.57-7.56 (m, 1H), 7.55 (d, J=4.6 Hz, 1H), 7.44 (dd, J=8.9, 2.5 Hz, 1H), 5.38 (s, 2H), 4.29 (dd, J=12.5, 4.7 Hz, 1H), 2.82 (ddd, J=17.6, 12.9, 5.3 Hz, 1H), 2.66 (s, 3H), 2.60 (dd, J=4.2, 2.9 Hz, 1H), 2.45-2.38 (m, 1H), 2.13 (dtd, J=13.1, 5.2, 3.0 Hz, 1H).

### Example 13: Synthesis of 3-(7-(((5-(3-chloro-4-methylphenyl)-1,3,4-oxadiazol-2-yl)amino)methyl)-2-methylquinolin-3-yl)piperidine-2,6-dione (Compound 13)

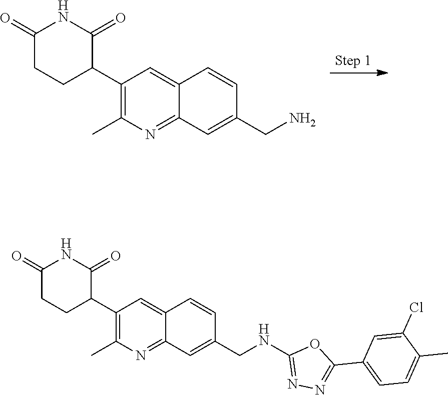 [SMILES: Cc1nc2cc(CN)ccc2cc1C1CCC(=O)NC1=O ; Cc1ccc(-c2nnc(NCc3ccc4cc(C5CCC(=O)NC5=O)c(C)nc4c3)o2)cc1Cl]

Step 1: 3-(7-(Aminomethyl)-2-methylquinolin-3-yl)piperidine-2,6-dione hydrochloride (10 mg, 0.031 mmol, 1 equiv) was dissolved in dry DMF (1 mL). DIPEA (5 equiv) was added followed by 5-(3-chloro-4-methylphenyl)-1,3,4-oxadiazol-2(3H)-one (3 equiv) in dry DMF (1 mL) and PyBOP (1.1 equiv). The resulting mixture was stirred at RT for 3 days. The reaction mixture was diluted with water and the product was extracted with ethyl acetate. The organic phase was dried with Na₂SO₄ and concentrated under reduced pressure. The crude product was purified by preparative HPLC to give 3-(7-(((5-(3-chloro-4-methylphenyl)-1,3,4-oxadiazol-2-yl)amino)methyl)-2-methylquinolin-3-yl)piperidine-2,6-dione (2.9 mg, 19% yield).

LCMS (ESI+) m/z 476.1 [M+H]⁺

¹H NMR (500 MHz, DMSO-d₆) δ 10.92 (s, 1H), 8.54 (t, J=6.2 Hz, 1H), 8.09 (s, 1H), 7.91-7.84 (m, 2H), 7.78 (d, J=1.8 Hz, 1H), 7.68 (dd, J=7.9, 1.8 Hz, 1H), 7.57-7.49 (m, 2H), 4.66 (d, J=6.2 Hz, 2H), 4.27 (dd, J=12.5, 4.7 Hz, 1H), 2.81 (m, 1H), 2.64 (m, 4H), 2.48-2.40 (m, 1H), 2.39 (s, 3H), 2.16-2.05 (m, 1H).

### Example 14: Synthesis of (3-(2,6-dioxopiperidin-3-yl)-2-methylquinolin-7-yl)methyl benzo[d][1,3]dioxol-5-ylcarbamate (Compound 14)

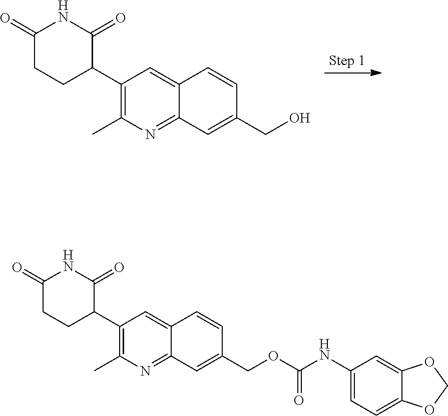 [SMILES: Cc1nc2cc(CO)ccc2cc1C1CCC(=O)NC1=O ; Cc1nc2cc(COC(=O)Nc3ccc4c(c3)OCO4)ccc2cc1C1CCC(=O)NC1=O]

Step 1: (3-(2,6-Dioxopiperidin-3-yl)-2-methylquinolin-7-yl)methyl benzo[d][1,3]dioxol-5-ylcarbamate was synthesized using the general procedure shown in Reaction Scheme 4 and Example Method 4, above (16% yield), using 3-(7-(hydroxymethyl)-2-methylquinolin-3-yl)piperidine-2,6-dione (12.0 mg, 0.042 mmol, 1 equiv) and phenyl benzo[d][1,3]dioxol-5-ylcarbamate (1.1 equiv) as starting materials, sodium hydride as base, and DMF as solvent.

LCMS (ESI+) m/z 448.2 [M+H]⁺

¹H NMR (500 MHz, DMSO-d₆): δ 10.93 (s, 1H), 9.73 (s, 1H), 8.12 (s, 1H), 7.97-7.93 (m, 1H), 7.90 (d, J=8.4 Hz, 1H), 7.54 (dd, J=8.3, 1.7 Hz, 1H), 7.14 (s, 1H), 6.88 (dd, J=8.4, 2.1 Hz, 1H), 6.83 (d, J=8.4 Hz, 1H), 5.96 (s, 2H), 5.33 (s, 2H), 4.29 (dd, J=12.5, 4.7 Hz, 1H), 2.82 (ddd, J=17.8, 12.9, 5.3 Hz, 1H), 2.69-2.55 (m, 4H), 2.47-2.37 (m, 1H), 2.13 (dtd, J=13.0, 5.2, 3.0 Hz, 1H).

### Example 15: Synthesis of (3-(2,6-dioxopiperidin-3-yl)-2-methylquinolin-7-yl)methyl (2,3-dihydrobenzo[b][1,4]dioxin-6-yl)carbamate (Compound 15)

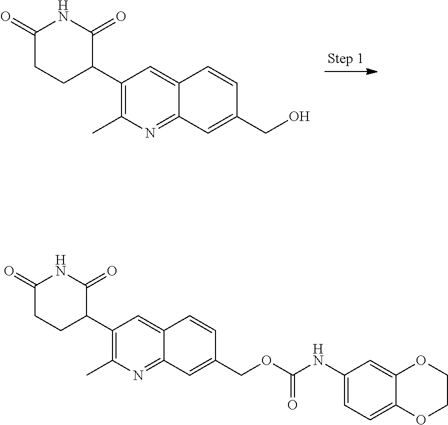 [SMILES: Cc1nc2cc(CO)ccc2cc1C1CCC(=O)NC1=O ; Cc1nc2cc(COC(=O)Nc3ccc4c(c3)OCCO4)ccc2cc1C1CCC(=O)NC1=O]

Step 1: (3-(2,6-Dioxopiperidin-3-yl)-2-methylquinolin-7-yl)methyl (2,3-dihydrobenzo[b][1,4]dioxin-6-yl)carbamate was synthesized using the general procedure shown in Reaction Scheme 3 and Example Method 3, above (24% yield), using 3-(7-(hydroxymethyl)-2-methylquinolin-3-yl)piperidine-2,6-dione (13.0 mg, 0.047 mmol, 1 equiv) and phenyl (2,3-dihydrobenzo[b][1,4]dioxin-6-yl)carbamate (1.1 equiv) as starting materials, TEA as base, and DMF as solvent.

LCMS (ESI+) m/z 462.1 [M+H]⁺

¹H NMR (500 MHz, DMSO-d₆): δ 10.93 (s, 1H), 9.65 (s, 1H), 8.12 (s, 1H), 7.96-7.92 (m, 1H), 7.90 (d, J=8.4 Hz, 1H), 7.54 (dd, J=8.4, 1.7 Hz, 1H), 7.10-7.01 (m, 1H), 6.89 (dd, J=8.5, 2.6 Hz, 1H), 6.76 (d, J=8.7 Hz, 1H), 5.33 (s, 2H), 4.29 (dd, J=12.5, 4.7 Hz, 1H), 4.22-4.16 (m, 4H), 2.88-2.76 (m, 1H), 2.66 (s, 3H), 2.62 (ddd, J=17.2, 4.1, 2.7 Hz, 1H), 2.46-2.37 (m, 1H), 2.12 (dtd, J=13.0, 5.1, 3.0 Hz, 1H).

### Example 16: Synthesis of (3-(2,6-dioxopiperidin-3-yl)-2-methylquinolin-7-yl)methyl (3-chloro-4-methyl-5-(morpholinomethyl)phenyl)carbamate (Compound 17)

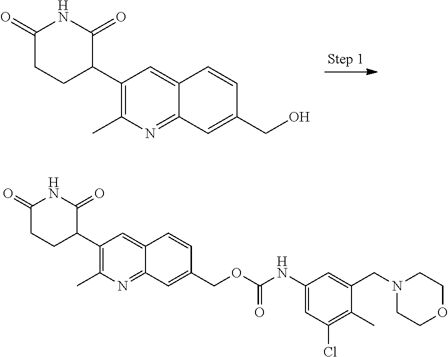 [SMILES: Cc1nc2cc(CO)ccc2cc1C1CCC(=O)NC1=O ; Cc1nc2cc(COC(=O)Nc3cc(Cl)c(C)c(CN4CCOCC4)c3)ccc2cc1C1CCC(=O)NC1=O]

Step 1: Sodium hydride (60% suspension in mineral oil, 3.8 mg, 0.094 mmol, 2 equiv) was suspended in dry DMF (1 mL) under argon and cooled in ice-water bath. 3-(7-(Hydroxymethyl)-2-methylquinolin-3-yl)piperidine-2,6-dione (14.7 mg, 0.052 mmol, 1.1 equiv) in DMF (1 mL) was added and the resulting mixture was stirred at 0° C. for 30 min. Phenyl (3-chloro-4-methyl-5-(morpholinomethyl)phenyl)carbamate (17 mg, 0.047 mmol, 1 equiv) was added, the reaction mixture was stirred at 0° C. for additional 30 min and warmed to RT. After completion, the reaction mixture was directly purified by flash column chromatography to give (3-(2,6-dioxopiperidin-3-yl)-2-methylquinolin-7-yl)methyl (3-chloro-4-methyl-5-(morpholinomethyl)phenyl)carbamate (2.2 mg, 8% yield).

The synthesis of phenyl (3-chloro-4-methyl-5-(morpholinomethyl)phenyl)carbamate was described in WO2022152821A1.

LCMS (ESI+) m/z 551.2 [M+H]⁺

¹H NMR (500 MHz, DMSO-d₆) δ 10.93 (s, 1H), 9.93 (s, 1H), 8.13 (s, 1H), 7.97-7.93 (m, 1H), 7.90 (d, J=8.4 Hz, 1H), 7.55 (dd, J=8.3, 1.7 Hz, 2H), 7.35 (d, J=2.3 Hz, 1H), 5.36 (s, 2H), 4.29 (dd, J=12.5, 4.7 Hz, 1H), 3.55 (t, J=4.6 Hz, 4H), 3.40 (s, 2H), 2.82 (ddd, J=17.7, 13.0, 5.3 Hz, 1H), 2.68-2.64 (m, 4H), 2.48-2.37 (m, 1H), 2.35 (s, 4H), 2.28 (s, 3H), 2.17-2.08 (m, 1H).

### Example 17: Synthesis of (3-(2,6-dioxopiperidin-3-yl)-2-methylquinolin-7-yl)methyl (3-chloro-4-(trifluoromethyl)phenyl)carbamate (Compound 18)

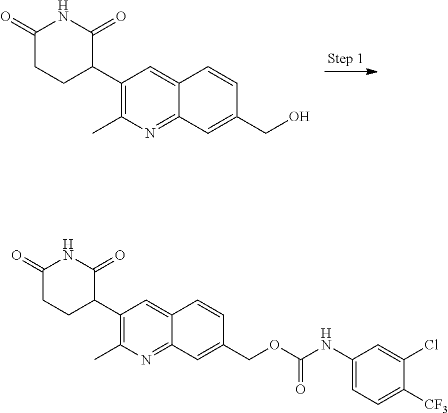 [SMILES: Cc1nc2cc(CO)ccc2cc1C1CCC(=O)NC1=O ; Cc1nc2cc(COC(=O)Nc3ccc(C(F)(F)F)c(Cl)c3)ccc2cc1C1CCC(=O)NC1=O]

Step 1: (3-(2,6-Dioxopiperidin-3-yl)-2-methylquinolin-7-yl)methyl (3-chloro-4-(trifluoromethyl)phenyl)carbamate was synthesized using the general procedure shown in Reaction Scheme 3 and Example Method 3, above (30% yield), using 3-(7-(hydroxymethyl)-2-methylquinolin-3-yl)piperidine-2,6-dione (12.0 mg, 0.042 mmol, 1 equiv) and phenyl (3-chloro-4-(trifluoromethyl)phenyl)carbamate (1.2 equiv) as starting materials, TEA as base, and DMF as solvent.

The synthesis of phenyl (3-chloro-4-(trifluoromethyl)phenyl)carbamate was described in WO2006067401A1.

LCMS (ESI+) m/z 506.1 [M+H]⁺

¹H NMR (500 MHz, DMSO-d₆): δ 10.93 (s, 1H), 10.49 (s, 1H), 8.13 (s, 1H), 7.99-7.96 (m, 1H), 7.91 (d, J=8.4 Hz, 1H), 7.85 (d, J=2.1 Hz, 1H), 7.79 (d, J=8.7 Hz, 1H), 7.62-7.55 (m, 2H), 5.41 (s, 2H), 4.30 (dd, J=12.5, 4.7 Hz, 1H), 2.87-2.78 (m, 1H), 2.66 (s, 3H), 2.64-2.59 (m, 1H), 2.42 (qd, J=12.9, 4.2 Hz, 1H), 2.13 (dtd, J=12.9, 5.2, 3.0 Hz, 1H).

### Example 18: Synthesis of (3-(2,6-dioxopiperidin-3-yl)-2-methylquinolin-7-yl)methyl (3-(trifluoromethoxy)phenyl)carbamate (Compound 19)

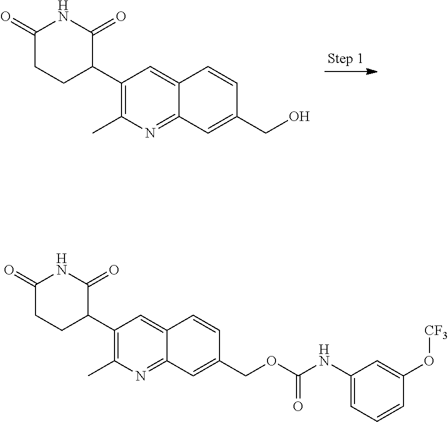 [SMILES: Cc1nc2cc(CO)ccc2cc1C1CCC(=O)NC1=O ; Cc1nc2cc(COC(=O)Nc3cccc(OC(F)(F)F)c3)ccc2cc1C1CCC(=O)NC1=O]

Step 1: (3-(2,6-Dioxopiperidin-3-yl)-2-methylquinolin-7-yl)methyl (3-(trifluoromethoxy)phenyl)carbamate was synthesized using the general procedure shown in Reaction Scheme 3 and Example Method 3, above (24% yield), using 3-(7-(hydroxymethyl)-2-methylquinolin-3-yl)piperidine-2,6-dione (12.0 mg, 0.042 mmol, 1 equiv) and phenyl (3-(trifluoromethoxy)phenyl)carbamate (1.2 equiv) as starting materials, TEA as base, and DMF as solvent.

The synthesis of phenyl (3-(trifluoromethoxy)phenyl)carbamate was described in WO2022152821A1.

LCMS (ESI+) m/z 488.2 [M+H]⁺

¹H NMR (500 MHz, DMSO-d₆): δ 10.93 (s, 1H), 10.19 (s, 1H), 8.13 (s, 1H), 7.99-7.94 (m, 1H), 7.93-7.88 (m, 1H), 7.63-7.58 (m, 1H), 7.56 (dd, J=8.3, 1.7 Hz, 1H), 7.46-7.39 (m, 2H), 7.01-6.96 (m, 1H), 5.38 (s, 2H), 4.30 (dd, J=12.5, 4.7 Hz, 1H), 2.82 (ddd, J=17.2, 12.9, 5.3 Hz, 1H), 2.66 (s, 3H), 2.64-2.59 (m, 1H), 2.42 (qd, J=12.9, 4.3 Hz, 1H), 2.13 (dtd, J=13.0, 5.2, 3.1 Hz, 1H).

### Example 19: Synthesis of (3-(2,6-dioxopiperidin-3-yl)-2-methylquinolin-7-yl)methyl (6-phenylpyridin-3-yl)carbamate (Compound 20)

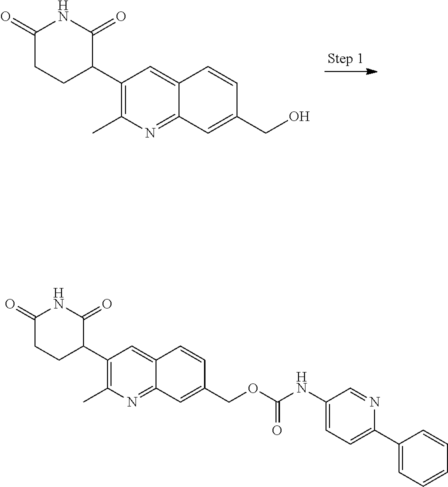 [SMILES: Cc1nc2cc(CO)ccc2cc1C1CCC(=O)NC1=O ; Cc1nc2cc(COC(=O)Nc3ccc(-c4ccccc4)nc3)ccc2cc1C1CCC(=O)NC1=O]

Step 1: (3-(2,6-Dioxopiperidin-3-yl)-2-methylquinolin-7-yl)methyl (6-phenylpyridin-3-yl)carbamate was synthesized using the general procedure shown in Reaction Scheme 3 and Example Method 3, above (33% yield), using 3-(7-(hydroxymethyl)-2-methylquinolin-3-yl)piperidine-2,6-dione (15.0 mg, 0.053 mmol, 1 equiv) and phenyl (6-phenylpyridin-3-yl)carbamate (1.2 equiv) as starting materials, TEA as base, and DMF as solvent.

The synthesis of phenyl (6-phenylpyridin-3-yl)carbamate was prepared as described in WO2022152821A1.

LCMS (ESI+) m/z 481.2 [M+H]⁺

¹H NMR (500 MHz, DMSO-d₆): δ 10.93 (s, 1H), 10.26-10.08 (m, 1H), 8.79-8.71 (m, 1H), 8.14 (s, 1H), 8.06-7.99 (m, 3H), 7.98 (dt, J=1.5, 0.8 Hz, 1H), 7.95-7.90 (m, 2H), 7.58 (dd, J=8.4, 1.7 Hz, 1H), 7.50-7.42 (m, 2H), 7.42-7.35 (m, 1H), 5.41 (s, 2H), 4.30 (dd, J=12.5, 4.7 Hz, 1H), 2.87-2.78 (m, 1H), 2.66 (s, 3H), 2.64-2.59 (m, 1H), 2.42 (qd, J=12.9, 4.3 Hz, 1H), 2.13 (ddq, J=10.0, 5.0, 2.6 Hz, 1H).

### Example 20: Synthesis of (3-(2,6-dioxopiperidin-3-yl)-2-methylquinolin-7-yl)methyl (5-chloro-2-methoxy-4-methylphenyl)carbamate (Compound 21)

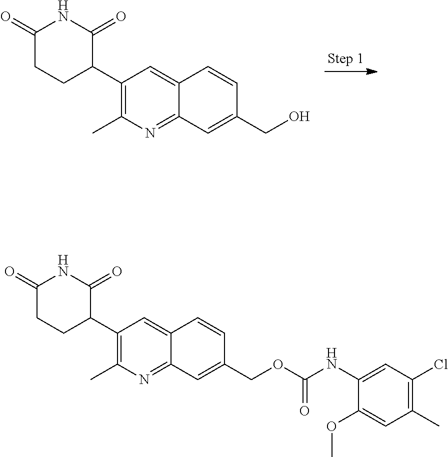 [SMILES: Cc1nc2cc(CO)ccc2cc1C1CCC(=O)NC1=O ; COc1cc(C)c(Cl)cc1NC(=O)OCc1ccc2cc(C3CCC(=O)NC3=O)c(C)nc2c1]

Step 1: (3-(2,6-Dioxopiperidin-3-yl)-2-methylquinolin-7-yl)methyl (5-chloro-2-methoxy-4-methylphenyl)carbamate was synthesized using the general procedure shown in Reaction Scheme 3 and Example Method 3, above (29% yield), using 3-(7-(hydroxymethyl)-2-methylquinolin-3-yl)piperidine-2,6-dione (15.0 mg, 0.053 mmol, 1 equiv) and phenyl (5-chloro-2-methoxy-4-methylphenyl)carbamate (1.2 equiv) as starting materials, TEA as base, and DMF as solvent.

The synthesis of phenyl (5-chloro-2-methoxy-4-methylphenyl)carbamate was described in WO2022152821A1.

LCMS (ESI+) m/z 482.2 [M+H]⁺

¹H NMR (500 MHz, DMSO-d₆): δ 10.93 (s, 1H), 8.89 (s, 1H), 8.12 (s, 1H), 7.98 (s, 1H), 7.89 (d, J=8.3 Hz, 1H), 7.72 (s, 1H), 7.54 (dd, J=8.4, 1.7 Hz, 1H), 7.06-7.01 (m, 1H), 5.34 (s, 2H), 4.29 (dd, J=12.5, 4.7 Hz, 1H), 3.81 (s, 3H), 2.82 (ddd, J=17.1, 12.9, 5.3 Hz, 1H), 2.66 (s, 3H), 2.65-2.58 (m, 1H), 2.42 (qd, J=12.9, 4.3 Hz, 1H), 2.29 (s, 3H), 2.13 (dtd, J=13.1, 5.2, 3.0 Hz, 1H).

### Example 21: Synthesis of 2-(4-(tert-butyl)piperidin-1-yl)-N-((3-(2,6-dioxopiperidin-3-yl)-2-methylquinolin-6-yl)methyl)-2-oxoacetamide (Compound 23)

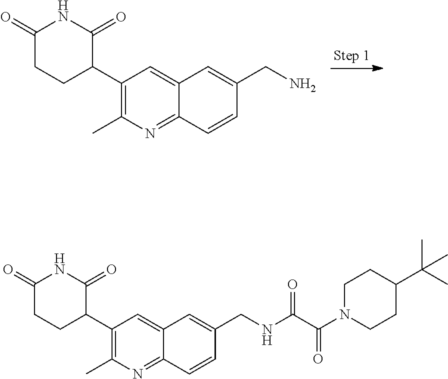 [SMILES: Cc1nc2ccc(CN)cc2cc1C1CCC(=O)NC1=O ; Cc1nc2ccc(CNC(=O)C(=O)N3CCC(C(C)(C)C)CC3)cc2cc1C1CCC(=O)NC1=O]

Step 1: 2-(4-(tert-Butyl)piperidin-1-yl)-N-((3-(2,6-dioxopiperidin-3-yl)-2-methylquinolin-6-yl)methyl)-2-oxoacetamide was synthesized using the general procedure shown in Reaction Scheme 1 and Example Method 1, above (8% yield), using 2-(4-(tert-butyl)piperidin-1-yl)-2-oxoacetic acid (8.3 mg, 0.039 mmol, 1 equiv) and 3-(6-(aminomethyl)-2-methylquinolin-3-yl)piperidine-2,6-dione trifluoroacetate (1 equiv) as starting materials, HATU (1.5 equiv), DIPEA as base, and DMF as solvent.

LCMS (ESI+) m/z 479.2 [M+H]⁺

¹H NMR (500 MHz, DMSO-d₆) δ 10.92 (s, 1H), 9.28 (td, J=6.2, 2.7 Hz, 1H), 8.06 (d, J=1.9 Hz, 1H), 7.89 (d, J=8.7 Hz, 1H), 7.72 (d, J=1.9 Hz, 1H), 7.61 (dd, J=8.6, 2.0 Hz, 1H), 4.49 (d, J=6.1 Hz, 2H), 4.43-4.33 (m, 1H), 4.28 (dd, J=12.5, 4.7 Hz, 1H), 3.83-3.75 (m, 1H), 3.01-2.91 (m, 1H), 2.83 (ddd, J=17.7, 13.0, 5.2 Hz, 1H), 2.67-2.61 (m, 4H), 2.62-2.53 (m, 1H), 2.47-2.38 (m, 1H), 2.17-2.07 (m, 1H), 1.72 (d, J=13.0 Hz, 1H), 1.69-1.61 (m, 1H), 1.31-1.22 (m, 1H), 1.20-1.01 (m, 2H), 0.83 (s, 9H).

### Example 22: Synthesis of 2-((3-chloro-4-methylphenyl)amino)-N-((3-(2,6-dioxopiperidin-3-yl)-2-methylquinolin-6-yl)methyl)acetamide (Compound 26)

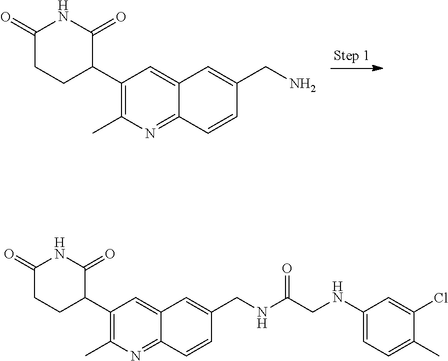 [SMILES: Cc1nc2ccc(CN)cc2cc1C1CCC(=O)NC1=O ; Cc1ccc(NCC(=O)NCc2ccc3nc(C)c(C4CCC(=O)NC4=O)cc3c2)cc1Cl]

Step 1: 2-(4-(tert-Butyl)piperidin-1-yl)-N-((3-(2,6-dioxopiperidin-3-yl)-2-methylquinolin-6-yl)methyl)-2-oxoacetamide was synthesized using the general procedure shown in Reaction Scheme 1 and Example Method 1, above (24% yield), using (3-chloro-4-methylphenyl)glycine (12.5 mg, 0.063 mmol, 1.2 equiv) and 3-(6-(aminomethyl)-2-methylquinolin-3-yl)piperidine-2,6-dione trifluoroacetate (20.7 mg, 0.052 mmol, 1 equiv) as starting materials, HATU (1.5 equiv), DIPEA as base, and DMF as solvent.

The synthesis of (3-chloro-4-methylphenyl)glycine was described in Takeda, A., J. Org. Chem. 1957, 22, 1096.

LCMS (ESI+) m/z 465.1 [M+H]⁺

¹H NMR (500 MHz, DMSO-d₆, 353K) δ 10.65 (s, 1H), 8.30-8.21 (m, 1H), 8.04 (s, 1H), 7.86 (d, J=8.6 Hz, 1H), 7.67 (s, 1H), 7.59 (dd, J=8.7, 2.0 Hz, 1H), 7.03 (dd, J=8.3, 0.8 Hz, 1H), 6.64 (d, J=2.4 Hz, 1H), 6.51 (dd, J=8.3, 2.4 Hz, 1H), 4.50 (d, J=6.0 Hz, 2H), 4.28 (dd, J=12.2, 4.9 Hz, 1H), 3.74 (s, 2H), 2.83 (ddd, J=17.7, 12.6, 5.4 Hz, 1H), 2.68 (s, 3H), 2.68-2.61 (m, 1H), 2.44-2.33 (m, 1H), 2.18 (s, 3H), 2.16 (td, J=5.0, 3.1 Hz, 1H).

### Example 23: Synthesis of (3-(2,4-dioxotetrahydropyrimidin-1(2H)-yl)-2-methylquinolin-7-yl)methyl (3-chloro-4-methylphenyl)carbamate (Compound 28)

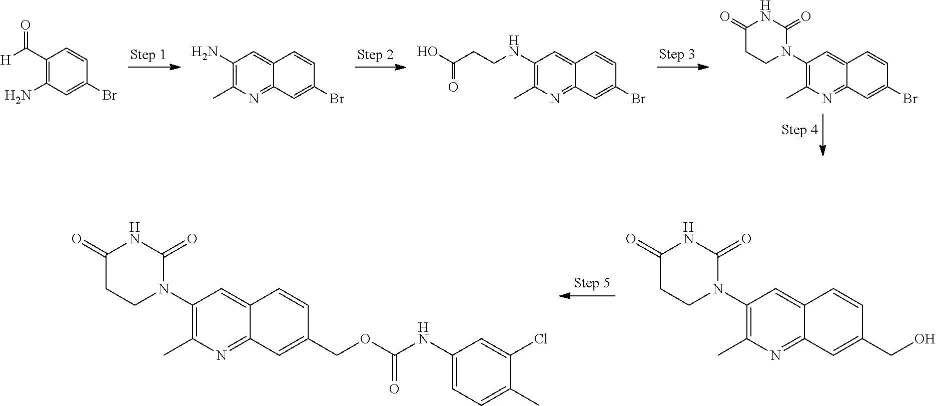 [SMILES: Nc1cc(Br)ccc1C=O ; Cc1nc2cc(Br)ccc2cc1N ; Cc1nc2cc(Br)ccc2cc1NCCC(=O)O ; Cc1nc2cc(Br)ccc2cc1N1CCC(=O)NC1=O ; Cc1ccc(NC(=O)OCc2ccc3cc(N4CCC(=O)NC4=O)c(C)nc3c2)cc1Cl ; Cc1nc2cc(CO)ccc2cc1N1CCC(=O)NC1=O]

Step 1: In a vial were placed 2-amino-4-bromobenzaldehyde (500 mg, 2.5 mmol, 1 equiv), 1-acetonylpyridinium chloride (1 equiv) and DMAP (0.02 equiv). n-Butanol (10 mL) and pyridine (0.7 equiv) were added and the reaction mixture was stirred at 100° C. for 16 h. Pyrrolidine (2.5 equiv) was then added and the reaction was stirred 100° C. for further 4 h. The volatiles were removed under reduced pressure and the residue was dissolved in DCM/NaHCO_{3 (aq)}. The product was extracted into DCM, the organic fraction was dried over Na₂SO₄ and concentrated. The crude product was purified by flash column chromatography to give 7-bromo-2-methylquinolin-3-amine (410 mg, 69% yield).

LCMS (ESI+) m/z 237.0 [M+H]⁺

Step 2: In a vial were placed 7-bromo-2-methylquinolin-3-amine (554 mg, 2.34 mmol, 1 equiv), water (1 mL) and acrylic acid (6 equiv). The reaction mixture was stirred at 70° C. for 20 h, the volatiles were removed under reduced pressure and crude product was purified by flash column chromatography to give 3-((7-bromo-2-methylquinolin-3-yl)amino)propanoic acid (720 mg, 87% yield).

LCMS (ESI+) m/z 309.0 [M+H]⁺

Step 3: In a vial 3-((7-bromo-2-methylquinolin-3-yl)amino)propanoic acid (720 mg, 2.026 mmol, 1 equiv) was dissolved in glacial acetic acid (7 mL). Urea (9 equiv) was added in several portions over stirring the mixture at 120° C. for 4 days. The mixture was cooled to RT, 10% HCl (20 mL) was added and the mixture was refluxed for 30 min. The volatiles were removed under reduced pressure and the crude product was purified by flash column chromatography to give 1-(7-bromo-2-methylquinolin-3-yl)dihydropyrimidine-2,4(1H,3H)-dione (500 mg, 74% yield).

LCMS (ESI+) m/z 334.0 [M+H]⁺

¹H NMR (500 MHz, DMSO-d₆) δ 10.55 (s, 1H), 8.34 (s, 1H), 8.18 (d, J=1.9 Hz, 1H), 7.91 (d, J=8.7 Hz, 1H), 7.73 (dd, J=8.7, 2.0 Hz, 1H), 3.94 (td, J=11.1, 4.8 Hz, 1H), 3.69 (dt, J=12.0, 5.8 Hz, 1H), 2.89 (ddd, J=16.5, 10.2, 6.2 Hz, 1H), 2.78-2.68 (m, 1H), 2.58 (s, 3H).

Step 4: 1-(7-(Hydroxymethyl)-2-methylquinolin-3-yl)dihydropyrimidine-2,4(1H,3H)-dione was synthesized using the general procedure shown in Reaction Scheme 7 and Example Method 7, above (60% yield), using 1-(7-bromo-2-methylquinolin-3-yl)dihydropyrimidine-2,4(1H,3H)-dione (50 mg, 0.15 mmol, 1 equiv) and (tributylstannyl)methanol (4 equiv) as starting materials, tetrakis(triphenylphosphine)palladium(0) (0.1 equiv) as catalyst and dioxane as solvent.

LCMS (ESI+) m/z 286.2 [M+H]⁺

Step 5: (3-(2,4-Dioxotetrahydropyrimidin-1(2H)-yl)-2-methylquinolin-7-yl)methyl (3-chloro-4-methylphenyl)carbamate was synthesized using the general procedure shown in Reaction Scheme 5 and Example Method 5, above (22% yield), using 1-(7-(hydroxymethyl)-2-methylquinolin-3-yl)dihydropyrimidine-2,4(1H,3H)-dione (16 mg, 0.05 mmol, 1 equiv) and 2-chloro-4-isocyanato-1-methylbenzene (2 equiv) as starting materials and TEA (3 equiv) as base.

LCMS (ESI+) m/z 452.1 [M+H]⁺

¹H NMR (500 MHz, DMSO-d₆) δ 10.53 (s, 1H), 9.98 (s, 1H), 8.30 (s, 1H), 8.02-7.97 (m, 1H), 7.95 (d, J=8.4 Hz, 1H), 7.63-7.58 (m, 2H), 7.31 (dd, J=8.3, 2.2 Hz, 1H), 7.26 (d, J=8.4 Hz, 1H), 5.38 (s, 2H), 3.94 (td, J=11.1, 10.2, 4.8 Hz, 1H), 3.68 (dt, J=12.1, 5.9 Hz, 1H), 2.88 (ddd, J=16.3, 10.1, 6.1 Hz, 1H), 2.77-2.69 (m, 1H), 2.57 (s, 3H), 2.25 (s, 3H).

### Example 24: Synthesis of (3-(2,6-dioxopiperidin-3-yl)-2-methylquinolin-7-yl)methyl (3-(tert-butyl)isoxazol-5-yl)carbamate (Compound 30)

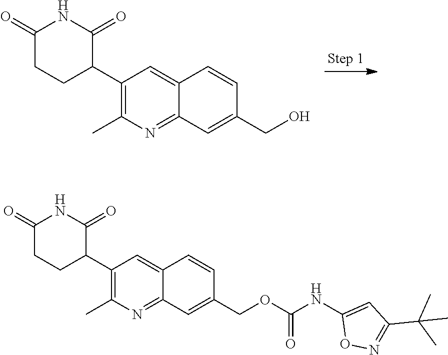 [SMILES: Cc1nc2cc(CO)ccc2cc1C1CCC(=O)NC1=O ; Cc1nc2cc(COC(=O)Nc3cc(C(C)(C)C)no3)ccc2cc1C1CCC(=O)NC1=O]

Step 1: (3-(2,6-Dioxopiperidin-3-yl)-2-methylquinolin-7-yl)methyl (3-(tert-butyl)isoxazol-5-yl)carbamate was synthesized using the general procedure shown in Reaction Scheme 3 and Example Method 3, above (17% yield), using 3-(7-(hydroxymethyl)-2-methylquinolin-3-yl)piperidine-2,6-dione (15.0 mg, 0.053 mmol, 1 equiv) and phenyl (3-(tert-butyl)isoxazol-5-yl)carbamate (1.2 equiv) as starting materials, TEA as base, and DMF as solvent.

The synthesis of phenyl (3-(tert-butyl)isoxazol-5-yl)carbamate was described in US20200390100A1.

LCMS (ESI+) m/z 451.4 [M+H]⁺

¹H NMR (500 MHz, DMSO-d₆): δ 11.43 (s, 1H), 10.93 (s, 1H), 8.13 (s, 1H), 7.96-7.93 (m, 1H), 7.91 (d, J=8.4 Hz, 1H), 7.54 (dd, J=8.4, 1.7 Hz, 1H), 6.02 (s, 1H), 5.40 (s, 2H), 4.30 (dd, J=12.5, 4.7 Hz, 1H), 2.82 (ddd, J=17.7, 12.9, 5.3 Hz, 1H), 2.66 (s, 3H), 2.64-2.58 (m, 1H), 2.42 (qd, J=12.9, 4.3 Hz, 1H), 2.12 (dtd, J=13.0, 5.2, 3.0 Hz, 1H), 1.24 (s, 9H).

### Example 25: Synthesis of (3-(2,6-dioxopiperidin-3-yl)-2-methylquinolin-7-yl)methyl (5-phenylthiophen-3-yl)carbamate (Compound 37)

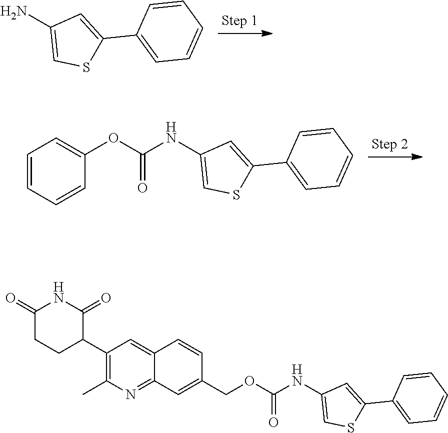 [SMILES: Nc1csc(-c2ccccc2)c1 ; O=C(Nc1csc(-c2ccccc2)c1)Oc1ccccc1 ; Cc1nc2cc(COC(=O)Nc3csc(-c4ccccc4)c3)ccc2cc1C1CCC(=O)NC1=O]

Step 1: Phenyl (5-phenylthiophen-3-yl)carbamate was synthesized using the general procedure shown in Reaction Scheme 6 and Example Method 6, above (41% yield), using 5-phenylthiophen-3-amine (50 mg, 0.285 mmol) as starting material.

LCMS (ESI+) m/z 296.1 [M+H]⁺

Step 2: Sodium hydride (60% suspension in mineral oil, 4.1 mg, 0.094 mmol, 2 equiv) was suspended in dry DMF (1 mL) under argon and cooled in ice-water bath. 3-(7-(Hydroxymethyl)-2-methylquinolin-3-yl)piperidine-2,6-dione (15.9 mg, 0.056 mmol, 1.1 equiv) in DMF (1 mL) was added and the resulting mixture was stirred at 0° C. for 30 min. Phenyl (5-phenylthiophen-3-yl)carbamate (15 mg, 0.051 mmol, 1 equiv) was added and the mixture was stirred at 0° C. for additional 30 min and allowed to warm to RT. The reaction mixture was directly purified by flash column chromatography to give (3-(2,6-dioxopiperidin-3-yl)-2-methylquinolin-7-yl)methyl (5-phenylthiophen-3-yl)carbamate (4.2 mg, 16.5% yield).

LCMS (ESI+) m/z 486.2 [M+H]⁺

¹H NMR (500 MHz, DMSO-d₆) δ 10.93 (s, 1H), 10.20 (s, 1H), 8.13 (s, 1H), 7.97-7.93 (m, 1H), 7.91 (d, J=8.4 Hz, 1H), 7.56 (m, 3H), 7.42 (t, J=7.8 Hz, 2H), 7.36 (d, J=1.5 Hz, 1H), 7.35-7.28 (m, 1H), 7.22 (s, 1H), 5.38 (s, 2H), 4.29 (dd, J=12.5, 4.7 Hz, 1H), 2.82 (ddd, J=17.8, 12.9, 5.3 Hz, 1H), 2.68-2.61 (m, 4H), 2.44-2.38 (m, 1H), 2.18-2.08 (m, 1H).

### Example 26: Synthesis of N-(3-chloro-4-methylphenyl)-3-(3-(2,6-dioxopiperidin-3-yl)-2-methylquinolin-7-yl)propanamide (Compound 32)

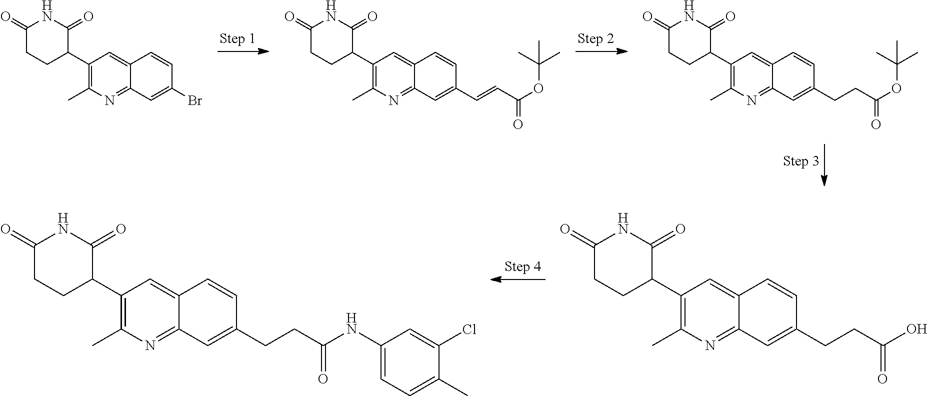 [SMILES: Cc1nc2cc(Br)ccc2cc1C1CCC(=O)NC1=O ; Cc1nc2cc(/C=C/C(=O)OC(C)(C)C)ccc2cc1C1CCC(=O)NC1=O ; Cc1nc2cc(CCC(=O)OC(C)(C)C)ccc2cc1C1CCC(=O)NC1=O ; Cc1ccc(NC(=O)CCc2ccc3cc(C4CCC(=O)NC4=O)c(C)nc3c2)cc1Cl ; Cc1nc2cc(CCC(=O)O)ccc2cc1C1CCC(=O)NC1=O]

Step 1: In a vial were placed 3-(7-bromo-2-methylquinolin-3-yl)piperidine-2,6-dione (30 mg, 0.09 mmol, 1 equiv), palladium(II) acetate (0.1 equiv) and tri-o-tolylphosphane (0.2 equiv) and purged with argon for 15 min. Dry DMF (2 mL) was added followed by TEA (5 equiv) and tert-butyl acrylate (3 equiv). The mixture was stirred at 120° C. for 18 h. The volatiles were removed under reduced pressure and the crude product was purified by flash column chromatography to give tert-butyl (E)-3-(3-(2,6-dioxopiperidin-3-yl)-2-methylquinolin-7-yl)acrylate (27.2 mg, 79% yield).

LCMS (ESI+) m/z 381.4 [M+H]⁺

Step 2: tert-Butyl (E)-3-(3-(2,6-dioxopiperidin-3-yl)-2-methylquinolin-7-yl)acrylate (27.2 mg, 0.071 mmol, 1 equiv) was dissolved in ethanol (5 mL) and palladium on activated charcoal (10 mg, 10% wt.) was added. The reaction mixture was stirred at RT under hydrogen atmosphere for 1 h. The solids were filtered off and the volatiles were removed under reduced pressure to give tert-butyl 3-(3-(2,6-dioxopiperidin-3-yl)-2-methylquinolin-7-yl)propanoate (27.2 mg, 99% yield).

LCMS (ESI+) m/z 382.9 [M+H]⁺

Step 3: tert-Butyl 3-(3-(2,6-dioxopiperidin-3-yl)-2-methylquinolin-7-yl)propanoate (27.2 mg, 0.071 mmol, 1 equiv) was dissolved in 18% aqueous HCl solution (3 mL) and stirred at RT for 10 min. The volatiles were removed under reduced pressure to give 3-(3-(2,6-dioxopiperidin-3-yl)-2-methylquinolin-7-yl)propanoic acid as hydrochloride salt (25.1 mg, 97% yield).

LCMS (ESI+) m/z 327.0 [M+H]⁺

Step 4: N-(3-Chloro-4-methylphenyl)-3-(3-(2,6-dioxopiperidin-3-yl)-2-methylquinolin-7-yl)propanamide was synthesized using the general procedure shown in Reaction Scheme 1 and Example Method 1, above (68% yield), using 3-(3-(2,6-dioxopiperidin-3-yl)-2-methylquinolin-7-yl)propanoic acid hydrochloride salt (13.8 mg, 0.038 mmol, 1 equiv) and 2-chloro-4-aminotoluene (1.2 equiv) as starting materials, DIPEA as base, and DMF as solvent.

LCMS (ESI+) m/z 450.1 [M+H]⁺

¹H NMR (500 MHz, DMSO-d₆) δ 10.91 (s, 1H), 10.03 (s, 1H), 8.05 (s, 1H), 7.82-7.77 (m, 2H), 7.76-7.73 (m, 1H), 7.43 (dd, J=8.4, 1.7 Hz, 1H), 7.33 (dd, J=8.3, 2.2 Hz, 1H), 7.24 (dd, J=8.3, 0.9 Hz, 1H), 4.26 (dd, J=12.4, 4.7 Hz, 1H), 3.11 (t, J=7.6 Hz, 2H), 2.81 (ddd, J=17.6, 12.8, 5.3 Hz, 1H), 2.74 (t, J=7.6 Hz, 2H), 2.63 (s, 3H), 2.62-2.57 (m, 1H), 2.45-2.36 (m, 1H), 2.25 (s, 3H), 2.11 (ddt, J=13.0, 5.0, 2.4 Hz, 1H).

### Example 27: Synthesis of (3-(2,4-dioxotetrahydropyrimidin-1(2H)-yl)-2-methylquinolin-6-yl)methyl (3-chloro-4-methylphenyl)carbamate (Compound 29)

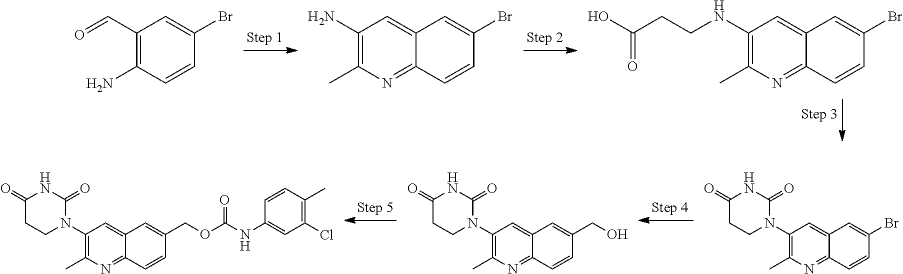 [SMILES: Nc1ccc(Br)cc1C=O ; Cc1nc2ccc(Br)cc2cc1N ; Cc1nc2ccc(Br)cc2cc1NCCC(=O)O ; Cc1ccc(NC(=O)OCc2ccc3nc(C)c(N4CCC(=O)NC4=O)cc3c2)cc1Cl ; Cc1nc2ccc(CO)cc2cc1N1CCC(=O)NC1=O ; Cc1nc2ccc(Br)cc2cc1N1CCC(=O)NC1=O]

Step 1: The mixture of 2-amino-5-bromobenzaldehyde (1 g, 5 mmol, 1 equiv), 1-(2-oxypropyl)pyridin-1-ium chloride (1 equiv) and DMAP (0.02 equiv) was purged with argon for 10 min and dissolved in n-butanol (20 mL). Pyridine (0.7 equiv) was added and the reaction mixture was stirred at 100° C. for 16 h. Pyrrolidine (2.5 equiv) was added and the reaction was further stirred at 100° C. for 4 h. The volatiles were removed under reduced pressure and the residue was taken up in aqueous NaHCO₃ solution. The product was extracted into DCM, the combined organic phases were dried over Na₂SO₄ and evaporated. 6-Bromo-2-methylquinolin-3-amine (865 mg, 73% yield) was purified by flash column chromatography.

LCMS (ESI+) m/z 237.3 [M+H]⁺

Step 2: In a vial were placed 6-bromo-2-methylquinolin-3-amine (865 mg, 3.65 mmol, 1 equiv), water (1.6 mL), acrylic acid (6 equiv), and the reaction mixture was stirred at 70° C. for 20 h. The volatiles were removed under reduced pressure and crude product was purified by flash column chromatography to give 3-((6-bromo-2-methylquinolin-3-yl)amino)propanoic acid (300 mg, 23% yield).

LCMS (ESI+) m/z 309.1 [M+H]⁺

Step 3: In a vial 3-((6-bromo-2-methylquinolin-3-yl)amino)propanoic acid (300 mg, 0.84 mmol, 1 equiv) was dissolved in glacial acetic acid (3 mL). Urea (12 equiv) was added in several portions over stirring the mixture at 120° C. for 3 days. The reaction mixture was cooled to RT, 10% HCl (20 mL) was added and the mixture was refluxed for 30 min. The volatiles were removed under reduced pressure and the crude product was purified by flash column chromatography to give 1-(6-bromo-2-methylquinolin-3-yl)dihydropyrimidine-2,4(1H,3H)-dione (100 mg, 35% yield).

LCMS (ESI+) m/z 334.1 [M+H]⁺

Step 4: 1-(6-(Hydroxymethyl)-2-methylquinolin-3-yl)dihydropyrimidine-2,4(1H,3H)-dione was synthesized using the general procedure shown in Reaction Scheme 7 and Example Method 7, above (68% yield), using 1-(6-bromo-2-methylquinolin-3-yl)dihydropyrimidine-2,4(1H,3H)-dione (25 mg, 0.075 mmol, 1 equiv) and (tributylstannyl)methanol (4 equiv) as starting materials, tetrakis(triphenylphosphine)palladium(0) (0.1 equiv) as catalyst and dioxane as solvent.

LCMS (ESI+) m/z 286.1 [M+H]⁺

Step 5: (3-(2,4-Dioxotetrahydropyrimidin-1(2H)-yl)-2-methylquinolin-6-yl)methyl (3-chloro-4-methylphenyl)carbamate was synthesized using the general procedure shown in Reaction Scheme 3 and Example Method 3, above (6.9% yield), using 1-(6-(hydroxymethyl)-2-methylquinolin-3-yl)dihydropyrimidine-2,4(1H,3H)-dione (15.0 mg, 0.051 mmol, 1 equiv) and phenyl (3-chloro-4-methylphenyl)carbamate (1.2 equiv) as starting materials, TEA as base, and DMF as solvent.

LCMS (ESI+) m/z 453.1 [M+H]⁺

¹H NMR (500 MHz, DMSO-d₆) δ 10.57 (s, 1H), 9.97 (s, 1H), 8.42 (s, 1H), 8.04 (d, J=8.7 Hz, 1H), 8.00 (d, J=1.9 Hz, 1H), 7.84 (dd, J=8.7, 2.0 Hz, 1H), 7.63 (d, J=2.1 Hz, 1H), 7.32 (dd, J=8.3, 2.2 Hz, 1H), 7.27 (d, J=8.4 Hz, 1H), 5.37 (s, 2H), 3.98 (td, J=11.6, 11.0, 4.8 Hz, 1H), 3.71 (dt, J=11.9, 5.7 Hz, 1H), 2.91 (ddd, J=16.4, 10.4, 6.3 Hz, 1H), 2.81-2.72 (m, 1H), 2.62 (s, 3H), 2.27 (s, 3H).

### Example 28: Synthesis of 1-(3-chloro-4-methylphenyl)-3-((3-(2,4-dioxotetrahydropyrimidin-1(2H)-yl)-2-methylquinolin-7-yl)methyl)urea (Compound 27)

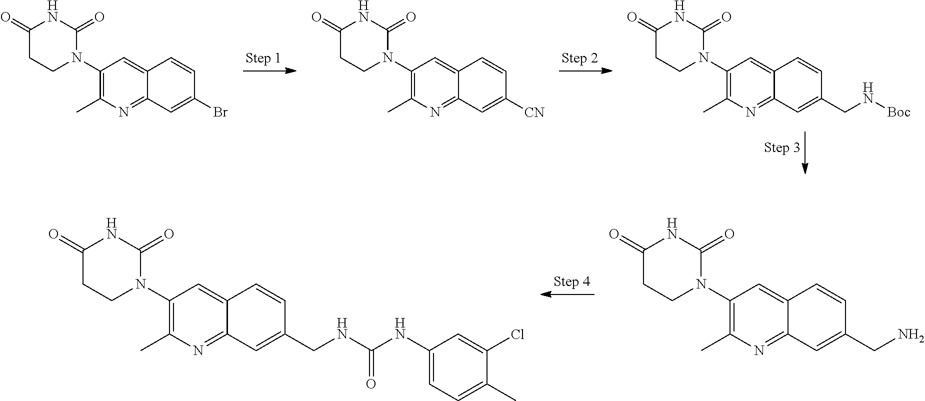 [SMILES: Cc1nc2cc(Br)ccc2cc1N1CCC(=O)NC1=O ; Cc1nc2cc(C#N)ccc2cc1N1CCC(=O)NC1=O ; Cc1nc2cc(CNC(=O)OC(C)(C)C)ccc2cc1N1CCC(=O)NC1=O ; Cc1ccc(NC(=O)NCc2ccc3cc(N4CCC(=O)NC4=O)c(C)nc3c2)cc1Cl ; Cc1nc2cc(CN)ccc2cc1N1CCC(=O)NC1=O]

Step 1: In a vial were placed 1-(7-bromo-2-methylquinolin-3-yl)dihydropyrimidine-2,4(1H,3H)-dione (250 mg, 0.748 mmol, 1 equiv), zinc cyanide (2 equiv), tetrakis(triphenylphosphine)palladium(0) (0.08 equiv), DMF (5 mL) and the reaction mixture was stirred at 120° C. for 18 h. The volatiles were removed under reduced pressure and the crude product was purified by flash column chromatography to give 3-(2,4-dioxotetrahydropyrimidin-1(2H)-yl)-2-methylquinoline-7-carbonitrile (190 mg, 79% yield).

LCMS (ESI+) m/z 281.1 [M+H]⁺

Step 2: To a solution of 3-(2,4-dioxotetrahydropyrimidin-1(2H)-yl)-2-methylquinoline-7-carbonitrile (190 mg, 0.678 mmol, 1 equiv) and di-tert-butyl dicarbonate (2 equiv) in DMF (4 mL) was added Raney Nickel (2 equiv) in THF (5 mL). The reaction mixture was stirred at RT under hydrogen atmosphere for 18 h, filtered through Celite® and the filtrates were concentrated under reduced pressure. The crude product was purified by flash column chromatography to give tert-butyl ((3-(2,4-dioxotetrahydropyrimidin-1(2H)-yl)-2-methylquinolin-7-yl)methyl)carbamate (47 mg, 18% yield).

LCMS (ESI+) m/z 385.4 [M+H]⁺

Step 3: tert-Butyl ((3-(2,4-dioxotetrahydropyrimidin-1(2H)-yl)-2-methylquinolin-7-yl)methyl)carbamate (22 mg, 0.057 mmol, 1 equiv) was dissolved in TFA (1 mL) and the solution was stirred at RT for 1 h. The volatiles were removed under reduced pressure to give 1-(7-(aminomethyl)-2-methylquinolin-3-yl)dihydropyrimidine-2,4(1H,3H)-dione trifluoroacetate (22.7 mg, 100% yield).

LCMS (ESI+) m/z 285.0 [M+H]⁺

Step 4: 1-(7-(Aminomethyl)-2-methylquinolin-3-yl)dihydropyrimidine-2,4(1H,3H)-dione trifluoroacetate (22.7 mg, 0.057 mmol, 1 equiv) was dissolved in DMF (0.55 mL). TEA (3 equiv) was added followed by 2-chloro-4-isocyanato-1-methylbenzene (2 equiv) and the resulting solution was stirred at RT for 2 h. The volatiles were removed under reduced pressure and the crude product was purified by preparative HPLC to give 1-(3-chloro-4-methylphenyl)-3-((3-(2,4-dioxotetrahydropyrimidin-1(2H)-yl)-2-methylquinolin-7-yl)methyl)urea (10 mg, 39% yield).

LCMS (ESI+) m/z 452.1 [M+H]⁺

¹H NMR (500 MHz, DMSO-d₆) δ 10.51 (s, 1H), 8.79 (s, 1H), 8.25 (s, 1H), 7.88 (d, J=8.4 Hz, 1H), 7.85-7.82 (m, 1H), 7.67 (d, J=2.1 Hz, 1H), 7.51 (dd, J=8.4, 1.7 Hz, 1H), 7.19 (d, J=8.4 Hz, 1H), 7.15 (dd, J=8.2, 2.1 Hz, 1H), 6.87 (t, J=6.1 Hz, 1H), 4.51 (d, J=5.9 Hz, 2H), 3.92 (ddd, J=12.3, 10.0, 5.1 Hz, 1H), 3.67 (dt, J=12.0, 5.9 Hz, 1H), 2.87 (ddd, J=16.3, 9.5, 6.1 Hz, 1H), 2.77-2.69 (m, 1H), 2.56 (s, 3H), 2.23 (s, 3H).

### Example 29: Synthesis of (3-(2,6-dioxopiperidin-3-yl)-5-fluoro-2-methylquinolin-7-yl)methyl (3-chloro-4-methylphenyl)carbamate (Compound 22)

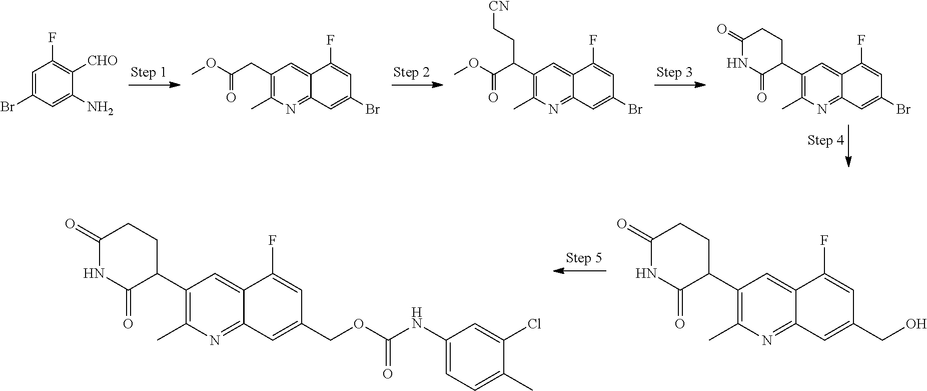 [SMILES: Nc1cc(Br)cc(F)c1C=O ; COC(=O)Cc1cc2c(F)cc(Br)cc2nc1C ; COC(=O)C(CCC#N)c1cc2c(F)cc(Br)cc2nc1C ; Cc1nc2cc(Br)cc(F)c2cc1C1CCC(=O)NC1=O ; Cc1ccc(NC(=O)OCc2cc(F)c3cc(C4CCC(=O)NC4=O)c(C)nc3c2)cc1Cl ; Cc1nc2cc(CO)cc(F)c2cc1C1CCC(=O)NC1=O]

Step 1: To a solution of 2-amino-4-bromo-6-fluorobenzaldehyde (1.46 g, 7 mmol, 1 equiv) and 4-oxopentanoic acid (1.1 equiv) in methanol (40 mL) was added 2M sodium hydroxide solution (4 mL, 8 mmol, 1.2 equiv) and the reaction was stirred at 75° C. for 18 h. The mixture was acidified with acetic acid volatiles were concentrated under reduced pressure. The precipitated product was filtered, washed with water and dried. The crude product was dissolved in methanol (40 mL), thionyl chloride (0.977 mL, 13 mmol, 2 equiv) was added dropwise, and the solution was refluxed for 18 h. Methanol was removed under reduced pressure, the residue was diluted with ethyl acetate and saturated NaHCO₃ was added. The organic phase was washed with brine, dried over MgSO₄ and concentrated under reduced pressure to give methyl 2-(7-bromo-5-fluoro-2-methylquinolin-3-yl)acetate (0.89 g, 43% yield).

LCMS (ESI+) m/z 312.1 [M+H]⁺

Step 2: Methyl 2-(7-bromo-5-fluoro-2-methylquinolin-3-yl)-4-cyanobutanoate was synthesized using the general procedure shown in Reaction Scheme 8 and Example Method 8, above (77% yield), using methyl 2-(7-bromo-5-fluoro-2-methylquinolin-3-yl)acetate (890 mg, 3 mmol, 1 equiv) and acrylonitrile (1 equiv) as starting materials.

LCMS (ESI+) m/z 365.1 [M+H]⁺

Step 3: 3-(7-Bromo-5-fluoro-2-methylquinolin-3-yl)piperidine-2,6-dione was synthesized using the general procedure shown in Reaction Scheme 9 and Example Method 9, above (57% yield), using methyl 2-(7-bromo-5-fluoro-2-methylquinolin-3-yl)-4-cyanobutanoate (100 mg, 0.274 mmol, 1 equiv) as starting material.

LCMS (ESI+) m/z 351.2 [M+H]⁺

Step 4: 3-(5-Fluoro-7-(hydroxymethyl)-2-methylquinolin-3-yl)piperidine-2,6-dione was synthesized using the general procedure shown in Reaction Scheme 7 and Example Method 7, above (37% yield), using 3-(7-bromo-5-fluoro-2-methylquinolin-3-yl)piperidine-2,6-dione (55 mg, 0.157 mmol, 1 equiv) and (tributylstannyl)methanol (1.5 equiv) as starting materials, tetrakis(triphenylphosphine)palladium(0) (0.08 equiv) as catalyst and dioxane as solvent.

LCMS (ESI+) m/z 303.2 [M+H]⁺

Step 5: (3-(2,6-Dioxopiperidin-3-yl)-5-fluoro-2-methylquinolin-7-yl)methyl (3-chloro-4-methylphenyl)carbamate was synthesized using the general procedure shown in Reaction Scheme 3 and Example Method 3, above (18% yield), using 3-(5-fluoro-7-(hydroxymethyl)-2-methylquinolin-3-yl)piperidine-2,6-dione (18 mg, 0.06 mmol, 1 equiv) and phenyl (3-chloro-4-methylphenyl)carbamate (1.7 equiv) as starting materials, TEA as base, and DMF as solvent.

LCMS (ESI+) m/z 470.1 [M+H]⁺

¹H NMR (500 MHz, DMSO-d₆) δ 10.96 (s, 1H), 10.01 (s, 1H), 8.24 (s, 1H), 7.85 (d, J=1.3 Hz, 1H), 7.63 (d, J=2.2 Hz, 1H), 7.44 (dd, J=10.7, 1.5 Hz, 1H), 7.33 (dd, J=8.3, 2.2 Hz, 1H), 7.28 (dd, J=8.3, 0.9 Hz, 1H), 5.37 (s, 2H), 4.38 (dd, J=12.7, 4.7 Hz, 1H), 2.84 (ddd, J=17.6, 13.0, 5.3 Hz, 1H), 2.70 (s, 3H), 2.65-2.59 (m, 1H), 2.59-2.52 (m, 1H), 2.28 (s, 3H), 2.16-2.09 (m, 1H).

### Example 30: Synthesis of N-(3-chloro-4-methylphenyl)-2-((3-(2,6-dioxopiperidin-3-yl)-2-methylquinolin-7-yl)oxy)propenamide (Compound 34)

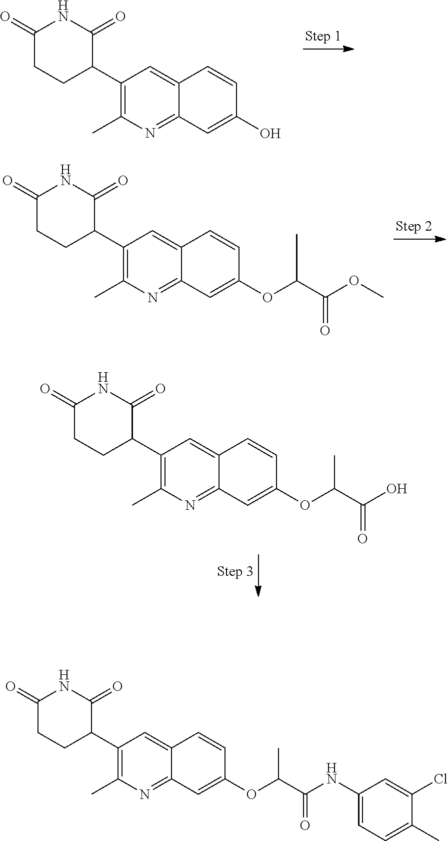 [SMILES: Cc1nc2cc(O)ccc2cc1C1CCC(=O)NC1=O ; COC(=O)C(C)Oc1ccc2cc(C3CCC(=O)NC3=O)c(C)nc2c1 ; Cc1nc2cc(OC(C)C(=O)O)ccc2cc1C1CCC(=O)NC1=O ; Cc1ccc(NC(=O)C(C)Oc2ccc3cc(C4CCC(=O)NC4=O)c(C)nc3c2)cc1Cl]

Step 1: In a vial were placed 3-(7-hydroxy-2-methylquinolin-3-yl)piperidine-2,6-dione (50 mg, 0.185 mmol, 1 equiv), potassium iodide (1 equiv), potassium hydrogencarbonate (3 equiv) and DMF (0.5 mL). The solution of methyl 2-bromopropanoate (1 equiv) in DMF (0.5 mL) was added dropwise and the reaction mixture was stirred at 60° C. for 48 h. The volatiles were removed under reduced pressure and the crude product was purified by flash column chromatography to give methyl 2-((3-(2,6-dioxopiperidin-3-yl)-2-methylquinolin-7-yl)oxy)propanoate (48 mg, 73% yield).

LCMS (ESI+) m/z 357.0 [M+H]⁺

Step 2: In a vial were placed methyl 2-((3-(2,6-dioxopiperidin-3-yl)-2-methylquinolin-7-yl)oxy)propanoate (48 mg, 0.135 mmol, 1 equiv), trimethyltin hydroxide (2 equiv), DCE (0.6 mL) and the reaction mixture was stirred at 85° C. for 24 h. The volatiles were removed under reduced pressure and the crude product was purified by flash column chromatography to give 2-((3-(2,6-dioxopiperidin-3-yl)-2-methylquinolin-7-yl)oxy)propanoic acid (44.5 mg, 96% yield).

LCMS (ESI+) m/z 343.3 [M+H]⁺

Step 3: N-(3-Chloro-4-methylphenyl)-2-((3-(2,6-dioxopiperidin-3-yl)-2-methylquinolin-7-yl)oxy)propenamide was synthesized using the general procedure shown in Reaction Scheme 1 and Example Method 1, above (40% yield), using 2-((3-(2,6-dioxopiperidin-3-yl)-2-methylquinolin-7-yl)oxy)propanoic acid (10 mg, 0.029 mmol, 1 equiv) and 2-chloro-4-aminotoluene (1.2 equiv) as starting materials, DIPEA as base, and DMF as solvent.

LCMS (ESI+) m/z 466.2 [M+H]⁺

¹H NMR (500 MHz, DMSO-d₆) δ 10.90 (s, 1H), 10.34 (d, J=5.6 Hz, 1H), 8.02 (d, J=1.6 Hz, 1H), 7.84-7.78 (m, 2H), 7.45 (dt, J=8.3, 2.3 Hz, 1H), 7.31-7.20 (m, 3H), 5.05 (qd, J=6.6, 1.8 Hz, 1H), 4.26-4.18 (m, 1H), 2.86-2.75 (m, 1H), 2.62-2.55 (m, 4H), 2.41-2.31 (m, 1H), 2.26 (d, J=1.1 Hz, 3H), 2.12-2.04 (m, 1H), 1.62 (dd, J=6.6, 1.7 Hz, 3H).

### Example 31: Synthesis of N-cyclohexyl-2-((3-(2,6-dioxopiperidin-3-yl)-2-methylquinolin-7-yl)oxy)propenamide (Compound 36)

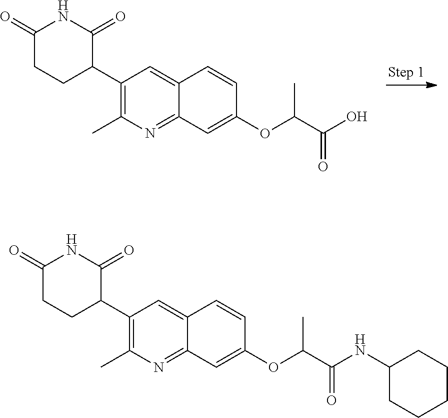 [SMILES: Cc1nc2cc(OC(C)C(=O)O)ccc2cc1C1CCC(=O)NC1=O ; Cc1nc2cc(OC(C)C(=O)NC3CCCCC3)ccc2cc1C1CCC(=O)NC1=O]

Step 1: N-Cyclohexyl-2-((3-(2,6-dioxopiperidin-3-yl)-2-methylquinolin-7-yl)oxy)propanamide was synthesized using the general procedure shown in Reaction Scheme 1 and Example Method 1, above (40% yield), using 2-((3-(2,6-dioxopiperidin-3-yl)-2-methylquinolin-7-yl)oxy)propanoic acid (10 mg, 0.029 mmol, 1 equiv) and cyclohexylamine (1.2 equiv) as starting materials, DIPEA as base, and DMF as solvent.

LCMS (ESI+) m/z 424.2 [M+H]⁺

¹H NMR (500 MHz, DMSO-d₆) δ 10.92 (s, 1H), 8.05 (dd, J=8.1, 2.7 Hz, 1H), 8.03 (s, 1H), 7.79 (d, J=8.9 Hz, 1H), 7.24 (d, J=2.5 Hz, 1H), 7.21 (dd, J=8.8, 2.5 Hz, 1H), 4.89-4.82 (m, 1H), 4.25 (dd, J=12.5, 4.8 Hz, 1H), 3.56 (s, 1H), 2.83 (ddd, J=17.6, 12.8, 5.2 Hz, 1H), 2.62 (s, 4H), 2.46-2.34 (m, 1H), 2.16-2.08 (m, 1H), 1.76-1.59 (m, 4H), 1.50 (d, J=6.6 Hz, 3H), 1.30-1.17 (m, 6H).

### Example 32: Synthesis of (3-(2,6-dioxopiperidin-3-yl)-2-methylquinolin-7-yl)methyl (6-morpholinopyridin-3-yl)carbamate (Compound 42)

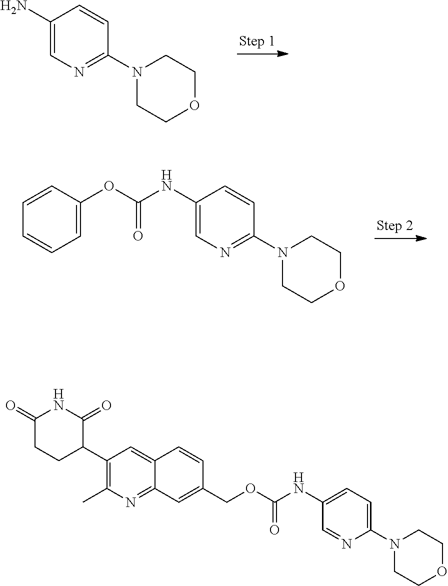 [SMILES: Nc1ccc(N2CCOCC2)nc1 ; O=C(Nc1ccc(N2CCOCC2)nc1)Oc1ccccc1 ; Cc1nc2cc(COC(=O)Nc3ccc(N4CCOCC4)nc3)ccc2cc1C1CCC(=O)NC1=O]

Step 1: Phenyl (6-morpholinopyridin-3-yl)carbamate was synthesized using the general procedure shown in Reaction Scheme 6 and Example Method 6, above (37% yield), using 6-morpholinopyridin-3-amine (300 mg, 1.67 mmol) as starting material.

LCMS (ESI+) m/z 300.8 [M+H]⁺

Step 2: (3-(2,6-Dioxopiperidin-3-yl)-2-methylquinolin-7-yl)methyl (6-morpholinopyridin-3-yl)carbamate was synthesized using the general procedure shown in Reaction Scheme 3 and Example Method 3, above (28% yield), using 3-(7-(hydroxymethyl)-2-methylquinolin-3-yl)piperidine-2,6-dione (15 mg, 0.053 mmol, 1 equiv) and phenyl (6-morpholinopyridin-3-yl)carbamate (1.2 equiv) as starting materials, TEA as base and DMF as solvent.

LCMS (ESI+) m/z 490.3 [M+H]⁺

¹H NMR (500 MHz, DMSO-d₆) δ 10.93 (s, 1H), 9.65 (s, 1H), 8.22 (s, 1H), 8.13 (s, 1H), 7.94 (s, 1H), 7.90 (d, J=8.4 Hz, 1H), 7.76-7.63 (m, 1H), 7.54 (dd, J=8.4, 1.7 Hz, 1H), 6.82 (d, J=9.1 Hz, 1H), 5.34 (s, 2H), 4.29 (dd, J=12.5, 4.7 Hz, 1H), 3.73-3.65 (m, 4H), 3.36-3.33 (m, 4H), 2.87-2.78 (m, 1H), 2.66 (s, 3H), 2.64-2.58 (m, 1H), 2.42 (qd, J=12.9, 4.3 Hz, 1H), 2.13 (dtd, J=12.9, 5.1, 3.0 Hz, 1H).

### Example 33: Synthesis of (3-(2,6-dioxopiperidin-3-yl)-2,4-dimethylquinolin-7-yl)methyl (3-chloro-4-methylphenyl)carbamate (Compound 44)

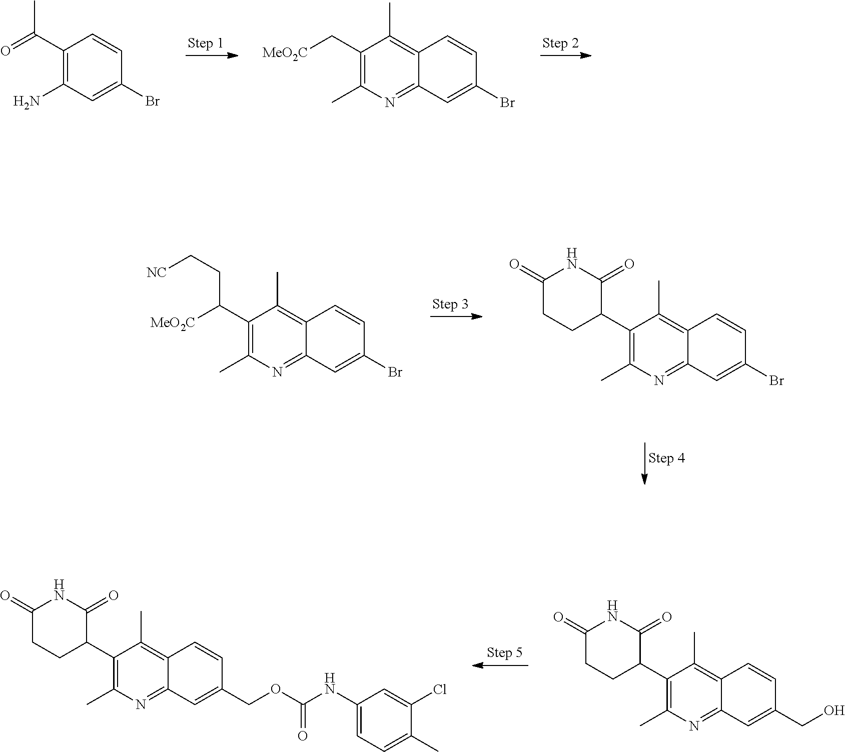 [SMILES: CC(=O)c1ccc(Br)cc1N ; COC(=O)Cc1c(C)nc2cc(Br)ccc2c1C ; COC(=O)C(CCC#N)c1c(C)nc2cc(Br)ccc2c1C ; Cc1nc2cc(Br)ccc2c(C)c1C1CCC(=O)NC1=O ; Cc1ccc(NC(=O)OCc2ccc3c(C)c(C4CCC(=O)NC4=O)c(C)nc3c2)cc1Cl ; Cc1nc2cc(CO)ccc2c(C)c1C1CCC(=O)NC1=O]

Step 1: To a solution of 1-(2-amino-4-bromophenyl)ethan-1-one (1.5 g, 7 mmol, 1 equiv) and 4-oxopentanoic acid (1.1 equiv) in dry DMF (7 ml) was added chlorotrimethylsilane (4 equiv) and the mixture was stirred under microwave irradiation at 100° C. for 2 h. The reaction was cooled, quenched with water and extracted with 20% isopropanol in DCM and 20% methanol in ethyl acetate. The combined organic fractions were dried over Na₂SO₄ and evaporated.

Crude 2-(7-bromo-2,4-dimethylquinolin-3-yl)acetic acid was dissolved in methanol (30 ml) and thionyl chloride (5 equiv) was slowly added. The reaction mixture was stirred at 70° C. for 18 h and concentrated under reduced pressure. DCM was added to the oily residue and the precipitated product was collected by filtration. The filtrate was concentrated in vacuo and the residue was submitted to flash column chromatography to provide additional portion of the product. The combined batches gave methyl 2-(7-bromo-2,4-dimethylquinolin-3-yl)acetate (1.37 g, 3.97 mmol) with 57% yield.

LCMS (ESI+) m/z 308.2 [M+H]⁺

Step 2: Methyl 2-(7-bromo-2,4-dimethylquinolin-3-yl)-4-cyanobutanoate was synthesized using the general procedure shown in Reaction Scheme 8 and Example Method 8, above (39% yield), using methyl 2-(7-bromo-2,4-dimethyl quinolin-3-yl)acetate (35 mg, 0.114 mmol, 1 equiv) and acrylonitrile (4 equiv) as starting materials.

LCMS (ESI+) m/z 360.9 [M+H]⁺

Step 3: 3-(7-Bromo-2,4-dimethyl quinolin-3-yl)piperidine-2,6-dione was synthesized using the general procedure shown in Reaction Scheme 9 and Example Method 9, above (15% yield), using methyl 2-(7-bromo-2,4-dimethylquinolin-3-yl)-4-cyanobutanoate (13 mg, 0.036 mmol) as starting material.

LCMS (ESI+) m/z 347.0 [M+H]⁺

Step 4: 3-(7-(Hydroxymethyl)-2,4-dimethylquinolin-3-yl)piperidine-2,6-dione was synthesized using the general procedure shown in Reaction Scheme 7 and Example Method 7, above (51% yield), using 3-(7-bromo-2,4-dimethylquinolin-3-yl)piperidine-2,6-dione (18.9 mg, 0.054 mmol, 1 equiv) and (tributylstannyl)methanol (1.14 equiv) as starting materials, tetrakis(triphenylphosphine)palladium(0) (0.079 equiv) as catalyst and dioxane as solvent.

LCMS (ESI+) m/z 299.1 [M+H]⁺

Step 5: (3-(2,6-Dioxopiperidin-3-yl)-2,4-dimethylquinolin-7-yl)methyl (3-chloro-4-methylphenyl)carbamate was synthesized using the general procedure shown in Reaction Scheme 5 and Example Method 5, above (31% yield), using 3-(7-(hydroxymethyl)-2,4-dimethylquinolin-3-yl)piperidine-2,6-dione (15 mg, 0.045 mmol, 1 equiv) and 2-chloro-4-isocyanato-1-methylbenzene (5 equiv) as starting materials, and DIPEA (3 equiv) as base.

LCMS (ESI+) m/z 466.2 [M+H]⁺

¹H NMR (500 MHz, DMSO-d₆, 353K) δ 10.66 (s, 1H), 9.66 (s, 1H), 8.08 (s, 1H), 7.92 (dd, J=1.9, 0.8 Hz, 1H), 7.60 (d, J=2.2 Hz, 1H), 7.56 (dd, J=8.7, 1.9 Hz, 1H), 7.32 (dd, J=8.3, 2.2 Hz, 1H), 7.23 (dd, J=8.3, 0.8 Hz, 1H), 5.37 (s, 2H), 4.44 (br s, 1H), 2.91 (ddd, J=16.9, 13.6, 5.9 Hz, 1H), 2.80-2.65 (br s, 3H), 2.62 (ddd, J=17.0, 4.3, 2.1 Hz, 1H), 2.50-2.40 (br s, 3H), 2.30-2.20 (m, 4H), 2.12-2.02 (br s, 1H).

### Example 34: Synthesis of (3-(2,6-dioxopiperidin-3-yl)-2-methylquinolin-7-yl)methyl (6-methoxypyridazin-3-yl)carbamate (Compound 45)

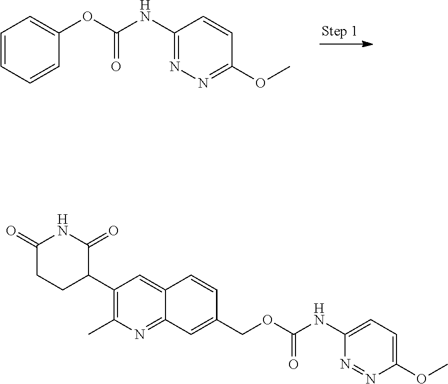 [SMILES: COc1ccc(NC(=O)Oc2ccccc2)nn1 ; COc1ccc(NC(=O)OCc2ccc3cc(C4CCC(=O)NC4=O)c(C)nc3c2)nn1]

Step 1: (3-(2,6-Dioxopiperidin-3-yl)-2-methylquinolin-7-yl)methyl (6-methoxypyridazin-3-yl)carbamate was synthesized using the general procedure shown in Reaction Scheme 3 and Example Method 3, above (22% yield), using 3-(7-(hydroxymethyl)-2-methylquinolin-3-yl)piperidine-2,6-dione (15 mg, 0.053 mmol, 1 equiv) and phenyl (6-methoxypyridazin-3-yl)carbamate (1.2 equiv) as starting materials, TEA as base and DMF as solvent.

The synthesis of phenyl (6-methoxypyridazin-3-yl)carbamate was described in US2008261941A1.

LCMS (ESI+) m/z 436.2 [M+H]⁺

¹H NMR (500 MHz, DMSO-d₆) δ 10.93 (s, 1H), 10.78 (s, 1H), 8.13 (s, 1H), 8.04 (d, J=9.5 Hz, 1H), 8.00-7.95 (m, 1H), 7.90 (d, J=8.3 Hz, 1H), 7.56 (dd, J=8.4, 1.7 Hz, 1H), 7.25 (dd, J=9.5, 0.5 Hz, 1H), 5.40 (s, 2H), 4.29 (dd, J=12.5, 4.8 Hz, 1H), 3.97 (s, 3H), 2.87-2.78 (m, 1H), 2.66 (s, 3H), 2.64-2.59 (m, 1H), 2.46-2.38 (m, 1H), 2.16-2.08 (m, 1H).

### Example 35: Synthesis of (3-(2,6-dioxopiperidin-3-yl)-2-methylquinolin-7-yl)methyl [1,1′-biphenyl]-3-ylcarbamate (Compound 46)

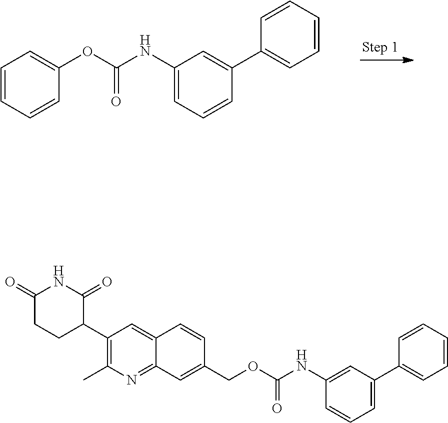 [SMILES: O=C(Nc1cccc(-c2ccccc2)c1)Oc1ccccc1 ; Cc1nc2cc(COC(=O)Nc3cccc(-c4ccccc4)c3)ccc2cc1C1CCC(=O)NC1=O]

Step 1: (3-(2,6-Dioxopiperidin-3-yl)-2-methylquinolin-7-yl)methyl [1,1′-biphenyl]-3-ylcarbamate was synthesized using the general procedure shown in Reaction Scheme 3 and Example Method 3, above (36% yield), using 3-(7-(hydroxymethyl)-2-methylquinolin-3-yl)piperidine-2,6-dione (15 mg, 0.053 mmol, 1 equiv) and phenyl [1,1′-biphenyl]-3-ylcarbamate (1.2 equiv) as starting materials, TEA as base and DMF as solvent.

The synthesis of phenyl [1,1′-biphenyl]-3-ylcarbamate was described in WO2005044810A1.

LCMS (ESI+) m/z 480.2 [M+H]⁺

¹H NMR (500 MHz, DMSO-d₆) δ 10.93 (s, 1H), 9.96 (s, 1H), 8.13 (s, 1H), 8.01-7.94 (m, 1H), 7.91 (d, J=8.3 Hz, 1H), 7.85-7.75 (m, 1H), 7.64-7.54 (m, 3H), 7.53-7.41 (m, 3H), 7.41-7.33 (m, 2H), 7.29 (ddd, J=7.7, 1.8, 1.1 Hz, 1H), 5.38 (s, 2H), 4.30 (dd, J=12.5, 4.7 Hz, 1H), 2.87-2.77 (m, 1H), 2.66 (s, 3H), 2.62 (dt, J=17.2, 2.9 Hz, 1H), 2.42 (qd, J=12.9, 4.3 Hz, 1H), 2.13 (dtd, J=12.9, 5.1, 3.0 Hz, 1H).

### Example 36: Synthesis of (3-(2,6-dioxopiperidin-3-yl)-2-methylquinolin-7-yl)methyl (1-phenylpiperidin-4-yl)carbamate (Compound 47)

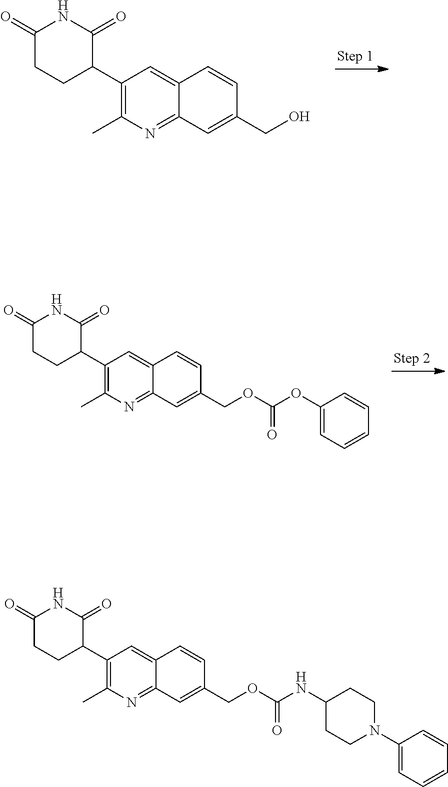 [SMILES: Cc1nc2cc(CO)ccc2cc1C1CCC(=O)NC1=O ; Cc1nc2cc(COC(=O)Oc3ccccc3)ccc2cc1C1CCC(=O)NC1=O ; Cc1nc2cc(COC(=O)NC3CCN(c4ccccc4)CC3)ccc2cc1C1CCC(=O)NC1=O]

Step 1: To a solution of 3-(7-(hydroxymethyl)-2-methylquinolin-3-yl)piperidine-2,6-dione (50 mg, 0.176 mmol, 1 equiv) and pyridine (5 equiv) in DMF (1.5 mL), cooled to 0° C., was added phenyl chloroformate (2 equiv). The reaction mixture was stirred at RT for 18 h. After completion the volatiles were removed in vacuo and the crude product was directly used in the next step.

LCMS (ESI+) m/z 405.3 [M+H]⁺

Step 2: tert-Butyl (1-phenylpiperidin-4-yl)carbamate (47.8 mg, 0.173 mmol, 2 equiv) was dissolved in TFA (0.5 mL) and the solution was stirred for 30 min at RT. The volatiles were removed in vacuo and the residue was redissolved in DMF (1 mL). TEA (10 equiv) and (3-(2,6-dioxopiperidin-3-yl)-2-methylquinolin-7-yl)methyl phenyl carbonate (1 equiv) were added and the reaction mixture was stirred at RT for 18 h, then at 50° C. for 18 h. (3-(2,6-Dioxopiperidin-3-yl)-2-methylquinolin-7-yl)methyl (1-phenylpiperidin-4-yl)carbamate (5.8 mg, 0.012 mmol, 13% yield) was purified by preparative HPLC and repurified twice by preparative TLC.

The synthesis of tert-butyl (1-phenylpiperidin-4-yl)carbamate was described in WO2016094824A1.

LCMS (ESI+) m/z 487.2 [M+H]⁺

¹H NMR (500 MHz, DMSO-d₆) δ 10.93 (s, 1H), 8.11 (s, 1H), 7.91-7.84 (m, 2H), 7.49 (dd, J=8.4, 1.7 Hz, 1H), 7.42 (d, J=7.7 Hz, 1H), 7.22-7.14 (m, 2H), 6.96-6.88 (m, 2H), 6.73 (tt, J=7.2, 1.0 Hz, 1H), 5.23 (s, 2H), 4.29 (dd, J=12.4, 4.8 Hz, 1H), 3.64 (d, J=12.6 Hz, 2H), 3.50 (dd, J=7.2, 3.7 Hz, 1H), 2.86-2.80 (m, 1H), 2.79-2.72 (m, 2H), 2.65 (s, 3H), 2.60 (dd, J=4.2, 2.9 Hz, 1H), 2.46-2.39 (m, 1H), 2.12 (dtd, J=13.0, 5.2, 3.0 Hz, 1H), 1.85 (d, J=11.9 Hz, 2H), 1.56-1.46 (m, 2H).

### Example 37: Synthesis of (3-(2,6-dioxopiperidin-3-yl)-6-fluoro-2-methylquinolin-7-yl)methyl (3-chloro-4-methylphenyl)carbamate (Compound 49)

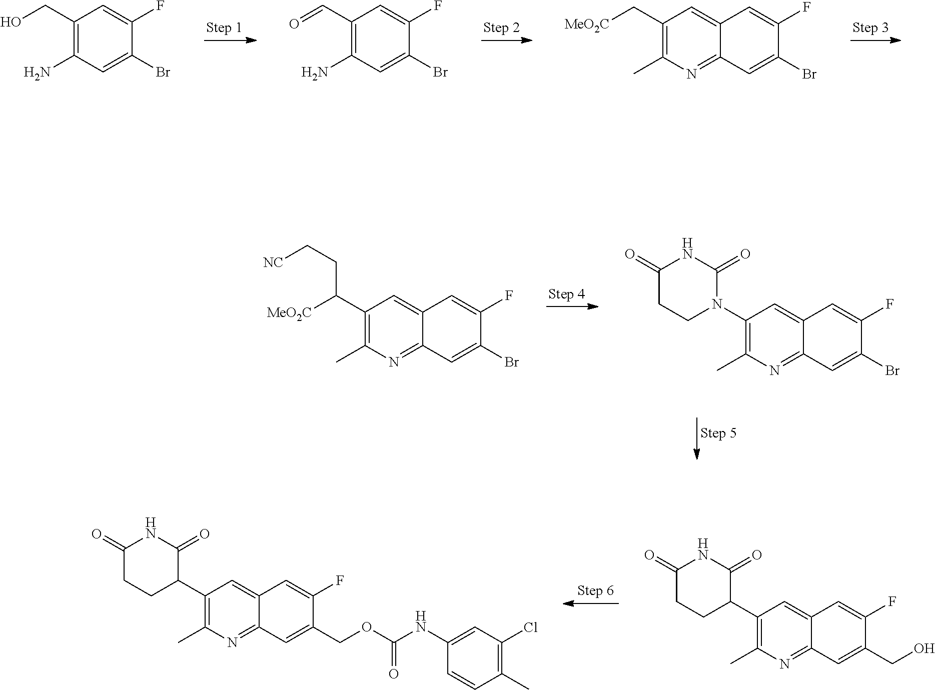

Step 1: (2-Amino-4-bromo-5-fluorophenyl)methanol (1.8 g, 8 mmol, 1 equiv) and MnO₂ (5 equiv) were suspended in DCM (30 mL) and stirred at RT for 6 h. After completion, the solid particles were filtered off on Celite® and concentrated. 2-Amino-4-bromo-5-fluorobenzaldehyde (1.2 g, 6 mmol, 67% yield) was purified by flash column chromatography.

The synthesis of (2-amino-4-bromo-5-fluorophenyl)methanol was described in WO2021247969A1.

LCMS (ESI+) m/z 217.9 [M+H]⁺

Step 2: 2-Amino-4-bromo-5-fluorobenzaldehyde (700 mg, 3.21 mmol, 1 equiv) and 4-oxopentanoic acid (1.1 equiv) were dissolved in methanol (9.6 mL) and purged with argon for 15 min. Sodium hydroxide (2M solution, 1.9 mL, 1.2 equiv) was added and the reaction mixture was stirred at 75° C. for 18 h. The reaction was then cooled, acidified with acetic acid, diluted with water and concentrated in vacuo to remove methanol. The crude 2-(7-bromo-6-fluoro-2-methylquinolin-3-yl)acetic acid was filtered, washed with water and vacuum-dried. The product was redissolved in methanol (9.6 mL) and thionyl chloride (0.47 mL, 6 mmol, 2 equiv) was added dropwise. The reaction mixture was refluxed for 18 h. After completion, the reaction mixture was concentrated, diluted with ethyl acetate and saturated solution of NaHCO₃. The product was extracted with ethyl acetate, combined organic fractions were dried over MgSO₄ and concentrated. Methyl 2-(7-bromo-6-fluoro-2-methylquinolin-3-yl)acetate (600 mg, 1.92 mmol, 60% yield) was purified by flash column chromatography.

LCMS (ESI+) m/z 312.5 [M+H]⁺

Step 3: Methyl 2-(7-bromo-6-fluoro-2-methylquinolin-3-yl)-4-cyanobutanoate was synthesized using the general procedure shown in Reaction Scheme 8 and Example Method 8, above (67% yield), using methyl 2-(7-bromo-6-fluoro-2-methylquinolin-3-yl)acetate (1 g, 3.2 mmol, 1 equiv) and acrylonitrile (1 equiv) as starting materials.

LCMS (ESI+) m/z 365.2 [M+H]⁺

Step 4: 3-(7-Bromo-6-fluoro-2-methylquinolin-3-yl)piperidine-2,6-dione was synthesized using the general procedure shown in Reaction Scheme 9 and Example Method 9, above (42% yield), using methyl 2-(7-bromo-6-fluoro-2-methylquinolin-3-yl)-4-cyanobutanoate (100 mg, 0.274 mmol) as starting material.

LCMS (ESI+) m/z 350.9 [M+H]⁺

Step 5: 3-(6-Fluoro-7-(hydroxymethyl)-2-methylquinolin-3-yl)piperidine-2,6-dione was synthesized using the general procedure shown in Reaction Scheme 7 and Example Method 7, above (30% yield), using 3-(7-bromo-6-fluoro-2-methylquinolin-3-yl)piperidine-2,6-dione (31 mg, 0.088 mmol, 1 equiv) and (tributylstannyl)methanol (3 equiv) as starting materials, tetrakis(triphenylphosphine)palladium(0) (0.09 equiv) as catalyst and dioxane as solvent.

LCMS (ESI+) m/z 303.2 [M+H]⁺

Step 6: (3-(2,6-Dioxopiperidin-3-yl)-6-fluoro-2-methylquinolin-7-yl)methyl (3-chloro-4-methylphenyl)carbamate was synthesized using the general procedure shown in Reaction Scheme 5 and Example Method 5, above (30% yield), using 3-(6-fluoro-7-(hydroxymethyl)-2-methylquinolin-3-yl)piperidine-2,6-dione (15 mg, 0.05 mmol, 1 equiv) and 2-chloro-4-isocyanato-1-methylbenzene (2 equiv) as starting materials and TEA (3 equiv) as base.

LCMS (ESI+) m/z 470.1 [M+H]⁺

¹H NMR (500 MHz, DMSO-d₆) δ 10.95 (s, 1H), 10.00 (s, 1H), 8.13 (s, 1H), 8.07 (d, J=7.1 Hz, 1H), 7.74 (d, J=10.5 Hz, 1H), 7.60 (d, J=2.2 Hz, 1H), 7.31 (dd, J=8.4, 2.2 Hz, 1H), 7.25 (d, J=8.4 Hz, 1H), 5.39 (s, 2H), 4.31 (dd, J=12.5, 4.7 Hz, 1H), 2.82 (ddd, J=17.7, 12.8, 5.3 Hz, 1H), 2.65 (s, 3H), 2.60 (d, J=3.7 Hz, 1H), 2.41 (dd, J=12.8, 4.1 Hz, 1H), 2.25 (s, 3H), 2.18-2.09 (m, 1H).

### Example 38: Synthesis of (3-(2,6-dioxopiperidin-3-yl)-8-fluoro-2-methylquinolin-7-yl)methyl (3-chloro-4-methylphenyl)carbamate (Compound 50)

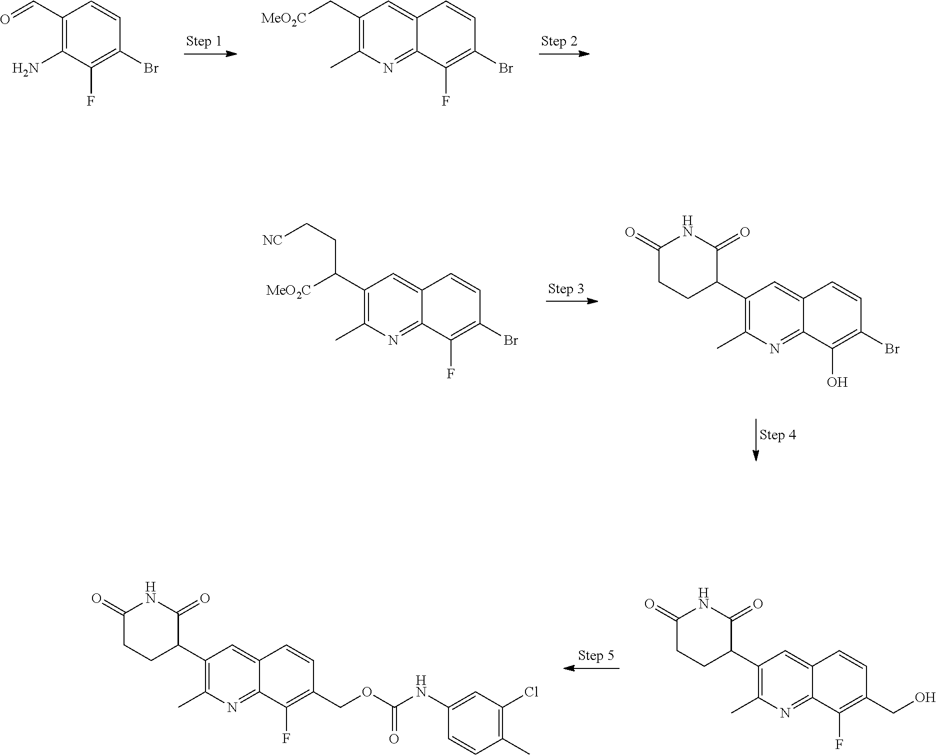 [SMILES: Nc1c(C=O)ccc(Br)c1F ; COC(=O)Cc1cc2ccc(Br)c(F)c2nc1C ; COC(=O)C(CCC#N)c1cc2ccc(Br)c(F)c2nc1C ; Cc1nc2c(O)c(Br)ccc2cc1C1CCC(=O)NC1=O ; Cc1ccc(NC(=O)OCc2ccc3cc(C4CCC(=O)NC4=O)c(C)nc3c2F)cc1Cl ; Cc1nc2c(F)c(CO)ccc2cc1C1CCC(=O)NC1=O]

Step 1: 2-Amino-4-bromo-3-fluorobenzaldehyde (860 mg, 3.95 mmol, 1 equiv) and 4-oxopentanoic acid (1.1 equiv) were dissolved in methanol (11.8 mL) and purged with argon for 15 min. Sodium hydroxide (2M solution, 2.4 mL, 1.2 equiv) was added and the reaction mixture was stirred at 75° C. for 18 h. The reaction was then cooled, acidified with acetic acid, diluted with water and concentrated in vacuo to remove methanol. The crude 2-(7-bromo-8-fluoro-2-methylquinolin-3-yl)acetic acid was filtered, washed with water and vacuum-dried. The product was redissolved in methanol (11.8 mL) and thionyl chloride (0.58 mL, 7.89 mmol, 2 equiv) was added dropwise. The reaction mixture was refluxed for 18 h. After completion, the reaction mixture was concentrated, diluted with ethyl acetate and saturated solution of NaHCO₃. The product was extracted with ethyl acetate, combined organic fractions were dried over MgSO₄ and concentrated. Methyl 2-(7-bromo-8-fluoro-2-methylquinolin-3-yl)acetate (431 mg, 1.38 mmol, 35% yield) was purified by flash column chromatography.

LCMS (ESI+) m/z 311.6 [M+H]⁺

Step 2: Methyl 2-(7-bromo-8-fluoro-2-methylquinolin-3-yl)-4-cyanobutanoate was synthesized using the general procedure shown in Reaction Scheme 8 and Example Method 8, above (36% yield), using methyl 2-(7-bromo-8-fluoro-2-methylquinolin-3-yl)acetate (430 mg, 1.38 mmol, 1 equiv) and acrylonitrile (1 equiv) as starting materials.

LCMS (ESI+) m/z 365.9 [M+H]⁺

Step 3: 3-(7-Bromo-8-fluoro-2-methylquinolin-3-yl)piperidine-2,6-dione was synthesized using the general procedure shown in Reaction Scheme 9 and Example Method 9, above (52% yield), using methyl 2-(7-bromo-8-fluoro-2-methylquinolin-3-yl)-4-cyanobutanoate (79 mg, 0.218 mmol) as starting material.

LCMS (ESI+) m/z 350.9 [M+H]⁺

Step 4: 3-(8-Fluoro-7-(hydroxymethyl)-2-methylquinolin-3-yl)piperidine-2,6-dione was synthesized using the general procedure shown in Reaction Scheme 7 and Example Method 7, above (57% yield), using 3-(7-bromo-8-fluoro-2-methylquinolin-3-yl)piperidine-2,6-dione (19 mg, 0.054 mmol, 1 equiv) and (tributylstannyl)methanol (3 equiv) as starting materials, tetrakis(triphenylphosphine)palladium(0) (0.09 equiv) as catalyst and dioxane as solvent.

LCMS (ESI+) m/z 302.8 [M+H]⁺

Step 5: (3-(2,6-Dioxopiperidin-3-yl)-8-fluoro-2-methylquinolin-7-yl)methyl (3-chloro-4-methylphenyl)carbamate was synthesized using the general procedure shown in Reaction Scheme 5 and Example Method 5, above (53% yield), using 3-(8-fluoro-7-(hydroxymethyl)-2-methylquinolin-3-yl)piperidine-2,6-dione (9.7 mg, 0.032 mmol, 1 equiv) and 2-chloro-4-isocyanato-1-methylbenzene (2 equiv) as starting materials and TEA (3 equiv) as base.

LCMS (ESI+) m/z 470.2 [M+H]⁺

¹H NMR (500 MHz, DMSO-d₆) δ 10.98 (s, 1H), 9.96 (s, 1H), 8.26-8.20 (m, 1H), 7.77 (d, J=8.4 Hz, 1H), 7.67-7.56 (m, 2H), 7.33-7.24 (m, 2H), 5.43 (s, 2H), 4.36 (dd, J=12.5, 4.7 Hz, 1H), 2.85 (ddd, J=17.7, 13.0, 5.3 Hz, 1H), 2.72 (s, 3H), 2.48-2.41 (m, 2H), 2.27 (s, 3H), 2.19-2.12 (m, 1H)

### Example 39: Synthesis of 1-(3-chloro-4-methylphenyl)-3-((3-(2,4-dioxotetrahydropyrimidin-1(2H)-yl)-2-methylquinolin-6-yl)methyl)urea (Compound 51)

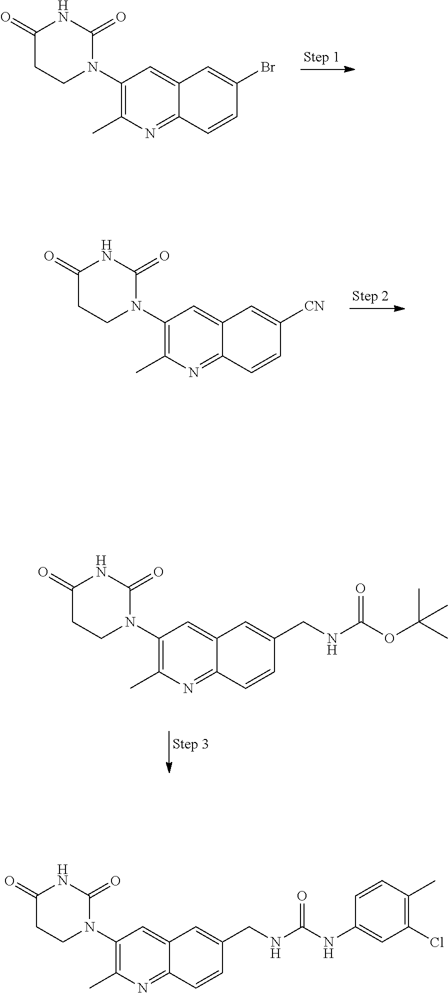 [SMILES: Cc1nc2ccc(Br)cc2cc1N1CCC(=O)NC1=O ; Cc1nc2ccc(C#N)cc2cc1N1CCC(=O)NC1=O ; Cc1nc2ccc(CNC(=O)OC(C)(C)C)cc2cc1N1CCC(=O)NC1=O ; Cc1ccc(NC(=O)NCc2ccc3nc(C)c(N4CCC(=O)NC4=O)cc3c2)cc1Cl]

Step 1: 1-(6-Bromo-2-methylquinolin-3-yl)dihydropyrimidine-2,4(1H,3H)-dione (50 mg, 0.15 mmol, 1 equiv), zinc cyanide (8 equiv) and tetrakis(triphenylphosphine)palladium(0) (0.15 equiv) were suspended in dry DMF under argon and the slurry was bubbled with argon for 15 min. The reaction was then carried out in a sealed tube at 120° C. for 18 h. After completion, the reaction mixture was concentrated and the crude product was purified by flash column chromatography to give 3-(2,4-Dioxotetrahydropyrimidin-1(2H)-yl)-2-methylquinoline-6-carbonitrile (30 mg, 71% yield).

LCMS (ESI+) m/z 280.4 [M+H]⁺

Step 2: To a solution of 3-(2,4-dioxotetrahydropyrimidin-1(2H)-yl)-2-methylquinoline-6-carbonitrile (40 mg, 0.143 mmol, 1 equiv) in DMF (0.84 mL) and THF (1.1 mL) were added Raney Nickel (2 equiv) and di-tert-butyl dicarbonate (2 equiv). The slurry was stirred at RT for 18 h under hydrogen atmosphere (balloon). After completion, the solid particles were filtered off on Celite® and activated carbon and washed with ethanol. The collected filtrate was concentrated in vacuo and tert-butyl ((3-(2,4-dioxotetrahydropyrimidin-1(2H)-yl)-2-methylquinolin-6-yl)methyl)carbamate was purified by flash column chromatography.

LCMS (ESI+) m/z 385.2 [M+H]⁺

Step 3: tert-Butyl ((3-(2,4-dioxotetrahydropyrimidin-1(2H)-yl)-2-methylquinolin-6-yl)methyl)carbamate (23 mg, 0.06 mmol, 1 equiv) was dissolved in TFA (0.3 mL) and stirred for 1 h at RT. The volatiles were removed in vacuo and the residue was redissolved in DMF (0.58 mL). TEA (3 equiv) was added followed by 2-chloro-4-isocyanato-1-methylbenzene (2 equiv). The reaction was carried out at RT for 24 h. 1-(3-Chloro-4-methylphenyl)-3-((3-(2,4-dioxotetrahydropyrimidin-1(2H)-yl)-2-methylquinolin-6-yl)methyl)urea (11 mg, 0.024 mmol, 40% yield) was purified by preparative HPLC.

LCMS (ESI+) m/z 452.1 [M+H]⁺

¹H NMR (500 MHz, DMSO-d₆) δ 10.51 (s, 1H), 8.76 (s, 1H), 8.27 (s, 1H), 7.93 (d, J=8.6 Hz, 1H), 7.77 (d, J=1.9 Hz, 1H), 7.68 (dd, J=8.5, 2.0 Hz, 2H), 7.21-7.12 (m, 2H), 6.83 (t, J=6.0 Hz, 1H), 4.48 (d, J=5.9 Hz, 2H), 3.93 (td, J=11.4, 4.9 Hz, 1H), 3.67 (dt, J=11.9, 5.7 Hz, 1H), 2.88 (ddd, J=16.3, 10.0, 6.1 Hz, 1H), 2.73 (dt, J=16.8, 5.4 Hz, 1H), 2.56 (s, 3H), 2.23 (s, 3H).

### Example 40: Synthesis of 1-cyclohexyl-3-((3-(2,4-dioxotetrahydropyrimidin-1(2H)-yl)-2-methylquinolin-6-yl)methyl)urea (Compound 52)

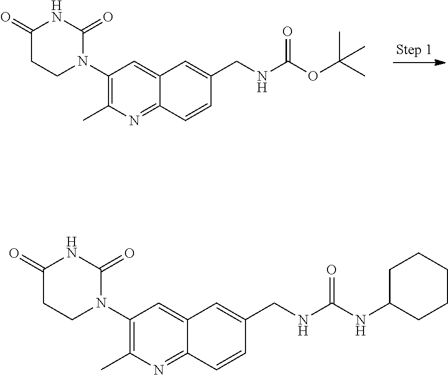 [SMILES: Cc1nc2ccc(CNC(=O)OC(C)(C)C)cc2cc1N1CCC(=O)NC1=O ; Cc1nc2ccc(CNC(=O)NC3CCCCC3)cc2cc1N1CCC(=O)NC1=O]

Step 1: tert-Butyl ((3-(2,4-dioxotetrahydropyrimidin-1(2H)-yl)-2-methylquinolin-6-yl)methyl)carbamate (23 mg, 0.06 mmol, 1 equiv) was dissolved in TFA (0.3 mL) and stirred for 1 h at RT. The volatiles were removed in vacuo and the residue was redissolved in DMF (0.58 mL). TEA (3 equiv) was added followed by isocyanatocyclohexane (2 equiv). The reaction was carried out at RT for 24 h. 1-Cyclohexyl-3-((3-(2,4-dioxotetrahydropyrimidin-1(2H)-yl)-2-methylquinolin-6-yl)methyl)urea (6 mg, 0.015 mmol, 25% yield) was purified by preparative HPLC.

LCMS (ESI+) m/z 410.2 [M+H]⁺

¹H NMR (500 MHz, DMSO-d₆) δ 10.51 (s, 1H), 8.24 (s, 1H), 7.90 (d, J=8.6 Hz, 1H), 7.70 (d, J=1.9 Hz, 1H), 7.62 (dd, J=8.6, 2.0 Hz, 1H), 6.32 (t, J=6.1 Hz, 1H), 5.88 (d, J=8.0 Hz, 1H), 4.38 (d, J=6.0 Hz, 2H), 4.00-3.87 (m, 1H), 3.67 (dt, J=12.1, 5.8 Hz, 1H), 3.45-3.36 (m, 1H), 2.88 (ddd, J=16.3, 10.3, 6.1 Hz, 1H), 2.74 (dt, J=11.4, 5.4 Hz, 1H), 2.56 (s, 3H), 1.77 (dd, J=12.6, 4.0 Hz, 2H), 1.64 (dt, J=13.0, 4.0 Hz, 2H), 1.57-1.48 (m, 1H), 1.31-1.22 (m, 2H), 1.17-1.06 (m, 3H).

### Example 41: Synthesis of (3-(2,6-dioxopiperidin-3-yl)-2-methylquinolin-7-yl)methyl (2,3-dichloro-4-methylphenyl)carbamate (Compound 54)

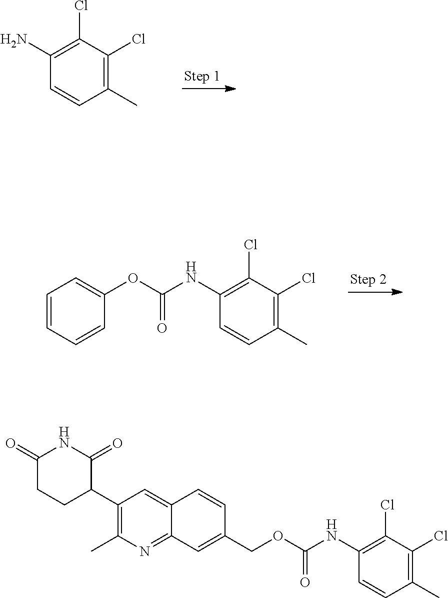

Step 1: Phenyl (2,3-dichloro-4-methylphenyl)carbamate was synthesized using the general procedure shown in Reaction Scheme 6 and Example Method 6, above (45% yield), using 2,3-dichloro-4-methylaniline (200 mg, 1.14 mmol) as starting material.

LCMS (ESI+) m/z 296.1 [M+H]⁺

Step 2: (3-(2,6-Dioxopiperidin-3-yl)-2-methylquinolin-7-yl)methyl (2,3-dichloro-4-methylphenyl)carbamate was synthesized using the general procedure shown in Reaction Scheme 3 and Example Method 3, above (10% yield), using 3-(7-(hydroxymethyl)-2-methylquinolin-3-yl)piperidine-2,6-dione (10 mg, 0.035 mmol, 1 equiv) and phenyl (2,3-dichloro-4-methylphenyl)carbamate (2 equiv) as starting materials, TEA as base and DMF as solvent.

LCMS (ESI+) m/z 486.1 [M+H]⁺

¹H NMR (500 MHz, DMSO-d₆) δ 10.95 (s, 1H), 9.45 (s, 1H), 8.15 (s, 1H), 7.99 (s, 1H), 7.92 (d, J=8.4 Hz, 1H), 7.56 (dd, J=8.4, 1.7 Hz, 1H), 7.50 (d, J=8.3 Hz, 1H), 7.35 (d, J=8.4 Hz, 1H), 5.38 (s, 2H), 4.32 (dd, J=12.5, 4.8 Hz, 1H), 2.85 (ddd, J=17.7, 12.9, 5.3 Hz, 1H), 2.68 (s, 3H), 2.65 (s, 1H), 2.49-2.41 (m, 1H), 2.38 (s, 3H), 2.15 (dtd, J=13.0, 5.2, 3.0 Hz, 1H).

### Example 42: Synthesis of (3-(2,6-dioxopiperidin-3-yl)-2-methylquinolin-7-yl)methyl (4-(tert-butyl)cyclohexyl)carbamate (Compound 55)

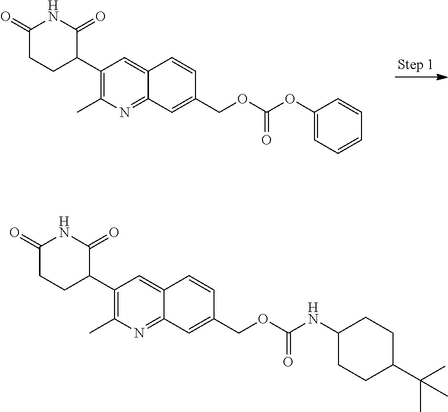 [SMILES: Cc1nc2cc(COC(=O)Oc3ccccc3)ccc2cc1C1CCC(=O)NC1=O ; Cc1nc2cc(COC(=O)NC3CCC(C(C)(C)C)CC3)ccc2cc1C1CCC(=O)NC1=O]

Step 1: To a solution of (3-(2,6-dioxopiperidin-3-yl)-2-methylquinolin-7-yl)methyl phenyl carbonate (35 mg, 0.087 mmol, 1 equiv) and TEA (16 equiv) in DMF (1 mL) was added 4-(tert-butyl)cyclohexan-1-amine (3 equiv) and the resulting mixture was stirred at 50° C. until full conversion was indicated by LCMS. (3-(2,6-Dioxopiperidin-3-yl)-2-methylquinolin-7-yl)methyl (4-(tert-butyl)cyclohexyl)carbamate (18.1 mg, 0.039 mmol, 45% yield) was purified by preparative HPLC.

LCMS (ESI+) m/z 466.3 [M+H]⁺

¹H NMR (500 MHz, DMSO-d₆) δ 10.97 (s, 1H), 8.31 (s, 1H), 7.98-7.90 (m, 2H), 7.57 (t, J=7.7 Hz, 1H), 7.45-7.25 (m, 1H), 5.27-5.22 (m, 2H), 4.34 (dd, J=12.5, 4.6 Hz, 1H), 3.75-3.65 (m, 1H), 2.83 (ddd, J=17.7, 13.0, 5.3 Hz, 1H), 2.72 (s, 3H), 2.63 (d, J=17.1 Hz, 1H), 2.43 (td, J=12.8, 4.2 Hz, 1H), 2.14 (dtd, J=13.0, 5.2, 2.8 Hz, 1H), 1.91-1.84 (m, 1H), 1.75 (dd, J=26.3, 12.5 Hz, 2H), 1.49-1.37 (m, 2H), 1.35-1.22 (m, 1H), 1.15 (qd, J=12.4, 3.1 Hz, 1H), 1.05-0.89 (m, 2H), 0.83 (d, J=4.0 Hz, 9H).

### Example 43: Synthesis of (3-(2,6-dioxopiperidin-3-yl)-2-methylquinolin-7-yl)methyl (5-chloro-6-methylpyridin-3-yl)carbamate (Compound 58)

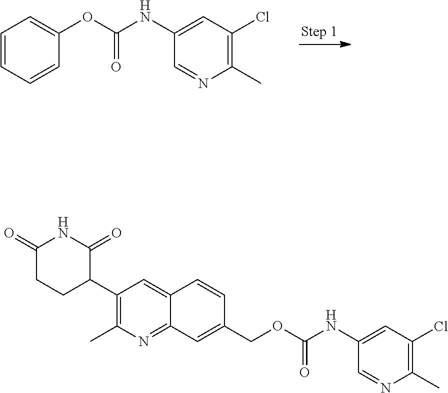 [SMILES: Cc1ncc(NC(=O)Oc2ccccc2)cc1Cl ; Cc1ncc(NC(=O)OCc2ccc3cc(C4CCC(=O)NC4=O)c(C)nc3c2)cc1Cl]

Step 1: (3-(2,6-Dioxopiperidin-3-yl)-2-methylquinolin-7-yl)methyl (5-chloro-6-methylpyridin-3-yl)carbamate was synthesized using the general procedure shown in Reaction Scheme 3 and Example Method 3, above (21% yield), using 3-(7-(hydroxymethyl)-2-methylquinolin-3-yl)piperidine-2,6-dione (15 mg, 0.053 mmol, 1 equiv) and phenyl (5-chloro-6-methylpyridin-3-yl)carbamate (1.2 equiv) as starting materials, TEA as base and DMF as solvent.

The synthesis of phenyl (5-chloro-6-methylpyridin-3-yl)carbamate was described in WO2021067905A1.

LCMS (ESI+) m/z 453.1 [M+H]⁺

¹H NMR (500 MHz, DMSO-d₆) δ 10.93 (s, 1H), 10.21 (s, 1H), 8.47 (d, J=2.3 Hz, 1H), 8.13 (s, 1H), 8.00 (d, J=2.2 Hz, 1H), 7.97-7.93 (m, 1H), 7.91 (d, J=8.4 Hz, 1H), 7.56 (dd, J=8.4, 1.7 Hz, 1H), 5.38 (s, 2H), 4.30 (dd, J=12.5, 4.7 Hz, 1H), 2.88-2.75 (m, 1H), 2.66 (s, 3H), 2.62 (dt, J=17.0, 3.3 Hz, 1H), 2.47 (s, 3H), 2.41 (td, J=12.8, 4.3 Hz, 1H), 2.13 (dtd, J=12.9, 5.2, 3.1 Hz, 1H).

### Example 44: Synthesis of (3-(2,6-dioxopiperidin-3-yl)-2-methylquinolin-7-yl)methyl (5-methoxypyrazin-2-yl)carbamate (Compound 59)

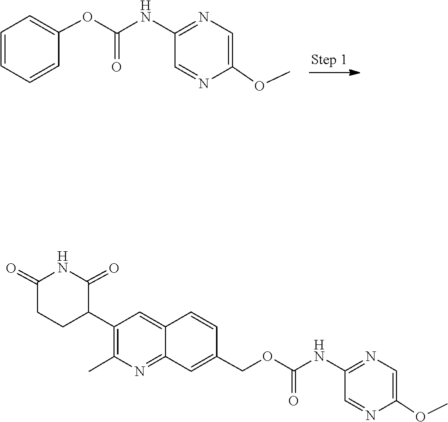 [SMILES: COc1cnc(NC(=O)Oc2ccccc2)cn1 ; COc1cnc(NC(=O)OCc2ccc3cc(C4CCC(=O)NC4=O)c(C)nc3c2)cn1]

Step 1: (3-(2,6-Dioxopiperidin-3-yl)-2-methylquinolin-7-yl)methyl (5-methoxypyrazin-2-yl)carbamate was synthesized using the general procedure shown in Reaction Scheme 3 and Example Method 3, above (21% yield), using 3-(7-(hydroxymethyl)-2-methylquinolin-3-yl)piperidine-2,6-dione (12 mg, 0.049 mmol, 1 equiv) and phenyl (5-methoxypyrazin-2-yl)carbamate (1.3 equiv) as starting materials, TEA as base and DMF as solvent.

The synthesis of phenyl (5-methoxypyrazin-2-yl)carbamate was described in WO2009127944A1.

LCMS (ESI+) m/z 436.1 [M+H]⁺

¹H NMR (500 MHz, DMSO-d₆) δ 10.93 (s, 1H), 10.41 (s, 1H), 8.60 (d, J=1.5 Hz, 1H), 8.13 (s, 1H), 8.07 (d, J=1.4 Hz, 1H), 7.96 (s, 1H), 7.90 (d, J=8.3 Hz, 1H), 7.55 (dd, J=8.4, 1.7 Hz, 1H), 5.39 (s, 2H), 4.29 (dd, J=12.5, 4.7 Hz, 1H), 3.88 (s, 3H), 2.82 (ddd, J=18.0, 12.9, 5.3 Hz, 1H), 2.66 (s, 3H), 2.62 (ddd, J=17.2, 3.7, 2.4 Hz, 1H), 2.42 (qd, J=12.9, 4.3 Hz, 1H), 2.13 (ddq, J=9.9, 5.0, 2.6 Hz, 1H).

### Example 45: Synthesis of (3-(2,4-dioxotetrahydropyrimidin-1(2H)-yl)-2-methylquinolin-6-yl)methyl cyclohexylcarbamate (Compound 60)

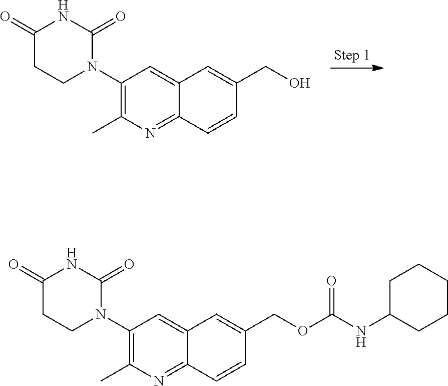 [SMILES: Cc1nc2ccc(CO)cc2cc1N1CCC(=O)NC1=O ; Cc1nc2ccc(COC(=O)NC3CCCCC3)cc2cc1N1CCC(=O)NC1=O]

Step 1: (3-(2,4-Dioxotetrahydropyrimidin-1(2H)-yl)-2-methylquinolin-6-yl)methyl cyclohexylcarbamate was synthesized using the general procedure shown in Reaction Scheme 5 and Example Method 5, above (96% yield), using 1-(6-(hydroxymethyl)-2-methylquinolin-3-yl)dihydropyrimidine-2,4(1H,3H)-dione (8.7 mg, 0.03 mmol, 1 equiv) and isocyanatocyclohexane (2 equiv) as starting materials, TEA (3 equiv) and 1-methyl-1H-imidazole (1 equiv) as bases.

LCMS (ESI+) m/z 411.1 [M+H]⁺

¹H NMR (500 MHz, DMSO-d₆) δ 10.52 (s, 1H), 8.27 (s, 1H), 7.95 (d, J=8.6 Hz, 1H), 7.86 (s, 1H), 7.70 (dd, J=8.7, 1.9 Hz, 1H), 7.24 (d, J=7.9 Hz, 1H), 5.18 (s, 2H), 4.00-3.90 (m, 1H), 3.72-3.64 (m, 1H), 2.93-2.84 (m, 1H), 2.79-2.69 (m, 1H), 2.57 (s, 3H), 1.80-1.74 (m, 2H), 1.70-1.64 (m, 2H), 1.58-1.50 (m, 1H), 1.27-1.03 (m, 6H).

### Example 46: Synthesis of 1-(3-(tert-butyl)isoxazol-5-yl)-3-((3-(2,4-dioxotetrahydropyrimidin-1(2H)-yl)-2-methylquinolin-7-yl)methyl)urea (Compound 64)

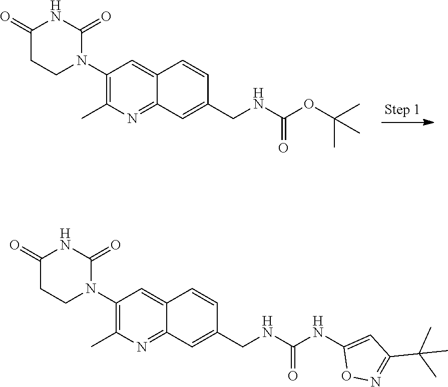 [SMILES: Cc1nc2cc(CNC(=O)OC(C)(C)C)ccc2cc1N1CCC(=O)NC1=O ; Cc1nc2cc(CNC(=O)Nc3cc(C(C)(C)C)no3)ccc2cc1N1CCC(=O)NC1=O]

Step 1: 1-(3-(tert-Butyl)isoxazol-5-yl)-3-((3-(2,4-dioxotetrahydropyrimidin-1(2H)-yl)-2-methylquinolin-7-yl)methyl)urea was synthesized using the general procedure shown in Reaction Scheme 10 and Example Method 10, above (41% yield), using tert-butyl ((3-(2,4-dioxotetrahydropyrimidin-1(2H)-yl)-2-methylquinolin-7-yl)methyl)carbamate (20 mg, 0.052 mmol, 1 equiv) and phenyl (3-(tert-butyl)isoxazol-5-yl)carbamate (1.5 equiv) as starting material, 1-methyl-1H-imidazole (3 equiv) as base and DMF as solvent.

LCMS (ESI+) m/z 451.2 [M+H]⁺

¹H NMR (500 MHz, DMSO-d₆) δ 10.51 (s, 1H), 10.21 (s, 1H), 8.25 (s, 1H), 7.88 (d, J=8.4 Hz, 1H), 7.83-7.80 (m, 1H), 7.50 (dd, J=8.4, 1.7 Hz, 1H), 7.09 (t, J=6.0 Hz, 1H), 5.95 (s, 1H), 4.52 (d, J=6.0 Hz, 2H), 3.92 (td, J=11.1, 10.2, 4.9 Hz, 1H), 3.67 (dt, J=11.9, 5.8 Hz, 1H), 2.88 (ddd, J=16.3, 10.0, 6.1 Hz, 1H), 2.73 (dt, J=16.6, 5.2 Hz, 1H), 2.56 (s, 3H), 1.23 (s, 9H).

### Example 47: Synthesis of (3-(2,4-dioxotetrahydropyrimidin-1(2H)-yl)-2-methylquinolin-6-yl)methyl (3-(tert-butyl)isoxazol-5-yl)carbamate (Compound 66)

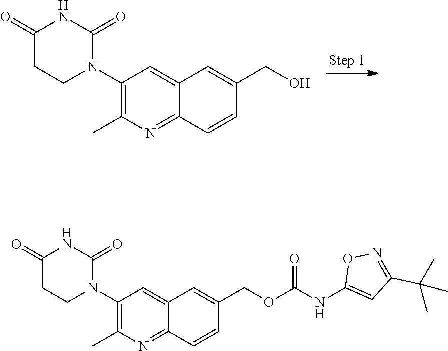 [SMILES: Cc1nc2ccc(CO)cc2cc1N1CCC(=O)NC1=O ; Cc1nc2ccc(COC(=O)Nc3cc(C(C)(C)C)no3)cc2cc1N1CCC(=O)NC1=O]

Step 1: (3-(2,4-Dioxotetrahydropyrimidin-1(2H)-yl)-2-methylquinolin-6-yl)methyl (3-(tert-butyl)isoxazol-5-yl)carbamate was synthesized using the general procedure shown in Reaction Scheme 3 and Example Method 3, above (17% yield), using 1-(6-(hydroxymethyl)-2-methylquinolin-3-yl)dihydropyrimidine-2,4(1H,3H)-dione (7.0 mg, 0.025 mmol, 1 equiv) and phenyl (3-(tert-butyl)isoxazol-5-yl)carbamate (1.2 equiv) as starting materials, 1-methyl-1H-imidazole (1 equiv) as base and NMP as solvent.

LCMS (ESI+) m/z 452.1 [M+H]⁺

¹H NMR (500 MHz, DMSO-d₆) δ 11.41 (s, 1H), 10.53 (s, 1H), 8.31 (s, 1H), 7.98 (d, J=8.6 Hz, 1H), 7.93 (d, J=1.9 Hz, 1H), 7.76 (dd, J=8.7, 2.0 Hz, 1H), 6.01 (s, 1H), 5.38 (s, 2H), 4.01-3.89 (m, 1H), 3.74-3.61 (m, 1H), 2.94-2.82 (m, 1H), 2.81-2.68 (m, 1H), 2.58 (s, 3H), 1.23 (s, 9H).

### Example 48: Synthesis of 1-(3-(tert-butyl)isoxazol-5-yl)-3-((3-(2,4-dioxotetrahydropyrimidin-1(2H)-yl)-2-methylquinolin-6-yl)methyl)urea (Compound 67)

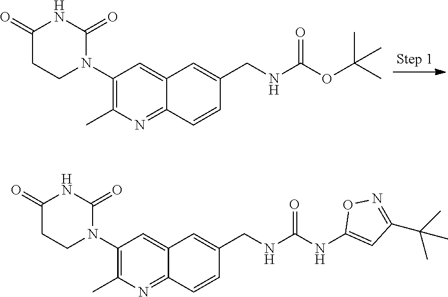 [SMILES: Cc1nc2ccc(CNC(=O)OC(C)(C)C)cc2cc1N1CCC(=O)NC1=O ; Cc1nc2ccc(CNC(=O)Nc3cc(C(C)(C)C)no3)cc2cc1N1CCC(=O)NC1=O]

Step 1: tert-Butyl ((3-(2,4-dioxotetrahydropyrimidin-1(2H)-yl)-2-methylquinolin-6-yl)methyl)carbamate (12.8 mg, 0.033 mmol, 1 equiv) was solubilized in TFA (0.5 mL) and stirred at RT for 1 h. The volatiles were then removed in vacuo. To the resulting residue were added NMP (1 mL), 1-methyl-1H-imidazole (3 equiv) and phenyl (3-(tert-butyl)isoxazol-5-yl)carbamate (1.2 equiv). The reaction was carried out at RT for 18 h. 1-(3-(tert-Butyl)isoxazol-5-yl)-3-((3-(2,4-dioxotetrahydropyrimidin-1(2H)-yl)-2-methylquinolin-6-yl)methyl)urea (3.9 mg, 26% yield) was purified by preparative HPLC, then by preparative TLC and again by preparative HPLC.

LCMS (ESI+) m/z 451.1 [M+H]⁺

¹H NMR (500 MHz, DMSO-d₆) δ 10.51 (s, 1H), 10.24 (s, 1H), 8.27 (s, 1H), 7.93 (d, J=8.7 Hz, 1H), 7.75 (d, J=1.9 Hz, 1H), 7.67 (dd, J=8.7, 2.0 Hz, 1H), 7.11 (t, J=6.1 Hz, 1H), 5.94 (s, 1H), 4.49 (d, J=6.0 Hz, 2H), 3.98-3.88 (m, 1H), 3.72-3.62 (m, 1H), 2.94-2.84 (m, 1H), 2.79-2.69 (m, 1H), 2.56 (s, 3H), 1.22 (s, 9H).

### Example 49: Synthesis of (3-(2,6-dioxopiperidin-3-yl)-2,6-dimethylquinolin-7-yl)methyl (3-chloro-4-methylphenyl)carbamate (Compound 68)

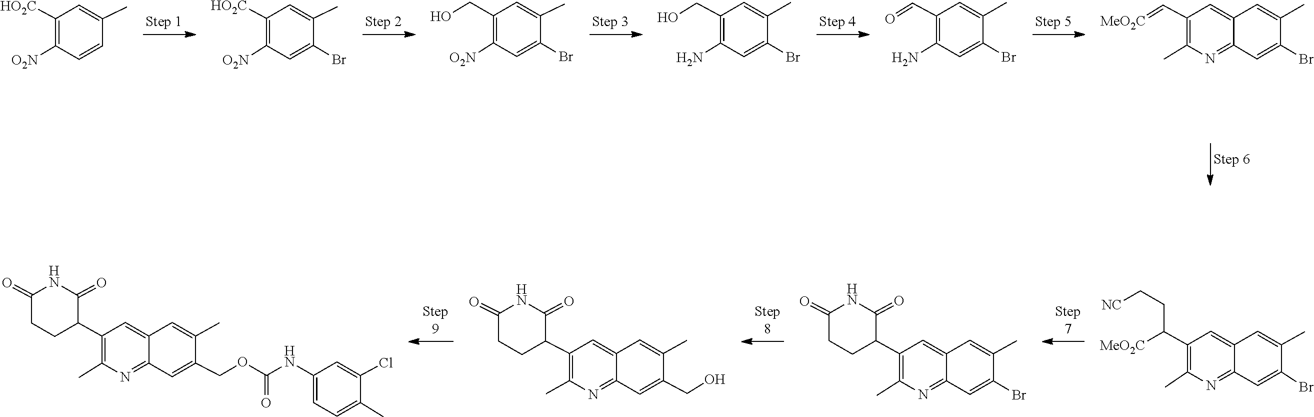

Step 1: To a solution of 5-methyl-2-nitrobenzoic acid (6 g, 33 mmol, 1 equiv) in concentrated sulfuric acid (20 mL) was added NBS (1.3 equiv) portionwise over the period of 150 min at 60° C. and the mixture was held at this temperature for additional 30 min. After that time the reaction mixture was poured onto crushed ice and 4-bromo-5-methyl-2-nitrobenzoic acid (6.5 g, 21 mmol, 65% yield) was collected by filtration, air-dried and directly forwarded into the next step.

LCMS (ESI+) m/z not detected.

Step 2: To a solution of 4-bromo-5-methyl-2-nitrobenzoic acid (1.5 g, 5.77 mmol, 1 equiv) in THF (15 mL) borane dimethyl sulfide complex (1.6 mL, 17.3 mmol, 3 equiv) was slowly added at 0° C. The reaction was stirred at 60° C. for 16 h. The reaction was cooled to 0° C. and quenched with methanol.

The resulting solution was washed with 2M sodium hydroxide solution, brine and organic phase was dried over Na₂SO₄ and evaporated. The crude (4-bromo-5-methyl-2-nitrophenyl)methanol (1.4 g, 5.54 mmol, 96% yield) was used directly for the next step.

LCMS (ESI+) m/z not detected.

Step 3: (4-Bromo-5-methyl-2-nitrophenyl)methanol (1.5 g, 6 mmol, 1 equiv) was taken up in ethanol (16 mL) and water (2 mL). Iron (5 equiv) and NH₄Cl (5 equiv) were added and the reaction was stirred in a sealed tube at 80° C. for 18 h. After completion, the solid particles were filtered and ethanol was evaporated. The residue was extracted with ethyl acetate, organic phase was dried over Na₂SO₄ and evaporated. (2-Amino-4-bromo-5-methylphenyl)methanol (850 mg, 4 mmol, 61% yield) was purified by flash column chromatography.

LCMS (ESI+) m/z not detected.

Step 4: (2-Amino-4-bromo-5-methylphenyl)methanol (800 mg, 3.7 mmol, 1 equiv) and MnO₂ (3 equiv) in DCM (28 mL) were stirred at RT for 20 h. After completion, the reaction was diluted with water and extracted with ethyl acetate. The combined organic fractions were dried over Na₂SO₄ and evaporated to yield crude 2-amino-4-bromo-5-methylbenzaldehyde (400 mg, 1.869 mmol, 50% yield) was directly forwarded into the next step.

LCMS (ESI+) m/z 213.9 [M+H]⁺

Step 5: 2-Amino-4-bromo-5-methylbenzaldehyde (380 mg, 1.775 mmol, 1 equiv) and 4-oxopentanoic acid (1 equiv) were dissolved in methanol (5.3 mL), purged with argon for 15 min. Sodium hydroxide (2M solution, 1.1 mL, 1.2 equiv) was added and the reaction mixture was stirred at 75° C. for 18 h. The reaction was then cooled, acidified with acetic acid, diluted with water and concentrated in vacuo to remove methanol. The crude 2-(7-bromo-2,6-dimethylquinolin-3-yl)acetic acid was filtered, washed with water and vacuum-dried. The product was redissolved in methanol (5.3 mL) and thionyl chloride (0.26 mL, 3.55 mmol, 2 equiv) was added dropwise. The reaction mixture was refluxed for 18 h. After completion, the reaction mixture was concentrated, diluted with DCM and saturated solution of NaHCO₃. The product was extracted with DCM, combined organic fractions were dried over Na₂SO₄ and concentrated. Methyl 2-(7-bromo-2,6-dimethylquinolin-3-yl)acetate (250 mg, 0.789 mmol, 44% yield) was purified by flash column chromatography.

LCMS (ESI+) m/z 309.7 [M+H]⁺

Step 6: Methyl 2-(7-bromo-2,6-dimethylquinolin-3-yl)-4-cyanobutanoate was synthesized using the general procedure shown in Reaction Scheme 8 and Example Method 8, above (13% yield), using methyl 2-(7-bromo-2,6-dimethylquinolin-3-yl)acetate (250 mg, 0.81 mmol, 1 equiv) and acrylonitrile (1 equiv) as starting materials.

LCMS (ESI+) m/z not detected

Step 7: 3-(7-Bromo-2,6-dimethylquinolin-3-yl)piperidine-2,6-dione was synthesized using the general procedure shown in Reaction Scheme 9 and Example Method 9, above (84% yield), using methyl 2-(7-bromo-2,6-dimethylquinolin-3-yl)-4-cyanobutanoate (52 mg, 0.144 mmol) as starting material.

LCMS (ESI+) m/z 347.1 [M+H]⁺

Step 8: 3-(7-(Hydroxymethyl)-2,6-dimethylquinolin-3-yl)piperidine-2,6-dione was synthesized using the general procedure shown in Reaction Scheme 7 and Example Method 7, above (26% yield), using 3-(7-bromo-2,6-dimethylquinolin-3-yl)piperidine-2,6-dione (45 mg, 0.13 mmol 1 equiv) and (tributylstannyl)methanol (1.05 equiv) as starting materials and tetrakis(triphenylphosphine)palladium(0) (0.1 equiv) as catalyst and dioxane as solvent.

LCMS (ESI+) m/z 299.1 [M+H]⁺

Step 9: (3-(2,6-Dioxopiperidin-3-yl)-2,6-dimethylquinolin-7-yl)methyl (3-chloro-4-methylphenyl)carbamate was synthesized using the general procedure shown in Reaction Scheme 3 and Example Method 3, above (16% yield), using 3-(7-(hydroxymethyl)-2,6-dimethylquinolin-3-yl)piperidine-2,6-dione (15 mg, 0.05 mmol, 1 equiv) and phenyl (3-chloro-4-methylphenyl)carbamate (1 equiv) as starting materials, TEA as base, and DMF as solvent.

LCMS (ESI+) m/z 466.1 [M+H]⁺

¹H NMR (500 MHz, DMSO-d₆) δ 10.92 (s, 1H), 9.99 (s, 1H), 8.02 (s, 1H), 7.93 (s, 1H), 7.72-7.66 (m, 1H), 7.65-7.58 (m, 1H), 7.32 (dd, J=8.3, 2.2 Hz, 1H), 7.26 (d, J=8.4 Hz, 1H), 5.35 (s, 2H), 4.27 (dd, J=12.4, 4.8 Hz, 1H), 2.87-2.75 (m, 1H), 2.64-2.59 (m, 4H), 2.48 (s, 3H), 2.43-2.38 (m, 1H), 2.26 (s, 3H), 2.14-2.09 (m, 1H).

### Example 50: Synthesis of (3-(2,6-dioxopiperidin-3-yl)-2-methylquinolin-7-yl)methyl (3-(trifluoromethyl)-1H-pyrazol-5-yl)carbamate (Compound 69)

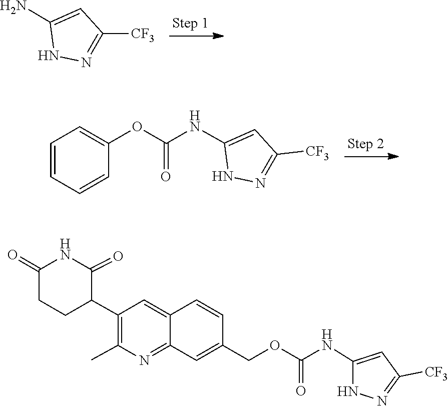 [SMILES: Nc1cc(C(F)(F)F)n[nH]1 ; O=C(Nc1cc(C(F)(F)F)n[nH]1)Oc1ccccc1 ; Cc1nc2cc(COC(=O)Nc3cc(C(F)(F)F)n[nH]3)ccc2cc1C1CCC(=O)NC1=O]

Step 1: Phenyl (3-(trifluoromethyl)-1H-pyrazol-5-yl)carbamate was synthesized using the general procedure shown in Reaction Scheme 6 and Example Method 6, above (48% yield), using 3-(trifluoromethyl)-1H-pyrazol-5-amine (300 mg, 1.99 mmol) as starting material.

LCMS (ESI+) m/z 272.0 [M+H]⁺

Step 2: (3-(2,6-Dioxopiperidin-3-yl)-2-methylquinolin-7-yl)methyl (3-(trifluoromethyl)-1H-pyrazol-5-yl)carbamate was synthesized using the general procedure shown in Reaction Scheme 3 and Example Method 3, above (8% yield), using phenyl (3-(trifluoromethyl)-1H-pyrazol-5-yl)carbamate (17 mg, 0.063 mmol, 1 equiv) and 3-(7-(hydroxymethyl)-2-methylquinolin-3-yl)piperidine-2,6-dione (1.2 equiv), TEA as base and DMF as solvent.

LCMS (ESI+) m/z 462.1 [M+H]⁺

¹H NMR (500 MHz, DMSO-d₆) δ 13.43 (s, 1H), 10.93 (s, 1H), 10.69 (s, 1H), 8.13 (s, 1H), 7.95 (d, J=1.8 Hz, 1H), 7.91 (d, J=8.4 Hz, 1H), 7.55 (dd, J=8.4, 1.7 Hz, 1H), 6.35 (s, 1H), 5.39 (s, 2H), 4.30 (dd, J=12.5, 4.7 Hz, 1H), 2.82 (ddd, J=17.7, 12.9, 5.3 Hz, 1H), 2.66 (s, 3H), 2.64-2.59 (m, 1H), 2.46-2.38 (m, 1H), 2.13 (dtd, J=13.0, 5.2, 2.8 Hz, 1H).

### Example 51: Synthesis of (3-(2,6-dioxopiperidin-3-yl)-2-methylquinolin-7-yl)methyl (5-(tert-butyl)isoxazol-3-yl)carbamate (Compound 70)

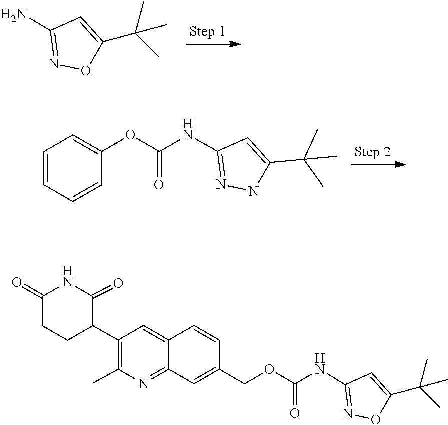

Step 1: Phenyl (5-(tert-butyl)isoxazol-3-yl)carbamate was synthesized using the general procedure shown in Reaction Scheme 6 and Example Method 6, above (20% yield), using 5-(tert-butyl)isoxazol-3-amine (500 mg, 3.57 mmol) as starting material.

LCMS (ESI+) m/z 261.2 [M+H]⁺

Step 2: (3-(2,6-Dioxopiperidin-3-yl)-2-methylquinolin-7-yl)methyl (5-(tert-butyl)isoxazol-3-yl)carbamate was synthesized using the general procedure shown in Reaction Scheme 3 and Example Method 3, above (5% yield), using phenyl (5-(tert-butyl)isoxazol-3-yl)carbamate (17 mg, 0.065 mmol, 1 equiv) and 3-(7-(hydroxymethyl)-2-methylquinolin-3-yl)piperidine-2,6-dione (1.2 equiv), TEA as base and DMF as solvent.

LCMS (ESI+) m/z 451.1 [M+H]⁺

¹H NMR (500 MHz, DMSO-d₆) δ 10.93 (s, 1H), 10.78 (s, 1H), 8.13 (s, 1H), 7.96-7.91 (m, 1H), 7.90 (d, J=8.4 Hz, 1H), 7.53 (dd, J=8.4, 1.7 Hz, 1H), 6.44 (s, 1H), 5.38 (s, 2H), 4.29 (dd, J=12.5, 4.7 Hz, 1H), 2.82 (ddd, J=17.7, 12.9, 5.3 Hz, 1H), 2.66 (s, 3H), 2.64-2.59 (m, 1H), 2.45-2.38 (m, 1H), 2.12 (dtd, J=12.9, 5.1, 2.9 Hz, 1H), 1.28 (s, 9H).

### Example 52: Synthesis of (3-(2,6-dioxopiperidin-3-yl)-2-methylquinolin-7-yl)methyl (3-(trifluoromethyl)isoxazol-5-yl)carbamate (Compound 71)

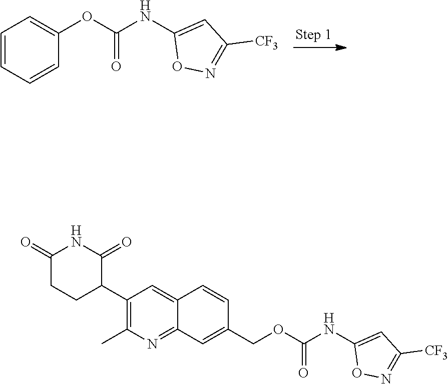 [SMILES: O=C(Nc1cc(C(F)(F)F)no1)Oc1ccccc1 ; Cc1nc2cc(COC(=O)Nc3cc(C(F)(F)F)no3)ccc2cc1C1CCC(=O)NC1=O]

Step 1: (3-(2,6-Dioxopiperidin-3-yl)-2-methylquinolin-7-yl)methyl (3-(trifluoromethyl)isoxazol-5-yl)carbamate was synthesized using the general procedure shown in Reaction Scheme 3 and Example Method 3, above (7% yield), using phenyl (3-(trifluoromethyl)isoxazol-5-yl)carbamate (17 mg, 0.062 mmol, 1 equiv) and 3-(7-(hydroxymethyl)-2-methylquinolin-3-yl)piperidine-2,6-dione (1.2 equiv), TEA as base and DMF as solvent.

The synthesis of phenyl (3-(trifluoromethyl)isoxazol-5-yl)carbamate was described in Rowbottom, M. W. et al., J. Med. Chem. 2012, 55, 1082.

LCMS (ESI+) m/z 463.0 [M+H]⁺

¹H NMR (500 MHz, DMSO-d₆) δ 12.25 (s, 1H), 10.93 (s, 1H), 8.13 (s, 1H), 7.96-7.92 (m, 1H), 7.90 (d, J=8.4 Hz, 1H), 7.55 (dd, J=8.4, 1.7 Hz, 1H), 6.49-6.25 (m, 1H), 5.40 (s, 2H), 4.29 (dd, J=12.5, 4.7 Hz, 1H), 2.87-2.77 (m, 1H), 2.66 (s, 3H), 2.63-2.59 (m, 1H), 2.45-2.37 (m, 1H), 2.13 (dtd, J=13.0, 5.2, 3.3 Hz, 1H).

### Example 53: Synthesis of (3-(2,4-dioxotetrahydropyrimidin-1(2H)-yl)-2-methylquinolin-7-yl)methyl (3-(tert-butyl)isoxazol-5-yl)carbamate (Compound 72)

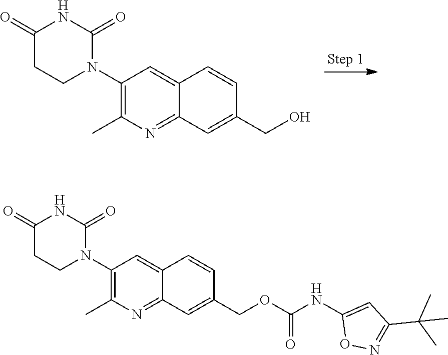 [SMILES: Cc1nc2cc(CO)ccc2cc1N1CCC(=O)NC1=O ; Cc1nc2cc(COC(=O)Nc3cc(C(C)(C)C)no3)ccc2cc1N1CCC(=O)NC1=O]

Step 1: (3-(2,4-Dioxotetrahydropyrimidin-1(2H)-yl)-2-methylquinolin-7-yl)methyl (3-(tert-butyl)isoxazol-5-yl)carbamate was synthesized using the general procedure shown in Reaction Scheme 3 and Example Method 3, above (14% yield), using 1-(7-(hydroxymethyl)-2-methylquinolin-3-yl)dihydropyrimidine-2,4(1H,3H)-dione (25.9 mg, 0.091 mmol, 1 equiv) and phenyl (3-(tert-butyl)isoxazol-5-yl)carbamate (1.5 equiv) as starting materials, 1-methyl-1H-imidazole (5 equiv) as base and NMP as solvent.

LCMS (ESI+) m/z 452.2 [M+H]⁺

¹H NMR (500 MHz, DMSO-d₆) δ 11.46 (s, 1H), 10.55 (s, 1H), 8.38 (s, 1H), 8.02-7.96 (m, 2H), 7.62 (dd, J=8.4, 1.7 Hz, 1H), 6.03 (s, 1H), 5.44 (s, 2H), 3.99-3.91 (m, 1H), 3.72-3.65 (m, 1H), 2.94-2.85 (m, 1H), 2.77-2.70 (m, 1H), 2.60 (s, 3H), 1.24 (s, 9H).

### Example 54: Synthesis of (3-(3-fluoro-2,6-dioxopiperidin-3-yl)-2-methylquinolin-7-yl)methyl (3-chloro-4-methylphenyl)carbamate (Compound 74)

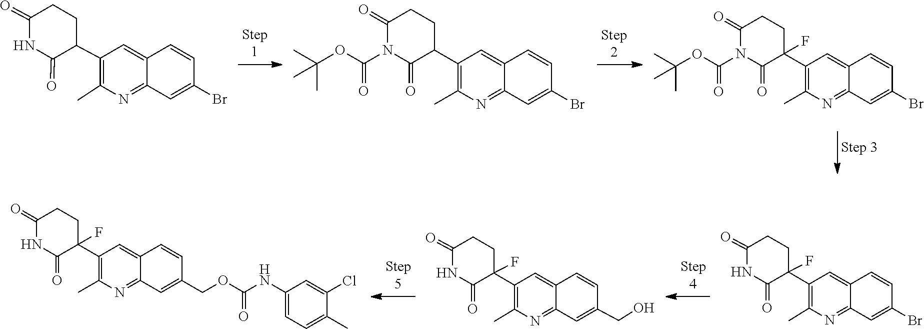 [SMILES: Cc1nc2cc(Br)ccc2cc1C1CCC(=O)NC1=O ; Cc1nc2cc(Br)ccc2cc1C1CCC(=O)N(C(=O)OC(C)(C)C)C1=O ; Cc1nc2cc(Br)ccc2cc1C1(F)CCC(=O)N(C(=O)OC(C)(C)C)C1=O ; Cc1ccc(NC(=O)OCc2ccc3cc(C4(F)CCC(=O)NC4=O)c(C)nc3c2)cc1Cl ; Cc1nc2cc(CO)ccc2cc1C1(F)CCC(=O)NC1=O ; Cc1nc2cc(Br)ccc2cc1C1(F)CCC(=O)NC1=O]

Step 1: 3-(7-Bromo-2-methylquinolin-3-yl)piperidine-2,6-dione (300 mg, 0.9 mmol, 1 equiv), DMAP (0.1 equiv) and di-tert-butyl dicarbonate (3.5 equiv) were dissolved in dioxane (29 mL) and stirred in a sealed tube at RT for 18 h. After completion the volatiles were removed in vacuo and tert-butyl 3-(7-bromo-2-methylquinolin-3-yl)-2,6-dioxopiperidine-1-carboxylate (298 mg, 76% yield) was purified by flash column chromatography.

LCMS (ESI+) m/z 432.8 [M+H]⁺

Step 2: To a solution of tert-butyl 3-(7-bromo-2-methylquinolin-3-yl)-2,6-dioxopiperidine-1-carboxylate (100 mg, 0.231 mmol, 1 equiv) in dry THF (5 mL), cooled to −78° C., was added 1M solution of LiHMDS in THF (0.36 mL, 0.36 mmol, 1.2 equiv) and the reaction was stirred at the same temperature for 2 h. Then the solution of NFSI (1.3 equiv) in THF (10 mL) was added dropwise and the resulting mixture was stirred overnight at RT. After completion, the reaction was quenched with saturated NH₄Cl solution and extracted with ethyl acetate. The combined organic fractions were dried over Na₂SO₄ and evaporated. tert-Butyl 3-(7-bromo-2-methylquinolin-3-yl)-3-fluoro-2,6-dioxopiperidine-1-carboxylate (65 mg, 48% yield) was purified by flash column chromatography.

LCMS (ESI+) m/z 450.9 [M+H]⁺

Step 3: tert-Butyl 3-(7-bromo-2-methylquinolin-3-yl)-3-fluoro-2,6-dioxopiperidine-1-carboxylate (65 mg, 0.144 mmol, 1 equiv) was dissolved in dry DCM (2.2 mL) and TFA (20 equiv) was added at RT. The reaction was carried out at RT for 1 h. After completion the reaction mixture was diluted with ethyl acetate, washed with saturated NaHCO₃ solution, dried over Na₂SO₄ and evaporated. Crude 3-(7-bromo-2-methylquinolin-3-yl)-3-fluoropiperidine-2,6-dione (43.7 mg) was directly forwarded into the next step.

LCMS (ESI+) m/z 350.8 [M+H]⁺

Step 4: 3-Fluoro-3-(7-(hydroxymethyl)-2-methylquinolin-3-yl)piperidine-2,6-dione was synthesized using the general procedure shown in Reaction Scheme 7 and Example Method 7, above (63% yield), using 3-(7-bromo-2-methylquinolin-3-yl)-3-fluoropiperidine-2,6-dione (23 mg, 0.065 mmol, 1 equiv, crude from the earlier step) and (tributylstannyl)methanol (4 equiv) as starting materials, tetrakis(triphenylphosphine)palladium(0) (0.1 equiv) as catalyst and dioxane as solvent.

LCMS (ESI+) m/z 302.8 [M+H]⁺

Step 5: (3-(3-Fluoro-2,6-dioxopiperidin-3-yl)-2-methylquinolin-7-yl)methyl (3-chloro-4-methylphenyl)carbamate was synthesized using the general procedure shown in Reaction Scheme 5 and Example Method 5, above (25% yield), using 3-fluoro-3-(7-(hydroxymethyl)-2-methylquinolin-3-yl)piperidine-2,6-dione (10 mg, 0.033 mmol, 1 equiv) and 2-chloro-4-isocyanato-1-methylbenzene (2 equiv) as starting materials, TEA (3 equiv) and 1-methyl-1H-imidazole (1 equiv) as bases, DMF as solvent.

LCMS (ESI+) m/z 470.1 [M+H]⁺

¹H NMR (500 MHz, DMSO-d₆) δ 11.30 (s, 1H), 9.97 (s, 1H), 8.25 (s, 1H), 7.96 (s, 1H), 7.88 (d, J=8.4 Hz, 1H), 7.64-7.59 (m, 1H), 7.56 (dd, J=8.4, 1.7 Hz, 1H), 7.31 (dd, J=8.3, 2.2 Hz, 1H), 7.25 (d, J=8.3 Hz, 1H), 5.59 (ddd, J=47.3, 12.4, 5.8 Hz, 1H), 5.36 (s, 2H), 4.53 (dd, J=13.5, 4.2 Hz, 1H), 2.89-2.78 (m, 1H), 2.67 (s, 3H), 2.62-2.55 (m, 1H), 2.25 (s, 3H).

### Example 55: Synthesis of (3-(2,6-dioxopiperidin-3-yl)-2-methylquinolin-7-yl)methyl (3-(tert-butyl)-1H-pyrazol-5-yl)carbamate (Compound 75)

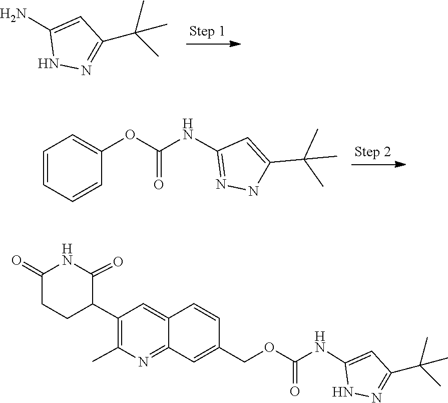

Step 1: Phenyl (3-(tert-butyl)-1H-pyrazol-5-yl)carbamate was synthesized using the general procedure shown in Reaction Scheme 6 and Example Method 6, above (23% yield), using 3-(tert-butyl)-1H-pyrazol-5-amine (200 mg, 1.44 mmol) as starting material.

LCMS (ESI+) m/z 260.1 [M+H]⁺

Step 2: (3-(2,6-Dioxopiperidin-3-yl)-2-methylquinolin-7-yl)methyl (3-(tert-butyl)-1H-pyrazol-5-yl)carbamate was synthesized using the general procedure shown in Reaction Scheme 3 and Example Method 3, above (6% yield), using 3-(7-(hydroxymethyl)-2-methylquinolin-3-yl)piperidine-2,6-dione (20 mg, 0.07 mmol, 1 equiv) and phenyl (3-(tert-butyl)-1H-pyrazol-5-yl)carbamate (1 equiv) as starting materials, 1-methyl-1H-imidazole (2 equiv) as base and NMP as solvent.

LCMS (ESI+) m/z 450.2 [M+H]⁺

¹H NMR (500 MHz, DMSO-d₆) δ 11.97 (s, 1H), 10.95 (s, 1H), 9.96 (s, 1H), 8.14 (s, 1H), 7.94-7.92 (m, 1H), 7.91 (d, J=8.4 Hz, 1H), 7.53 (dd, J=8.4, 1.7 Hz, 1H), 6.11 (s, 1H), 5.34 (s, 2H), 4.31 (dd, J=12.5, 4.7 Hz, 1H), 2.84 (ddd, J=17.7, 12.9, 5.3 Hz, 1H), 2.68 (s, 3H), 2.65-2.60 (m, 1H), 2.44 (m, 1H), 2.19-2.07 (m, 1H), 1.26 (s, 9H).

### Example 56: Synthesis of (3-(2,6-dioxopiperidin-3-yl)-2-methylquinolin-7-yl)methyl (3-(tert-butyl)-1,2,4-oxadiazol-5-yl)carbamate (Compound 80)

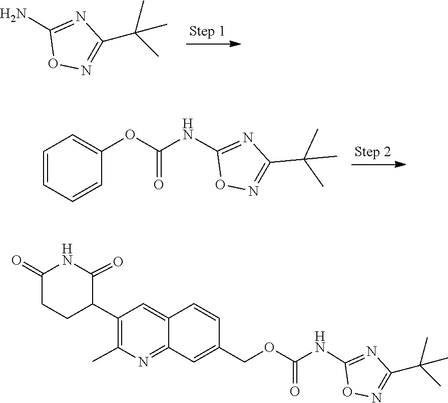

Step 1: Phenyl (3-(tert-butyl)-1,2,4-oxadiazol-5-yl)carbamate was synthesized using the general procedure shown in Reaction Scheme 6 and Example Method 6, above (52% yield), using 3-(tert-butyl)-1,2,4-oxadiazol-5-amine (34 mg, 0.24 mmol) as starting material.

LCMS (ESI+) m/z 261.8 [M+H]⁺

Step 2: (3-(2,6-Dioxopiperidin-3-yl)-2-methylquinolin-7-yl)methyl (3-(tert-butyl)-1,2,4-oxadiazol-5-yl)carbamate was synthesized using the general procedure shown in Reaction Scheme 4 and Example Method 4, above (9% yield), using 3-(7-(hydroxymethyl)-2-methylquinolin-3-yl)piperidine-2,6-dione (25 mg, 0.088 mmol, 1 equiv) and phenyl (3-(tert-butyl)-1,2,4-oxadiazol-5-yl)carbamate (1.3 equiv) as starting materials, sodium hydride as base and DMF as solvent.

LCMS (ESI+) m/z 452.1 [M+H]⁺

¹H NMR (500 MHz, DMSO-d₆) δ 12.21 (s, 1H), 10.88 (s, 1H), 8.12 (s, 1H), 7.96 (s, 1H), 7.90 (d, J=8.4 Hz, 1H), 7.54 (dd, J=8.4, 1.7 Hz, 1H), 5.39 (s, 2H), 4.29 (dd, J=12.5, 4.7 Hz, 1H), 2.82 (ddd, J=17.7, 12.9, 5.3 Hz, 1H), 2.66 (s, 3H), 2.65-2.60 (m, 1H), 2.42 (qd, J=12.8, 4.2 Hz, 1H), 2.17-2.10 (m, 1H), 1.27 (s, 9H).

### Example 57: Synthesis of (3-(2,6-dioxopiperidin-3-yl)-2-methylquinolin-7-yl)methyl (3-(1-(trifluoromethyl)cyclopropyl)isoxazol-5-yl)carbamate (Compound 81)

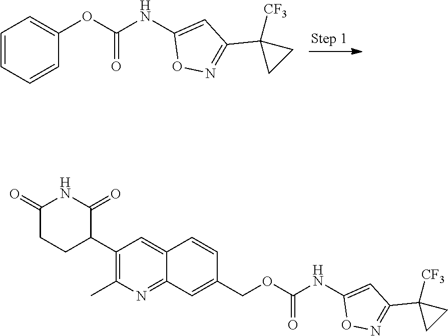 [SMILES: O=C(Nc1cc(C2(C(F)(F)F)CC2)no1)Oc1ccccc1 ; Cc1nc2cc(COC(=O)Nc3cc(C4(C(F)(F)F)CC4)no3)ccc2cc1C1CCC(=O)NC1=O]

Step 1: (3-(2,6-Dioxopiperidin-3-yl)-2-methylquinolin-7-yl)methyl (3-(1-(trifluoromethyl)cyclopropyl)isoxazol-5-yl)carbamate was synthesized using the general procedure shown in Reaction Scheme 3 and Example Method 3, above (55% yield), using 3-(7-(hydroxymethyl)-2-methylquinolin-3-yl)piperidine-2,6-dione (30.4 mg, 0.107 mmol, 1 equiv) and phenyl (3-(1-(trifluoromethyl)cyclopropyl)isoxazol-5-yl)carbamate (1.2 equiv) as starting materials, 1-methyl-1H-imidazole (2 equiv) as base and DMF as solvent.

The synthesis of phenyl (3-(1-(trifluoromethyl)cyclopropyl)isoxazol-5-yl)carbamate was described in U.S. Ser. No. 11/161,852B1.

LCMS (ESI+) m/z 503.2 [M+H]⁺

¹H NMR (500 MHz, DMSO-d₆) δ 11.73 (s, 1H), 10.93 (s, 1H), 8.13 (s, 1H), 7.96-7.93 (m, 1H), 7.91 (d, J=8.3, 1H), 7.54 (dd, J=8.4, 1.7, 1H), 6.11 (s, 1H), 5.41 (s, 2H), 4.30 (dd, J=12.5, 4.7, 1H), 2.82 (ddd, J=17.7, 12.9, 5.3, 1H), 2.66 (s, 3H), 2.63-2.57 (m, 1H), 2.42 (qd, J=12.9, 4.3, 1H), 2.16-2.09 (m, 1H), 1.46-1.40 (m, 2H), 1.40-1.33 (m, 2H).

### Example 58: Synthesis of (3-(2,6-dioxopiperidin-3-yl)-2-methylquinolin-7-yl)methyl-d₂ (3-(tert-butyl)isoxazol-5-yl)carbamate (Compound 82)

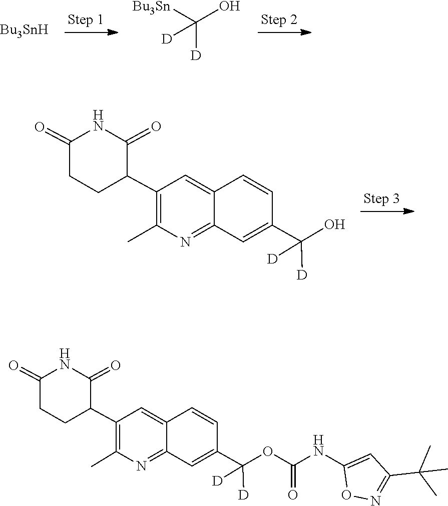

Step 1: To a stirred solution of LDA (221 mg, 2.06 mmol, 1.5 equiv) in THF (1M solution, 2 mL), cooled to 0° C., was added solution of tributylstannane (400 mg, 1.37 mmol, 1 equiv) in dry THF (2 mL) under argon. The resulting mixture was stirred at 0° C. for 20 min. Then the suspension of paraformaldehyde-d₂ (1.5 equiv) in dry THF was slowly added under argon purge and the reaction mixture was stirred at RT for 1 h. The reaction was quenched with water, extracted with ethyl acetate, the combined organic fractions were dried over Na₂SO₄ and evaporated. (Tributylstannyl)methan-d₂-ol (270 mg, 56% yield) was purified by flash column chromatography.

LCMS (ESI+) m/z not detected.

Step 2: 3-(7-(Hydroxymethyl-d₂)-2-methylquinolin-3-yl)piperidine-2,6-dione was synthesized using the general procedure shown in Reaction Scheme 7 and Example Method 7, above (55% yield), using 3-(7-bromo-2-methylquinolin-3-yl)piperidine-2,6-dione (51 mg, 0.153 mmol, 1 equiv) and (tributylstannyl)methan-d₂-ol (1.04 equiv) as starting materials, tetrakis(triphenylphosphine)palladium(0) (0.07 equiv) as catalyst and dioxane as solvent.

LCMS (ESI+) m/z 286.9 [M+H]⁺

Step 3: To a solution of bis(trichloromethyl) carbonate (67.5 mg, 0.23 mmol, 2.1 equiv) in dry ACN (2 mL) was added tetraethylammonium chloride (0.5 equiv) and the mixture was stirred at 50° C. for 3.5 h, then cooled. A solution of 3-(tert-butyl)isoxazol-5-amine (6 equiv) in dry ACN (2 mL) was added dropwise to the reaction mixture and stirred at RT for 30 min. Then the solution of 3-(7-(hydroxymethyl-d₂)-2-methylquinolin-3-yl)piperidine-2,6-dione (31 mg, 0.108 mmol, 1 equiv) and DIPEA (1 equiv) in dry DMF (1 mL) and dry ACN (1 mL) was added to the reaction mixture and further stirred at RT for 60 h. The volatiles were then removed in vacuo. (3-(2,6-Dioxopiperidin-3-yl)-2-methylquinolin-7-yl)methyl-d₂ (3-(tert-butyl)isoxazol-5-yl)carbamate (10 mg, 0.015 mmol, 14% yield) was purified by flash column chromatography and repurified by preparative HPLC.

LCMS (ESI+) m/z 453.2 [M+H]⁺

¹H NMR (500 MHz, DMSO-d₆) δ 11.45 (s, 1H), 10.95 (s, 1H), 8.15 (s, 1H), 7.96 (d, J=1.6 Hz, 1H), 7.93 (d, J=8.4 Hz, 1H), 7.56 (dd, J=8.4, 1.7 Hz, 1H), 6.05 (s, 1H), 4.32 (dd, J=12.5, 4.7 Hz, 1H), 2.84 (ddd, J=17.9, 12.9, 5.3 Hz, 1H), 2.68 (s, 3H), 2.61 (m, 1H), 2.44 (qd, J=12.9, 4.3 Hz, 1H), 2.15 (dtd, J=13.0, 5.1, 2.9 Hz, 1H), 1.26 (s, 9H).

### Example 59: Separation of enantiomers of (3-(2,6-dioxopiperidin-3-yl)-2-methylquinolin-7-yl)methyl (3-chloro-4-methylphenyl)carbamate

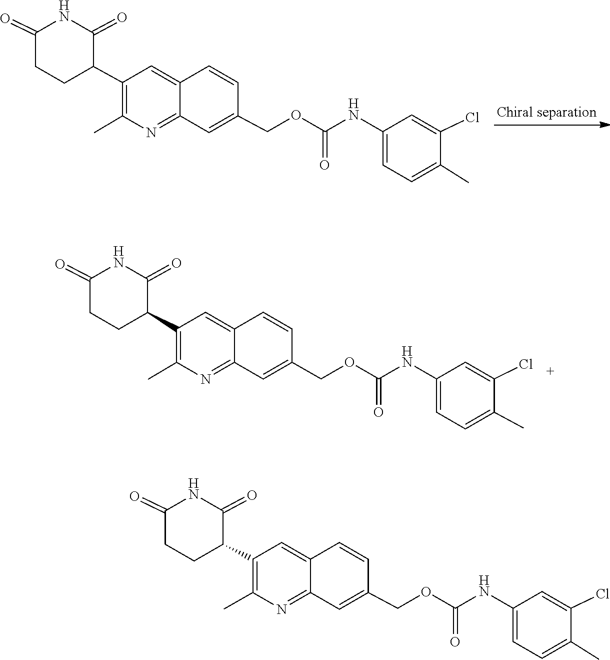 [SMILES: Cc1ccc(NC(=O)OCc2ccc3cc(C4CCC(=O)NC4=O)c(C)nc3c2)cc1Cl ; Cc1ccc(NC(=O)OCc2ccc3cc([C@H]4CCC(=O)NC4=O)c(C)nc3c2)cc1Cl ; Cc1ccc(NC(=O)OCc2ccc3cc([C@@H]4CCC(=O)NC4=O)c(C)nc3c2)cc1Cl]

Enantiomers of (3-(2,6-dioxopiperidin-3-yl)-2-methylquinolin-7-yl)methyl (3-chloro-4-methylphenyl)carbamate were separated using Thermo Scientific Vanquish UHPLC instrument and Regis REFLECT I-Cellulose C 5 mm, 250×10 mm column, and 1:1 ACN/water as eluent.

### Isomer 1 of (3-(2,6-dioxopiperidin-3-yl)-2-methylquinolin-7-yl)methyl (3-chloro-4-methylphenyl)carbamate (Compound 39)

Rₜ=19.74 min (Regis REFLECT I-Cellulose C, 5 mm, 250×4.6 mm, 1:1 ACN/water)

LCMS (ESI+) m/z 452.1 [M+H]⁺

### Isomer 2 of (3-(2,6-dioxopiperidin-3-yl)-2-methylquinolin-7-yl)methyl (3-chloro-4-methylphenyl)carbamate (Compound 38)

Rₜ=16.03 min (Regis REFLECT I-Cellulose C, 5 m, 250×4.6 mm, 1:1 ACN/water)

LCMS (ESI+) m/z 452.1 [M+H]⁺

### Example 60: CRBN Displacement TR-FRET Assay

CRBN/DDB1 protein complex was mixed with Thalidomide-Red and a compound to be tested (the “test compound”). The test solution contained PPI Europium detection buffer, 1 mM DTT, 6.8 nM Thalidomide-Red (the tracer), 5 nM 6×His-CRBN/DDB1 protein, 0.5 nM Anti-6× His-Eu cryptate, 1% DMSO. The test solution was added to a 384-well assay plate.

The plate was spun-down (1 min, 1000 rpm, 22° C.) and then shaken using a VibroTurbulator for 30 sec at room temperature (20-25° C.), with the frequency set to level 10. The assay plate with protein and the tracer was incubated for 180 min at room temperature (20-25° C.) prior to read-out with a plate reader. The read-out was performed with plate reader (Pherastar, BMG Labtech) in time resolved fluorescence mode. Filter settings: TR 337 665 620.

The CRBN displacement TR-FRET experiment was carried out with various concentrations of the test compounds in order to measure Kᵢ values.

The Kᵢ values of competitive inhibitors were calculated using the equation based on the IC₅₀ values of relationship between compound concentration and measured fluorescence polarization, the K_{d} value of the Thalidomide-Red and CRBN/DDB1 complex, and the concentrations of the protein and the tracer in the displacement assay (as described by Cheng and Prusoff, Biochem Pharmacol 1973; 22:3099-3108).

### CRBN Displacement TR-FRET Assay—Results

Compounds are categorized based on their affinity to CRBN defined as Kᵢ. As reported in Table 1 below.

CRBN binding Kᵢ [μM] is indicated as follows:
A<0.5 μM
0.5 μM≤B≤1 μM
C>1 μM

TABLE 1
CRBN displacement TR-FRET assay.

| Compound No. | CRBN binding Kᵢ [μM] |
|---|---|
| 1 | A |
| 2 | B |
| 3 | C |
| 4 | B |
| 5 | A |
| 6 | C |
| 7 | A |
| 8 | A |
| 9 | C |
| 10 | A |
| 11 | C |
| 12 | C |
| 13 | A |
| 14 | C |
| 15 | C |
| 17 | A |
| 18 | B |
| 19 | C |
| 20 | A |
| 21 | B |
| 22 | C |
| 23 | A |
| 26 | B |
| 27 | A |
| 28 | B |
| 29 | A |
| 30 | B |
| 32 | B |
| 34 | B |
| 36 | A |
| 37 | B |
| 38 | C |
| 39 | B |
| 42 | A |
| 44 | A |
| 45 | C |
| 46 | B |
| 47 | A |
| 49 | C |
| 50 | B |
| 51 | A |
| 52 | C |
| 54 | B |
| 55 | B |
| 58 | B |
| 59 | A |
| 60 | B |
| 64 | A |
| 66 | A |
| 67 | A |
| 68 | C |
| 69 | C |
| 70 | A |
| 71 | C |
| 72 | B |
| 74 | C |
| 75 | C |
| 80 | B |
| 81 | A |
| 82 | A |

### Example 61: Ternary Complex Formation Assay

The effect of the molecular glue compounds of the invention on the formation of a ternary complex composed of [GSPT1]-[compound of formula (I)]-[CRBN/DDB1] was investigated with two methods: AlphaLISA dose response assay or HTRF ternary complex assay.

### AlphaLISA Assay:

Two types of protein solution were prepared:
200 nM biotinylated GSPT1 (Table 3), 40 μg/ml AlphaScreen Streptavidin-coated Donor Beads in HBS (10 mM HEPES, 150 mM NaCL, pH 7.4) buffer with 0.1% Tween-20 and 1 mM DTT,
200 nM 6×His-CRBN/DDB1, 40 μg/ml AlphaLISA Anti-6×His Acceptor beads in HBS buffer with 0.1% Tween-20 and 1 mM DTT.

The prepared solutions were incubated at room temperature for 30 min and then the solution containing the donor beads was mixed with the solution containing the acceptor beads.

The tested compounds were dispensed onto a white 384-well AlphaPlate 384 SW. DMSO was backfilled to all wells, resulting in a final DMSO content of 2%. Wells containing only DMSO served as background. Next, 10 μl of solution with donor and acceptor beads was added to the wells.

The plate was sealed with transparent film and shaken using a VibroTurbulator for 60 sec at room temperature, level 3. The plate was then spun down shortly (10 sec, 1000 rcf, room temperature) and incubated at 25° C. for 30 min.

The read-out was performed with PerkinElmer Enspire Multimode Plate Reader (method for AlphaLISA 384-well low volume, Filterset: λexc=680 nm, λem=615 nm).

The results were analyzed as follows:
1) an average of luminescence for background signal was calculated and used as a negative control;
2) average of the maximum measured luminescence for reference compound (structure shown in Table 3 below) was calculated and used as an internal positive control;
3) raw luminescence values were normalized against positive and negative controls;
4) Normalized responses to positive control were determined.

As illustrated in Table 2, the compounds of the present invention have the capability to induce the formation of the [GSPT1]-[compound of formula (I)]-[CRBN/DDB1] complex.

### HTRF Ternary Complex Assay:

The effect of the molecular glue compounds of the invention on the formation of a ternary complex composed of [GSPT1]-[compound of formula (I)]-[CRBN/DDB1] was investigated.

Mix solution of proteins and reagents was prepared:
48 nM GSPT1, 52.8 nM 6×His-CRBN/DDB1, 3 nM of Streptavidin-Eu cryptate (acceptor) and 6.67 nM of Anti-6×his-d2 (donor) was prepared in PPI Europium detection buffer (purchased from Cisbio) with 1 mM DTT.

The tested compounds in dose-response were dispensed onto a white 384-well low volume plate (Greiner, 784075). DMSO was backfilled to all wells, resulting in a final DMSO content of 0.5%. Wells containing only DMSO served as background.

The plate was sealed with transparent film and shaken using a VibroTurbulator for 60 sec at level 3.

The plate was then spun down shortly (10 sec, 1000 rcf) and incubated at 25° C. for 180 min.

The read-out was performed with plate reader (Pherastar, BMG Labtech) in time resolved fluorescence mode. Filter settings: TR 337 665 620.

The results were analyzed as follows:
1) an average of fluorescence for background signal was calculated and used as a negative control;
2) raw fluorescence values for tested compounds were normalized against negative controls;
3) saturation curve was fitted to specific binding with Hill slope model;
4) EC50 and pEC50 values were determined.

As illustrated in Table 2, the compounds of the present invention have the capability to induce the formation of the [GSPT1]-[compound of formula (I)]-[CRBN/DDB1] complex.

TABLE 2
Ternary complex assay results for the compounds of the invention

| | Normalized AlphaLISA | HTRF ternary complex |
|---|---|---|
| | ternary complex average | Average pEC50 by |
| Compound No. | signal at 1 μM molecule | molecule |
| 1 | A | +++ |
| 2 | B | + |
| 3 | B | + |
| 4 | A | ++ |
| 5 | A | + |
| 6 | B | ++ |
| 7 | B | + |
| 8 | A | ++ |
| 9 | B | ++ |
| 10 | B | +++ |
| 11 | A | ++ |
| 12 | B | ++ |
| 13 | B | + |
| 14 | B | ++ |
| 15 | B | ++ |
| 17 | B | +++ |
| 18 | B | ++ |
| 19 | B | + |
| 20 | B | +++ |
| 21 | A | ++ |
| 22 | A | ++ |
| 23 | B | + |
| 26 | B | ++ |
| 27 | B | +++ |
| 28 | B | ++ |
| 29 | B | + |
| 30 | A | ++ |
| 32 | A | ++ |
| 34 | C | + |
| 36 | C | + |
| 37 | B | ++ |
| 38 | C | ++ |
| 39 | A | ++ |
| 42 | A | ++ |
| 44 | B | +++ |
| 45 | B | ++ |
| 46 | B | ++ |
| 47 | B | + |
| 49 | B | ++ |
| 50 | A | ++ |
| 51 | B | +++ |
| 52 | B | ++ |
| 54 | A | ++ |
| 55 | A | ++ |
| 58 | A | ++ |
| 59 | A | +++ |
| 60 | B | ++ |
| 64 | B | +++ |
| 66 | A | ++ |
| 67 | A | +++ |
| 68 | C | + |
| 69 | A | ++ |
| 70 | A | +++ |
| 71 | A | + |
| 72 | B | ++ |
| 74 | B | + |
| 75 | A | ++ |
| 80 | A | ++ |
| 81 | A | +++ |
| 82 | A | +++ |

### AlphaLISA Ternary Complex Level Description:

A>50%
10%≤B≤50%
C<10%

### HTRF Ternary Complex Activity Description:

+++ pEC50>6.5
++ 5.5 pEC50<6.5
+ pEC50<5.5

### AlphaLISA Assay for Off-Target Recruitment Detection:

The ability of the molecular glue compounds of the invention to mediate the proximity of CRBN with its known neo-substrates (shown in Table 3) was assessed using AlphaLISA to detect the formation of a [neo-substrate]-[compound of formula (I)]-[CRBN/DDB1] ternary complex.

Two types of protein solution were prepared:
neo-substrate, 40 μg/ml AlphaScreen Donor Beads (Table 3) in PBS (10 mM phosphate buffer, 137 mM NaCL, 2.7 mM KCl, pH 7.4) buffer supplemented with Tween-20 (Table 3) and 1 mM DTT,
200 nM 6×His-CRBN/DDB1, 40 μg/ml AlphaLISA Anti-6×His Acceptor beads in PBS buffer with Tween-20 (Table 3) and 1 mM DTT.

The prepared solutions were incubated at room temperature for 30 min and then the solution containing the donor beads was mixed with the solution containing the acceptor beads.

The tested compounds were dispensed onto a white 384-well AlphaPlate 384 SW. DMSO was backfilled to all wells, resulting in a final DMSO content of 2%. Wells containing only DMSO served as background. Next, 10 μl of solution with donor and acceptor beads was added to the wells.

The plate was sealed with transparent film and shaken using a VibroTurbulator for 60 sec at room temperature, level 3. The plate was then spun down shortly (10 sec, 1000 rcf, room temperature) and incubated at 25° C. for 30 min.

The read-out was performed with PerkinElmer Enspire Multimode Plate Reader (method for AlphaLISA 384-well low volume, Filterset: λexc=680 nm, λem=615 nm).

The results were analyzed as follows:
1) an average of luminescence for background signal was calculated and used as a negative control;
2) an average of the maximum measured luminescence for reference compound (Table 3) was calculated and used as an internal positive control;
3) raw luminescence values were normalized against positive and negative controls;
4) Normalized responses to reference molecular glue were determined.

As illustrated in Table 4, the compounds of the present invention have low capability to induce the formation of the [neo-substrate]-[compound of formula (I)]-[CRBN/DDB1] complex, which indicates their high selectivity.

TABLE 3
Utilized neo-substrate proteins and AlphaLISA assay conditions.

| Neo- | Protein | Donor bead | Tween-20 | Reference | |
|---|---|---|---|---|---|
| substrate | [nM] | type | [%] | compound | Structure |
| GSPT1 | 200 | Streptavidin- coated | 0.1 | CC-885; Nature 2016, 14, 535(7611): 252-7. doi: 10.1038/ nature18611 | 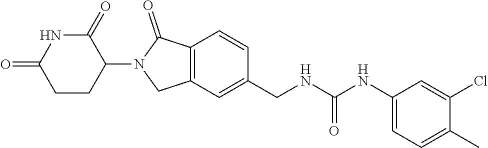 [SMILES: Cc1ccc(NC(=O)NCc2ccc3c(c2)CN(C2CCC(=O)NC2=O)C3=O)cc1Cl] |
| SALL4 ZF2 | 800 | Strep-Tactin- coated | 0.1 | Lenalidomide | 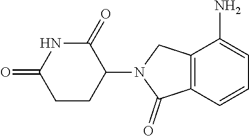 [SMILES: Nc1cccc2c1CN(C1CCC(=O)NC1=O)C2=O] |
| IKZF1 ZF2 | 800 | Strep-Tactin- coated | 0.1 | Lenalidomide | 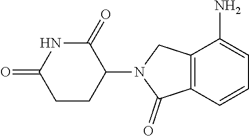 [SMILES: Nc1cccc2c1CN(C1CCC(=O)NC1=O)C2=O] |
| IKZF2 ZF2 | 800 | Strep-Tactin- coated | 0.1 | Helios modulator; Novartis; WO 2019079569; WO 2019038717 | 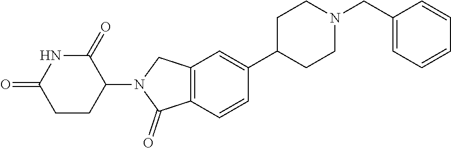 [SMILES: O=C1CCC(N2Cc3cc(C4CCN(Cc5ccccc5)CC4)ccc3C2=O)C(=O)N1] |
| CK1a | 40 | Streptavidin- coated | 0.01 | Ikaros & CK1a degrader; FPFT-2216; WO 2019146773, PNAS 2018, 115, 11802-11807 | 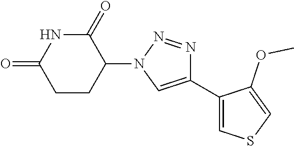 [SMILES: COc1cscc1-c1cn(C2CCC(=O)NC2=O)nn1] |

TABLE 4
The Normalized ternary complex responses observed for various neo-
substrates in the presence of compounds of invention or GSPT1-positive
control. Presented data were determined at 1 μM compound.

| Compound | | | | |
|---|---|---|---|---|
| No. | SALL4 ZF2 | IKZF1 ZF2 | IKZF2 ZF2 | CK1α |
| CC-885 | A | A | A | C |
| 1 | A | B | C | D |
| 2 | D | D | D | D |
| 3 | D | D | D | D |
| 4 | D | D | D | D |
| 5 | B | B | C | D |
| 6 | D | D | D | D |
| 7 | A | B | D | D |
| 8 | D | D | D | D |
| 9 | D | D | D | D |
| 10 | D | D | D | D |
| 11 | D | D | D | D |
| 12 | D | D | D | D |
| 13 | D | D | D | D |
| 14 | D | D | D | D |
| 15 | D | D | D | D |
| 17 | D | D | D | D |
| 18 | D | D | D | D |
| 19 | D | D | D | D |
| 20 | D | D | D | D |
| 21 | D | D | D | D |
| 22 | D | D | D | D |
| 23 | B | D | D | D |
| 26 | B | D | D | D |
| 27 | D | D | D | D |
| 28 | D | D | D | D |
| 29 | B | C | D | D |
| 30 | D | D | D | D |
| 32 | D | D | D | D |
| 34 | D | D | D | D |
| 36 | D | D | D | D |
| 37 | D | D | D | D |
| 38 | D | D | D | D |
| 39 | D | D | D | D |
| 42 | D | D | D | D |
| 44 | D | D | D | D |
| 45 | D | D | D | D |
| 46 | D | D | D | D |
| 47 | D | D | D | D |
| 49 | D | D | D | D |
| 50 | D | D | D | D |
| 51 | A | B | C | D |
| 52 | A | B | C | D |
| 54 | D | D | D | D |
| 55 | D | D | D | D |
| 58 | D | D | D | D |
| 59 | D | D | D | D |
| 60 | A | C | D | D |
| 64 | D | D | D | D |
| 66 | B | B | C | C |
| 67 | B | A | B | C |
| 68 | D | D | D | D |
| 69 | D | D | D | D |
| 70 | D | D | D | D |
| 71 | D | D | D | D |
| 72 | D | D | D | D |
| 74 | D | D | D | D |
| 75 | D | D | D | D |
| 80 | D | D | D | D |
| 81 | D | D | D | D |
| 82 | D | D | D | D |

### AlphaLISA Ternary Complex Level Description:

A≥50%
25%≤B<50%
10%≤C<25%
D<10%

### Example 62: GSPT1 Degradation Assay—Hep3B Cell Line

The effect of selected compounds of the invention on GSPT1 protein levels in the Hep3B cell line was investigated, using the degradation assay protocol below.

Hep3B cells (ATCC, cat. no HB-8064) were maintained in EMEM medium, supplemented with penicillin/streptomycin and 10% Fetal Bovine Serum (FBS). Compounds were stored frozen as 20 mM DMSO stocks. Cells were seeded on 60 mm culture dishes to achieve around 70% confluency on the day of treatment and incubated overnight. Following media exchange to a fresh one, cells were treated with the test compound (prediluted in DMSO and added directly to the media) and vehicle only (DMSO) control. Final DMSO concentration in all conditions was 0.25%. After 6- and 24-hours incubation (37° C., 5% CO₂), culture dishes were placed on ice, media removed and collected. Cells were washed with ice-cold DPBS, and the washing solution was transferred to the previously collected culture media. RIPA lysis buffer (50 mM Tris-HCl pH 7.4, 150 mM NaCl, 1% NP-40, 0.25% sodium deoxycholate, 0.1% SDS, 1 mM EDTA) supplemented with protease and phosphatase inhibitors (Roche, cat. no 05056489001 and ThermoFisher Scientific, cat. no 78426, respectively) was added to the plates, cells were scraped, transferred into microtube and placed on ice. Media and washing solution were centrifuged (5 min, 230×g), supernatant was removed, pellet washed with DPBS and centrifuged as previously. Supernatant was discarded and the resulting pellet was combined with the respective cell lysate collected from the culture dishes. Samples were snap frozen using liquid nitrogen and stored at −20° C. before proceeding. Cell lysates were thawed on ice, centrifuged at 19 000×g at 4° C. for 15 minutes, and supernatants (cleared lysates) were collected.

Total protein concentration was determined using BCA assay (ThermoFisher Scientific, 23225), protein concentrations of the samples were adjusted using RIPA lysis buffer and SDS-PAGE samples were prepared by adding appropriate amount of 5× sample buffer. Finally, samples were briefly spun down, boiled for 5 min at 95° C., cooled down, spun down again and vortex mixed.

Equal quantities of the samples were then loaded on the precast polyacrylamide gel (4-20% TGX Stain-Free™ Protein Gel, Bio-Rad), alongside molecular mass marker (Precision Plus Protein Dual Color Standards, Bio-Rad, cat. no 1610374). Following separation, proteins were transferred onto nitrocellulose membrane using Trans-Blot® Turbo™ system (Bio-Rad) and the appropriate manufacturer's pre-set transfer program.

Membrane was blocked for 60 min in 5% NFM (non-fat milk) TBS-T solution. Anti-GSPT1 primary antibody (ThermoFisher Scientific, cat. no PA5-28256) was prepared as 1:1000 dilution in 5% NFM TBS-T solution and incubated with the membrane at 4° C. overnight. Next, membrane was washed with TBS-T and incubated with the HRP-conjugated secondary antibody (anti-rabbit, ThermoFisher Scientific, cat. no 31466) prepared as 1:2500 dilution in 5% NFM TBS-T solution for 60 min in RT. Membrane was washed in TBS-T solution followed by addition of the chemiluminescence substrate (SuperSignal West Pico PLUS, ThermoFisher Scientific, 34578) and incubated for 5 min. Chemiluminescence signal was detected and image acquired using Chemi Doc MP imager (Bio-Rad).

Following TBS-T wash, membrane was incubated with HRP-conjugated anti-ß-actin (Abcam, cat. no ab20272, 1:10000 dilution in 5% NFM TBS-T) antibody for 60 min in RT. Membrane was then washed, signal was acquired as described above.

For image analysis “Image Lab” software was used. The obtained GSPT1 signal values were normalized to the ß-actin loading control and relative GSPT1 levels were calculated as % of the DMSO control. The half-maximal degradation concentration (DC₅₀) was calculated using a four-parameter non-linear regression curve fitting model (GraphPad Prism Software), using % degradation values (100%−% relative GSPT1 level).

The tested compound demonstrated high degradation potency towards GSPT1 with DC50<10 nM after 6 and 24 hours of treatment in the HEP3B cell line.

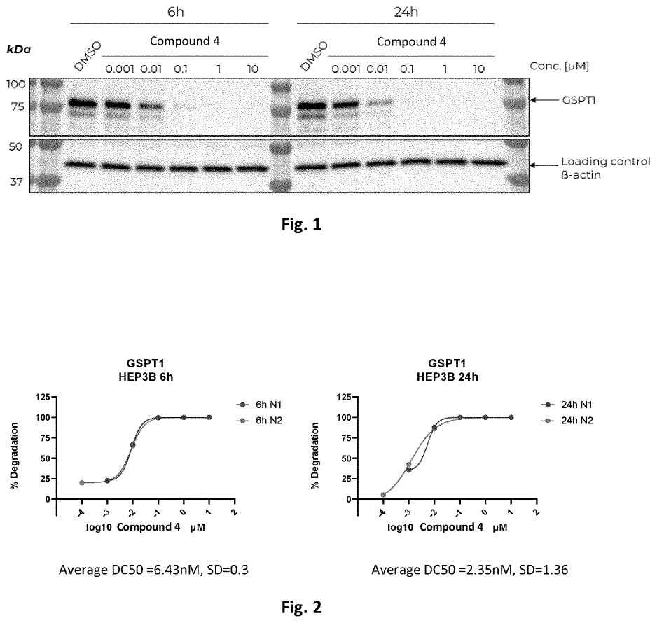

FIG. 1 shows the immunoblot analysis of Hep3B cells treated with DMSO or Compound 4, for 6 and 24 hours as indicated. Representative data shown.

FIG. 2 is a graphical representation of the dose response curves showing % of GSPT1 protein degradation induced by Compound 4 in HEP3B cells. For each timepoint the average DC₅₀ value is reported.

### Example 63: Cell Viability in Kelly, NCI-H929, KG-1, Hep3B and Hep3B GSPT1 G575N Mut. Cell Lines

The effect of selected compounds of the invention on cell viability in various cell lines was investigated using the Cell Viability—CTG Assay Protocol below.

Hep3B, Hep3B GSPT1 G575N mut., Kelly, KG-1 and NCI-H929 cells were maintained in respective cell medium (see Table 5, below). Cells were seeded in appropriate density (see Table 6, below) onto 384-white plates (Greiner, cat. no. 781080 or 781098) and incubated overnight (Hep3B, Hep3B GSPT1 G575N, Kelly) or treated with compounds immediately after seeding (KG-1, NCI-H929). Tested compounds and DMSO (vehicle control) were dispensed to the plates using Echo 555 Liquid Handler. 12 data-point compound titration curve with concentrations ranging from 50 μM to 1 nM was used. Final DMSO concentration was kept constant at 0.25% v/v across the assay plate. After 72 h incubation (37° C., 5% CO₂), cell viability was assessed using CellTiter-Glo® assay kit (Promega, cat. no G7573). Luminescence was measured using CLARIOstar multimode plate reader (BMG LABTECH). The raw data (the relative luminescence unit values, RLU) were uploaded under the relevant protocol and analysed in CDDVault data management platform. Luminescence (RLU) values were normalized to the controls, and reported absolute IC₅₀ values were calculated in CDDVault using non-linear regression and appropriate equations.

Exemplified compounds of the present invention potently inhibit growth of several cancer types: hepatocellular carcinoma (HEP3B), neuroblastoma (Kelly), leukemia (KG-1) and multiple myeloma (H929), demonstrating the potential anti-cancer effect. Lack of cytotoxic effect of the compounds on the Hep3B cell line with GSPT1 mutation G575N (Hep3B GSPT1 G575N mut.) conferring resistance to compound induced degradation, confirms GSPT1 dependent mechanism.

TABLE 5
Cell lines and culture media.

| Cell line | Source (ref#) | Culture medium |
|---|---|---|
| Hep3B | ATCC (HB-8064) | 89% Eagle's Minimum Essential Medium (EMEM) |
| | | 10% Fetal Bovine Serum, qualified, heat inactivated |
| | | 1% Penicillin/Streptomycin |
| Hep3B GSPT1 | ATCC (HB-8064) | 89% Eagle's Minimum Essential Medium (EMEM) |
| G575N | | 10% Fetal Bovine Serum, qualified, heat inactivated |
| | | 1% Penicillin/Streptomycin |
| Kelly | DSMZ (ACC355) | 89% RPMI 1640 Medium |
| | | 10% Fetal Bovine Serum, qualified, heat inactivated |
| | | 1% Penicillin/Streptomycin |
| NCI-H929 | ATCC (CRL-9068) | 89% RPMI 1640 (ATCC modification) |
| | | 10% Fetal Bovine Serum, qualified, heat inactivated |
| | | 1% Penicillin/Streptomycin |
| | | 0.05 mM β-Mercaptoethanol |
| KG-1 | DMSZ (ACC14) | 89% RPMI 1640 Medium |
| | | 10% Fetal Bovine Serum, qualified, heat inactivated |
| | | 1% Penicillin/Streptomycin |

TABLE 6
Cell seeding density

| | Seeding density for 72 h treatment |
|---|---|
| Cell line | (cells/50 μL/well) |
| Kelly | 2500 |
| NCI-H929 | 3000 |
| KG-1 | 3000 |
| Hep3B | 1250 |
| Hep3B GSPT1 G575N | 1250 |

TABLE 7
Effect of selected compounds on cell viability
following 72 hours treatment

| | | | | | Hep3B |
|---|---|---|---|---|---|
| | | | NCI- | | GSPT1 |
| | KG-1 | Kelly | H929 | Hep3B | G575N |
| Com- | Geomean | Geomean | Geomean | Geomean | Geomean |
| pound | absolute | absolute | absolute | absolute | absolute |
| No. | IC₅₀ [μM] | IC₅₀ [μM] | IC₅₀ [μM] | IC₅₀ [μM] | IC₅₀ [μM] |
| 2 | C | C | D | D | D |
| 4 | A | A | A | A | D |
| 5 | A | A | A | A | D |

A represents IC₅₀ ≤ 100 nM,
B represents 1 μM ≥ IC₅₀ > 100 nM,
C represents 5 μM ≥ IC₅₀ > 1 μM,
D represents IC₅₀ > 5 μM

### Example 64: Chemical Stability

Chemical stability of the compounds has been tested in MV-4-11 medium (IMDM medium, supplemented with penicillin/streptomycin and 10% Fetal Bovine Serum (FBS)). Compounds stock solutions (1 mM or 10 mM in DMSO) were dissolved 1000-fold in MV-4-11 medium (e.g. 1.6 μL of 10 mM stock solution was added to 1600 μL of MV-4-11 medium), to give the final concentrations of approximately 1 μM or 10 μM, and mixed. Next, 500 μL of each solution was transferred to the 96-deep-well plate. Three replicates for each compound were performed. Plate was afterwards incubated at 37° C. with shaking (220 rpm). 50 μL of sample were withdrawn for the each timepoint (0, 1, 3, 6, 24, 48, 72, and 168 hours) and immediately mixed with 50 μL of acetonitrile. Next, samples were centrifuged at 3900 rpm, 10° C. for 20 minutes. 40 μL of supernatant was diluted with 120 μL of acetonitrile containing internal standard (IS) (10 μM), mixed, and analyzed by LC-MS/MS.

Thermo Fisher Scientific Altis+ coupled with a Vanquish system were used for UHPLC-MS/MS analysis. Analytes were separated on a Kinetex XB-C18 2.6 μm, 50×2.1 mm column, using gradient of water with 0.1% (v/v) formic acid (solvent A) and acetonitrile with 0.1% (v/v) formic acid (solvent B): 0.0 min 5% B, 0.5 min 5% B, 1.0 min 70% B, 3.0 min 95% B, 3.5 min 95% B, 3.7 min 5% B, 5 min 5% B. The mobile phase flow rate was set to 0.3 mL/min and the column was kept in 40° C. 1 μL of samples were injected. The MS/MS analysis was performed using SRM mode in positive ionization. Source parameters were set as follow: Ion Source Type H-ESI, Spray Voltage 3800 V, Sheath Gas 50 Arb, Aux Gas 10 Arb, Sweep Gas 1 Arb, Ion Transfer Tube Temp 325° C., Vaporizer Temp 350° C., Collision Gas Pressure 1.5 mTorr, Q1/Q3 Resolution (FWHM) 0.7. Three transitions were monitored for each analyte and for the internal standard:

TABLE 8
SRM transitions

| | | Quantitative | | Qualitative | | Qualitative | |
|---|---|---|---|---|---|---|---|
| | | transition | | transition | | transition | |
| | | Product | | Product | | Product | |
| | Precursor | ion | CE | ion | CE | ion | CE |
| Compound | Ion (m/z) | (m/z) | (V) | (m/z) | (V) | (m/z) | (V) |
| lenalidomide | 260.0 | 149.0 | 16 | 106.0 | 35 | 187.0 | 25 |
| pomalidomide | 274.0 | 201.0 | 32 | 173.4 | 18 | — | — |
| avadomide | 287.0 | 176.0 | 25 | 106.9 | 45 | 133.0 | 35 |
| eragidomide | 460.1 | 298.1 | 18 | 243.0 | 24 | 397.0 | 18 |
| 4 | 452.1 | 285.1 | 33 | 200.0 | 51 | 267.0 | 28 |

Quantitation was based on the peak area ratio between the test compound and IS. The amount of compound remaining in the sample was calculated as follow:

$$\% remaining = ( Area ratio analyte / IS at time ) \times 100 / ( Area ratio analyte / IS at time 0 ) .$$

The slope of logarithmic curve of percent remaining versus time has been used for calculation of half-life (t_half). First, the elimination constant (k) was calculated using the equation:

$$elimination constant ( k ) = ( - slope of logarithmic curve )$$

Next, half-life (t_half) was calculated using equation:

$$T\_half = ln ( 2 ) / k$$

Exemplified compound of the present invention demonstrates improved chemical stability in comparison to the known IMiDs, which is evidenced by higher values of t_half at 24 hours. Eragidomide, a GSPT1 degrader, is much less stable than exemplified compound no 4.

TABLE 9
Chemical stability of selected compound and
known IMiDs in MV-4-11 medium at 24 hours

| | Concentration | % remaining at | t_half |
|---|---|---|---|
| Compound | [μM] | 24 hrs | [hours] |
| lenalidomide | 10 | e | D |
| pomalidomide | 10 | e | D |
| avadomide | 10 | d | C |
| eragidomide | 10 | e | D |
| 4 | 10 | b | A |
| 4 | 1 | a | A |

### Description of % Remaining Compounds at 24 Hrs:

a≥50%
25%≤b<50%
10%≤c<25%
5%≤d<10%
e<5%
   Description of t_Half:
A≥15 hrs
10 hrs≤B<15 hrs
5 hrs≤C<10 hrs
D<5 hrs

### Example 65: GSPT1 Degradation Assay Using HiBiT System—GSPT1-HiBiT HEK293 Cell Line

The effect of selected compounds of the invention on GSPT1 protein levels was investigated, using the GSPT1-HiBiT HEK293 cell line and the Nano-Glo HiBiT degradation assay protocol below.

GSPT1-HiBiT HEK293 cells were generated in-house using the HEK293 parental cells (ATCC, cat. no 70016364) and the CRISPR/Cas9 system. HEK293 cells were transformed with pSpCas9-BB-2A-Puro v2.0 plasmid carrying gRNA targeting the C-terminus of GSPT1 and ssODN template containing the HiBiT tag sequence with flanking homology sequences. Neon Transfection System (Thermo Fisher Scientific) was used for electroporation. HEK293 GSPT1-HiBiT cells were cultured with DMEM Glutamax (Gibco) supplemented with 10% heat inactivated FBS (Gibco). After transfection the culture medium was changed to DMEM Glutamax with 10% heat-inactivated FBS and 1% Penicillin-Streptomycin (Biowest) supplemented with Puromycin (2 μg/ml; Invivogen) for clonal selection. In order to isolate single cell clones for further validation and analysis, limiting dilution cloning in 96-well plates was performed. When the single clones reached confluency, HiBiT Lytic Assay (Promega) was performed in order to identify HiBiT positive clones. The clone selected for further studies was verified and validated using genotyping and HiBiT Blotting (Promega).

Cells were maintained in DMEM Glutamax medium, supplemented with 1% penicillin/streptomycin and 10% Fetal Bovine Serum (FBS) at 37° C., 5% CO₂. Compounds were stored frozen as 20 mM DMSO stocks.

For the Nano-Glo HiBiT assay, cells were seeded onto 384-well white solid bottom plate at 2×10³ cells/well in 40 μL using the Agilent BioTek MultiFlo FX Multimode Dispenser and incubated overnight. Following overnight incubation (37° C., 5% CO₂) cells were treated with the test compounds and vehicle only (DMSO) control using the Echo 555 Liquid Handler. Twelve data-point compound titration curve with concentrations ranging from 10 μM to 0.03 nM was used. Final DMSO concentration was kept constant at 0.25% v/v across the assay plate. After 6 hours of incubation (37° C., 5% CO₂), plates were removed from the incubator and let to equilibrate to RT. Luminescent signal detection was conducted using the Nano-Glo HiBiT Lytic Detection System (Promega, cat. nr N3050). Nano-Glo HiBiT Lytic Reagent was prepared freshly by diluting the LgBiT Protein (1:100 vol/vol) and the Nano-Glo HiBiT Lytic Substrate (1:50 vol/vol) in the relevant volume of the Nano-Glo HiBiT Lytic Buffer. 40 μL of the HiBiT Lytic Reagent was added per well, plates were shaken to facilitate lysis (350 rpm, 5 min, RT) and incubated in the dark for the following 10 min. Luminescence was read using the PHERAstar multimode plate reader (BMG LABTECH). The raw data (the relative luminescence unit values, RLU) were uploaded under the relevant protocol and analysed in CDDVault data management platform. Luminescence (RLU) values were normalized to the controls, and reported absolute DC₅₀ values and Dₘₐₓ values were calculated in CDDVault using non-linear regression and appropriate equations.

In parallel, the Cell Viability-CTG assay was performed to ensure that the potential decrease of the luminescent signal observed in the Nano-Glo HiBiT assay is a result of protein degradation and not a result of decreasing cell viability. For CTG assay accompanying the Nano-Glo HiBiT assay, cells were cultured, seeded and treated as described above. After 6 hours of incubation (37° C., 5% CO₂), plates were removed from the incubator and let to equilibrate to RT. Luminescent signal detection was conducted using the CellTiter-Glo Lumiescent Cell Viability Assay (Promega, cat. nr G7573). 10 μL of the Cell Titer Glo reagent was added per well, plates were shaken to facilitate lysis (460 rpm, 4 min, RT) and incubated in the dark for the following 8 min. Luminescence was read using the CLARIOstar multimode plate reader (BMG LABTECH). The raw data (the relative luminescence unit values, RLU) were uploaded under the relevant protocol and analysed in CDDVault data management platform, in a manner similar to the description above. The Mean Minimum values were calculated in CDDVault using non-linear regression and appropriate equations.

The tested compounds demonstrated high degradation potency towards GSPT1 with majority of the compounds reaching the absolute DC₅₀ at low nanomolar concentrations and being able to degrade vast majority of the target protein during 6 hours of treatment, without affecting the viability of the HiBiT-GSPT1 HEK293 cells.

TABLE 10
Effect of selected compounds on GSPT1 protein using
the Nano-Glo HiBiT assay and on cell viability following
6 hours treatment of GSPT1-HiBiT HEK293 cells.

| Compound | HiBiT lytic assay | | CTG assay |
|---|---|---|---|
| No. | Geomean absolute DC₅₀ | Mean Dₘₐₓ | Mean minimum |
| 4 | B | a | ++++ |
| 6 | B | a | ++++ |
| 8 | A | a | ++++ |
| 9 | B | a | ++++ |
| 10 | A | a | ++++ |
| 11 | A | a | ++++ |
| 12 | B | a | ++++ |
| 14 | B | a | ++++ |
| 15 | A | a | ++++ |
| 18 | B | a | ++++ |
| 17 | A | a | ++++ |
| 19 | A | a | ++++ |
| 20 | A | a | ++++ |
| 21 | B | a | ++++ |
| 22 | B | a | ++++ |
| 27 | C | a | ++++ |
| 28 | B | a | ++++ |
| 29 | B | a | ++++ |
| 30 | A | a | ++++ |
| 32 | B | a | ++++ |
| 37 | B | a | ++++ |
| 38 | C | a | ++++ |
| 39 | A | a | ++++ |
| 42 | B | a | ++++ |
| 44 | A | a | ++++ |
| 45 | B | a | ++++ |
| 46 | B | a | ++++ |
| 49 | C | a | ++++ |
| 50 | B | a | ++++ |
| 51 | B | a | ++++ |
| 54 | B | a | ++++ |
| 55 | B | a | ++++ |
| 58 | A | a | ++++ |
| 59 | B | a | ++++ |
| 64 | D | a | ++++ |
| 66 | B | a | ++++ |
| 67 | B | a | ++++ |
| 69 | B | a | ++++ |
| 70 | A | a | ++++ |
| 71 | C | a | ++++ |
| 72 | B | a | ++++ |
| 74 | D | a | ++++ |
| 75 | B | a | ++++ |
| 80 | B | a | ++++ |
| 81 | A | a | ++++ |
| 82 | A | a | ++++ |

### Geomean Absolute DC₅₀ Level Description:

A 10 nM
10 nM<B≤50 nM
50 nM<C≤100 nm
D>100 nM

### Mean Dₘₐₓ Level Description:

a≥75% of GSPT1-HiBiT protein degraded
50%≤b<75% of GSPT1-HiBiT protein degraded
25%≤c<50% of GSPT1-HiBiT protein degraded
d<25% of GSPT1-HiBiT protein degraded

### Mean Minimum Level Description:

+<25% of viability of the HiBiT-GSPT1 HEK293 cells
25%≤++<50% of viability of the HiBiT-GSPT1 HEK293 cells
50%≤+++<75% of viability of the HiBiT-GSPT1 HEK293 cells
++++≥75% of viability of the HiBiT-GSPT1 HEK293 cells

The invention is further described with reference to the following clauses:

1. A compound of formula (I):

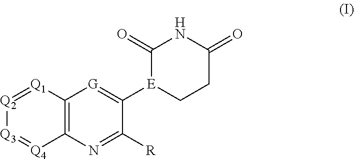

wherein
   R⁶ is hydrogen, unsubstituted C₁-C₄ alkyl, haloalkyl, —OR¹ or —N(R¹)₂;
   E is CH, CD, CF, C₁-C₃ alkyl or N;
   G is CR¹ or N;
   one of Q₁, Q₂, Q₃, and Q₄ is CR⁵ and the other three of Q₁, Q₂, Q₃, and Q₄ are each independently N or CR⁴; wherein when G is CR¹ then at least one of Q₁, Q₂, Q₃, and Q₄ is CR⁴; and
when G is N then at least two of Q₁, Q₂, Q₃, and Q₄ are CR⁴;
   each R¹ is independently hydrogen, unsubstituted C₁-C₄ alkyl, or C₁-C₄ haloalkyl;
   each R⁴ is independently hydrogen, unsubstituted alkyl, haloalkyl, halogen, —CN, —OR¹ or —N(R¹)₂;
   R⁵ is selected from:

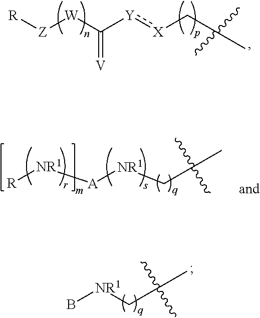

wherein [symbol] is a single bond or a double bond, wherein
when [symbol] is a single bond, then X and Y are each independently O, NR¹ or CHR¹;
wherein when one of X and Y is O or NR¹, then the other of X and Y is CHR¹; and
   when [symbol] is a double bond, then X and Y are each CR¹;
W is CH₂ or C═O;
Z is C═O, NR¹ or C(R²)₂; wherein each R² is independently hydrogen, unsubstituted alkyl, halogen, OR¹ or N(R¹)₂;
V is O or S;
each R is independently cycloalkyl, heterocycloalkyl, aryl, heteroaryl, alkyl, or benzyl wherein each cycloalkyl, heterocycloalkyl, aryl, heteroaryl, alkyl or benzyl is optionally substituted with one or more R³, wherein each R³ is independently selected from halogen, R⁷, —CH₂-heterocycloalkyl, —CN, —OH, OR⁷, —NH₂, —NR¹R⁷, —NHC(O)R⁷, —NHC(O)OR⁷, —NHC(O)NR¹R⁷ and —C(O)R⁷; or wherein two R³ together with the carbon atoms to which they are attached form a cycloalkyl, heterocycloalkyl or heteroaryl ring;
or wherein R and R′, together with the N atom to which they are attached when Z is NR¹, form a heterocycloalkyl or heteroaryl ring which is optionally substituted with one or more groups selected from halogen, unsubstituted alkyl, haloalkyl, —OH, —O(haloalkyl), —O(unsubstituted alkyl) and —N(R¹)₂;
each R⁷ is independently alkyl, haloalkyl, heteroaryl, aryl, benzyl, cycloalkyl or heterocycloalkyl;
A is heteroaryl, heterocycloalkyl, cycloalkyl or cycloalkenyl;
B is —SO₂NHR or 8-12 membered bicyclic heteroaryl substituted with one or more R⁸, wherein each R⁸ is independently selected from halogen, alkyl, —O(haloalkyl), —O(unsubstituted alkyl), aryl, and CH₂-heterocycloalkyl;
n is 0 or 1;
m is 1 or 2;
r is 0 or 1;
s is 0 or 1;
each p is independently 0, 1, 2 or 3; and
each q is independently 1, 2, 3 or 4;
wherein:
   one or more CH₂ groups of any cycloalkyl, heterocycloalkyl or cycloalkenyl are optionally replaced by a C═O group;
   in E, R³, R⁷ and R⁸, each alkyl is independently optionally substituted with one or more groups selected from halogen, —OH, —O(haloalkyl), —O(unsubstituted alkyl) and —N(R¹)₂; and
   in A, R³, R⁷ and R⁸, each cycloalkyl, heterocycloalkyl, cycloalkenyl, aryl and heteroaryl is independently optionally substituted with one or more groups selected from halogen, unsubstituted alkyl, haloalkyl, —OH, —O(haloalkyl), —O(unsubstituted alkyl) and —N(R¹)₂;
   provided that:
(a) when R⁵ is

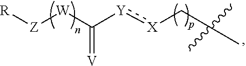

W is C═O, n is 1 and p is 0; then Z is NR¹ or C(R²)₂; and
(b) when R⁵ is

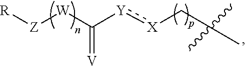

X is CHR¹, Y is NH, V is O, n is 0, p is 0, Z is NH or C(halogen)₂, and R is aryl; then at least one of E and G is N.

2. The compound of clause 1, wherein when R⁵ is

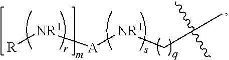

then E is CH, CD, CF or C₁-C₃ alkyl.

3. The compound of clause 2, wherein when R⁵ is

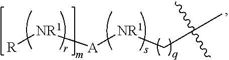

then E is CH.

4. The compound of any preceding clause, wherein at least one of r and s is 1.

5. The compound of any preceding clause, wherein r is 1.

6. The compound of any preceding clause, wherein s is 1.

7. The compound of any preceding clause, wherein when [symbol] is a single bond and one of X and Y is CHR¹, then the other of X and Y is O or NR¹.

8. The compound of any preceding clause, wherein in E, R³, R⁷ and R⁸, each alkyl is independently optionally substituted with one or more groups selected from halogen, —OH, —O(haloalkyl), and —O(unsubstituted alkyl).

9. The compound of clause 8, wherein in E, R³, R⁷ and R⁸, each alkyl is independently optionally substituted with one or more groups selected from halogen and —OH.

10. The compound of any one of clauses 1-9, wherein in E, R³, R⁷ and R⁸, each alkyl is unsubstituted.

11. The compound of any preceding clause, wherein the C₁-C₃ alkyl of E is unsubstituted C₁-C₃ alkyl.

12. The compound of any one of clauses 1-7, wherein E is CH, CD or N.

13. The compound of clause 12, wherein E is CH or N.

14. The compound of clause 13, wherein E is N.

15. The compound of any one of clauses 1-11, wherein E is CH, CD, CF or C₁-C₃ alkyl.

16. The compound of clause 15, wherein E is CH, CD or C₁-C₃ alkyl.

17. The compound of clause 16, wherein E is CH.

18. The compound of any preceding clause, wherein in A, R³, R⁷ and R⁸, any cycloalkyl, heterocycloalkyl, cycloalkenyl, aryl and heteroaryl is optionally substituted with one or more groups selected from halogen, unsubstituted alkyl, haloalkyl, —O(haloalkyl) and —O(unsubstituted alkyl).

19. The compound of any preceding clause, wherein the heterocycloalkyl or heteroaryl ring formed by R and R¹, together with the N atom to which they are attached, is optionally substituted with one or more groups selected from halogen, unsubstituted alkyl, haloalkyl, —O(haloalkyl) and —O(unsubstituted alkyl).

20. The compound of any preceding clause, wherein each R³ is independently selected from halogen, R⁷, —CH₂-heterocycloalkyl, —CN, —OH, —OR⁷, —NH₂, —NR¹R⁷, —NHC(O)R⁷, —NHC(O)OR⁷, —NHC(O)NR¹R⁷ and —C(O)R⁷.

21. The compound of clause 20, wherein each R³ is independently selected from halogen, R⁷, —CH₂-heterocycloalkyl, —CN, —OR⁷, —NH₂, —NHC(O)R⁷ and —C(O)R⁷.

22. The compound of clause 21, wherein each R³ is independently selected from halogen, R⁷, —CH₂-heterocycloalkyl, —CN, —OR⁷, —NH₂, —NHC(O)alkyl and —C(O)alkyl.

23. The compound of clause 22, wherein each R³ is independently selected from halogen, R⁷, —CH₂-heterocycloalkyl, —CN, —O(haloalkyl), —O(alkyl), —NH₂, —NHC(O)alkyl and —C(O)alkyl;

24. The compound of any preceding clause, wherein each R¹ is independently hydrogen or unsubstituted C₁-C₄ alkyl.

25. The compound of any preceding clause, wherein each R⁴ is independently hydrogen, unsubstituted alkyl, haloalkyl, halogen, —OR¹ or —N(R¹)₂

26. The compound of clause 25, wherein each R⁴ is independently hydrogen, unsubstituted alkyl, halogen, —OR¹ or —N(R¹)₂

27. The compound of any preceding clause, wherein R⁶ is unsubstituted alkyl, haloalkyl, OR¹ or N(R¹)₂.

28. The compound of clause 27, wherein R⁶ is OR¹ or N(R¹)₂.

29. The compound of clause 27, wherein R⁶ is unsubstituted alkyl or haloalkyl.

30. The compound of clause 29, wherein R⁶ is unsubstituted C₁-C₄ alkyl or C₁-C₄ haloalkyl.

31. The compound of clause 30, wherein R⁶ is unsubstituted C₁-C₄ alkyl.

32. The compound of clause 31, wherein R⁶ is methyl.

33. The compound of any preceding clause, wherein when R⁶ is

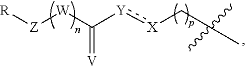

X is CHR¹, Y is NR¹, V is O, n is 0, p is 0, Z is NR¹ or C(halogen)₂, and R is aryl; then at least one of E and G is N.

34. The compound of any preceding clause, wherein G is CR¹.

35. The compound of clause 34, wherein G is CH.

36. The compound of any one of clauses 1-33, wherein G is N.

37. The compound of any preceding clause, wherein at least two of Q₁, Q₂, Q₃, and Q₄ are CR⁴.

38. The compound of any preceding clause, wherein three of Q₁, Q₂, Q₃, and Q₄ are CR⁴.

39. The compound of clause 38, wherein:
(a) Q₁ is CR⁵ and Q₂, Q₃, and Q₄ are each independently CR⁴; or
(b) Q₂ is CR⁵ and Q₁, Q₃, and Q₄ are each independently CR⁴; or
(c) Q₃ is CR⁵ and Q₁, Q₂, and Q₄ are each independently CR⁴; or
(d) Q₄ is CR⁵ and Q₁, Q₂, and Q₃ are each independently CR⁴.

40. The compound of any preceding clause, wherein R⁵ is

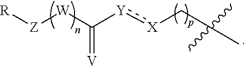

41. The compound of clause 40, wherein the compound is selected from:

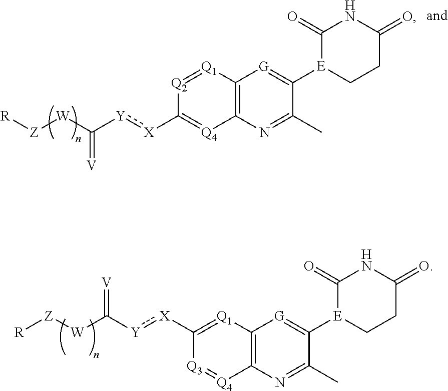

42. The compound of any one of clauses 40-41, wherein R⁵ is

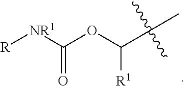

43. The compound of clause 42, wherein R⁵ is selected from

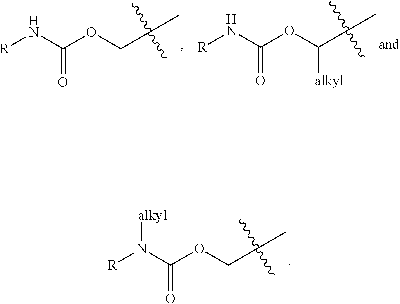

44. The compound of clause 42 or 43, wherein G and E are CH.

45. The compound of clause 44, wherein the compound is selected from:

| Compound No. | Structure |
|---|---|
| 4 | 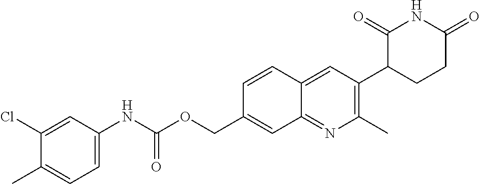 [SMILES: Cc1ccc(NC(=O)OCc2ccc3cc(C4CCC(=O)NC4=O)c(C)nc3c2)cc1Cl] |
| 5 | 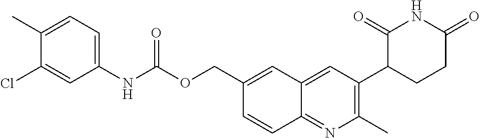 [SMILES: Cc1ccc(NC(=O)OCc2ccc3nc(C)c(C4CCC(=O)NC4=O)cc3c2)cc1Cl] |
| 6 | 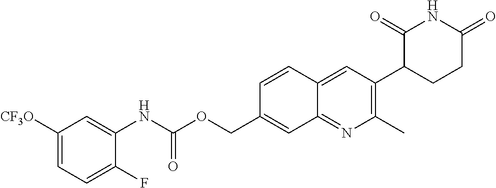 |
| 7 | 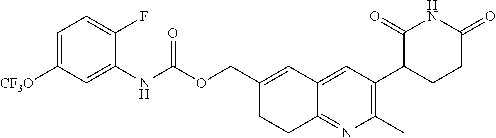 |
| 8 | 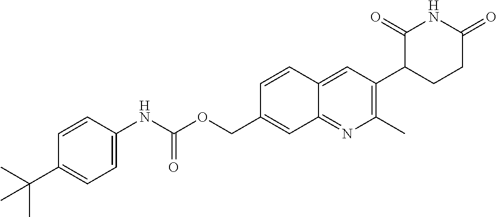 [SMILES: Cc1nc2cc(COC(=O)Nc3ccc(C(C)(C)C)cc3)ccc2cc1C1CCC(=O)NC1=O] |
| 9 | 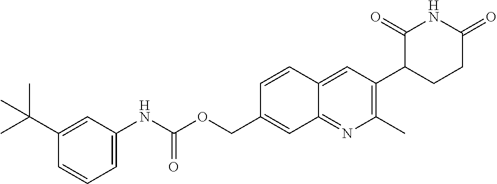 [SMILES: Cc1nc2cc(COC(=O)Nc3cccc(C(C)(C)C)c3)ccc2cc1C1CCC(=O)NC1=O] |
| 10 | 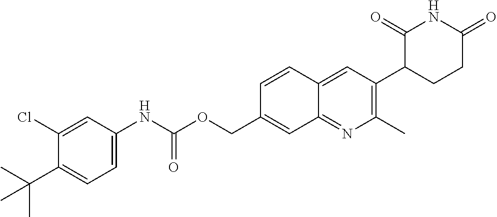 [SMILES: Cc1nc2cc(COC(=O)Nc3ccc(C(C)(C)C)c(Cl)c3)ccc2cc1C1CCC(=O)NC1=O] |
| 11 | 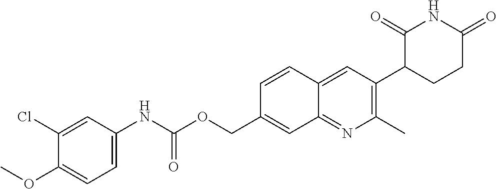 [SMILES: COc1ccc(NC(=O)OCc2ccc3cc(C4CCC(=O)NC4=O)c(C)nc3c2)cc1Cl] |
| 12 | 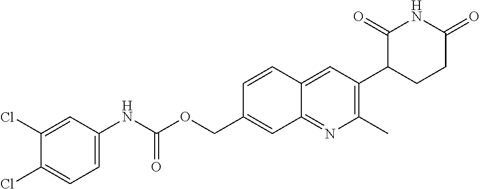 [SMILES: Cc1nc2cc(COC(=O)Nc3ccc(Cl)c(Cl)c3)ccc2cc1C1CCC(=O)NC1=O] |
| 14 | 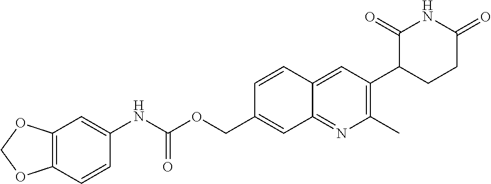 [SMILES: Cc1nc2cc(COC(=O)Nc3ccc4c(c3)OCO4)ccc2cc1C1CCC(=O)NC1=O] |
| 15 | 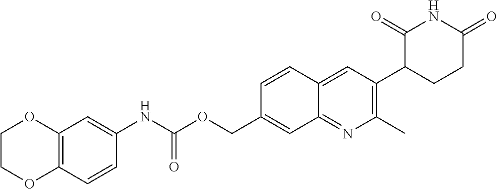 [SMILES: Cc1nc2cc(COC(=O)Nc3ccc4c(c3)OCCO4)ccc2cc1C1CCC(=O)NC1=O] |
| 17 | 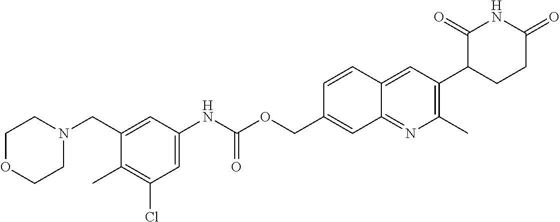 [SMILES: Cc1nc2cc(COC(=O)Nc3cc(Cl)c(C)c(CN4CCOCC4)c3)ccc2cc1C1CCC(=O)NC1=O] |
| 18 | 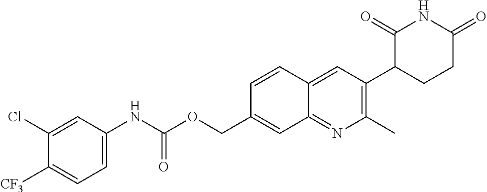 [SMILES: Cc1nc2cc(COC(=O)Nc3ccc(C(F)(F)F)c(Cl)c3)ccc2cc1C1CCC(=O)NC1=O] |
| 19 | 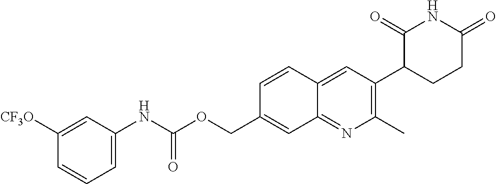 |
| 20 | 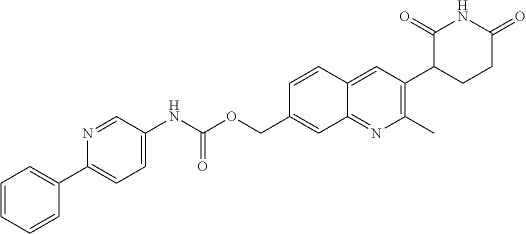 [SMILES: Cc1nc2cc(COC(=O)Nc3ccc(-c4ccccc4)nc3)ccc2cc1C1CCC(=O)NC1=O] |
| 21 | 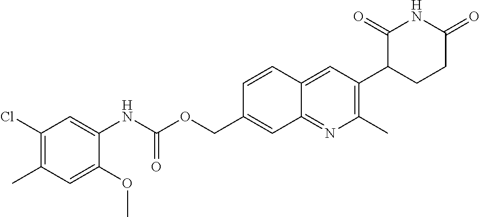 [SMILES: COc1cc(C)c(Cl)cc1NC(=O)OCc1ccc2cc(C3CCC(=O)NC3=O)c(C)nc2c1] |
| 22 | 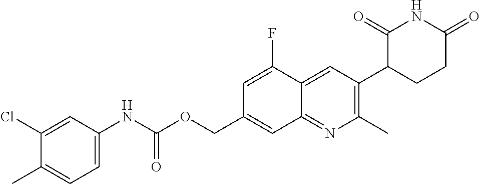 [SMILES: Cc1ccc(NC(=O)OCc2cc(F)c3cc(C4CCC(=O)NC4=O)c(C)nc3c2)cc1Cl] |
| 28 | 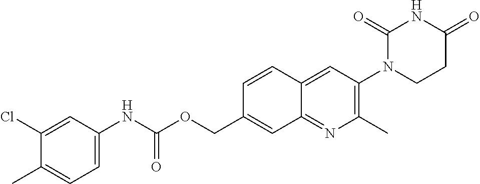 [SMILES: Cc1ccc(NC(=O)OCc2ccc3cc(N4CCC(=O)NC4=O)c(C)nc3c2)cc1Cl] |
| 29 | 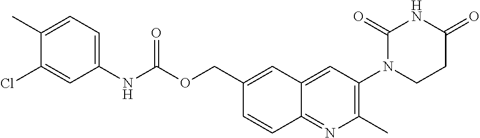 [SMILES: Cc1ccc(NC(=O)OCc2ccc3nc(C)c(N4CCC(=O)NC4=O)cc3c2)cc1Cl] |
| 30 | 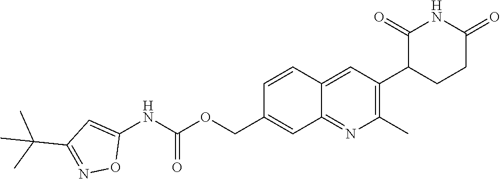 [SMILES: Cc1nc2cc(COC(=O)Nc3cc(C(C)(C)C)no3)ccc2cc1C1CCC(=O)NC1=O] |
| 37 | 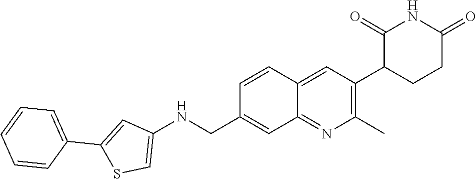 [SMILES: Cc1nc2cc(CNc3csc(-c4ccccc4)c3)ccc2cc1C1CCC(=O)NC1=O] |

46. The compound of clause 44, wherein the compound is selected from

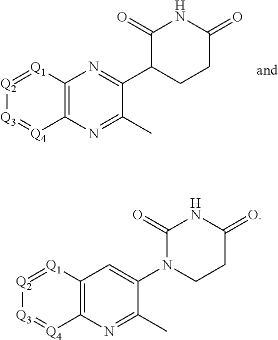

47. The compound of any one of clauses 1-41, wherein R⁵ is

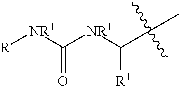

and wherein the compound is selected from

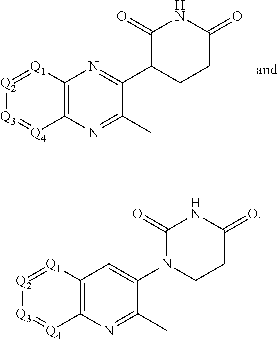

48. The compound of clause 47, wherein the compound is:

| Compound No. | Structure |
|---|---|
| 27 | 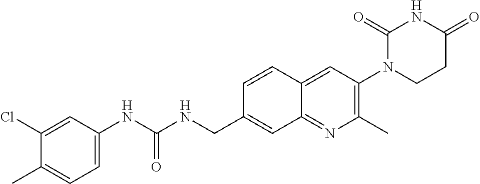 [SMILES: Cc1ccc(NC(=O)NCc2ccc3cc(N4CCC(=O)NC4=O)c(C)nc3c2)cc1Cl] |

49. The compound of any one of clauses 1-41, wherein R⁵ is

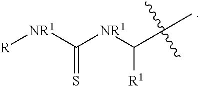

50. The compound of clause 49, wherein the compound is:

| Compound No. | Structure |
|---|---|
| 2 | 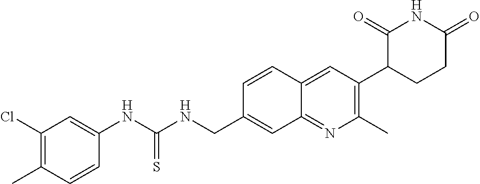 [SMILES: Cc1ccc(NC(=S)NCc2ccc3cc(C4CCC(=O)NC4=O)c(C)nc3c2)cc1Cl] |

51. The compound of any one of clauses 1-41, wherein R⁵ is

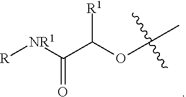

52. The compound of clause 51, wherein R⁵ is selected from:

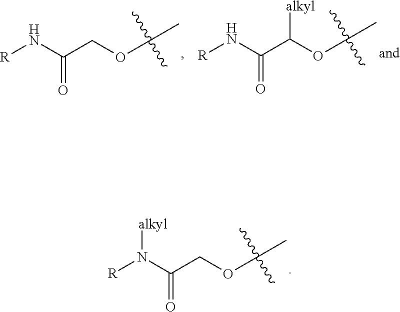

53. The compound of clause 51 or 52, wherein G and E are CH.

54. The compound of clause 53, wherein the compound is selected from:

| Compound No. | Structure |
|---|---|
| 33 | 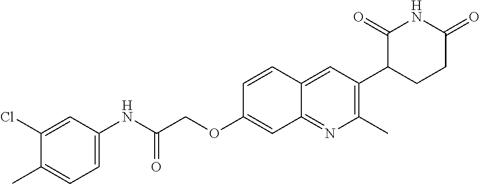 [SMILES: Cc1ccc(NC(=O)COc2ccc3cc(C4CCC(=O)NC4=O)c(C)nc3c2)cc1Cl] |
| 34 | 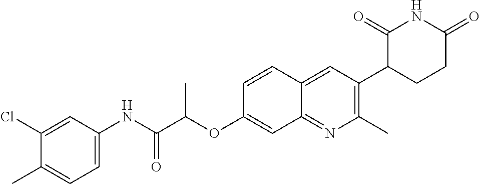 [SMILES: Cc1ccc(NC(=O)C(C)Oc2ccc3cc(C4CCC(=O)NC4=O)c(C)nc3c2)cc1Cl] |
| 35 | 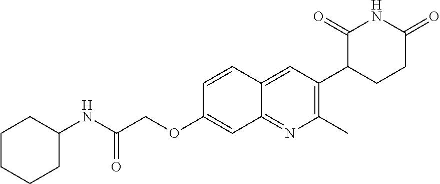 [SMILES: Cc1nc2cc(OCC(=O)NC3CCCCC3)ccc2cc1C1CCC(=O)NC1=O] |
| 36 | 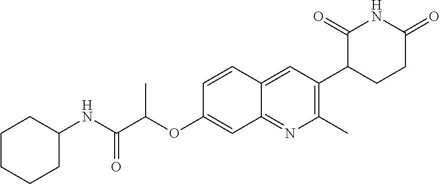 [SMILES: Cc1nc2cc(OC(C)C(=O)NC3CCCCC3)ccc2cc1C1CCC(=O)NC1=O] |

55. The compound of any one of clauses 1-41, wherein R⁵ is:

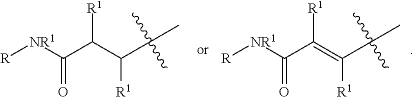

56. The compound of clause 55, wherein R⁵ is:

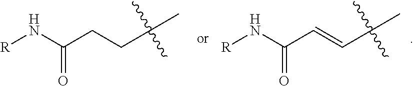

57. The compound of clause 56, wherein the compound is selected from:

| Compound No. | Structure |
|---|---|
| 31 | 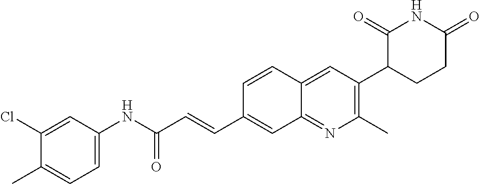 [SMILES: Cc1ccc(NC(=O)/C=C/c2ccc3cc(C4CCC(=O)NC4=O)c(C)nc3c2)cc1Cl] |
| 32 | 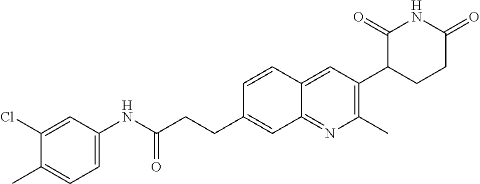 [SMILES: Cc1ccc(NC(=O)CCc2ccc3cc(C4CCC(=O)NC4=O)c(C)nc3c2)cc1Cl] |

58. The compound of any one of clauses 1-41, wherein R⁵ is

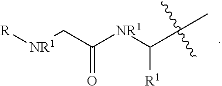

59. The compound of clause 58, wherein R⁵ is

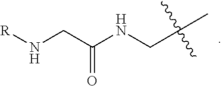

60. The compound of clause 59, wherein the compound is selected from:

| Compound No. | Structure |
|---|---|
| 25 | 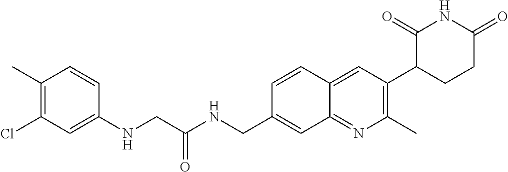 [SMILES: Cc1ccc(NCC(=O)NCc2ccc3cc(C4CCC(=O)NC4=O)c(C)nc3c2)cc1Cl] |
| 26 | 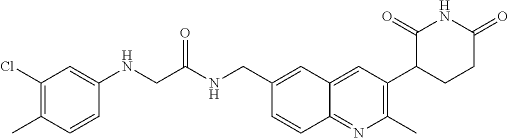 [SMILES: Cc1ccc(NCC(=O)NCc2ccc3nc(C)c(C4CCC(=O)NC4=O)cc3c2)cc1Cl] |

61. The compound of any one of clauses 1-41, wherein R⁵ is

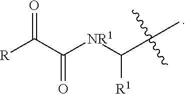

62. The compound of clause 61, wherein the compound is selected from:

| Compound No. | Structure |
|---|---|
| 1 | 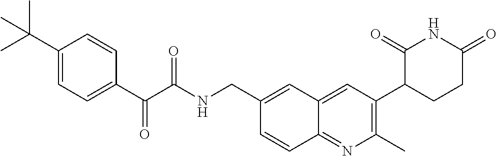 [SMILES: Cc1nc2ccc(CNC(=O)C(=O)c3ccc(C(C)(C)C)cc3)cc2cc1C1CCC(=O)NC1=O] |
| 16 | 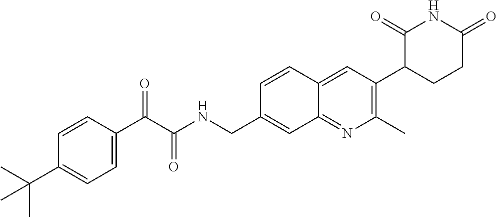 [SMILES: Cc1nc2cc(CNC(=O)C(=O)c3ccc(C(C)(C)C)cc3)ccc2cc1C1CCC(=O)NC1=O] |
| 23 | 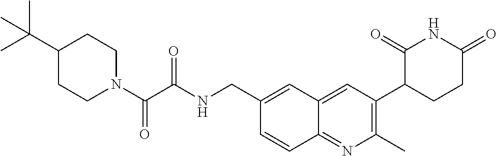 [SMILES: Cc1nc2ccc(CNC(=O)C(=O)N3CCC(C(C)(C)C)CC3)cc2cc1C1CCC(=O)NC1=O] |
| 24 | 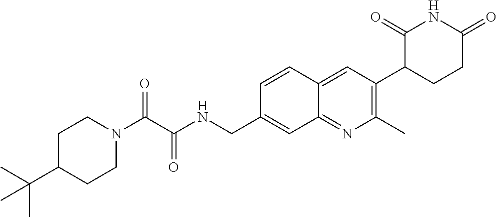 [SMILES: Cc1nc2cc(CNC(=O)C(=O)N3CCC(C(C)(C)C)CC3)ccc2cc1C1CCC(=O)NC1=O] |

63. The compound of any one of clauses 1-39, wherein R⁵ is

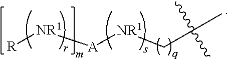

64. The compound of clause 63, wherein the compound is selected from:

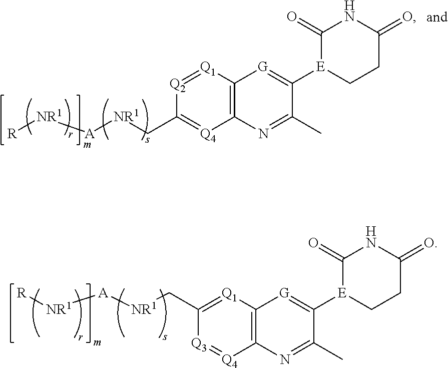

65. The compound of clause 63 or 64, wherein R⁵ is

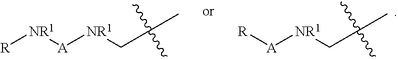

66. The compound of any one of clauses 1-39 and 63-65, wherein A is 5- or 6-membered heteroaryl having 1-3 heteroatoms, 5- or 6-membered heterocycloalkyl having 1-3 heteroatoms, or 4- to 6-membered cycloalkenyl; wherein one or more CH₂ groups of the heterocycloalkyl or cycloalkenyl are optionally replaced by a C═O group.

67. The compound of clause 66, wherein A is 5-membered heteroaryl having 2 or 3 heteroatoms.

68. The compound of clause 66, wherein A is 4-membered cycloalkenyl in which one or more CH₂ groups are replaced by a C═O group.

69. The compound of clause 66, wherein the compound is selected from:

| Compound No. | Structure |
|---|---|
| 3 | 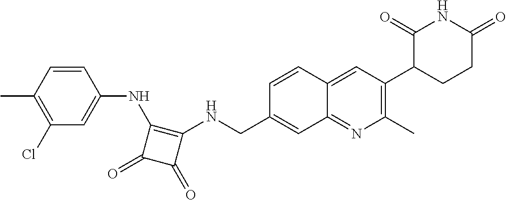 [SMILES: Cc1ccc(Nc2c(NCc3ccc4cc(C5CCC(=O)NC5=O)c(C)nc4c3)c(=O)c2=O)cc1Cl] |
| 13 | 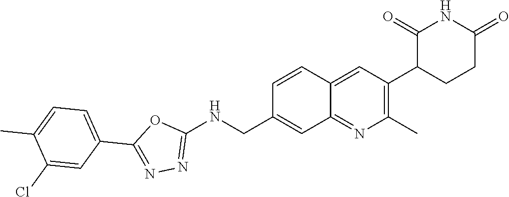 [SMILES: Cc1ccc(-c2nnc(NCc3ccc4cc(C5CCC(=O)NC5=O)c(C)nc4c3)o2)cc1Cl] |

70. The compound of any one of clauses 1-39 and 46, wherein R⁵ is

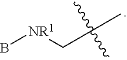

71. The compound of any preceding clause, wherein B is —SO₂NHR or 8-12 membered bicyclic heteroaryl substituted with one or more groups selected from Cl, Me, ^{t}Bu, OCF₃, OMe, CH₂-morpholino, and phenyl.

72. The compound of clause 71, wherein B is 8-12 membered bicyclic heteroaryl substituted with one or more groups selected from Cl, Me, ^{t}Bu, OCF₃, OMe, CH₂-morpholino, and phenyl.

73. The compound of any preceding clause, wherein R is cycloalkyl, heterocycloalkyl, aryl or heteroaryl optionally substituted with one or more R³, or wherein two R³ together with the carbon atoms to which they are attached form a cycloalkyl, heterocycloalkyl or heteroaryl ring.

74. The compound of any preceding clause, wherein each R³ is independently selected from Cl, Me, ^{t}Bu, CF₃, OCF₃, OMe, CH₂-heterocycloalkyl, phenyl, CN, F, NH₂, NHC(O)Me, C(O)Me, cyclopropyl, cyclopropyl substituted with haloalkyl, morpholino, benzyl, pyridyl or ethyl; or wherein two R³ together with the carbon atoms to which they are attached form a cycloalkyl, heterocycloalkyl or heteroaryl ring.

75. The compound of any preceding clause, wherein each R³ is independently selected from Cl, Me, ^{t}Bu, CF₃, OCF₃, OMe, CH₂-heterocycloalkyl or phenyl; or wherein two R³ together with the carbon atoms to which they are attached form a heterocycloalkyl ring.

76. The compound of clause 75 wherein each R³ is independently selected from Cl, Me, ^{t}Bu, CF₃, OCF₃, OMe, —CH₂-morpholino or phenyl; or wherein two R³ together with the carbon atoms to which they are attached form a heterocycloalkyl ring.

77. The compound of any preceding clause, wherein R is selected from:

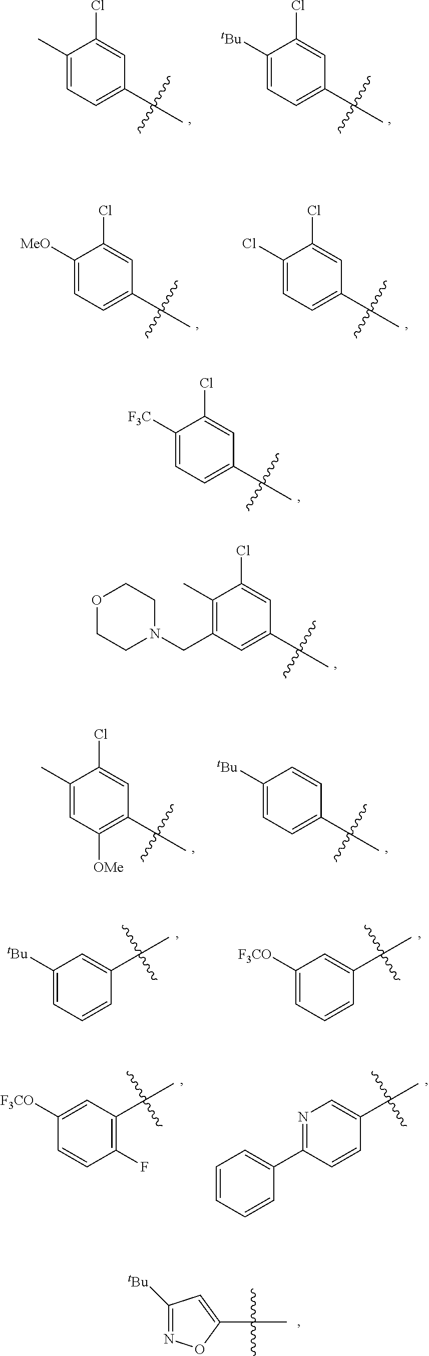

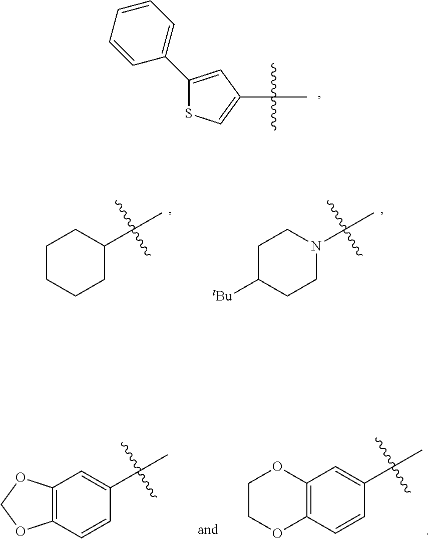 [SMILES: *c1csc(-c2ccccc2)c1 ; *C1CCCCC1 ; *N1CCC(C(C)(C)C)CC1 ; *c1ccc2c(c1)OCO2 ; *c1ccc2c(c1)OCCO2]

78. The compound of any preceding clause, wherein each R² is independently hydrogen or halogen.

79. The compound of any preceding clause, wherein each R⁴ is independently hydrogen, halogen or alkyl.

80. The compound of any preceding clause, wherein each R¹ is independently hydrogen or methyl

81. The compound of any preceding clause, wherein each NR¹ is NH.

82. The compound of clause 1, wherein the compound is selected from:

| Compound No. | Structure |
|---|---|
| 1 | 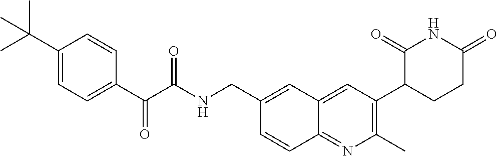 [SMILES: Cc1nc2ccc(CNC(=O)C(=O)c3ccc(C(C)(C)C)cc3)cc2cc1C1CCC(=O)NC1=O] |
| 2 | 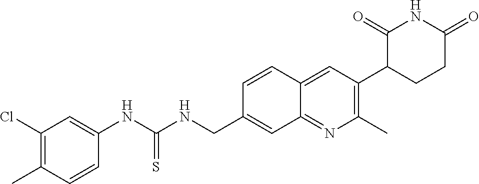 [SMILES: Cc1ccc(NC(=S)NCc2ccc3cc(C4CCC(=O)NC4=O)c(C)nc3c2)cc1Cl] |
| 3 | 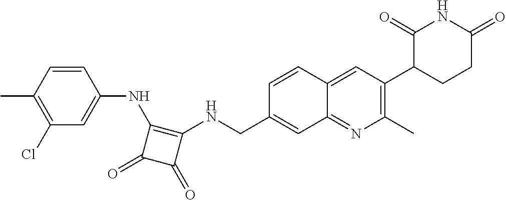 [SMILES: Cc1ccc(Nc2c(NCc3ccc4cc(C5CCC(=O)NC5=O)c(C)nc4c3)c(=O)c2=O)cc1Cl] |
| 4 | 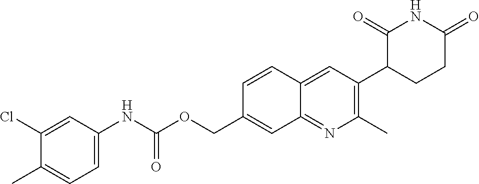 [SMILES: Cc1ccc(NC(=O)OCc2ccc3cc(C4CCC(=O)NC4=O)c(C)nc3c2)cc1Cl] |
| 5 | 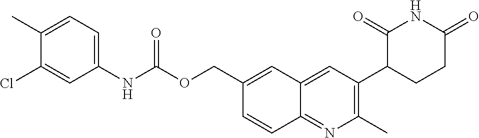 [SMILES: Cc1ccc(NC(=O)OCc2ccc3nc(C)c(C4CCC(=O)NC4=O)cc3c2)cc1Cl] |
| 6 | 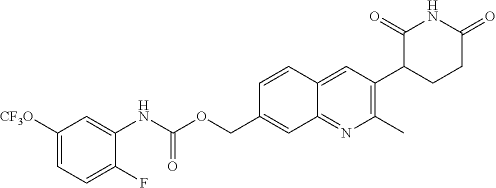 |
| 7 | 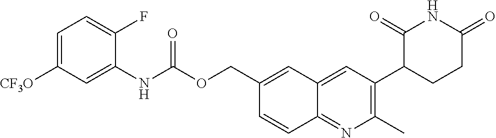 |
| 8 | 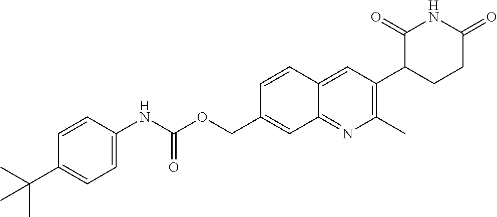 [SMILES: Cc1nc2cc(COC(=O)Nc3ccc(C(C)(C)C)cc3)ccc2cc1C1CCC(=O)NC1=O] |
| 9 | 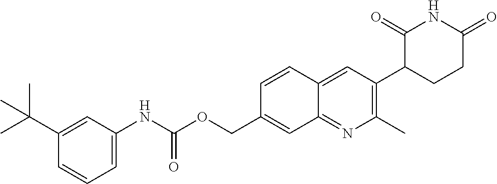 [SMILES: Cc1nc2cc(COC(=O)Nc3cccc(C(C)(C)C)c3)ccc2cc1C1CCC(=O)NC1=O] |
| 10 | 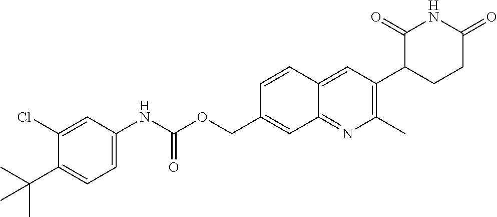 [SMILES: Cc1nc2cc(COC(=O)Nc3ccc(C(C)(C)C)c(Cl)c3)ccc2cc1C1CCC(=O)NC1=O] |
| 11 | 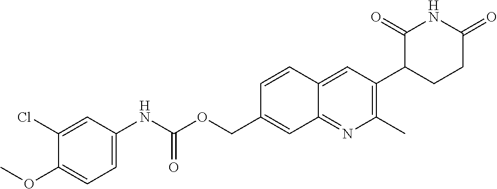 [SMILES: COc1ccc(NC(=O)OCc2ccc3cc(C4CCC(=O)NC4=O)c(C)nc3c2)cc1Cl] |
| 12 | 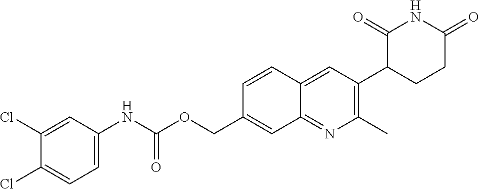 [SMILES: Cc1nc2cc(COC(=O)Nc3ccc(Cl)c(Cl)c3)ccc2cc1C1CCC(=O)NC1=O] |
| 13 | 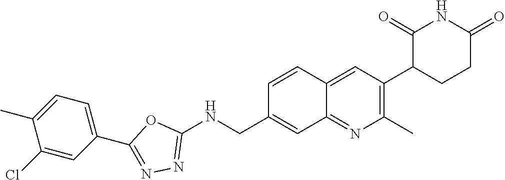 [SMILES: Cc1ccc(-c2nnc(NCc3ccc4cc(C5CCC(=O)NC5=O)c(C)nc4c3)o2)cc1Cl] |
| 14 | 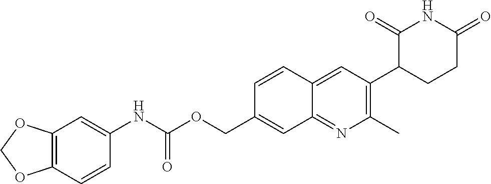 [SMILES: Cc1nc2cc(COC(=O)Nc3ccc4c(c3)OCO4)ccc2cc1C1CCC(=O)NC1=O] |
| 15 | 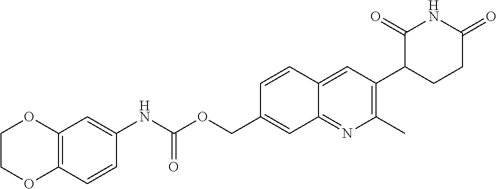 [SMILES: Cc1nc2cc(COC(=O)Nc3ccc4c(c3)OCCO4)ccc2cc1C1CCC(=O)NC1=O] |
| 16 | 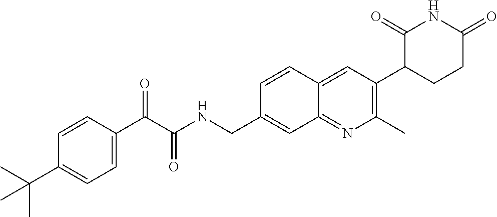 [SMILES: Cc1nc2cc(CNC(=O)C(=O)c3ccc(C(C)(C)C)cc3)ccc2cc1C1CCC(=O)NC1=O] |
| 17 | 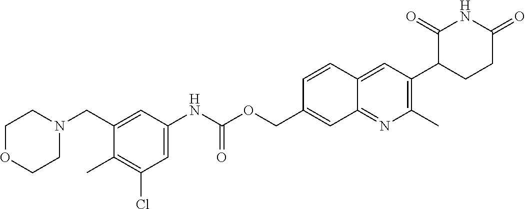 [SMILES: Cc1nc2cc(COC(=O)Nc3cc(Cl)c(C)c(CN4CCOCC4)c3)ccc2cc1C1CCC(=O)NC1=O] |
| 18 | 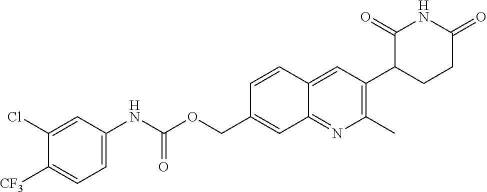 [SMILES: Cc1nc2cc(COC(=O)Nc3ccc(C(F)(F)F)c(Cl)c3)ccc2cc1C1CCC(=O)NC1=O] |
| 19 | 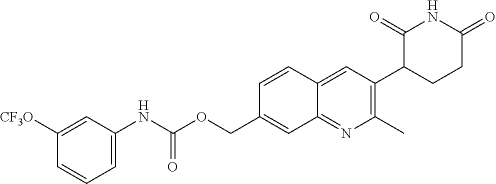 |
| 20 | 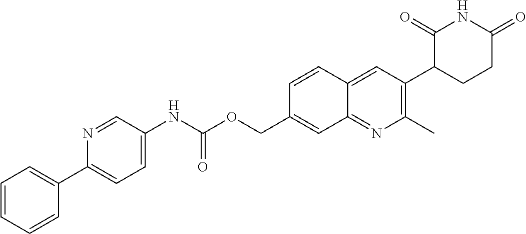 [SMILES: Cc1nc2cc(COC(=O)Nc3ccc(-c4ccccc4)nc3)ccc2cc1C1CCC(=O)NC1=O] |
| 21 | 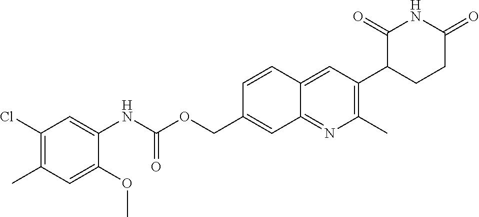 [SMILES: COc1cc(C)c(Cl)cc1NC(=O)OCc1ccc2cc(C3CCC(=O)NC3=O)c(C)nc2c1] |
| 22 | 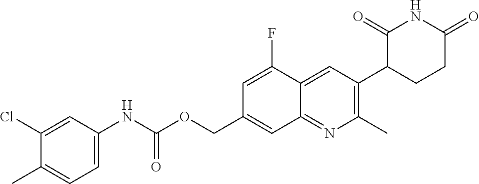 [SMILES: Cc1ccc(NC(=O)OCc2cc(F)c3cc(C4CCC(=O)NC4=O)c(C)nc3c2)cc1Cl] |
| 23 | 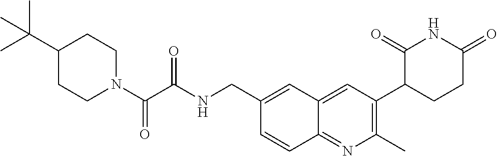 [SMILES: Cc1nc2ccc(CNC(=O)C(=O)N3CCC(C(C)(C)C)CC3)cc2cc1C1CCC(=O)NC1=O] |
| 24 | 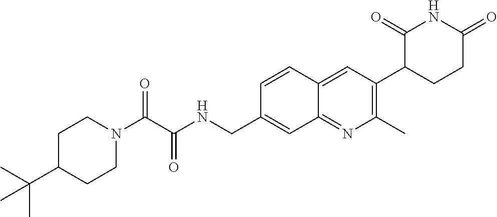 [SMILES: Cc1nc2cc(CNC(=O)C(=O)N3CCC(C(C)(C)C)CC3)ccc2cc1C1CCC(=O)NC1=O] |
| 25 | 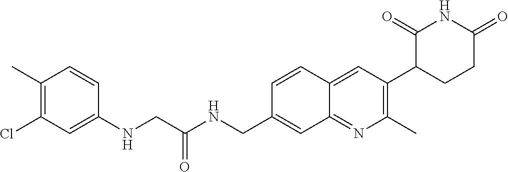 [SMILES: Cc1ccc(NCC(=O)NCc2ccc3cc(C4CCC(=O)NC4=O)c(C)nc3c2)cc1Cl] |
| 26 | 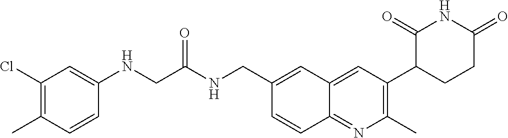 [SMILES: Cc1ccc(NCC(=O)NCc2ccc3nc(C)c(C4CCC(=O)NC4=O)cc3c2)cc1Cl] |
| 27 | 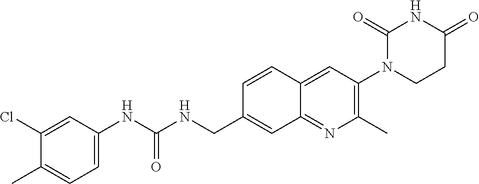 [SMILES: Cc1ccc(NC(=O)NCc2ccc3cc(N4CCC(=O)NC4=O)c(C)nc3c2)cc1Cl] |
| 28 | 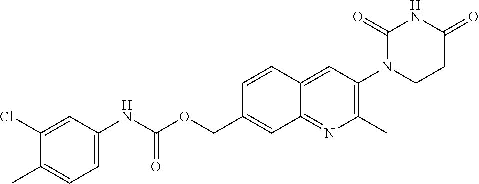 [SMILES: Cc1ccc(NC(=O)OCc2ccc3cc(N4CCC(=O)NC4=O)c(C)nc3c2)cc1Cl] |
| 29 | 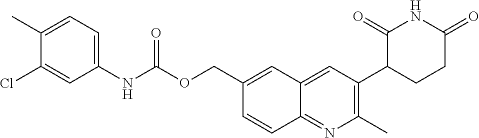 [SMILES: Cc1ccc(NC(=O)OCc2ccc3nc(C)c(C4CCC(=O)NC4=O)cc3c2)cc1Cl] |
| 30 | 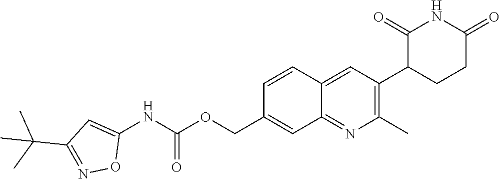 [SMILES: Cc1nc2cc(COC(=O)Nc3cc(C(C)(C)C)no3)ccc2cc1C1CCC(=O)NC1=O] |
| 31 | 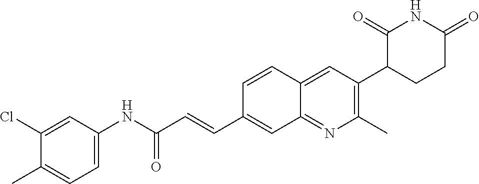 [SMILES: Cc1ccc(NC(=O)/C=C/c2ccc3cc(C4CCC(=O)NC4=O)c(C)nc3c2)cc1Cl] |
| 32 | 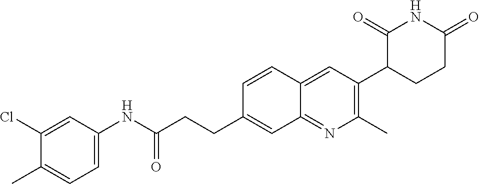 [SMILES: Cc1ccc(NC(=O)CCc2ccc3cc(C4CCC(=O)NC4=O)c(C)nc3c2)cc1Cl] |
| 33 | 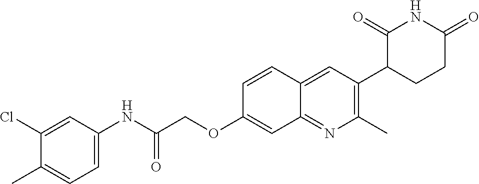 [SMILES: Cc1ccc(NC(=O)COc2ccc3cc(C4CCC(=O)NC4=O)c(C)nc3c2)cc1Cl] |
| 34 | 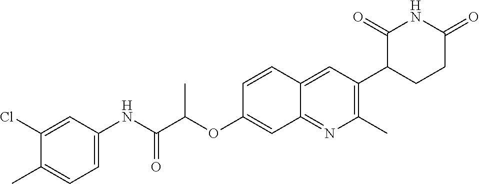 [SMILES: Cc1ccc(NC(=O)C(C)Oc2ccc3cc(C4CCC(=O)NC4=O)c(C)nc3c2)cc1Cl] |
| 35 | 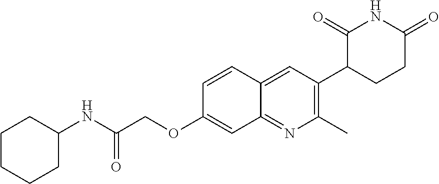 [SMILES: Cc1nc2cc(OCC(=O)NC3CCCCC3)ccc2cc1C1CCC(=O)NC1=O] |
| 36 | 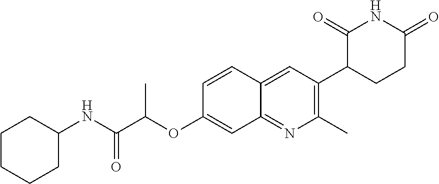 [SMILES: Cc1nc2cc(OC(C)C(=O)NC3CCCCC3)ccc2cc1C1CCC(=O)NC1=O] |
| 37 | 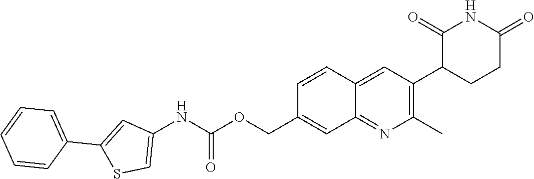 [SMILES: Cc1nc2cc(COC(=O)Nc3csc(-c4ccccc4)c3)ccc2cc1C1CCC(=O)NC1=O] |

83. A pharmaceutical composition comprising a compound of any one of clauses 1-82.

84. The compound of any one of clauses 1-82 or the pharmaceutical composition of clause 83 for use in medicine.

85. The compound of any one of clauses 1-82 or the pharmaceutical composition of clause 83 for use in the treatment of cancer.

86. The compound or pharmaceutical composition for use of clause 85, wherein the cancer is hepatocellular carcinoma, neuroblastoma, leukemia, acute myeloid leukemia (AML), acute promyelocytic leukemia (APL), multiple myeloma, breast cancer, prostate cancer, bladder cancer, kidney cancer, muscle cancer, ovarian cancer, skin cancer, pancreatic cancer, colon cancer, hematological cancer, cancer of connective tissue, placental cancer, bone cancer, uterine cancer, cervical cancer, choriocarcinoma, endometrial cancer, gastric cancer, or lung cancer.

1. A compound of formula (I): 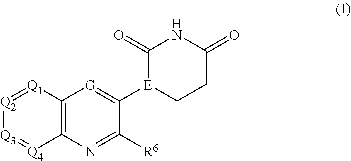
wherein
R⁶ is hydrogen, unsubstituted C₁-C₄ alkyl, haloalkyl, —OR¹ or —N(R¹)₂;
E is CH, CD, CF, C—(C₁-C₃ alkyl) or N;
G is CR¹ or N;
one of Q₁, Q₂, Q₃, and Q₄ is CR⁵ and the other three of Q₁, Q₂, Q₃, and Q₄ are each independently N or CR⁴; wherein when G is CR¹ then at least one of Q₁, Q₂, Q₃, and Q₄ is CR⁴; and
when G is N then at least two of Q₁, Q₂, Q₃, and Q₄ are CR⁴;
each R¹ is independently hydrogen, unsubstituted C₁-C₄ alkyl, or C₁-C₄ haloalkyl;
each R⁴ is independently hydrogen, unsubstituted alkyl, haloalkyl, halogen, —CN, —OR¹ or —N(R¹)₂;
R⁵ is selected from: 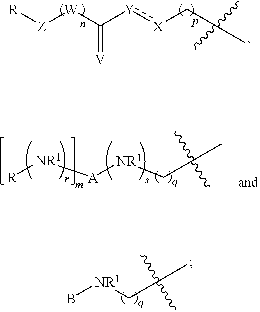
wherein [symbol] is a single bond or a double bond, wherein
when [symbol] is a single bond, then X and Y are each independently O, NR¹, CHR¹ or CD₂;
wherein when one of X and Y is O or NR¹, then the other of X and Y is CHR¹ or CD₂; and
when [symbol] is a double bond, then X and Y are each CR¹;
W is CH₂ or C═O;
Z is C═O, NR¹ or C(R²)₂; wherein each R² is independently hydrogen, unsubstituted alkyl, halogen, OR¹ or N(R¹)₂;
V is O or S;
each R is independently cycloalkyl, heterocycloalkyl, aryl, heteroaryl, alkyl, or benzyl wherein each cycloalkyl, heterocycloalkyl, aryl, heteroaryl, alkyl or benzyl is optionally substituted with one or more R³, wherein each R³ is independently selected from halogen, R⁷, —CH₂-heterocycloalkyl, —CN, —OH, OR⁷, —NH₂, —NR¹R⁷, —NHC(O)R⁷, —NHC(O)OR⁷, —NHC(O)NR¹R⁷ and —C(O)R⁷; or wherein two R³ together with the carbon atoms to which they are attached form a cycloalkyl, heterocycloalkyl or heteroaryl ring;
or wherein R and R¹, together with the N atom to which they are attached when Z is NR¹, form a heterocycloalkyl or heteroaryl ring which is optionally substituted with one or more groups selected from halogen, unsubstituted alkyl, haloalkyl, —OH, —O(haloalkyl), —O(unsubstituted alkyl) and —N(R¹)₂;
each R⁷ is independently alkyl, haloalkyl, heteroaryl, aryl, benzyl, cycloalkyl or heterocycloalkyl;
A is heteroaryl, heterocycloalkyl, cycloalkyl or cycloalkenyl;
B is —SO₂NHR or 8-12 membered bicyclic heteroaryl substituted with one or more R⁸, wherein each R⁸ is independently selected from halogen, alkyl, —O(haloalkyl), —O(unsubstituted alkyl), aryl, and CH₂-heterocycloalkyl;
n is 0 or 1;
m is 1 or 2;
r is 0 or 1;
s is 0 or 1;
each p is independently 0, 1, 2 or 3; and
each q is independently 1, 2, 3 or 4;
wherein:
one or more CH₂ groups of any cycloalkyl, heterocycloalkyl or cycloalkenyl are optionally replaced by a C═O group;
in E, R³, R⁷ and R⁸, each alkyl is independently optionally substituted with one or more groups selected from halogen, —OH, —O(haloalkyl), —O(unsubstituted alkyl) and —N(R¹)₂; and
in A, R³, R⁷ and R⁸, each cycloalkyl, heterocycloalkyl, cycloalkenyl, aryl and heteroaryl is independently optionally substituted with one or more groups selected from halogen, unsubstituted alkyl, haloalkyl, —OH, —O(haloalkyl), —O(unsubstituted alkyl) and —N(R¹)₂;
provided that:
(a) when R⁵ is 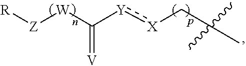
W is C═O, n is 1 and p is 0; then Z is NR¹ or C(R²)₂; and
(b) when R⁵ is 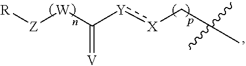
X is CHR¹, Y is NH, V is O, n is 0, p is 0, Z is NH or C(halogen)₂, and R is aryl; then at least one of E and G is N.

2. The compound of claim 1, wherein when R⁵ is 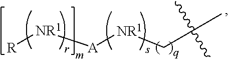
then E is CH, CD, CF or C—(C₁-C₃ alkyl).

3. The compound of claim 2, wherein when R⁵ is 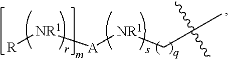
then E is CH.

4. The compound of any preceding claim, wherein at least one of r and s is 1.

5. The compound of any preceding claim, wherein r is 1.

6. The compound of any preceding claim, wherein s is 1.

7. The compound of any preceding claim, wherein when [symbol] is a single bond and one of X and Y is CHR¹, then the other of X and Y is O or NR¹.

8. The compound of any preceding claim, wherein in E, R³, R⁷ and R⁸, each alkyl is independently optionally substituted with one or more groups selected from halogen, —OH, —O(haloalkyl), and —O(unsubstituted alkyl).

9. The compound of claim 8, wherein in E, R³, R⁷ and R⁸, each alkyl is independently optionally substituted with one or more groups selected from halogen and —OH.

10. The compound of any one of claims 1-9, wherein in E, R³, R⁷ and R⁸, each alkyl is unsubstituted.

11. The compound of any preceding claim, wherein the C₁-C₃ alkyl of E is unsubstituted C₁-C₃ alkyl.

12. The compound of any one of claims 1-7, wherein E is CH, CD or N.

13. The compound of claim 12, wherein E is CH or N.

14. The compound of claim 13, wherein E is N.

15. The compound of any one of claims 1-11, wherein E is CH, CD, CF or C—(C₁-C₃ alkyl).

16. The compound of claim 15, wherein E is CH, CD or C—(C₁-C₃ alkyl).

17. The compound of claim 15, wherein E is CH or CF; optionally wherein E is CH.

18. The compound of any preceding claim, wherein in A, R³, R⁷ and R⁸, any cycloalkyl, heterocycloalkyl, cycloalkenyl, aryl and heteroaryl is optionally substituted with one or more groups selected from halogen, unsubstituted alkyl, haloalkyl, —O(haloalkyl) and —O(unsubstituted alkyl).

19. The compound of any preceding claim, wherein the heterocycloalkyl or heteroaryl ring formed by R and R¹, together with the N atom to which they are attached when Z is NR¹, is optionally substituted with one or more groups selected from halogen, unsubstituted alkyl, haloalkyl, —O(haloalkyl) and —O(unsubstituted alkyl).

20. The compound of any preceding claim, wherein each R³ is independently selected from halogen, R⁷, —CH₂-heterocycloalkyl, —CN, —OH, —OR⁷, —NH₂, —NR¹R⁷, —NHC(O)R⁷, —NHC(O)OR⁷, —NHC(O)NR¹R⁷ and —C(O)R⁷.

21. The compound of claim 20, wherein each R³ is independently selected from halogen, R⁷, —CH₂-heterocycloalkyl, —CN, —OR⁷, —NH₂, —NHC(O)R⁷ and —C(O)R⁷.

22. The compound of claim 21, wherein each R³ is independently selected from halogen, R⁷, —CH₂-heterocycloalkyl, —CN, —OR⁷, —NH₂, —NHC(O)alkyl and —C(O)alkyl.

23. The compound of claim 22, wherein each R³ is independently selected from halogen, R⁷, —CH₂-heterocycloalkyl, —CN, —O(haloalkyl), —O(alkyl), —NH₂, —NHC(O)alkyl and —C(O)alkyl;

24. The compound of any preceding claim, wherein each R¹ is independently hydrogen or unsubstituted C₁-C₄ alkyl.

25. The compound of any preceding claim, wherein each R⁴ is independently hydrogen, unsubstituted alkyl, haloalkyl, halogen, —OR¹ or —N(R¹)₂

26. The compound of claim 25, wherein each R⁴ is independently hydrogen, unsubstituted alkyl, halogen, —OR¹ or —N(R¹)₂

27. The compound of any preceding claim, wherein R⁶ is unsubstituted C₁-C₄ alkyl, haloalkyl, OR¹ or N(R¹)₂.

28. The compound of claim 27, wherein R⁶ is OR¹ or N(R¹)₂.

29. The compound of claim 27, wherein R⁶ is unsubstituted C₁-C₄ alkyl or haloalkyl.

30. The compound of claim 29, wherein R⁶ is unsubstituted C₁-C₄ alkyl or C₁-C₄ haloalkyl.

31. The compound of claim 30, wherein R⁶ is unsubstituted C₁-C₄ alkyl, optionally methyl.

32. The compound of claim 31, wherein R⁶ is methyl.

33. The compound of any preceding claim, wherein when R⁵ is 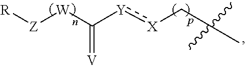
X is CHR¹, Y is NR¹, V is O, n is 0, p is 0, Z is NR¹ or C(halogen)₂, and R is aryl; then at least one of E and G is N.

34. The compound of any preceding claim, wherein G is CR¹.

35. The compound of claim 34, wherein G is CH.

36. The compound of any one of claims 1-33, wherein G is N.

37. The compound of any preceding claim, wherein at least two of Q₁, Q₂, Q₃, and Q₄ are CR⁴.

38. The compound of any preceding claim, wherein three of Q₁, Q₂, Q₃, and Q₄ are CR⁴.

39. The compound of claim 38, wherein:
(a) Q₁ is CR⁵ and Q₂, Q₃, and Q₄ are each independently CR⁴; or
(b) Q₂ is CR⁵ and Q₁, Q₃, and Q₄ are each independently CR⁴; or
(c) Q₃ is CR⁵ and Q₁, Q₂, and Q₄ are each independently CR⁴; or
(d) Q₄ is CR⁵ and Q₁, Q₂, and Q₃ are each independently CR⁴.

40. The compound of any preceding claim, wherein R⁵ is

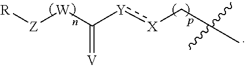

41. The compound of claim 40, wherein the compound is selected from: 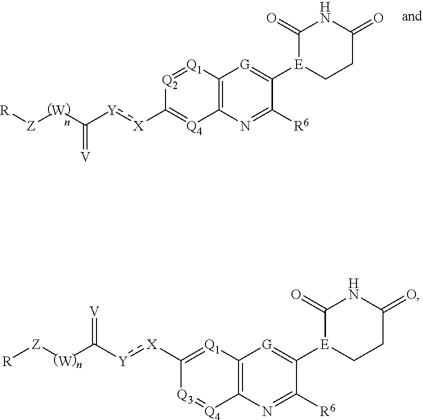
optionally wherein R⁶ is methyl.

42. The compound of any one of claims 40-41, wherein R⁵ is

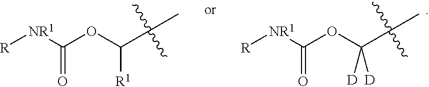

43. The compound of claim 42, wherein R⁵ is selected from

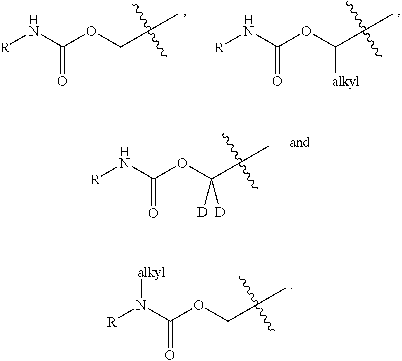

44. The compound of claim 42 or 43, wherein G is CH and E is CH, CD, CF or C—(C₁-C₃ alkyl).

45. The compound of claim 44, wherein the compound is selected from:

| Compound No. | Structure |
|---|---|
| 4 | 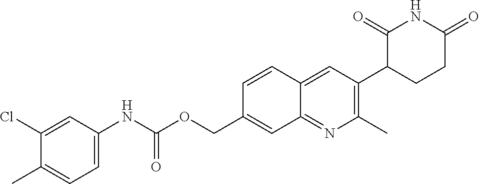 [SMILES: Cc1ccc(NC(=O)OCc2ccc3cc(C4CCC(=O)NC4=O)c(C)nc3c2)cc1Cl] |
| 5 | 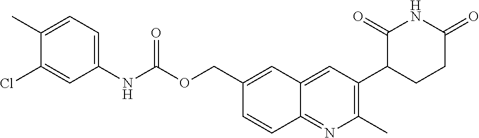 [SMILES: Cc1ccc(NC(=O)OCc2ccc3nc(C)c(C4CCC(=O)NC4=O)cc3c2)cc1Cl] |
| 6 | 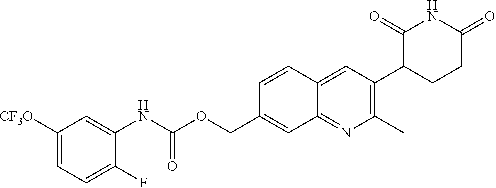 |
| 7 | 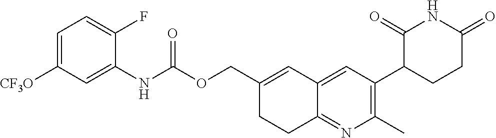 |
| 8 | 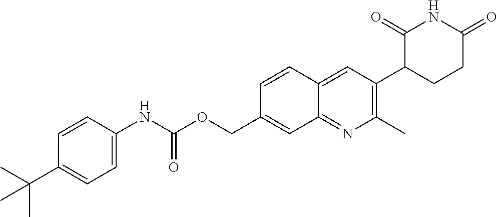 [SMILES: Cc1nc2cc(COC(=O)Nc3ccc(C(C)(C)C)cc3)ccc2cc1C1CCC(=O)NC1=O] |
| 9 | 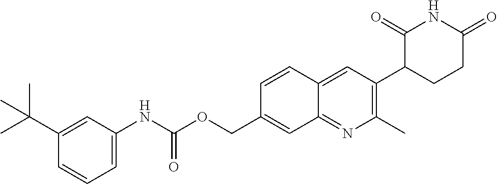 [SMILES: Cc1nc2cc(COC(=O)Nc3cccc(C(C)(C)C)c3)ccc2cc1C1CCC(=O)NC1=O] |
| 10 | 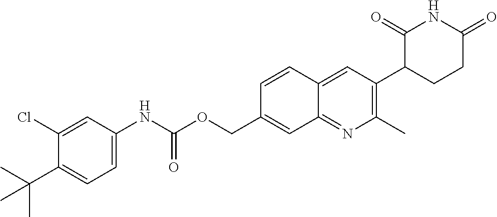 [SMILES: Cc1nc2cc(COC(=O)Nc3ccc(C(C)(C)C)c(Cl)c3)ccc2cc1C1CCC(=O)NC1=O] |
| 11 | 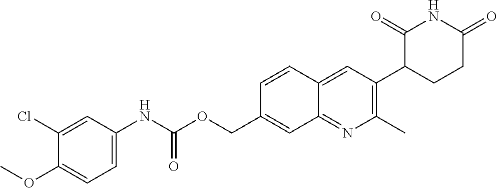 [SMILES: COc1ccc(NC(=O)OCc2ccc3cc(C4CCC(=O)NC4=O)c(C)nc3c2)cc1Cl] |
| 12 | 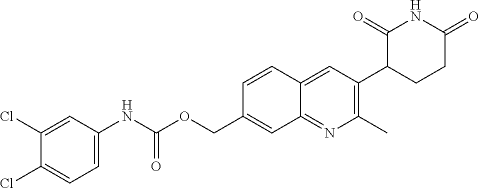 [SMILES: Cc1nc2cc(COC(=O)Nc3ccc(Cl)c(Cl)c3)ccc2cc1C1CCC(=O)NC1=O] |
| 14 | 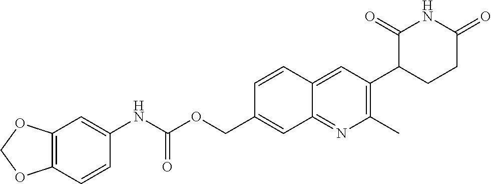 [SMILES: Cc1nc2cc(COC(=O)Nc3ccc4c(c3)OCO4)ccc2cc1C1CCC(=O)NC1=O] |
| 15 | 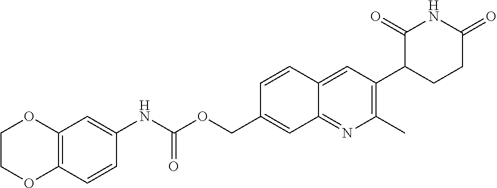 [SMILES: Cc1nc2cc(COC(=O)Nc3ccc4c(c3)OCCO4)ccc2cc1C1CCC(=O)NC1=O] |
| 17 | 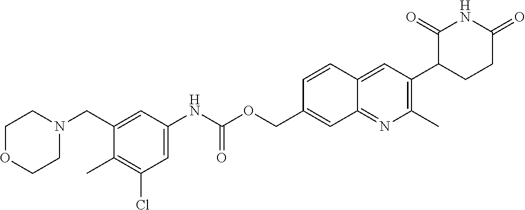 [SMILES: Cc1nc2cc(COC(=O)Nc3cc(Cl)c(C)c(CN4CCOCC4)c3)ccc2cc1C1CCC(=O)NC1=O] |
| 18 | 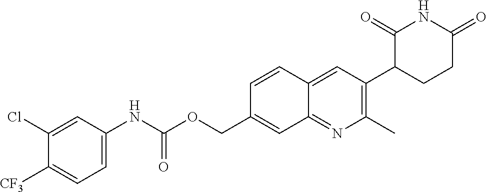 [SMILES: Cc1nc2cc(COC(=O)Nc3ccc(C(F)(F)F)c(Cl)c3)ccc2cc1C1CCC(=O)NC1=O] |
| 19 | 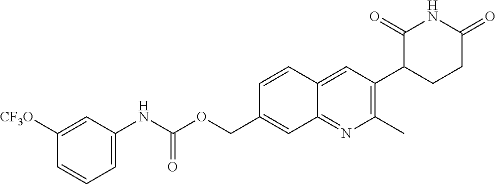 |
| 20 | 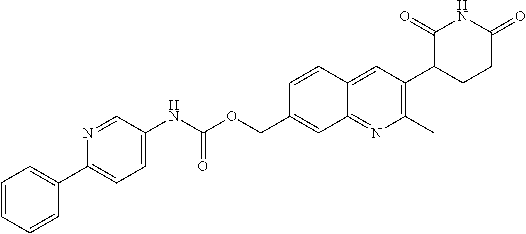 [SMILES: Cc1nc2cc(COC(=O)Nc3ccc(-c4ccccc4)nc3)ccc2cc1C1CCC(=O)NC1=O] |
| 21 | 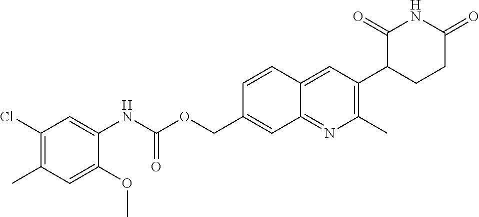 [SMILES: COc1cc(C)c(Cl)cc1NC(=O)OCc1ccc2cc(C3CCC(=O)NC3=O)c(C)nc2c1] |
| 22 | 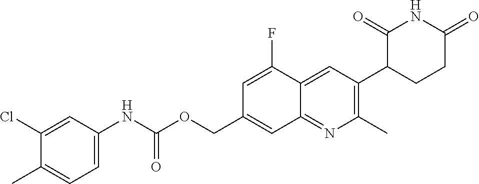 [SMILES: Cc1ccc(NC(=O)OCc2cc(F)c3cc(C4CCC(=O)NC4=O)c(C)nc3c2)cc1Cl] |
| 28 | 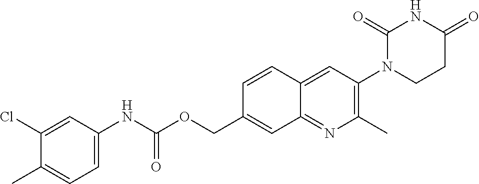 [SMILES: Cc1ccc(NC(=O)OCc2ccc3cc(N4CCC(=O)NC4=O)c(C)nc3c2)cc1Cl] |
| 29 | 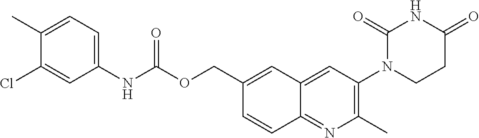 [SMILES: Cc1ccc(NC(=O)OCc2ccc3nc(C)c(N4CCC(=O)NC4=O)cc3c2)cc1Cl] |
| 30 | 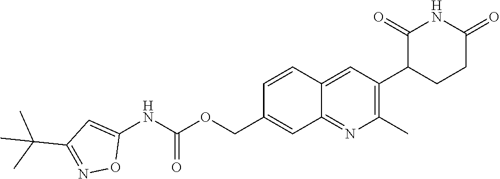 [SMILES: Cc1nc2cc(COC(=O)Nc3cc(C(C)(C)C)no3)ccc2cc1C1CCC(=O)NC1=O] |
| 37 | 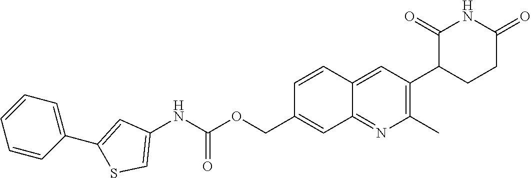 [SMILES: Cc1nc2cc(COC(=O)Nc3csc(-c4ccccc4)c3)ccc2cc1C1CCC(=O)NC1=O] |
| 38 | 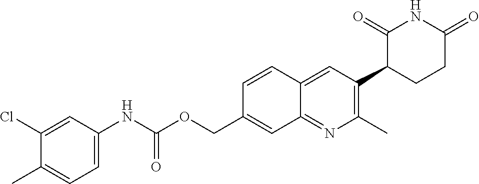 [SMILES: Cc1ccc(NC(=O)OCc2ccc3cc([C@@H]4CCC(=O)NC4=O)c(C)nc3c2)cc1Cl] |
| 39 | 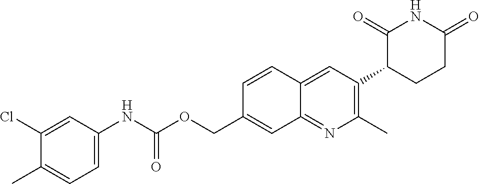 [SMILES: Cc1ccc(NC(=O)OCc2ccc3cc([C@H]4CCC(=O)NC4=O)c(C)nc3c2)cc1Cl] |
| 42 | 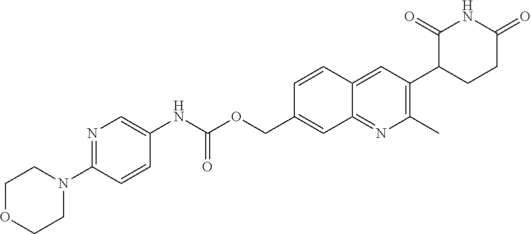 [SMILES: Cc1nc2cc(COC(=O)Nc3ccc(N4CCOCC4)nc3)ccc2cc1C1CCC(=O)NC1=O] |
| 44 | 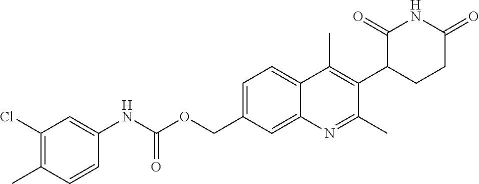 [SMILES: Cc1ccc(NC(=O)OCc2ccc3c(C)c(C4CCC(=O)NC4=O)c(C)nc3c2)cc1Cl] |
| 45 | 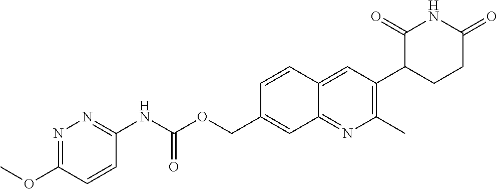 [SMILES: COc1ccc(NC(=O)OCc2ccc3cc(C4CCC(=O)NC4=O)c(C)nc3c2)nn1] |
| 46 | 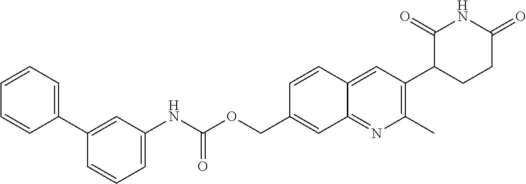 [SMILES: Cc1nc2cc(COC(=O)Nc3cccc(-c4ccccc4)c3)ccc2cc1C1CCC(=O)NC1=O] |
| 47 | 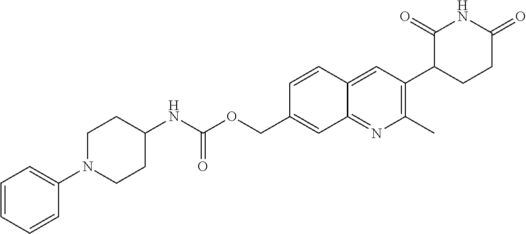 [SMILES: Cc1nc2cc(COC(=O)NC3CCN(c4ccccc4)CC3)ccc2cc1C1CCC(=O)NC1=O] |
| 49 | 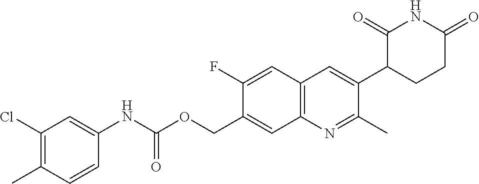 [SMILES: Cc1ccc(NC(=O)OCc2cc3nc(C)c(C4CCC(=O)NC4=O)cc3cc2F)cc1Cl] |
| 50 | 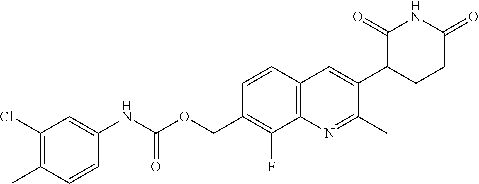 [SMILES: Cc1ccc(NC(=O)OCc2ccc3cc(C4CCC(=O)NC4=O)c(C)nc3c2F)cc1Cl] |
| 54 | 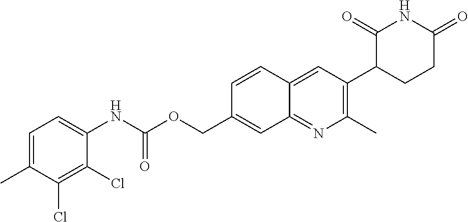 [SMILES: Cc1ccc(NC(=O)OCc2ccc3cc(C4CCC(=O)NC4=O)c(C)nc3c2)c(Cl)c1Cl] |
| 55 | 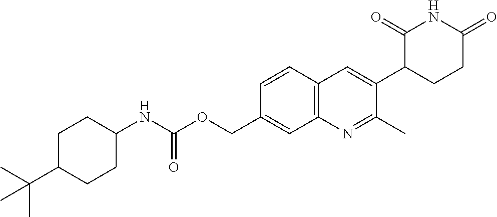 [SMILES: Cc1nc2cc(COC(=O)NC3CCC(C(C)(C)C)CC3)ccc2cc1C1CCC(=O)NC1=O] |
| 58 | 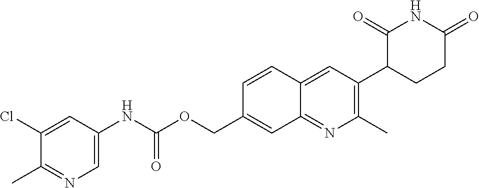 [SMILES: Cc1ncc(NC(=O)OCc2ccc3cc(C4CCC(=O)NC4=O)c(C)nc3c2)cc1Cl] |
| 59 | 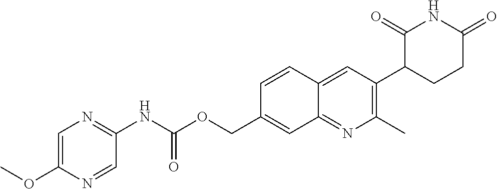 [SMILES: COc1cnc(NC(=O)OCc2ccc3cc(C4CCC(=O)NC4=O)c(C)nc3c2)cn1] |
| 60 | 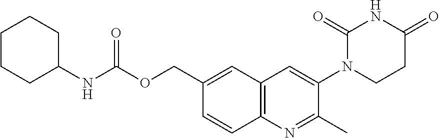 [SMILES: Cc1nc2ccc(COC(=O)NC3CCCCC3)cc2cc1N1CCC(=O)NC1=O] |
| 68 | 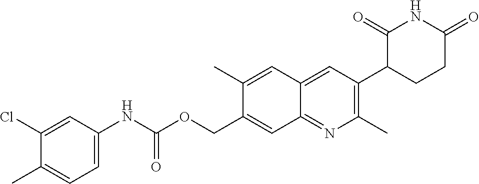 [SMILES: Cc1ccc(NC(=O)OCc2cc3nc(C)c(C4CCC(=O)NC4=O)cc3cc2C)cc1Cl] |
| 69 | 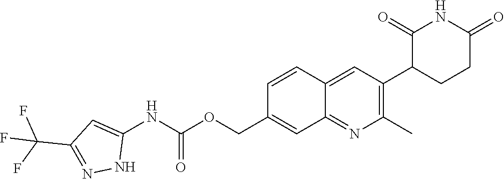 [SMILES: Cc1nc2cc(COC(=O)Nc3cc(C(F)(F)F)n[nH]3)ccc2cc1C1CCC(=O)NC1=O] |
| 70 | 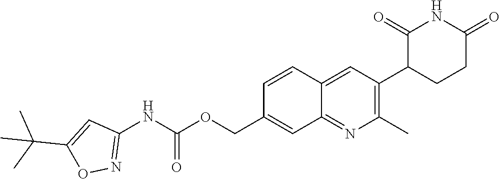 [SMILES: Cc1nc2cc(COC(=O)Nc3cc(C(C)(C)C)on3)ccc2cc1C1CCC(=O)NC1=O] |
| 71 | 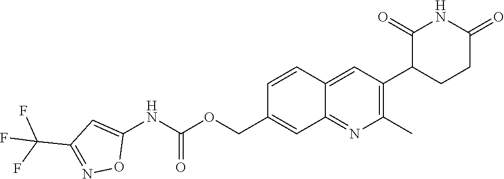 [SMILES: Cc1nc2cc(COC(=O)Nc3cc(C(F)(F)F)no3)ccc2cc1C1CCC(=O)NC1=O] |
| 74 | 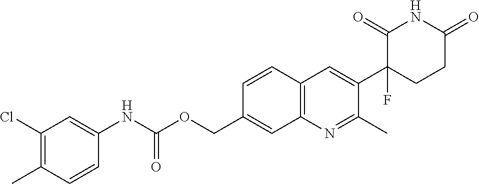 [SMILES: Cc1ccc(NC(=O)OCc2ccc3cc(C4(F)CCC(=O)NC4=O)c(C)nc3c2)cc1Cl] |
| 75 | 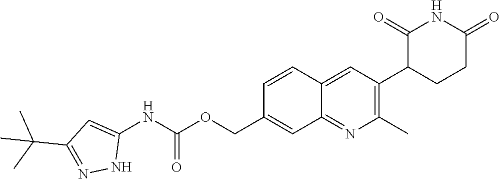 [SMILES: Cc1nc2cc(COC(=O)Nc3cc(C(C)(C)C)n[nH]3)ccc2cc1C1CCC(=O)NC1=O] |
| 80 | 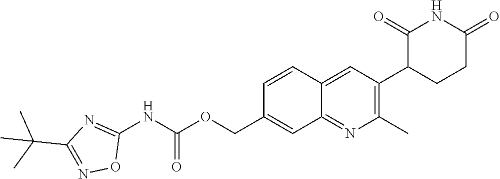 [SMILES: Cc1nc2cc(COC(=O)Nc3nc(C(C)(C)C)no3)ccc2cc1C1CCC(=O)NC1=O] |
| 81 | 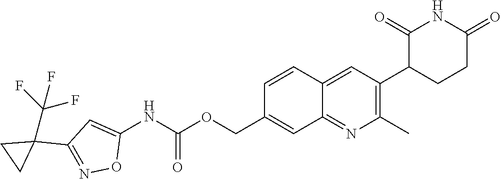 [SMILES: Cc1nc2cc(COC(=O)Nc3cc(C4(C(F)(F)F)CC4)no3)ccc2cc1C1CCC(=O)NC1=O] |
| 82 | 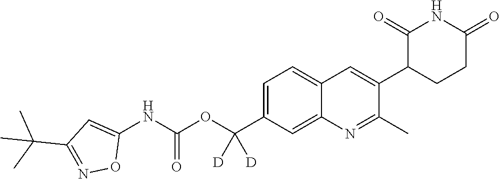 [SMILES: [2H]C([2H])(OC(=O)Nc1cc(C(C)(C)C)no1)c1ccc2cc(C3CCC(=O)NC3=O)c(C)nc2c1] |

46. The compound of any one of claims 1-41, wherein R⁵ is

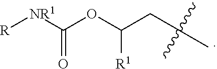

47. The compound of claim 46, wherein R⁵ is

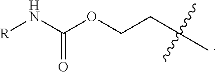

48. The compound of any one of claims 1-43, 46 and 47, wherein the compound is selected from 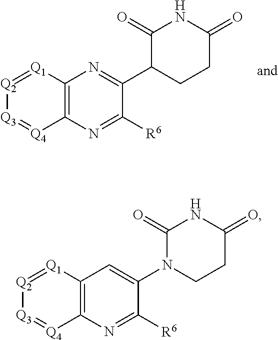
optionally wherein R⁶ is methyl.

49. The compound of claim 48, wherein the compound is selected from

| Compound No. | Structure |
|---|---|
| 66 | 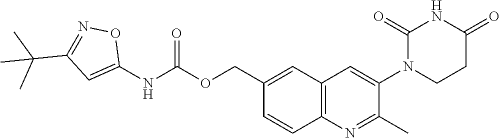 [SMILES: Cc1nc2ccc(COC(=O)Nc3cc(C(C)(C)C)no3)cc2cc1N1CCC(=O)NC1=O] |
| 72 | 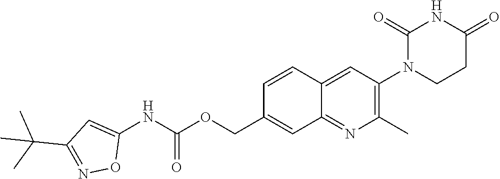 [SMILES: Cc1nc2cc(COC(=O)Nc3cc(C(C)(C)C)no3)ccc2cc1N1CCC(=O)NC1=O] |

50. The compound of any one of claims 1-41, wherein R⁵ is 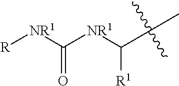
and wherein the compound is selected from

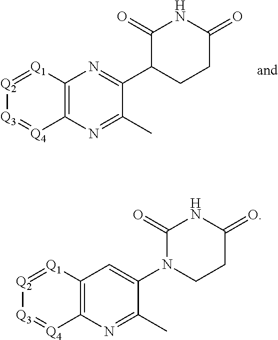

51. The compound of claim 50, wherein the compound is:

| Compound No. | Structure |
|---|---|
| 27 | 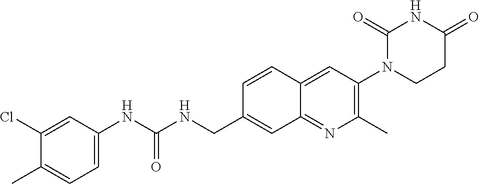 [SMILES: Cc1ccc(NC(=O)NCc2ccc3cc(N4CCC(=O)NC4=O)c(C)nc3c2)cc1Cl] |
| 41 | 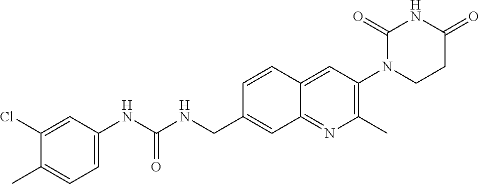 [SMILES: Cc1ccc(NC(=O)NCc2ccc3cc(N4CCC(=O)NC4=O)c(C)nc3c2)cc1Cl] |
| 51 | 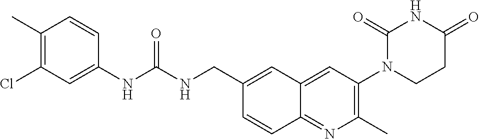 [SMILES: Cc1ccc(NC(=O)NCc2ccc3nc(C)c(N4CCC(=O)NC4=O)cc3c2)cc1Cl] |
| 52 | 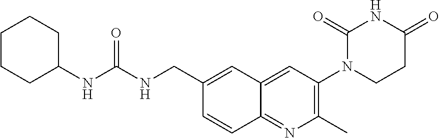 [SMILES: Cc1nc2ccc(CNC(=O)NC3CCCCC3)cc2cc1N1CCC(=O)NC1=O] |
| 64 | 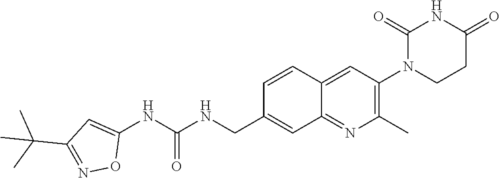 [SMILES: Cc1nc2cc(CNC(=O)Nc3cc(C(C)(C)C)no3)ccc2cc1N1CCC(=O)NC1=O] |
| 67 | 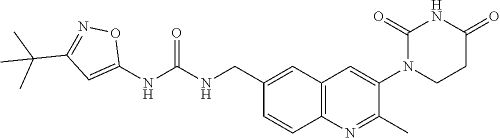 [SMILES: Cc1nc2ccc(CNC(=O)Nc3cc(C(C)(C)C)no3)cc2cc1N1CCC(=O)NC1=O] |

52. The compound of any one of claims 1-41, wherein R⁵ is

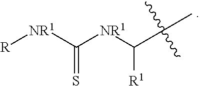

53. The compound of claim 52, wherein the compound is:

| Compound No. | Structure |
|---|---|
| 2 | 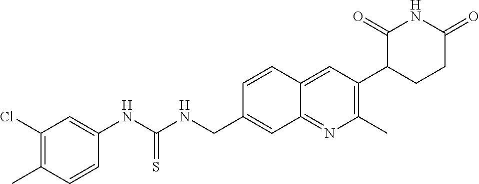 [SMILES: Cc1ccc(NC(=S)NCc2ccc3cc(C4CCC(=O)NC4=O)c(C)nc3c2)cc1Cl] |

54. The compound of any one of claims 1-41, wherein R⁵ is

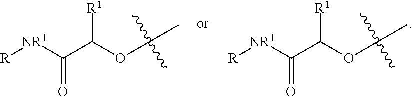

55. The compound of claim 54, wherein R⁵ is selected from:

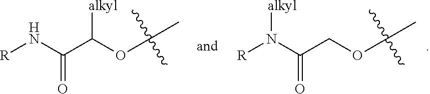

56. The compound of claim 54 or 55, wherein G and E are CH.

57. The compound of claim 56, wherein the compound is selected from:

| Compound No. | Structure |
|---|---|
| 34 | 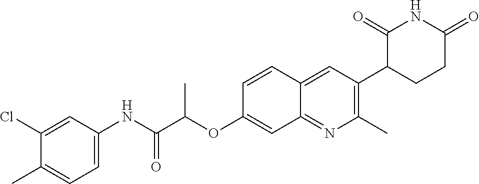 [SMILES: Cc1ccc(NC(=O)C(C)Oc2ccc3cc(C4CCC(=O)NC4=O)c(C)nc3c2)cc1Cl] |
| 36 | 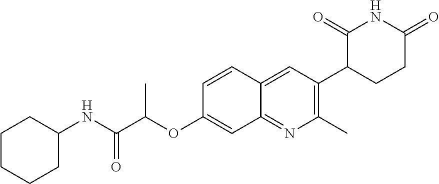 [SMILES: Cc1nc2cc(OC(C)C(=O)NC3CCCCC3)ccc2cc1C1CCC(=O)NC1=O] |

58. The compound of any one of claims 1-41, wherein R⁵ is:

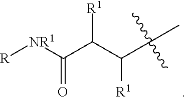

59. The compound of claim 58, wherein R⁵ is:

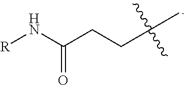

60. The compound of claim 59, wherein the compound is selected from:

| Compound No. | Structure |
|---|---|
| 32 | 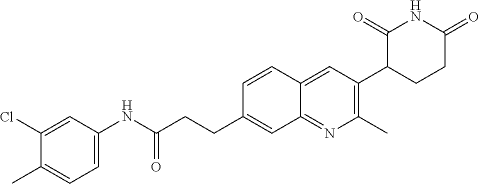 [SMILES: Cc1ccc(NC(=O)CCc2ccc3cc(C4CCC(=O)NC4=O)c(C)nc3c2)cc1Cl] |

61. The compound of any one of claims 1-41, wherein R⁵ is

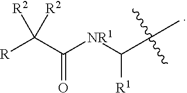

62. The compound of any one of claims 1-41, wherein R⁵ is

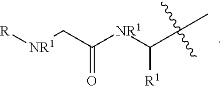

63. The compound of claim 62, wherein R⁵ is

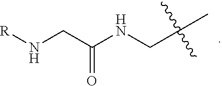

64. The compound of claim 63, wherein the compound is selected from:

| Compound No. | Structure |
|---|---|
| 26 | 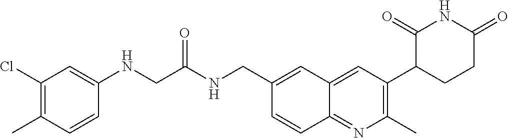 [SMILES: Cc1ccc(NCC(=O)NCc2ccc3nc(C)c(C4CCC(=O)NC4=O)cc3c2)cc1Cl] |

65. The compound of any one of claims 1-41, wherein R⁵ is

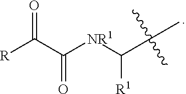

66. The compound of claim 65, wherein the compound is selected from:

| Compound No. | Structure |
|---|---|
| 1 | 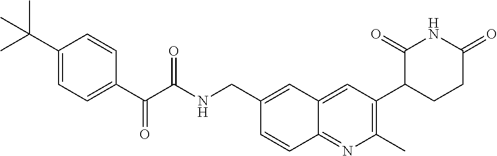 [SMILES: Cc1nc2ccc(CNC(=O)C(=O)c3ccc(C(C)(C)C)cc3)cc2cc1C1CCC(=O)NC1=O] |
| 23 | 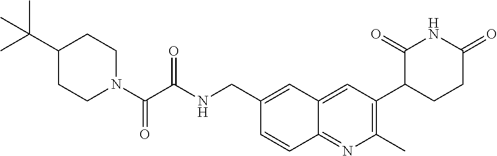 [SMILES: Cc1nc2ccc(CNC(=O)C(=O)N3CCC(C(C)(C)C)CC3)cc2cc1C1CCC(=O)NC1=O] |

67. The compound of any one of claims 1-39 and 49, wherein R⁵ is

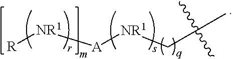

68. The compound of claim 67, wherein the compound is selected from:

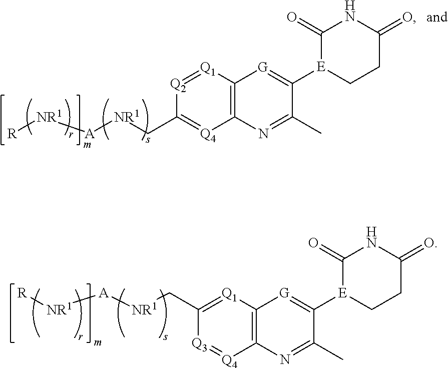

69. The compound of claim 67 or 68, wherein R⁵ is

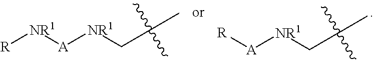

70. The compound of any one of claims 1-39 and 67-69, wherein A is 5- or 6-membered heteroaryl having 1-3 heteroatoms, 5- or 6-membered heterocycloalkyl having 1-3 heteroatoms, or 4- to 6-membered cycloalkenyl; wherein one or more CH₂ groups of the heterocycloalkyl or cycloalkenyl are optionally replaced by a C═O group.

71. The compound of claim 70, wherein A is 5-membered heteroaryl having 2 or 3 heteroatoms.

72. The compound of claim 70, wherein A is 4-membered cycloalkenyl in which one or more CH₂ groups are replaced by a C═O group.

73. The compound of claim 70, wherein the compound is selected from:

| Compound No. | Structure |
|---|---|
| 3 | 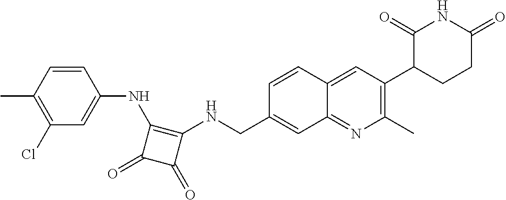 [SMILES: Cc1ccc(Nc2c(NCc3ccc4cc(C5CCC(=O)NC5=O)c(C)nc4c3)c(=O)c2=O)cc1Cl] |
| 13 | 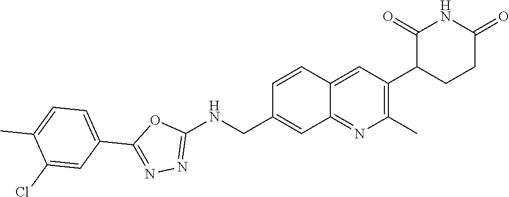 [SMILES: Cc1ccc(-c2nnc(NCc3ccc4cc(C5CCC(=O)NC5=O)c(C)nc4c3)o2)cc1Cl] |

74. The compound of any one of claims 1-39 and 49, wherein R⁵ is

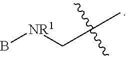

75. The compound of any preceding claim, wherein B is —SO₂NHR or 8-12 membered bicyclic heteroaryl substituted with one or more groups selected from Cl, Me, ^{t}Bu, OCF₃, OMe, CH₂-morpholino, and phenyl.

76. The compound of claim 75, wherein B is 8-12 membered bicyclic heteroaryl substituted with one or more groups selected from Cl, Me, ^{t}Bu, OCF₃, OMe, CH₂-morpholino, and phenyl.

77. The compound of any preceding claim, wherein R is cycloalkyl, heterocycloalkyl, aryl or heteroaryl optionally substituted with one or more R³, or wherein two R³ together with the carbon atoms to which they are attached form a cycloalkyl, heterocycloalkyl or heteroaryl ring.

78. The compound of any preceding claim, wherein each R³ is independently selected from Cl, Me, ^{t}Bu, CF₃, OCF₃, OMe, CH₂-heterocycloalkyl, phenyl, CN, F, NH₂, NHC(O)Me, C(O)Me, cyclopropyl, cyclopropyl substituted with haloalkyl, morpholino, benzyl, pyridyl or ethyl; or wherein two R³ together with the carbon atoms to which they are attached form a cycloalkyl, heterocycloalkyl or heteroaryl ring.

79. The compound of any preceding claim, wherein each R³ is independently selected from Cl, Me, ^{t}Bu, CF₃, OCF₃, OMe, CH₂-heterocycloalkyl or phenyl; or wherein two R³ together with the carbon atoms to which they are attached form a heterocycloalkyl ring.

80. The compound of claim 79 wherein each R³ is independently selected from Cl, Me, ^{t}Bu, CF₃, OCF₃, OMe, —CH₂-morpholino or phenyl; or wherein two R³ together with the carbon atoms to which they are attached form a heterocycloalkyl ring.

81. The compound of any preceding claim, wherein R is selected from:

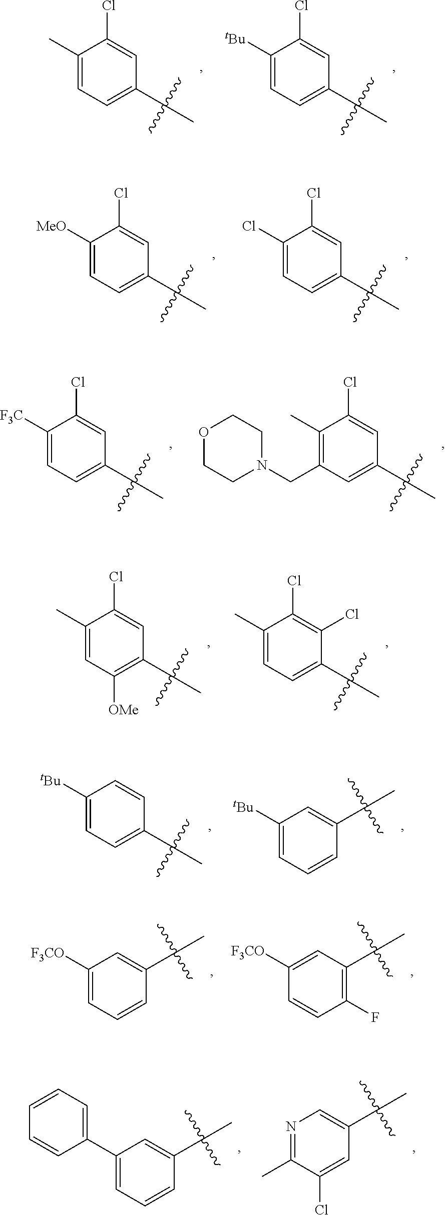

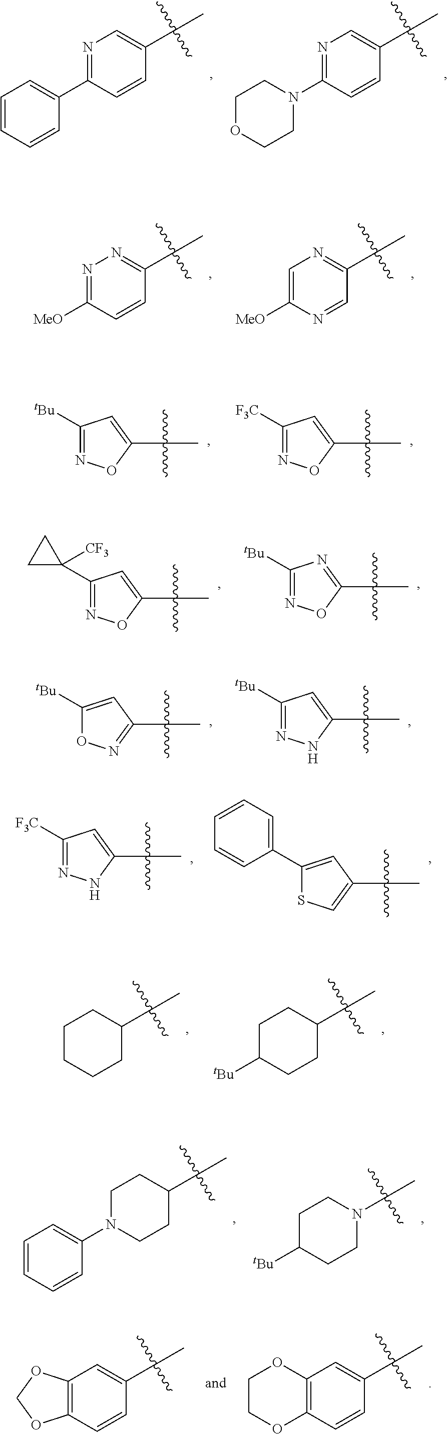

82. The compound of claim 81, wherein R is selected from:

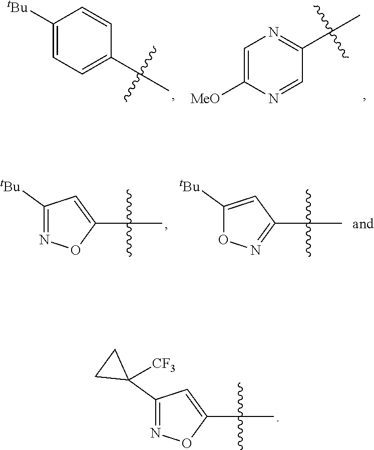 [SMILES: *c1ccc(C(C)(C)C)cc1 ; *c1cnc(OC)cn1 ; *c1cc(C(C)(C)C)no1 ; *c1cc(C(C)(C)C)on1 ; *c1cc(C2(C(F)(F)F)CC2)no1]

83. The compound of claim 82, wherein when R is 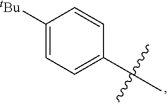 [SMILES: *c1ccc(C(C)(C)C)cc1]
then Q₂ is CR⁵ and Q₁, Q₃, and Q₄ are each independently CR⁴.

84. The compound of claim 82 or 83, wherein when R is 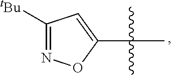 [SMILES: *c1cc(C(C)(C)C)no1]
Q₃ is CR⁵ and Q₁, Q₂, and Q₄ are each independently CR⁴; then R⁵ is

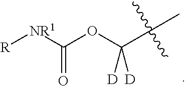

85. The compound of and one of claims 82-84, wherein when R is 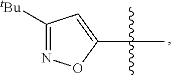 [SMILES: *c1cc(C(C)(C)C)no1]
Q₂ is CR⁵ and Q₁, Q₃, and Q₄ are each independently CR⁴; then R⁵ is

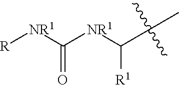

86. The compound of claim 82, wherein R is selected from:

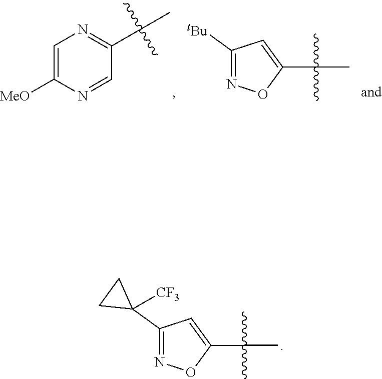 [SMILES: *c1cnc(OC)cn1 ; *c1cc(C(C)(C)C)no1 ; *c1cc(C2(C(F)(F)F)CC2)no1]

87. The compound of claim 82, wherein R is selected from:

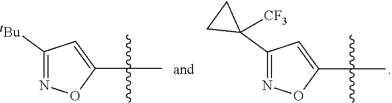 [SMILES: *c1cc(C(C)(C)C)no1 ; *c1cc(C2(C(F)(F)F)CC2)no1]

88. The compound of claim 82, wherein R is selected from:

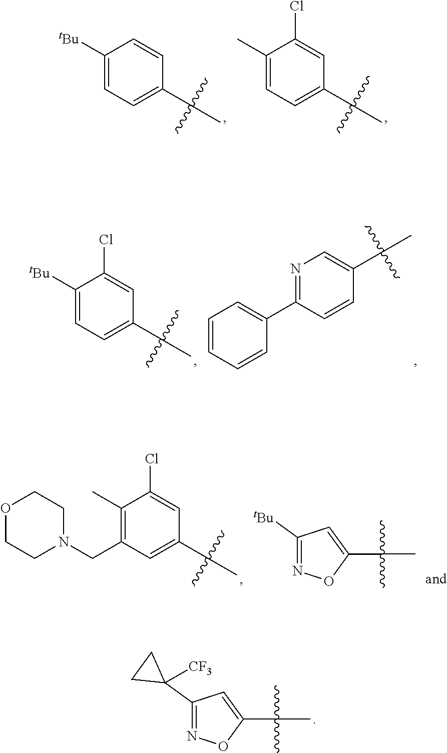

89. The compound of claim 88, wherein when R is 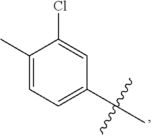 [SMILES: *c1ccc(C)c(Cl)c1]
then G is C-(unsubstituted C₁-C₄ alkyl).

90. The compound of any one of claims 86-89, wherein when R is 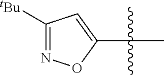 [SMILES: *c1cc(C(C)(C)C)no1]
then R⁵ is

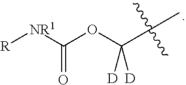

91. The compound of any preceding claim, wherein each R² is independently hydrogen or halogen.

92. The compound of any preceding claim, wherein each R⁴ is independently hydrogen, halogen or alkyl.

93. The compound of any preceding claim, wherein each R¹ is independently hydrogen or methyl.

94. The compound of any preceding claim, wherein each NR¹ is NH.

95. The compound of claim 1, wherein the compound is selected from:

| Compound No. | Structure |
|---|---|
| 1 | 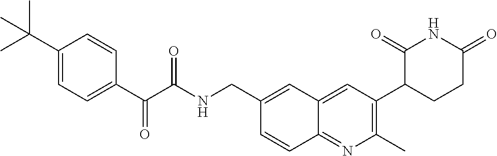 [SMILES: Cc1nc2ccc(CNC(=O)C(=O)c3ccc(C(C)(C)C)cc3)cc2cc1C1CCC(=O)NC1=O] |
| 2 | 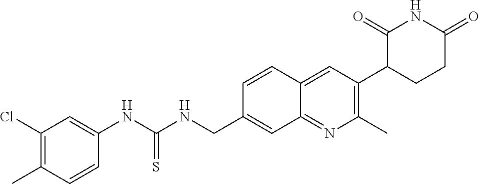 [SMILES: Cc1ccc(NC(=S)NCc2ccc3cc(C4CCC(=O)NC4=O)c(C)nc3c2)cc1Cl] |
| 3 | 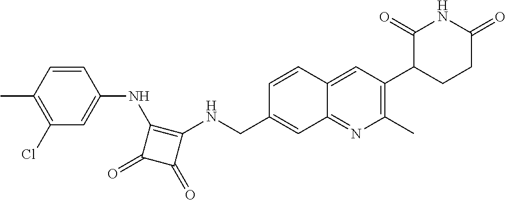 [SMILES: Cc1ccc(Nc2c(NCc3ccc4cc(C5CCC(=O)NC5=O)c(C)nc4c3)c(=O)c2=O)cc1Cl] |
| 4 | 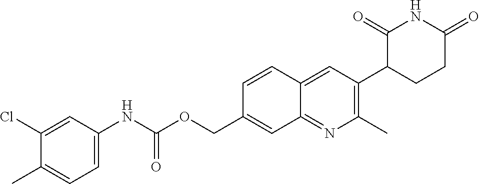 [SMILES: Cc1ccc(NC(=O)OCc2ccc3cc(C4CCC(=O)NC4=O)c(C)nc3c2)cc1Cl] |
| 5 | 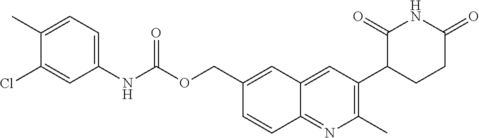 [SMILES: Cc1ccc(NC(=O)OCc2ccc3nc(C)c(C4CCC(=O)NC4=O)cc3c2)cc1Cl] |
| 6 | 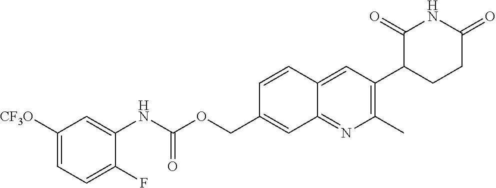 |
| 7 | 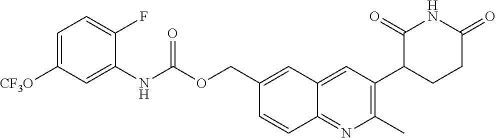 |
| 8 | 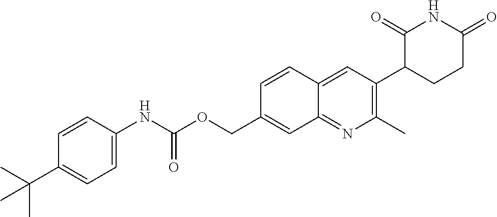 [SMILES: Cc1nc2cc(COC(=O)Nc3ccc(C(C)(C)C)cc3)ccc2cc1C1CCC(=O)NC1=O] |
| 9 | 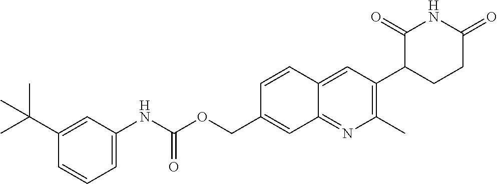 [SMILES: Cc1nc2cc(COC(=O)Nc3cccc(C(C)(C)C)c3)ccc2cc1C1CCC(=O)NC1=O] |
| 10 | 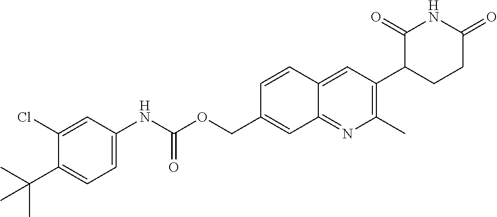 [SMILES: Cc1nc2cc(COC(=O)Nc3ccc(C(C)(C)C)c(Cl)c3)ccc2cc1C1CCC(=O)NC1=O] |
| 11 | 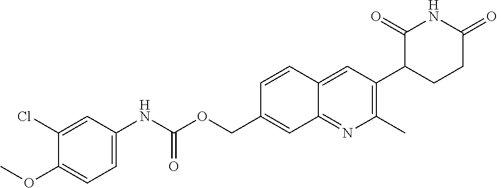 [SMILES: COc1ccc(NC(=O)OCc2ccc3cc(C4CCC(=O)NC4=O)c(C)nc3c2)cc1Cl] |
| 12 | 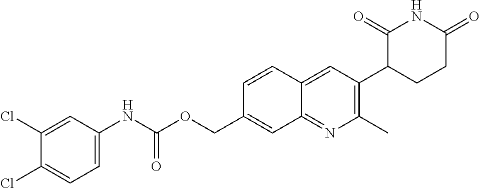 [SMILES: Cc1nc2cc(COC(=O)Nc3ccc(Cl)c(Cl)c3)ccc2cc1C1CCC(=O)NC1=O] |
| 13 | 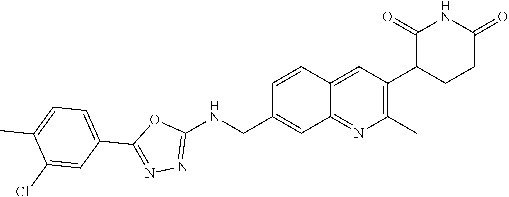 [SMILES: Cc1ccc(-c2nnc(NCc3ccc4cc(C5CCC(=O)NC5=O)c(C)nc4c3)o2)cc1Cl] |
| 14 | 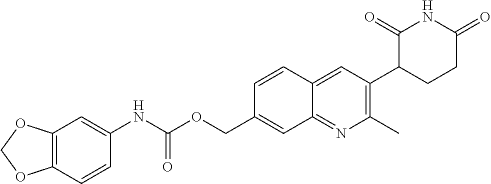 [SMILES: Cc1nc2cc(COC(=O)Nc3ccc4c(c3)OCO4)ccc2cc1C1CCC(=O)NC1=O] |
| 15 | 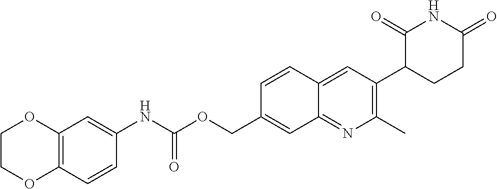 [SMILES: Cc1nc2cc(COC(=O)Nc3ccc4c(c3)OCCO4)ccc2cc1C1CCC(=O)NC1=O] |
| 17 | 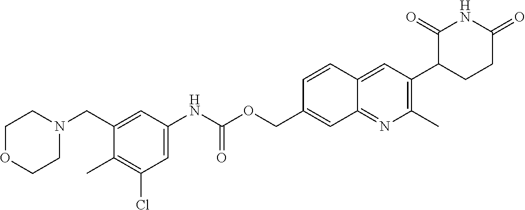 [SMILES: Cc1nc2cc(COC(=O)Nc3cc(Cl)c(C)c(CN4CCOCC4)c3)ccc2cc1C1CCC(=O)NC1=O] |
| 18 | 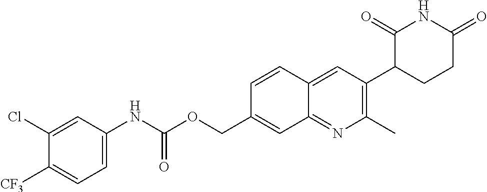 [SMILES: Cc1nc2cc(COC(=O)Nc3ccc(C(F)(F)F)c(Cl)c3)ccc2cc1C1CCC(=O)NC1=O] |
| 19 | 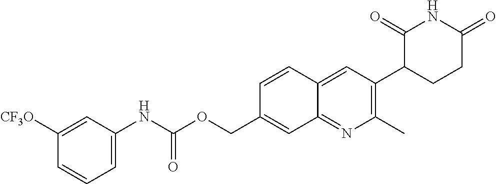 |
| 20 | 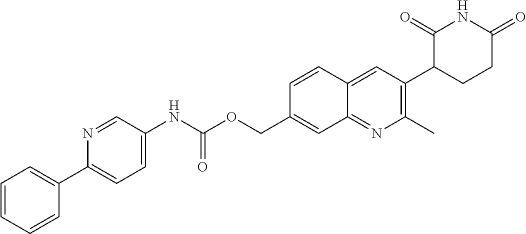 [SMILES: Cc1nc2cc(COC(=O)Nc3ccc(-c4ccccc4)nc3)ccc2cc1C1CCC(=O)NC1=O] |
| 21 | 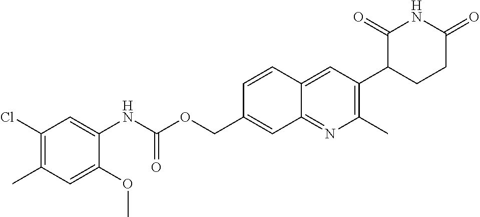 [SMILES: COc1cc(C)c(Cl)cc1NC(=O)OCc1ccc2cc(C3CCC(=O)NC3=O)c(C)nc2c1] |
| 22 | 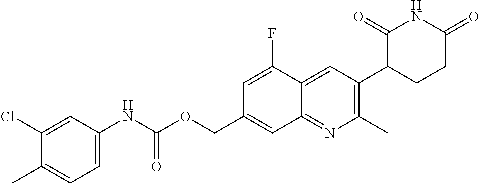 [SMILES: Cc1ccc(NC(=O)OCc2cc(F)c3cc(C4CCC(=O)NC4=O)c(C)nc3c2)cc1Cl] |
| 23 | 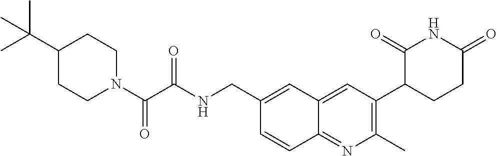 [SMILES: Cc1nc2ccc(CNC(=O)C(=O)N3CCC(C(C)(C)C)CC3)cc2cc1C1CCC(=O)NC1=O] |
| 26 | 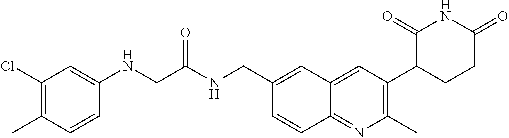 [SMILES: Cc1ccc(NCC(=O)NCc2ccc3nc(C)c(C4CCC(=O)NC4=O)cc3c2)cc1Cl] |
| 27 | 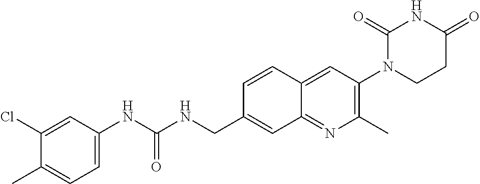 [SMILES: Cc1ccc(NC(=O)NCc2ccc3cc(N4CCC(=O)NC4=O)c(C)nc3c2)cc1Cl] |
| 28 | 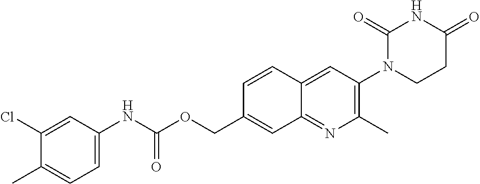 [SMILES: Cc1ccc(NC(=O)OCc2ccc3cc(N4CCC(=O)NC4=O)c(C)nc3c2)cc1Cl] |
| 29 | 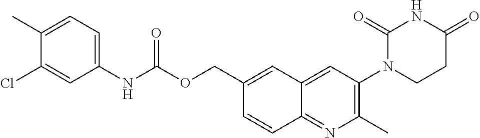 [SMILES: Cc1ccc(NC(=O)OCc2ccc3nc(C)c(N4CCC(=O)NC4=O)cc3c2)cc1Cl] |
| 30 | 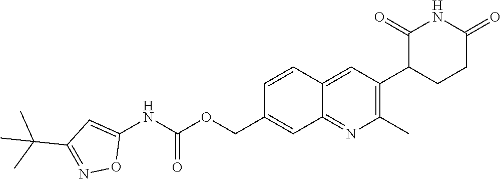 [SMILES: Cc1nc2cc(COC(=O)Nc3cc(C(C)(C)C)no3)ccc2cc1C1CCC(=O)NC1=O] |
| 32 | 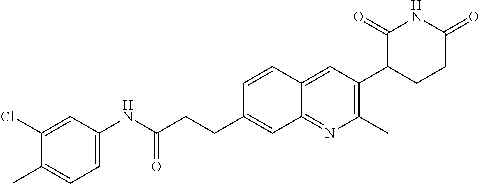 [SMILES: Cc1ccc(NC(=O)CCc2ccc3cc(C4CCC(=O)NC4=O)c(C)nc3c2)cc1Cl] |
| 34 | 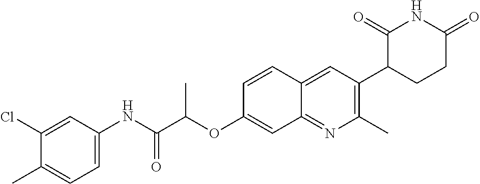 [SMILES: Cc1ccc(NC(=O)C(C)Oc2ccc3cc(C4CCC(=O)NC4=O)c(C)nc3c2)cc1Cl] |
| 36 | 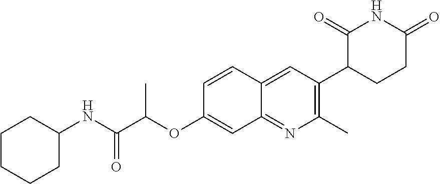 [SMILES: Cc1nc2cc(OC(C)C(=O)NC3CCCCC3)ccc2cc1C1CCC(=O)NC1=O] |
| 37 | 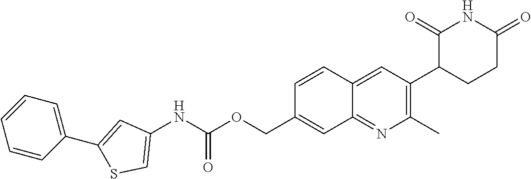 [SMILES: Cc1nc2cc(COC(=O)Nc3csc(-c4ccccc4)c3)ccc2cc1C1CCC(=O)NC1=O] |
| 38 | 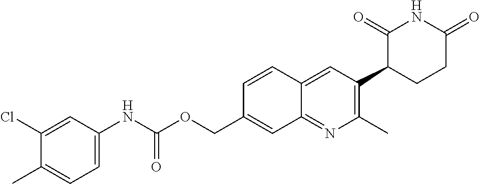 [SMILES: Cc1ccc(NC(=O)OCc2ccc3cc([C@@H]4CCC(=O)NC4=O)c(C)nc3c2)cc1Cl] |
| 39 | 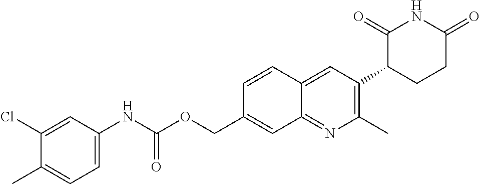 [SMILES: Cc1ccc(NC(=O)OCc2ccc3cc([C@H]4CCC(=O)NC4=O)c(C)nc3c2)cc1Cl] |
| 41 | 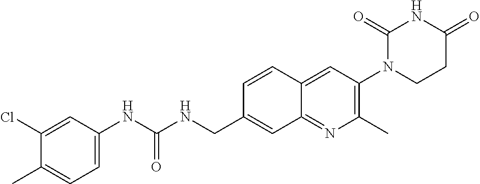 [SMILES: Cc1ccc(NC(=O)NCc2ccc3cc(N4CCC(=O)NC4=O)c(C)nc3c2)cc1Cl] |
| 42 | 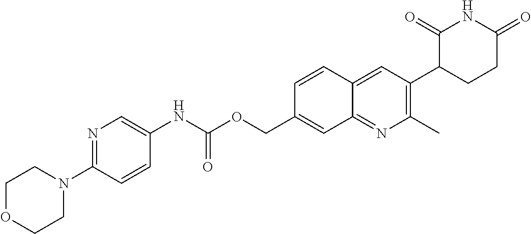 [SMILES: Cc1nc2cc(COC(=O)Nc3ccc(N4CCOCC4)nc3)ccc2cc1C1CCC(=O)NC1=O] |
| 44 | 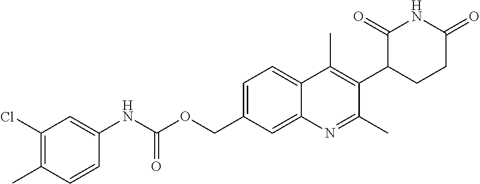 [SMILES: Cc1ccc(NC(=O)OCc2ccc3c(C)c(C4CCC(=O)NC4=O)c(C)nc3c2)cc1Cl] |
| 45 | 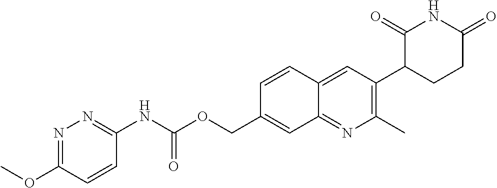 [SMILES: COc1ccc(NC(=O)OCc2ccc3cc(C4CCC(=O)NC4=O)c(C)nc3c2)nn1] |
| 46 | 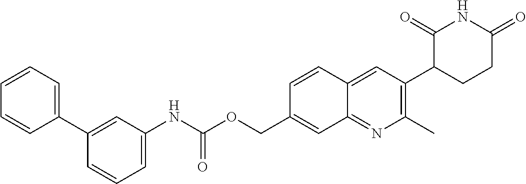 [SMILES: Cc1nc2cc(COC(=O)Nc3cccc(-c4ccccc4)c3)ccc2cc1C1CCC(=O)NC1=O] |
| 47 | 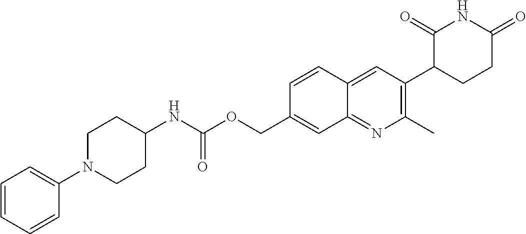 [SMILES: Cc1nc2cc(COC(=O)NC3CCN(c4ccccc4)CC3)ccc2cc1C1CCC(=O)NC1=O] |
| 49 | 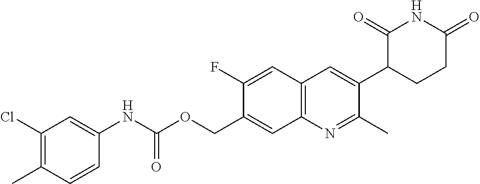 [SMILES: Cc1ccc(NC(=O)OCc2cc3nc(C)c(C4CCC(=O)NC4=O)cc3cc2F)cc1Cl] |
| 50 | 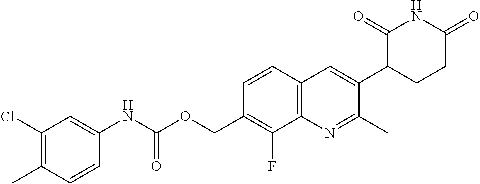 [SMILES: Cc1ccc(NC(=O)OCc2ccc3cc(C4CCC(=O)NC4=O)c(C)nc3c2F)cc1Cl] |
| 51 | 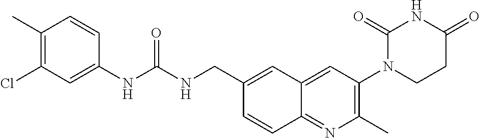 [SMILES: Cc1ccc(NC(=O)NCc2ccc3nc(C)c(N4CCC(=O)NC4=O)cc3c2)cc1Cl] |
| 52 | 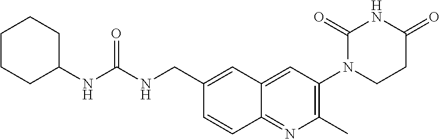 [SMILES: Cc1nc2ccc(CNC(=O)NC3CCCCC3)cc2cc1N1CCC(=O)NC1=O] |
| 54 | 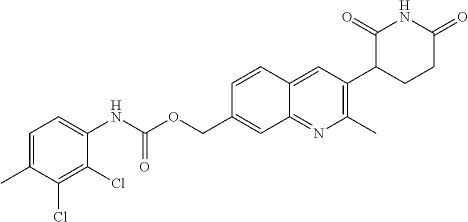 [SMILES: Cc1ccc(NC(=O)OCc2ccc3cc(C4CCC(=O)NC4=O)c(C)nc3c2)c(Cl)c1Cl] |
| 55 | 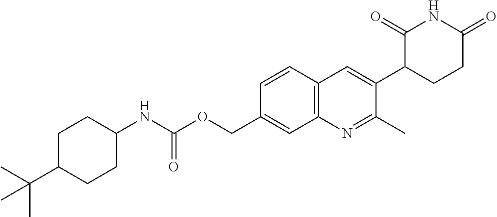 [SMILES: Cc1nc2cc(COC(=O)NC3CCC(C(C)(C)C)CC3)ccc2cc1C1CCC(=O)NC1=O] |
| 58 | 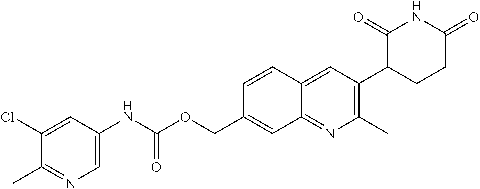 [SMILES: Cc1ncc(NC(=O)OCc2ccc3cc(C4CCC(=O)NC4=O)c(C)nc3c2)cc1Cl] |
| 59 | 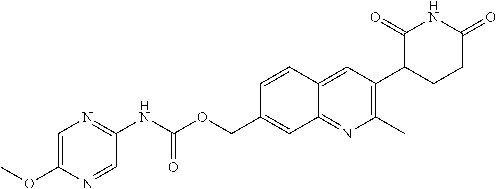 [SMILES: COc1cnc(NC(=O)OCc2ccc3cc(C4CCC(=O)NC4=O)c(C)nc3c2)cn1] |
| 60 | 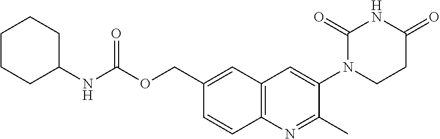 [SMILES: Cc1nc2ccc(COC(=O)NC3CCCCC3)cc2cc1N1CCC(=O)NC1=O] |
| 64 | 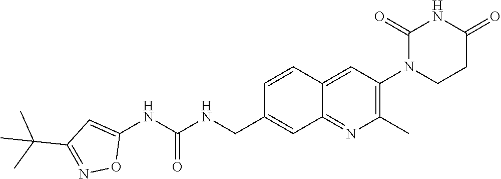 [SMILES: Cc1nc2cc(CNC(=O)Nc3cc(C(C)(C)C)no3)ccc2cc1N1CCC(=O)NC1=O] |
| 66 | 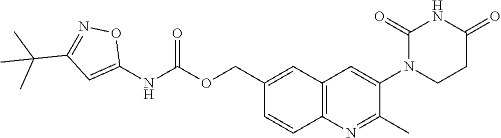 [SMILES: Cc1nc2ccc(COC(=O)Nc3cc(C(C)(C)C)no3)cc2cc1N1CCC(=O)NC1=O] |
| 67 | 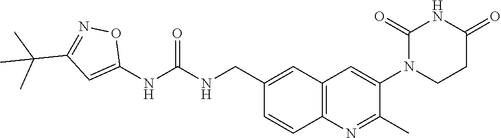 [SMILES: Cc1nc2ccc(CNC(=O)Nc3cc(C(C)(C)C)no3)cc2cc1N1CCC(=O)NC1=O] |
| 68 | 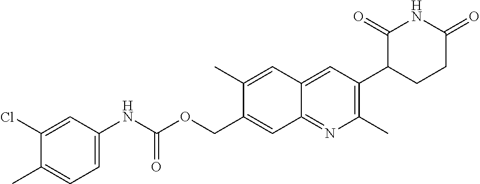 [SMILES: Cc1ccc(NC(=O)OCc2cc3nc(C)c(C4CCC(=O)NC4=O)cc3cc2C)cc1Cl] |
| 69 | 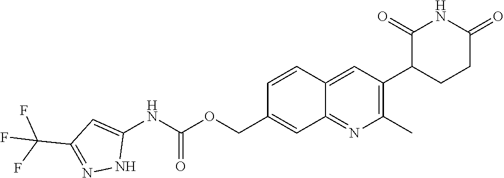 [SMILES: Cc1nc2cc(COC(=O)Nc3cc(C(F)(F)F)n[nH]3)ccc2cc1C1CCC(=O)NC1=O] |
| 70 | 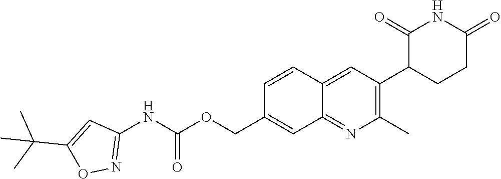 [SMILES: Cc1nc2cc(COC(=O)Nc3cc(C(C)(C)C)on3)ccc2cc1C1CCC(=O)NC1=O] |
| 71 | 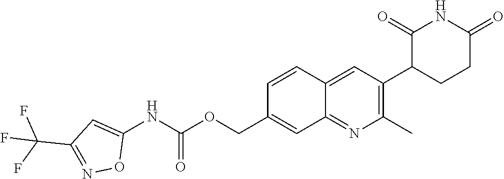 [SMILES: Cc1nc2cc(COC(=O)Nc3cc(C(F)(F)F)no3)ccc2cc1C1CCC(=O)NC1=O] |
| 72 | 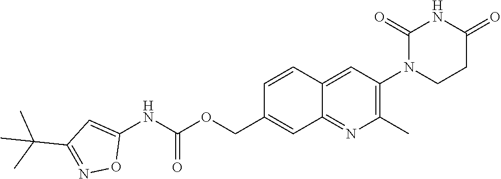 [SMILES: Cc1nc2cc(COC(=O)Nc3cc(C(C)(C)C)no3)ccc2cc1N1CCC(=O)NC1=O] |
| 74 | 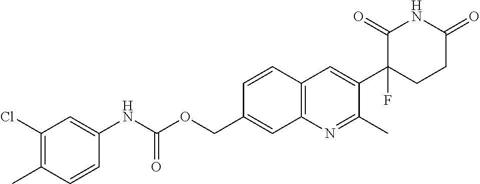 [SMILES: Cc1ccc(NC(=O)OCc2ccc3cc(C4(F)CCC(=O)NC4=O)c(C)nc3c2)cc1Cl] |
| 75 | 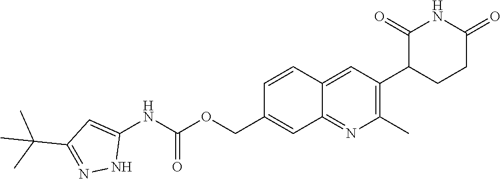 [SMILES: Cc1nc2cc(COC(=O)Nc3cc(C(C)(C)C)n[nH]3)ccc2cc1C1CCC(=O)NC1=O] |
| 80 | 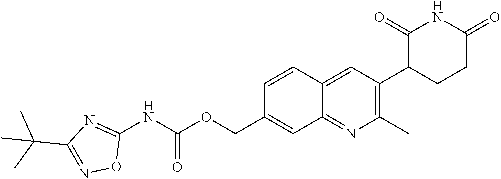 [SMILES: Cc1nc2cc(COC(=O)Nc3nc(C(C)(C)C)no3)ccc2cc1C1CCC(=O)NC1=O] |
| 81 | 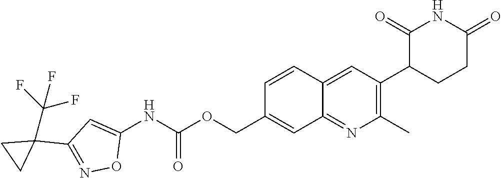 [SMILES: Cc1nc2cc(COC(=O)Nc3cc(C4(C(F)(F)F)CC4)no3)ccc2cc1C1CCC(=O)NC1=O] |
| 82 | 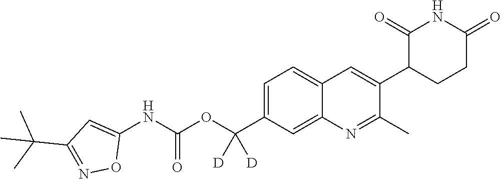 [SMILES: [2H]C([2H])(OC(=O)Nc1cc(C(C)(C)C)no1)c1ccc2cc(C3CCC(=O)NC3=O)c(C)nc2c1] |

96. The compound of claim 95, wherein the compound is selected from Compounds 1, 2, 4, 5, 7, 8, 10, 13, 17, 18, 20, 21, 23, 26, 27, 28, 29, 30, 32, 34, 36, 39, 42, 44, 46, 47, 50, 51, 54, 55, 58, 59, 60, 64, 66, 67, 70, 72, 80, 81 and 82

97. The compound of claim 96, wherein the compound is selected from Compounds 1, 5, 7, 8, 10, 13, 17, 20, 23, 27, 29, 36, 42, 44, 47, 51, 59, 64, 66, 67, 70, 81 and 82.

98. The compound of claim 95, wherein the compound is selected from Compounds 1, 4, 8, 11, 21, 22, 30, 32, 39, 42, 50, 54, 55, 58, 59, 66, 67, 69, 70, 80, 81 and 82.

99. The compound of claim 98, wherein the compound is selected from Compounds 1, 59, 67, 70, 81 and 82.

100. The compound of claim 95, wherein the compound is selected from Compounds 2, 3, 4, 6, 8, 9, 10, 11, 12, 13, 14, 15, 16, 17, 18, 19, 20, 21, 22, 27, 28, 30, 32, 34, 36, 37, 38, 39, 42, 44, 45, 46, 47, 49, 50, 54, 55, 58, 59, 64, 68, 72, 74, 75, 80, 81 and 82.

101. The compound of claim 95, wherein the compound is selected from Compounds 4, 6, 8, 9, 10, 11, 12, 14, 15, 17, 18, 19, 20, 21, 22, 28, 29, 30, 32, 37, 39, 42, 44, 45, 46, 50, 51, 54, 55, 58, 59, 66, 67, 69, 70, 72, 75, 80, 81 and 82.

102. The compound of claim 101, wherein the compound is selected from Compounds 8, 10, 11, 15, 17, 19, 20, 30, 39, 44, 58, 70, 81 and 82.

103. A pharmaceutical composition comprising a compound of any one of claims 1-102.

104. The compound of any one of claims 1-102 or the pharmaceutical composition of claim 103 for use in medicine.

105. The compound of any one of claims 1-102 or the pharmaceutical composition of claim 103 for use in the treatment of cancer.

106. The compound or pharmaceutical composition for use of claim 105, wherein the cancer is hepatocellular carcinoma, neuroblastoma, leukemia, acute myeloid leukemia (AML), acute promyelocytic leukemia (APL), multiple myeloma, breast cancer, prostate cancer, bladder cancer, kidney cancer, muscle cancer, ovarian cancer, skin cancer, pancreatic cancer, colon cancer, hematological cancer, cancer of connective tissue, placental cancer, bone cancer, uterine cancer, cervical cancer, choriocarcinoma, endometrial cancer, gastric cancer, or lung cancer.
